SEQUENCE LISTING (358 sequences)

SEQ ID NO: 1 - DNA, 105 bp, Homo sapiens
  modified_base (25)..(25): Potential to be methylated (5-methylcytosine)
  modified_base (27)..(27): Potential to be methylated (5-methylcytosine)
  modified_base (35)..(35): Potential to be methylated (5-methylcytosine)
  modified_base (37)..(37): Potential to be methylated (5-methylcytosine)
  modified_base (42)..(42): Potential to be methylated (5-methylcytosine)
  modified_base (45)..(45): Potential to be methylated (5-methylcytosine)
  modified_base (52)..(52): Potential to be methylated (5-methylcytosine)
  modified_base (61)..(61): Potential to be methylated (5-methylcytosine)
  modified_base (63)..(63): Potential to be methylated (5-methylcytosine)
  modified_base (66)..(66): Potential to be methylated (5-methylcytosine)
  modified_base (71)..(71): Potential to be methylated (5-methylcytosine)
cgtgaggttggtgggcaggcctagcgcggagatgcgcgccacgtcgccccccgagcactgcgcggcgtcccggaagacacacttgcaagctggcggacaggggaa

SEQ ID NO: 2 - DNA, 105 bp, Homo sapiens; identical to SEQ ID NO: 1
  modified_base (71)..(71): Potential to be methylated (5-methylcytosine)

SEQ ID NO: 3 - DNA, 105 bp, Homo sapiens; identical to SEQ ID NO: 1
  modified_base (66)..(66): Potential to be methylated (5-methylcytosine)

SEQ ID NO: 4 - DNA, 105 bp, Homo sapiens; identical to SEQ ID NO: 1
  modified_base (63)..(63): Potential to be methylated (5-methylcytosine)

SEQ ID NO: 5 - DNA, 105 bp, Homo sapiens; identical to SEQ ID NO: 1
  modified_base (61)..(61): Potential to be methylated (5-methylcytosine)

SEQ ID NO: 6 - DNA, 105 bp, Homo sapiens; identical to SEQ ID NO: 1
  modified_base (52)..(52): Potential to be methylated (5-methylcytosine)

SEQ ID NO: 7 - DNA, 105 bp, Homo sapiens; identical to SEQ ID NO: 1
  modified_base (45)..(45): Potential to be methylated (5-methylcytosine)

SEQ ID NO: 8 - DNA, 105 bp, Homo sapiens; identical to SEQ ID NO: 1
  modified_base (42)..(42): Potential to be methylated (5-methylcytosine)

SEQ ID NO: 9 - DNA, 105 bp, Homo sapiens; identical to SEQ ID NO: 1
  modified_base (37)..(37): Potential to be methylated (5-methylcytosine)

SEQ ID NO: 10 - DNA, 105 bp, Homo sapiens; identical to SEQ ID NO: 1
  modified_base (35)..(35): Potential to be methylated (5-methylcytosine)

SEQ ID NO: 11 - DNA, 105 bp, Homo sapiens; identical to SEQ ID NO: 1
  modified_base (27)..(27): Potential to be methylated (5-methylcytosine)

SEQ ID NO: 12 - DNA, 105 bp, Homo sapiens; identical to SEQ ID NO: 1
  modified_base (25)..(25): Potential to be methylated (5-methylcytosine)

SEQ ID NO: 13 - DNA, 140 bp, Homo sapiens
  modified_base (22)..(22): Potential to be methylated (5-methylcytosine)
  modified_base (24)..(24): Potential to be methylated (5-methylcytosine)
  modified_base (27)..(27): Potential to be methylated (5-methylcytosine)
  modified_base (31)..(31): Potential to be methylated (5-methylcytosine)
  modified_base (44)..(44): Potential to be methylated (5-methylcytosine)
  modified_base (55)..(55): Potential to be methylated (5-methylcytosine)
  modified_base (58)..(58): Potential to be methylated (5-methylcytosine)
  modified_base (60)..(60): Potential to be methylated (5-methylcytosine)
  modified_base (103)..(103): Potential to be methylated (5-methylcytosine)
  modified_base (108)..(108): Potential to be methylated (5-methylcytosine)
  modified_base (111)..(111): Potential to be methylated (5-methylcytosine)
ggggtgcggggaggttgagagcgcggcggccgctgccagcaatcgaggagccagcggcgcgtgtgctgagggcccagctagcaaaataaagagggttttcagcggagcggcggctcaggcgaggctgggggagccgggga

SEQ ID NO: 14 - DNA, 140 bp, Homo sapiens; identical to SEQ ID NO: 13
  modified_base (22)..(22): Potential to be methylated (5-methylcytosine)

SEQ ID NO: 15 - DNA, 140 bp, Homo sapiens; identical to SEQ ID NO: 13
  modified_base (24)..(24): Potential to be methylated (5-methylcytosine)

SEQ ID NO: 16 - DNA, 140 bp, Homo sapiens; identical to SEQ ID NO: 13
  modified_base (27)..(27): Potential to be methylated (5-methylcytosine)

SEQ ID NO: 17 - DNA, 140 bp, Homo sapiens; identical to SEQ ID NO: 13
  modified_base (31)..(31): Potential to be methylated (5-methylcytosine)

SEQ ID NO: 18 - DNA, 140 bp, Homo sapiens; identical to SEQ ID NO: 13
  modified_base (44)..(44): Potential to be methylated (5-methylcytosine)

SEQ ID NO: 19 - DNA, 140 bp, Homo sapiens; identical to SEQ ID NO: 13
  modified_base (55)..(55): Potential to be methylated (5-methylcytosine)

SEQ ID NO: 20 - DNA, 140 bp, Homo sapiens; identical to SEQ ID NO: 13
  modified_base (58)..(58): Potential to be methylated (5-methylcytosine)

SEQ ID NO: 21 - DNA, 140 bp, Homo sapiens; identical to SEQ ID NO: 13
  modified_base (60)..(60): Potential to be methylated (5-methylcytosine)

SEQ ID NO: 22 - DNA, 140 bp, Homo sapiens; identical to SEQ ID NO: 13
  modified_base (103)..(103): Potential to be methylated (5-methylcytosine)

SEQ ID NO: 23 - DNA, 140 bp, Homo sapiens; identical to SEQ ID NO: 13
  modified_base (108)..(108): Potential to be methylated (5-methylcytosine)

SEQ ID NO: 24 - DNA, 140 bp, Homo sapiens; identical to SEQ ID NO: 13
  modified_base (111)..(111): Potential to be methylated (5-methylcytosine)

SEQ ID NO: 25 - DNA, 127 bp, Homo sapiens
  modified_base (28)..(28): Potential to be methylated (5-methylcytosine)
  modified_base (42)..(42): Potential to be methylated (5-methylcytosine)
  modified_base (44)..(44): Potential to be methylated (5-methylcytosine)
  modified_base (48)..(48): Potential to be methylated (5-methylcytosine)
  modified_base (54)..(54): Potential to be methylated (5-methylcytosine)
  modified_base (60)..(60): Potential to be methylated (5-methylcytosine)
  modified_base (62)..(62): Potential to be methylated (5-methylcytosine)
  modified_base (68)..(68): Potential to be methylated (5-methylcytosine)
  modified_base (70)..(70): Potential to be methylated (5-methylcytosine)
  modified_base (79)..(79): Potential to be methylated (5-methylcytosine)
  modified_base (100)..(100): Potential to be methylated (5-methylcytosine)
ggcgctgaagctggagaggccatcctgcgcttgggaaaggccgcgggcgccaccgcctgcgcggtcccgcggtcagggcgctggagctggggggagccccgccttgccccaaggagaagagccccgg

SEQ ID NO: 26 - DNA, 127 bp, Homo sapiens; identical to SEQ ID NO: 25
  modified_base (28)..(28): Potential to be methylated (5-methylcytosine)

SEQ ID NO: 27 - DNA, 127 bp, Homo sapiens; identical to SEQ ID NO: 25
  modified_base (42)..(42): Potential to be methylated (5-methylcytosine)

SEQ ID NO: 28 - DNA, 127 bp, Homo sapiens; identical to SEQ ID NO: 25
  modified_base (44)..(44): Potential to be methylated (5-methylcytosine)

SEQ ID NO: 29 - DNA, 127 bp, Homo sapiens; identical to SEQ ID NO: 25
  modified_base (48)..(48): Potential to be methylated (5-methylcytosine)

SEQ ID NO: 30 - DNA, 127 bp, Homo sapiens; identical to SEQ ID NO: 25
  modified_base (54)..(54): Potential to be methylated (5-methylcytosine)

SEQ ID NO: 31 - DNA, 127 bp, Homo sapiens; identical to SEQ ID NO: 25
  modified_base (60)..(60): Potential to be methylated (5-methylcytosine)

SEQ ID NO: 32 - DNA, 127 bp, Homo sapiens; identical to SEQ ID NO: 25
  modified_base (62)..(62): Potential to be methylated (5-methylcytosine)

SEQ ID NO: 33 - DNA, 127 bp, Homo sapiens; identical to SEQ ID NO: 25
  modified_base (68)..(68): Potential to be methylated (5-methylcytosine)

SEQ ID NO: 34 - DNA, 127 bp, Homo sapiens; identical to SEQ ID NO: 25
  modified_base (70)..(70): Potential to be methylated (5-methylcytosine)

SEQ ID NO: 35 - DNA, 127 bp, Homo sapiens; identical to SEQ ID NO: 25
  modified_base (79)..(79): Potential to be methylated (5-methylcytosine)

SEQ ID NO: 36 - DNA, 127 bp, Homo sapiens; identical to SEQ ID NO: 25
  modified_base (100)..(100): Potential to be methylated (5-methylcytosine)

SEQ ID NO: 37 - DNA, 149 bp, Homo sapiens
  modified_base (34)..(34): Potential to be methylated (5-methylcytosine)
  modified_base (36)..(36): Potential to be methylated (5-methylcytosine)
  modified_base (40)..(40): Potential to be methylated (5-methylcytosine)
  modified_base (43)..(43): Potential to be methylated (5-methylcytosine)
  modified_base (45)..(45): Potential to be methylated (5-methylcytosine)
  modified_base (50)..(50): Potential to be methylated (5-methylcytosine)
  modified_base (54)..(54): Potential to be methylated (5-methylcytosine)
  modified_base (79)..(79): Potential to be methylated (5-methylcytosine)
  modified_base (93)..(93): Potential to be methylated (5-methylcytosine)
  modified_base (95)..(95): Potential to be methylated (5-methylcytosine)
  modified_base (99)..(99): Potential to be methylated (5-methylcytosine)
  modified_base (105)..(105): Potential to be methylated (5-methylcytosine)
  modified_base (111)..(111): Potential to be methylated (5-methylcytosine)
  modified_base (113)..(113): Potential to be methylated (5-methylcytosine)
  modified_base (119)..(119): Potential to be methylated (5-methylcytosine)
  modified_base (121)..(121): Potential to be methylated (5-methylcytosine)
tgcagcagggaagcttatagtccagttgtcatccgcggccgccgcgctccgggcgctgaagctggagaggccatcctgcgcttgggaaaggccgcgggcgccaccgcctgcgcggtcccgcggtcagggcgctggagctggggggagcc

SEQ ID NO: 38 - DNA, 149 bp, Homo sapiens; identical to SEQ ID NO: 37
  modified_base (34)..(34): Potential to be methylated (5-methylcytosine)

SEQ ID NO: 39 - DNA, 149 bp, Homo sapiens; identical to SEQ ID NO: 37
  modified_base (36)..(36): Potential to be methylated (5-methylcytosine)

SEQ ID NO: 40 - DNA, 149 bp, Homo sapiens; identical to SEQ ID NO: 37
  modified_base (40)..(40): Potential to be methylated (5-methylcytosine)

SEQ ID NO: 41 - DNA, 149 bp, Homo sapiens; identical to SEQ ID NO: 37
  modified_base (43)..(43): Potential to be methylated (5-methylcytosine)

SEQ ID NO: 42 - DNA, 149 bp, Homo sapiens; identical to SEQ ID NO: 37
  modified_base (45)..(45): Potential to be methylated (5-methylcytosine)

SEQ ID NO: 43 - DNA, 149 bp, Homo sapiens; identical to SEQ ID NO: 37
  modified_base (50)..(50): Potential to be methylated (5-methylcytosine)

SEQ ID NO: 44 - DNA, 149 bp, Homo sapiens; identical to SEQ ID NO: 37
  modified_base (54)..(54): Potential to be methylated (5-methylcytosine)

SEQ ID NO: 45 - DNA, 149 bp, Homo sapiens; identical to SEQ ID NO: 37
  modified_base (79)..(79): Potential to be methylated (5-methylcytosine)

SEQ ID NO: 46 - DNA, 149 bp, Homo sapiens; identical to SEQ ID NO: 37
  modified_base (93)..(93): Potential to be methylated (5-methylcytosine)

SEQ ID NO: 47 - DNA, 149 bp, Homo sapiens; identical to SEQ ID NO: 37
  modified_base (95)..(95): Potential to be methylated (5-methylcytosine)

SEQ ID NO: 48 - DNA, 149 bp, Homo sapiens; identical to SEQ ID NO: 37
  modified_base (99)..(99): Potential to be methylated (5-methylcytosine)

SEQ ID NO: 49 - DNA, 149 bp, Homo sapiens; identical to SEQ ID NO: 37
  modified_base (105)..(105): Potential to be methylated (5-methylcytosine)

SEQ ID NO: 50 - DNA, 149 bp, Homo sapiens; identical to SEQ ID NO: 37
  modified_base (111)..(111): Potential to be methylated (5-methylcytosine)

SEQ ID NO: 51 - DNA, 149 bp, Homo sapiens; identical to SEQ ID NO: 37
  modified_base (113)..(113): Potential to be methylated (5-methylcytosine)

SEQ ID NO: 52 - DNA, 149 bp, Homo sapiens; identical to SEQ ID NO: 37
  modified_base (119)..(119): Potential to be methylated (5-methylcytosine)

SEQ ID NO: 53 - DNA, 149 bp, Homo sapiens; identical to SEQ ID NO: 37
  modified_base (121)..(121): Potential to be methylated (5-methylcytosine)

SEQ ID NO: 54 - DNA, 124 bp, Homo sapiens
  modified_base (27)..(27): Potential to be methylated (5-methylcytosine)
  modified_base (51)..(51): Potential to be methylated (5-methylcytosine)
  modified_base (53)..(53): Potential to be methylated (5-methylcytosine)
  modified_base (61)..(61): Potential to be methylated (5-methylcytosine)
  modified_base (63)..(63): Potential to be methylated (5-methylcytosine)
  modified_base (68)..(68): Potential to be methylated (5-methylcytosine)
  modified_base (71)..(71): Potential to be methylated (5-methylcytosine)
  modified_base (78)..(78): Potential to be methylated (5-methylcytosine)
  modified_base (87)..(87): Potential to be methylated (5-methylcytosine)
  modified_base (89)..(89): Potential to be methylated (5-methylcytosine)
  modified_base (92)..(92): Potential to be methylated (5-methylcytosine)
  modified_base (97)..(97): Potential to be methylated (5-methylcytosine)
cggcccattccgaagagcaggatgtgcgtgaggttggtgggcaggcctagcgcggagatgcgcgccacgtcgccccccgagcactgcgcggcgtcccggaagacacacttgcaagctggcggac

SEQ ID NO: 55 - DNA, 124 bp, Homo sapiens; identical to SEQ ID NO: 54
  modified_base (27)..(27): Potential to be methylated (5-methylcytosine)

SEQ ID NO: 56 - DNA, 124 bp, Homo sapiens; identical to SEQ ID NO: 54
  modified_base (51)..(51): Potential to be methylated (5-methylcytosine)

SEQ ID NO: 57 - DNA, 124 bp, Homo sapiens; identical to SEQ ID NO: 54
  modified_base (53)..(53): Potential to be methylated (5-methylcytosine)

SEQ ID NO: 58 - DNA, 124 bp, Homo sapiens; identical to SEQ ID NO: 54
  modified_base (61)..(61): Potential to be methylated (5-methylcytosine)

SEQ ID NO: 59 - DNA, 124 bp, Homo sapiens; identical to SEQ ID NO: 54
  modified_base (63)..(63): Potential to be methylated (5-methylcytosine)

SEQ ID NO: 60 - DNA, 124 bp, Homo sapiens; identical to SEQ ID NO: 54
  modified_base (68)..(68): Potential to be methylated (5-methylcytosine)

SEQ ID NO: 61 - DNA, 124 bp, Homo sapiens; identical to SEQ ID NO: 54
  modified_base (71)..(71): Potential to be methylated (5-methylcytosine)

SEQ ID NO: 62 - DNA, 124 bp, Homo sapiens; identical to SEQ ID NO: 54
  modified_base (78)..(78): Potential to be methylated (5-methylcytosine)

SEQ ID NO: 63 - DNA, 124 bp, Homo sapiens; identical to SEQ ID NO: 54
  modified_base (87)..(87): Potential to be methylated (5-methylcytosine)

SEQ ID NO: 64 - DNA, 124 bp, Homo sapiens; identical to SEQ ID NO: 54
  modified_base (89)..(89): Potential to be methylated (5-methylcytosine)

SEQ ID NO: 65 - DNA, 124 bp, Homo sapiens; identical to SEQ ID NO: 54
  modified_base (92)..(92): Potential to be methylated (5-methylcytosine)

SEQ ID NO: 66 - DNA, 124 bp, Homo sapiens; identical to SEQ ID NO: 54
  modified_base (97)..(97): Potential to be methylated (5-methylcytosine)

SEQ ID NO: 67 - DNA, 108 bp, Homo sapiens
  modified_base (33)..(33): Potential to be methylated (5-methylcytosine)
  modified_base (36)..(36): Potential to be methylated (5-methylcytosine)
  modified_base (39)..(39): Potential to be methylated (5-methylcytosine)
  modified_base (49)..(49): Potential to be methylated (5-methylcytosine)
  modified_base (58)..(58): Potential to be methylated (5-methylcytosine)
  modified_base (77)..(77): Potential to be methylated (5-methylcytosine)
  modified_base (80)..(80): Potential to be methylated (5-methylcytosine)
gaacaacagataagggtggctggcagtaagcacgacgacgagcaaccccgtttccttcgcctaaccaggagtcagtcgccgggcttctggaatgcctgccccaggtga

SEQ ID NO: 68 - DNA, 108 bp, Homo sapiens; identical to SEQ ID NO: 67
  modified_base (33)..(33): Potential to be methylated (5-methylcytosine)

SEQ ID NO: 69 - DNA, 108 bp, Homo sapiens; identical to SEQ ID NO: 67
  modified_base (36)..(36): Potential to be methylated (5-methylcytosine)

SEQ ID NO: 70 - DNA, 108 bp, Homo sapiens; identical to SEQ ID NO: 67
  modified_base (39)..(39): Potential to be methylated (5-methylcytosine)

SEQ ID NO: 71 - DNA, 108 bp, Homo sapiens; identical to SEQ ID NO: 67
  modified_base (49)..(49): Potential to be methylated (5-methylcytosine)

SEQ ID NO: 72 - DNA, 108 bp, Homo sapiens; identical to SEQ ID NO: 67
  modified_base (58)..(58): Potential to be methylated (5-methylcytosine)

SEQ ID NO: 73 - DNA, 108 bp, Homo sapiens; identical to SEQ ID NO: 67
  modified_base (77)..(77): Potential to be methylated (5-methylcytosine)

SEQ ID NO: 74 - DNA, 108 bp, Homo sapiens; identical to SEQ ID NO: 67
  modified_base (80)..(80): Potential to be methylated (5-methylcytosine)

SEQ ID NO: 75 - DNA, 113 bp, Homo sapiens
  modified_base (36)..(36): Potential to be methylated (5-methylcytosine)
  modified_base (39)..(39): Potential to be methylated (5-methylcytosine)
  modified_base (42)..(42): Potential to be methylated (5-methylcytosine)
  modified_base (52)..(52): Potential to be methylated (5-methylcytosine)
  modified_base (61)..(61): Potential to be methylated (5-methylcytosine)
  modified_base (80)..(80): Potential to be methylated (5-methylcytosine)
  modified_base (83)..(83): Potential to be methylated (5-methylcytosine)
aaggaacaacagataagggtggctggcagtaagcacgacgacgagcaaccccgtttccttcgcctaaccaggagtcagtcgccgggcttctggaatgcctgccccaggtgagc

SEQ ID NO: 76 - DNA, 113 bp, Homo sapiens; identical to SEQ ID NO: 75
  modified_base (36)..(36): Potential to be methylated (5-methylcytosine)

SEQ ID NO: 77 - DNA, 113 bp, Homo sapiens; identical to SEQ ID NO: 75
  modified_base (39)..(39): Potential to be methylated (5-methylcytosine)

SEQ ID NO: 78 - DNA, 113 bp, Homo sapiens; identical to SEQ ID NO: 75
  modified_base (42)..(42): Potential to be methylated (5-methylcytosine)

SEQ ID NO: 79 - DNA, 113 bp, Homo sapiens; identical to SEQ ID NO: 75
  modified_base (52)..(52): Potential to be methylated (5-methylcytosine)

SEQ ID NO: 80 - DNA, 113 bp, Homo sapiens; identical to SEQ ID NO: 75
  modified_base (61)..(61): Potential to be methylated (5-methylcytosine)

SEQ ID NO: 81 - DNA, 113 bp, Homo sapiens; identical to SEQ ID NO: 75
  modified_base (80)..(80): Potential to be methylated (5-methylcytosine)

SEQ ID NO: 82 - DNA, 113 bp, Homo sapiens; identical to SEQ ID NO: 75
  modified_base (83)..(83): Potential to be methylated (5-methylcytosine)

SEQ ID NO: 83 - DNA, 118 bp, Homo sapiens
  modified_base (26)..(26): Potential to be methylated (5-methylcytosine)
  modified_base (32)..(32): Potential to be methylated (5-methylcytosine)
  modified_base (46)..(46): Potential to be methylated (5-methylcytosine)
  modified_base (84)..(84): Potential to be methylated (5-methylcytosine)
  modified_base (92)..(92): Potential to be methylated (5-methylcytosine)
ccgggacagcaccttgggagctgggcggagacgcttaaatcccaacgcttccagaaagaagtttgtgaagaaaaggtgaagagcgagttcccgcaggcaaattggatgggcgtctggc

SEQ ID NO: 84 - DNA, 118 bp, Homo sapiens; identical to SEQ ID NO: 83
  modified_base (26)..(26): Potential to be methylated (5-methylcytosine)

SEQ ID NO: 85 - DNA, 118 bp, Homo sapiens; identical to SEQ ID NO: 83
  modified_base (32)..(32): Potential to be methylated (5-methylcytosine)

SEQ ID NO: 86 - DNA, 118 bp, Homo sapiens; identical to SEQ ID NO: 83
  modified_base (46)..(46): Potential to be methylated (5-methylcytosine)

SEQ ID NO: 87 - DNA, 118 bp, Homo sapiens; identical to SEQ ID NO: 83
  modified_base (84)..(84): Potential to be methylated (5-methylcytosine)

SEQ ID NO: 88 - DNA, 118 bp, Homo sapiens; identical to SEQ ID NO: 83
  modified_base (92)..(92): Potential to be methylated (5-methylcytosine)

SEQ ID NO: 89 - DNA, 150 bp, Homo sapiens
  modified_base (27)..(27): Potential to be methylated (5-methylcytosine)
  modified_base (37)..(37): Potential to be methylated (5-methylcytosine)
  modified_base (65)..(65): Potential to be methylated (5-methylcytosine)
  modified_base (70)..(70): Potential to be methylated (5-methylcytosine)
  modified_base (81)..(81): Potential to be methylated (5-methylcytosine)
  modified_base (95)..(95): Potential to be methylated (5-methylcytosine)
  modified_base (115)..(115): Potential to be methylated (5-methylcytosine)
ttaaaaacccctctctcttccgggtgcggtggctcacgcctgtaatcccagcactttgggaggccgaggcgggtggatcacgaggtcaggagatcgagaccatcctggttaacacgatgaaaacccgtctctactaaaaaaaatacaaaa

SEQ ID NO: 90 - DNA, 150 bp, Homo sapiens; identical to SEQ ID NO: 89
  modified_base (27)..(27): Potential to be methylated (5-methylcytosine)

SEQ ID NO: 91 - DNA, 150 bp, Homo sapiens; identical to SEQ ID NO: 89
  modified_base (37)..(37): Potential to be methylated (5-methylcytosine)

SEQ ID NO: 92 - DNA, 150 bp, Homo sapiens; identical to SEQ ID NO: 89
  modified_base (65)..(65): Potential to be methylated (5-methylcytosine)

SEQ ID NO: 93 - DNA, 150 bp, Homo sapiens; identical to SEQ ID NO: 89
  modified_base (70)..(70): Potential to be methylated (5-methylcytosine)

SEQ ID NO: 94 - DNA, 150 bp, Homo sapiens; identical to SEQ ID NO: 89
  modified_base (81)..(81): Potential to be methylated (5-methylcytosine)

SEQ ID NO: 95 - DNA, 150 bp, Homo sapiens; identical to SEQ ID NO: 89
  modified_base (95)..(95): Potential to be methylated (5-methylcytosine)

SEQ ID NO: 96 - DNA, 150 bp, Homo sapiens; identical to SEQ ID NO: 89
  modified_base (115)..(115): Potential to be methylated (5-methylcytosine)

SEQ ID NO: 97 - DNA, 125 bp, Homo sapiens
  modified_base (23)..(23): Potential to be methylated (5-methylcytosine)
  modified_base (25)..(25): Potential to be methylated (5-methylcytosine)
  modified_base (32)..(32): Potential to be methylated (5-methylcytosine)
  modified_base (35)..(35): Potential to be methylated (5-methylcytosine)
  modified_base (38)..(38): Potential to be methylated (5-methylcytosine)
  modified_base (41)..(41): Potential to be methylated (5-methylcytosine)
  modified_base (46)..(46): Potential to be methylated (5-methylcytosine)
  modified_base (52)..(52): Potential to be methylated (5-methylcytosine)
  modified_base (66)..(66): Potential to be methylated (5-methylcytosine)
  modified_base (75)..(75): Potential to be methylated (5-methylcytosine)
  modified_base (78)..(78): Potential to be methylated (5-methylcytosine)
  modified_base (85)..(85): Potential to be methylated (5-methylcytosine)
  modified_base (90)..(90): Potential to be methylated (5-methylcytosine)
  modified_base (93)..(93): Potential to be methylated (5-methylcytosine)
gcctcctcacgaaagagcagctcgcgggtgacgccgtcgccgcctcggaagcggcctctgccccccgagccccccgccgcagctcgaagcggcgcaggatgaccgggtacctgcgagggcgagga

SEQ ID NO: 98 - DNA, 125 bp, Homo sapiens; identical to SEQ ID NO: 97
  modified_base (23)..(23): Potential to be methylated (5-methylcytosine)

SEQ ID NO: 99 - DNA, 125 bp, Homo sapiens; identical to SEQ ID NO: 97
  modified_base (25)..(25): Potential to be methylated (5-methylcytosine)

SEQ ID NO: 100 - DNA, 125 bp, Homo sapiens; identical to SEQ ID NO: 97
  modified_base (32)..(32): Potential to be methylated (5-methylcytosine)

SEQ ID NO: 101 - DNA, 125 bp, Homo sapiens; identical to SEQ ID NO: 97
  modified_base (35)..(35): Potential to be methylated (5-methylcytosine)

SEQ ID NO: 102 - DNA, 125 bp, Homo sapiens; identical to SEQ ID NO: 97
  modified_base (38)..(38): Potential to be methylated (5-methylcytosine)

SEQ ID NO: 103 - DNA, 125 bp, Homo sapiens; identical to SEQ ID NO: 97
  modified_base (41)..(41): Potential to be methylated (5-methylcytosine)

SEQ ID NO: 104 - DNA, 125 bp, Homo sapiens; identical to SEQ ID NO: 97
  modified_base (46)..(46): Potential to be methylated (5-methylcytosine)

SEQ ID NO: 105 - DNA, 125 bp, Homo sapiens; identical to SEQ ID NO: 97
  modified_base (52)..(52): Potential to be methylated (5-methylcytosine)

SEQ ID NO: 106 - DNA, 125 bp, Homo sapiens; identical to SEQ ID NO: 97
  modified_base (66)..(66): Potential to be methylated (5-methylcytosine)

SEQ ID NO: 107 - DNA, 125 bp, Homo sapiens; identical to SEQ ID NO: 97
  modified_base (75)..(75): Potential to be methylated (5-methylcytosine)

SEQ ID NO: 108 - DNA, 125 bp, Homo sapiens; identical to SEQ ID NO: 97
  modified_base (78)..(78): Potential to be methylated (5-methylcytosine)

SEQ ID NO: 109 - DNA, 125 bp, Homo sapiens; identical to SEQ ID NO: 97
  modified_base (85)..(85): Potential to be methylated (5-methylcytosine)

SEQ ID NO: 110 - DNA, 125 bp, Homo sapiens; identical to SEQ ID NO: 97
  modified_base (90)..(90): Potential to be methylated (5-methylcytosine)

SEQ ID NO: 111 - DNA, 125 bp, Homo sapiens; identical to SEQ ID NO: 97
  modified_base (93)..(93): Potential to be methylated (5-methylcytosine)

SEQ ID NO: 112 - DNA, 119 bp, Homo sapiens
  modified_base (36)..(36): Potential to be methylated (5-methylcytosine)
  modified_base (54)..(54): Potential to be methylated (5-methylcytosine)
  modified_base (68)..(68): Potential to be methylated (5-methylcytosine)
  modified_base (72)..(72): Potential to be methylated (5-methylcytosine)
  modified_base (74)..(74): Potential to be methylated (5-methylcytosine)
  modified_base (80)..(80): Potential to be methylated (5-methylcytosine)
  modified_base (89)..(89): Potential to be methylated (5-methylcytosine)
ggatccagggtggggatttgagatcaggtccctttcgggttttctttttgaagcgcccctctgcctccgcccgcgcctccgccaggctcgctgcgtcagcacctcaccggctttgcaca

SEQ ID NO: 113 - DNA, 119 bp, Homo sapiens; identical to SEQ ID NO: 112
  modified_base (36)..(36): Potential to be methylated (5-methylcytosine)

SEQ ID NO: 114 - DNA, 119 bp, Homo sapiens; identical to SEQ ID NO: 112
  modified_base (54)..(54): Potential to be methylated (5-methylcytosine)

SEQ ID NO: 115 - DNA, 119 bp, Homo sapiens; identical to SEQ ID NO: 112
  modified_base (68)..(68): Potential to be methylated (5-methylcytosine)

SEQ ID NO: 116 - DNA, 119 bp, Homo sapiens; identical to SEQ ID NO: 112
  modified_base (72)..(72): Potential to be methylated (5-methylcytosine)

SEQ ID NO: 117 - DNA, 119 bp, Homo sapiens; identical to SEQ ID NO: 112
  modified_base (74)..(74): Potential to be methylated (5-methylcytosine)

SEQ ID NO: 118 - DNA, 119 bp, Homo sapiens; identical to SEQ ID NO: 112
  modified_base (80)..(80): Potential to be methylated (5-methylcytosine)

SEQ ID NO: 119 - DNA, 119 bp, Homo sapiens; identical to SEQ ID NO: 112
  modified_base (89)..(89): Potential to be methylated (5-methylcytosine)

SEQ ID NO: 120 - DNA, 115 bp, Homo sapiens
  modified_base (37)..(37): Potential to be methylated (5-methylcytosine)
  modified_base (39)..(39): Potential to be methylated (5-methylcytosine)
  modified_base (56)..(56): Potential to be methylated (5-methylcytosine)
  modified_base (64)..(64): Potential to be methylated (5-methylcytosine)
  modified_base (71)..(71): Potential to be methylated (5-methylcytosine)
  modified_base (73)..(73): Potential to be methylated (5-methylcytosine)
  modified_base (76)..(76): Potential to be methylated (5-methylcytosine)
  modified_base (86)..(86): Potential to be methylated (5-methylcytosine)
ggcagagtgaagcacaagcaataatcctgtattattcgcgttcccagagtcccttcggatttgcgccatgcgcggcggggagaaccggcctcctgctcgagttcagagctcatct

SEQ ID NO: 121 - DNA, 115 bp, Homo sapiens; identical to SEQ ID NO: 120
  modified_base (37)..(37): Potential to be methylated (5-methylcytosine)

SEQ ID NO: 122 - DNA, 115 bp, Homo sapiens; identical to SEQ ID NO: 120
  modified_base (39)..(39): Potential to be methylated (5-methylcytosine)

SEQ ID NO: 123 - DNA, 115 bp, Homo sapiens; identical to SEQ ID NO: 120
  modified_base (56)..(56): Potential to be methylated (5-methylcytosine)

SEQ ID NO: 124 - DNA, 115 bp, Homo sapiens; identical to SEQ ID NO: 120
  modified_base (64)..(64): Potential to be methylated (5-methylcytosine)

SEQ ID NO: 125 - DNA, 115 bp, Homo sapiens; identical to SEQ ID NO: 120
  modified_base (71)..(71): Potential to be methylated (5-methylcytosine)

SEQ ID NO: 126 - DNA, 115 bp, Homo sapiens; identical to SEQ ID NO: 120
  modified_base (73)..(73): Potential to be methylated (5-methylcytosine)

SEQ ID NO: 127 - DNA, 115 bp, Homo sapiens; identical to SEQ ID NO: 120
  modified_base (76)..(76): Potential to be methylated (5-methylcytosine)

SEQ ID NO: 128 - DNA, 115 bp, Homo sapiens; identical to SEQ ID NO: 120
  modified_base (86)..(86): Potential to be methylated (5-methylcytosine)

SEQ ID NO: 129 - DNA, 102 bp, Homo sapiens
  modified_base (36)..(36): Potential to be methylated (5-methylcytosine)
  modified_base (38)..(38): Potential to be methylated (5-methylcytosine)
  modified_base (55)..(55): Potential to be methylated (5-methylcytosine)
  modified_base (63)..(63): Potential to be methylated (5-methylcytosine)
  modified_base (70)..(70): Potential to be methylated (5-methylcytosine)
  modified_base (72)..(72): Potential to be methylated (5-methylcytosine)
  modified_base (75)..(75): Potential to be methylated (5-methylcytosine)
gcagagtgaagcacaagcaataatcctgtattattcgcgttcccagagtcccttcggatttgcgccatgcgcggcggggagaaccggcctcctgctcgagtt

SEQ ID NO: 130 - DNA, 102 bp, Homo sapiens; identical to SEQ ID NO: 129
  modified_base (36)..(36): Potential to be methylated (5-methylcytosine)

SEQ ID NO: 131 - DNA, 102 bp, Homo sapiens; identical to SEQ ID NO: 129
  modified_base (38)..(38): Potential to be methylated (5-methylcytosine)

SEQ ID NO: 132 - DNA, 102 bp, Homo sapiens; identical to SEQ ID NO: 129
  modified_base (55)..(55): Potential to be methylated (5-methylcytosine)

SEQ ID NO: 133 - DNA, 102 bp, Homo sapiens; identical to SEQ ID NO: 129
  modified_base (63)..(63): Potential to be methylated (5-methylcytosine)

SEQ ID NO: 134 - DNA, 102 bp, Homo sapiens; identical to SEQ ID NO: 129
  modified_base (70)..(70): Potential to be methylated (5-methylcytosine)

SEQ ID NO: 135 - DNA, 102 bp, Homo sapiens; identical to SEQ ID NO: 129
  modified_base (72)..(72): Potential to be methylated (5-methylcytosine)

SEQ ID NO: 136 - DNA, 102 bp, Homo sapiens; identical to SEQ ID NO: 129
  modified_base (75)..(75): Potential to be methylated (5-methylcytosine)

SEQ ID NO: 137 - DNA, 95 bp, Homo sapiens
  modified_base (36)..(36): Potential to be methylated (5-methylcytosine)
  modified_base (38)..(38): Potential to be methylated (5-methylcytosine)
  modified_base (42)..(42): Potential to be methylated (5-methylcytosine)
  modified_base (44)..(44): Potential to be methylated (5-methylcytosine)
  modified_base (64)..(64): Potential to be methylated (5-methylcytosine)
  modified_base (66)..(66): Potential to be methylated (5-methylcytosine)
actccctcctcctgcacctcctgcagcccggctcccgcggccgcgcctggtgcccctctgtctcgcgccacctgagatgcccaggctggcctctg

SEQ ID NO: 138 - DNA, 95 bp, Homo sapiens; identical to SEQ ID NO: 137
  modified_base (36)..(36): Potential to be methylated (5-methylcytosine)

SEQ ID NO: 139 - DNA, 95 bp, Homo sapiens; identical to SEQ ID NO: 137
  modified_base (38)..(38): Potential to be methylated (5-methylcytosine)

SEQ ID NO: 140 - DNA, 95 bp, Homo sapiens; identical to SEQ ID NO: 137
  modified_base (42)..(42): Potential to be methylated (5-methylcytosine)

SEQ ID NO: 141 - DNA, 95 bp, Homo sapiens; identical to SEQ ID NO: 137
  modified_base (44)..(44): Potential to be methylated (5-methylcytosine)

SEQ ID NO: 142 - DNA, 95 bp, Homo sapiens; identical to SEQ ID NO: 137
  modified_base (64)..(64): Potential to be methylated (5-methylcytosine)

SEQ ID NO: 143 - DNA, 95 bp, Homo sapiens; identical to SEQ ID NO: 137
  modified_base (66)..(66): Potential to be methylated (5-methylcytosine)

SEQ ID NO: 144 - DNA, 110 bp, Homo sapiens
  modified_base (28)..(28): Potential to be methylated (5-methylcytosine)
  modified_base (50)..(50): Potential to be methylated (5-methylcytosine)
  modified_base (54)..(54): Potential to be methylated (5-methylcytosine)
  modified_base (56)..(56): Potential to be methylated (5-methylcytosine)
  modified_base (62)..(62): Potential to be methylated (5-methylcytosine)
  modified_base (65)..(65): Potential to be methylated (5-methylcytosine)
  modified_base (70)..(70): Potential to be methylated (5-methylcytosine)
  modified_base (72)..(72): Potential to be methylated (5-methylcytosine)
  modified_base (85)..(85): Potential to be methylated (5-methylcytosine)
ccagccccaagtcttgcgggcagttcccgaagaaaagatgggtttggggcggtcgcgaaagcggcgcctcgcgtgttttcctgccgttcccgggtccttatagcccggcc

SEQ ID NO: 145 - DNA, 110 bp, Homo sapiens; identical to SEQ ID NO: 144
  modified_base (28)..(28): Potential to be methylated (5-methylcytosine)

SEQ ID NO: 146 - DNA, 110 bp, Homo sapiens; identical to SEQ ID NO: 144
  modified_base (50)..(50): Potential to be methylated (5-methylcytosine)

SEQ ID NO: 147 - DNA, 110 bp, Homo sapiens; identical to SEQ ID NO: 144
  modified_base (54)..(54): Potential to be methylated (5-methylcytosine)

SEQ ID NO: 148 - DNA, 110 bp, Homo sapiens; identical to SEQ ID NO: 144
  modified_base (56)..(56): Potential to be methylated (5-methylcytosine)

SEQ ID NO: 149 - DNA, 110 bp, Homo sapiens; identical to SEQ ID NO: 144
  modified_base (62)..(62): Potential to be methylated (5-methylcytosine)

SEQ ID NO: 150 - DNA, 110 bp, Homo sapiens; identical to SEQ ID NO: 144
  modified_base (65)..(65): Potential to be methylated (5-methylcytosine)

SEQ ID NO: 151 - DNA, 110 bp, Homo sapiens; identical to SEQ ID NO: 144
  modified_base (70)..(70): Potential to be methylated (5-methylcytosine)

SEQ ID NO: 152 - DNA, 110 bp, Homo sapiens; identical to SEQ ID NO: 144
  modified_base (72)..(72): Potential to be methylated (5-methylcytosine)

SEQ ID NO: 153 - DNA, 110 bp, Homo sapiens; identical to SEQ ID NO: 144
  modified_base (85)..(85): Potential to be methylated (5-methylcytosine)

SEQ ID NO: 154 - DNA, 129 bp, Homo sapiens
  modified_base (29)..(29): Potential to be methylated (5-methylcytosine)
  modified_base (39)..(39): Potential to be methylated (5-methylcytosine)
  modified_base (56)..(56): Potential to be methylated (5-methylcytosine)
  modified_base (66)..(66): Potential to be methylated (5-methylcytosine)
  modified_base (87)..(87): Potential to be methylated (5-methylcytosine)
  modified_base (102)..(102): Potential to be methylated (5-methylcytosine)
  modified_base (104)..(104): Potential to be methylated (5-methylcytosine)
gccccacgagcctccgtccgttctggttcgggtttctccgagttttgctaccagccgaggctgtgcgggcaactgggtcagcctcccgtcaggagagaagccgcgtctgtgggacgaagaccgggcacc

SEQ ID NO: 155 - DNA, 129 bp, Homo sapiens; identical to SEQ ID NO: 154
  modified_base (29)..(29): Potential to be methylated (5-methylcytosine)

SEQ ID NO: 156 - DNA, 129 bp, Homo sapiens; identical to SEQ ID NO: 154
  modified_base (39)..(39): Potential to be methylated (5-methylcytosine)

SEQ ID NO: 157 - DNA, 129 bp, Homo sapiens; identical to SEQ ID NO: 154
  modified_base (56)..(56): Potential to be methylated (5-methylcytosine)

SEQ ID NO: 158 - DNA, 129 bp, Homo sapiens; identical to SEQ ID NO: 154
  modified_base (66)..(66): Potential to be methylated (5-methylcytosine)

SEQ ID NO: 159 - DNA, 129 bp, Homo sapiens; identical to SEQ ID NO: 154
  modified_base (87)..(87): Potential to be methylated (5-methylcytosine)

SEQ ID NO: 160 - DNA, 129 bp, Homo sapiens; identical to SEQ ID NO: 154
  modified_base (102)..(102): Potential to be methylated (5-methylcytosine)

SEQ ID NO: 161 - DNA, 129 bp, Homo sapiens; identical to SEQ ID NO: 154
  modified_base (104)..(104): Potential to be methylated (5-methylcytosine)

SEQ ID NO: 162 - DNA, 131 bp, Homo sapiens
  modified_base (34)..(34): Potential to be methylated (5-methylcytosine)
  modified_base (40)..(40): Potential to be methylated (5-methylcytosine)
  modified_base (54)..(54): Potential to be methylated (5-methylcytosine)
  modified_base (92)..(92): Potential to be methylated (5-methylcytosine)
  modified_base (100)..(100): Potential to be methylated (5-methylcytosine)
ctacaggtccgggacagcaccttgggagctgggcggagacgcttaaatcccaacgcttccagaaagaagtttgtgaagaaaaggtgaagagcgagttcccgcaggcaaattggatgggcgtctggccgccg

SEQ ID NO: 163 - DNA, 131 bp, Homo sapiens; identical to SEQ ID NO: 162
  modified_base (34)..(34): Potential to be methylated (5-methylcytosine)

SEQ ID NO: 164 - DNA, 131 bp, Homo sapiens; identical to SEQ ID NO: 162
  modified_base (40)..(40): Potential to be methylated (5-methylcytosine)

SEQ ID NO: 165 - DNA, 131 bp, Homo sapiens; identical to SEQ ID NO: 162
  modified_base (54)..(54): Potential to be methylated (5-methylcytosine)

SEQ ID NO: 166 - DNA, 131 bp, Homo sapiens; identical to SEQ ID NO: 162
  modified_base (92)..(92): Potential to be methylated (5-methylcytosine)

SEQ ID NO: 167 - DNA, 131 bp, Homo sapiens; identical to SEQ ID NO: 162
  modified_base (100)..(100): Potential to be methylated (5-methylcytosine)

SEQ ID NO: 168 - DNA, 127 bp, Homo sapiens
  modified_base (32)..(32): Potential to be methylated (5-methylcytosine)
  modified_base (34)..(34): Potential to be methylated (5-methylcytosine)
  modified_base (36)..(36): Potential to be methylated (5-methylcytosine)
  modified_base (43)..(43): Potential to be methylated (5-methylcytosine)
  modified_base (45)..(45): Potential to be methylated (5-methylcytosine)
  modified_base (55)..(55): Potential to be methylated (5-methylcytosine)
  modified_base (60)..(60): Potential to be methylated (5-methylcytosine)
  modified_base (67)..(67): Potential to be methylated (5-methylcytosine)
  modified_base (76)..(76): Potential to be methylated (5-methylcytosine)
  modified_base (79)..(79): Potential to be methylated (5-methylcytosine)
  modified_base (87)..(87): Potential to be methylated (5-methylcytosine)
  modified_base (99)..(99): Potential to be methylated (5-methylcytosine)
gtgtcctcctaaggcaagcacagatgaggggcgcgcggctggcgcgcacagacacgactcggagcacgaactaggcgccgtagctgcgtccccagaaccgggagacttaaggcatctttattgcggg

SEQ ID NO: 169 - DNA, 127 bp, Homo sapiens; identical to SEQ ID NO: 168
  modified_base (32)..(32): Potential to be methylated (5-methylcytosine)

SEQ ID NO: 170 - DNA, 127 bp, Homo sapiens; identical to SEQ ID NO: 168
  modified_base (34)..(34): Potential to be methylated (5-methylcytosine)

SEQ ID NO: 171 - DNA, 127 bp, Homo sapiens; identical to SEQ ID NO: 168
  modified_base (36)..(36): Potential to be methylated (5-methylcytosine)

SEQ ID NO: 172 - DNA, 127 bp, Homo sapiens; identical to SEQ ID NO: 168
  modified_base (43)..(43): Potential to be methylated (5-methylcytosine)

SEQ ID NO: 173 - DNA, 127 bp, Homo sapiens; identical to SEQ ID NO: 168
  modified_base (45)..(45): Potential to be methylated (5-methylcytosine)

SEQ ID NO: 174 - DNA, 127 bp, Homo sapiens; identical to SEQ ID NO: 168
  modified_base (55)..(55): Potential to be methylated (5-methylcytosine)

SEQ ID NO: 175 - DNA, 127 bp, Homo sapiens; identical to SEQ ID NO: 168
  modified_base (60)..(60): Potential to be methylated (5-methylcytosine)

SEQ ID NO: 176 - DNA, 127 bp, Homo sapiens; identical to SEQ ID NO: 168
  modified_base (67)..(67): Potential to be methylated (5-methylcytosine)

SEQ ID NO: 177 - DNA, 127 bp, Homo sapiens; identical to SEQ ID NO: 168
  modified_base (76)..(76): Potential to be methylated (5-methylcytosine)

SEQ ID NO: 178 - DNA, 127 bp, Homo sapiens; identical to SEQ ID NO: 168
  modified_base (79)..(79): Potential to be methylated (5-methylcytosine)

SEQ ID NO: 179 - DNA, 127 bp, Homo sapiens; identical to SEQ ID NO: 168
  modified_base (87)..(87): Potential to be methylated (5-methylcytosine)

SEQ ID NO: 180 - DNA, 127 bp, Homo sapiens; identical to SEQ ID NO: 168
  modified_base (99)..(99): Potential to be methylated (5-methylcytosine)

SEQ ID NO: 181 - DNA, 120 bp, Homo sapiens
  modified_base (36)..(36): Potential to be methylated (5-methylcytosine)
  modified_base (38)..(38): Potential to be methylated (5-methylcytosine)
  modified_base (40)..(40): Potential to be methylated (5-methylcytosine)
  modified_base (47)..(47): Potential to be methylated (5-methylcytosine)
  modified_base (49)..(49): Potential to be methylated (5-methylcytosine)
  modified_base (59)..(59): Potential to be methylated (5-methylcytosine)
  modified_base (64)..(64): Potential to be methylated (5-methylcytosine)
  modified_base (71)..(71): Potential to be methylated (5-methylcytosine)
  modified_base (80)..(80): Potential to be methylated (5-methylcytosine)
  modified_base (83)..(83): Potential to be methylated (5-methylcytosine)
  modified_base (91)..(91): Potential to be methylated (5-methylcytosine)
ctcagtgtcctcctaaggcaagcacagatgaggggcgcgcggctggcgcgcacagacacgactcggagcacgaactaggcgccgtagctgcgtccccagaaccgggagacttaaggcatc

SEQ ID NO: 182 - DNA, 120 bp, Homo sapiens; identical to SEQ ID NO: 181
  modified_base (36)..(36): Potential to be methylated (5-methylcytosine)

SEQ ID NO: 183 - DNA, 120 bp, Homo sapiens; identical to SEQ ID NO: 181
  modified_base (38)..(38): Potential to be methylated (5-methylcytosine)

SEQ ID NO: 184 - DNA, 120 bp, Homo sapiens; identical to SEQ ID NO: 181
  modified_base (40)..(40): Potential to be methylated (5-methylcytosine)

SEQ ID NO: 185 - DNA, 120 bp, Homo sapiens; identical to SEQ ID NO: 181
  modified_base (47)..(47): Potential to be methylated (5-methylcytosine)

SEQ ID NO: 186 - DNA, 120 bp, Homo sapiens; identical to SEQ ID NO: 181
  modified_base (49)..(49): Potential to be methylated (5-methylcytosine)

SEQ ID NO: 187 - DNA, 120 bp, Homo sapiens; identical to SEQ ID NO: 181
  modified_base (59)..(59): Potential to be methylated (5-methylcytosine)

SEQ ID NO: 188 - DNA, 120 bp, Homo sapiens; identical to SEQ ID NO: 181
  modified_base (64)..(64): Potential to be methylated (5-methylcytosine)

SEQ ID NO: 189 - DNA, 120 bp, Homo sapiens; identical to SEQ ID NO: 181
  modified_base (71)..(71): Potential to be methylated (5-methylcytosine)

SEQ ID NO: 190 - DNA, 120 bp, Homo sapiens; identical to SEQ ID NO: 181
  modified_base (80)..(80): Potential to be methylated (5-methylcytosine)

SEQ ID NO: 191 - DNA, 120 bp, Homo sapiens; identical to SEQ ID NO: 181
  modified_base (83)..(83): Potential to be methylated (5-methylcytosine)

SEQ ID NO: 192 - DNA, 120 bp, Homo sapiens; identical to SEQ ID NO: 181
  modified_base (91)..(91): Potential to be methylated (5-methylcytosine)

SEQ ID NO: 193 - DNA, 99 bp, Homo sapiens
  modified_base (25)..(25): Potential to be methylated (5-methylcytosine)
  modified_base (32)..(32): Potential to be methylated (5-methylcytosine)
  modified_base (34)..(34): Potential to be methylated (5-methylcytosine)
  modified_base (38)..(38): Potential to be methylated (5-methylcytosine)
  modified_base (40)..(40): Potential to be methylated (5-methylcytosine)
  modified_base (60)..(60): Potential to be methylated (5-methylcytosine)
  modified_base (62)..(62): Potential to be methylated (5-methylcytosine)
cctcctcctgcacctcctgcagcccggctcccgcggccgcgcctggtgcccctctgtctcgcgccacctgagatgcccaggctggcctctgccaggggc

SEQ ID NO: 194 - DNA, 99 bp, Homo sapiens; identical to SEQ ID NO: 193
  modified_base (25)..(25): Potential to be methylated (5-methylcytosine)

SEQ ID NO: 195 - DNA, 99 bp, Homo sapiens; identical to SEQ ID NO: 193
  modified_base (32)..(32): Potential to be methylated (5-methylcytosine)

SEQ ID NO: 196 - DNA, 99 bp, Homo sapiens; identical to SEQ ID NO: 193
  modified_base (34)..(34): Potential to be methylated (5-methylcytosine)

SEQ ID NO: 197 - DNA, 99 bp, Homo sapiens; identical to SEQ ID NO: 193
  modified_base (38)..(38): Potential to be methylated (5-methylcytosine)

SEQ ID NO: 198 - DNA, 99 bp, Homo sapiens; identical to SEQ ID NO: 193
  modified_base (40)..(40): Potential to be methylated (5-methylcytosine)

SEQ ID NO: 199 - DNA, 99 bp, Homo sapiens; identical to SEQ ID NO: 193
  modified_base (60)..(60): Potential to be methylated (5-methylcytosine)

SEQ ID NO: 200 - DNA, 99 bp, Homo sapiens; identical to SEQ ID NO: 193
  modified_base (62)..(62): Potential to be methylated (5-methylcytosine)

SEQ ID NO: 201 - DNA, 118 bp, Homo sapiens
  modified_base (30)..(30): Potential to be methylated (5-methylcytosine)
  modified_base (34)..(34): Potential to be methylated (5-methylcytosine)
  modified_base (36)..(36): Potential to be methylated (5-methylcytosine)
  modified_base (42)..(42): Potential to be methylated (5-methylcytosine)
  modified_base (45)..(45): Potential to be methylated (5-methylcytosine)
  modified_base (50)..(50): Potential to be methylated (5-methylcytosine)
  modified_base (52)..(52): Potential to be methylated (5-methylcytosine)
  modified_base (65)..(65): Potential to be methylated (5-methylcytosine)
  modified_base (71)..(71): Potential to be methylated (5-methylcytosine)
  modified_base (86)..(86): Potential to be methylated (5-methylcytosine)
  modified_base (90)..(90): Potential to be methylated (5-methylcytosine)
cagttcccgaagaaaagatgggtttggggcggtcgcgaaagcggcgcctcgcgtgttttcctgccgttcccgggtccttatagcccggccggagactccgctgagttgactcggcgcc

SEQ ID NO: 202 - DNA, 118 bp, Homo sapiens; identical to SEQ ID NO: 201
  modified_base (30)..(30): Potential to be methylated (5-methylcytosine)

SEQ ID NO: 203 - DNA, 118 bp, Homo sapiens; identical to SEQ ID NO: 201
  modified_base (34)..(34): Potential to be methylated (5-methylcytosine)

SEQ ID NO: 204 - DNA, 118 bp, Homo sapiens; identical to SEQ ID NO: 201
  modified_base (36)..(36): Potential to be methylated (5-methylcytosine)

SEQ ID NO: 205 - DNA, 118 bp, Homo sapiens; identical to SEQ ID NO: 201
  modified_base (42)..(42): Potential to be methylated (5-methylcytosine)

SEQ ID NO: 206 - DNA, 118 bp, Homo sapiens; identical to SEQ ID NO: 201
  modified_base (45)..(45): Potential to be methylated (5-methylcytosine)

SEQ ID NO: 207 - DNA, 118 bp, Homo sapiens; identical to SEQ ID NO: 201
  modified_base (50)..(50): Potential to be methylated (5-methylcytosine)

SEQ ID NO: 208 - DNA, 118 bp, Homo sapiens; identical to SEQ ID NO: 201
  modified_base (52)..(52): Potential to be methylated (5-methylcytosine)

SEQ ID NO: 209 - DNA, 118 bp, Homo sapiens; identical to SEQ ID NO: 201
  modified_base (65)..(65): Potential to be methylated (5-methylcytosine)

SEQ ID NO: 210 - DNA, 118 bp, Homo sapiens; identical to SEQ ID NO: 201
  modified_base (71)..(71): Potential to be methylated (5-methylcytosine)

SEQ ID NO: 211 - DNA, 118 bp, Homo sapiens; identical to SEQ ID NO: 201
  modified_base (86)..(86): Potential to be methylated (5-methylcytosine)

SEQ ID NO: 212 - DNA, 118 bp, Homo sapiens; identical to SEQ ID NO: 201
  modified_base (90)..(90): Potential to be methylated (5-methylcytosine)

SEQ ID NO: 213 - DNA, 123 bp, Homo sapiens
  modified_base (33)..(33): Potential to be methylated (5-methylcytosine)
  modified_base (37)..(37): Potential to be methylated (5-methylcytosine)
  modified_base (43)..(43): Potential to be methylated (5-methylcytosine)
  modified_base (62)..(62): Potential to be methylated (5-methylcytosine)
  modified_base (65)..(65): Potential to be methylated (5-methylcytosine)
  modified_base (71)..(71): Potential to be methylated (5-methylcytosine)
  modified_base (78)..(78): Potential to be methylated (5-methylcytosine)
  modified_base (86)..(86): Potential to be methylated (5-methylcytosine)
  modified_base (96)..(96): Potential to be methylated (5-methylcytosine)
  modified_base (99)..(99): Potential to be methylated (5-methylcytosine)
ggagccgctatggacgctgagctcctcagcttcgtccgtgtccgagtcaggggctgtgtggcggcggatacgggacacggcttctcgcagggccccggcgtagggccctggggtccgcgccca

SEQ ID NO: 214 - DNA, 123 bp, Homo sapiens; identical to SEQ ID NO: 213
  modified_base (33)..(33): Potential to be methylated (5-methylcytosine)

SEQ ID NO: 215 - DNA, 123 bp, Homo sapiens; identical to SEQ ID NO: 213
  modified_base (37)..(37): Potential to be methylated (5-methylcytosine)

SEQ ID NO: 216 - DNA, 123 bp, Homo sapiens; identical to SEQ ID NO: 213
  modified_base (43)..(43): Potential to be methylated (5-methylcytosine)

SEQ ID NO: 217 - DNA, 123 bp, Homo sapiens; identical to SEQ ID NO: 213
  modified_base (62)..(62): Potential to be methylated (5-methylcytosine)

SEQ ID NO: 218 - DNA, 123 bp, Homo sapiens; identical to SEQ ID NO: 213
  modified_base (65)..(65): Potential to be methylated (5-methylcytosine)

SEQ ID NO: 219 - DNA, 123 bp, Homo sapiens; identical to SEQ ID NO: 213
  modified_base (71)..(71): Potential to be methylated (5-methylcytosine)

SEQ ID NO: 220 - DNA, 123 bp, Homo sapiens; identical to SEQ ID NO: 213
  modified_base (78)..(78): Potential to be methylated (5-methylcytosine)

SEQ ID NO: 221 - DNA, 123 bp, Homo sapiens; identical to SEQ ID NO: 213
  modified_base (86)..(86): Potential to be methylated (5-methylcytosine)

SEQ ID NO: 222 - DNA, 123 bp, Homo sapiens; identical to SEQ ID NO: 213
  modified_base (96)..(96): Potential to be methylated (5-methylcytosine)

SEQ ID NO: 223 - DNA, 123 bp, Homo sapiens; identical to SEQ ID NO: 213
  modified_base (99)..(99): Potential to be methylated (5-methylcytosine)

SEQ ID NO: 224 - DNA, 141 bp, Homo sapiens
  modified_base (33)..(33): Potential to be methylated (5-methylcytosine)
  modified_base (42)..(42): Potential to be methylated (5-methylcytosine)
  modified_base (60)..(60): Potential to be methylated (5-methylcytosine)
  modified_base (64)..(64): Potential to be methylated (5-methylcytosine)
  modified_base (70)..(70): Potential to be methylated (5-methylcytosine)
  modified_base (89)..(89): Potential to be methylated (5-methylcytosine)
  modified_base (92)..(92): Potential to be methylated (5-methylcytosine)
  modified_base (98)..(98): Potential to be methylated (5-methylcytosine)
  modified_base (105)..(105): Potential to be methylated (5-methylcytosine)
  modified_base (113)..(113): Potential to be methylated (5-methylcytosine)
cggggcttctgtgtcgcttccatcagaggagccgctatggacgctgagctcctcagcttcgtccgtgtccgagtcaggggctgtgtggcggcggatacgggacacggcttctcgcagggccccggcgtagggccctggggt

SEQ ID NO: 225 - DNA, 141 bp, Homo sapiens; identical to SEQ ID NO: 224
  modified_base (33)..(33): Potential to be methylated (5-methylcytosine)

SEQ ID NO: 226 - DNA, 141 bp, Homo sapiens; identical to SEQ ID NO: 224
  modified_base (42)..(42): Potential to be methylated (5-methylcytosine)

SEQ ID NO: 227 - DNA, 141 bp, Homo sapiens; identical to SEQ ID NO: 224
  modified_base (60)..(60): Potential to be methylated (5-methylcytosine)

SEQ ID NO: 228 - DNA, 141 bp, Homo sapiens; identical to SEQ ID NO: 224
  modified_base (64)..(64): Potential to be methylated (5-methylcytosine)

SEQ ID NO: 229 - DNA, 141 bp, Homo sapiens; identical to SEQ ID NO: 224
  modified_base (70)..(70): Potential to be methylated (5-methylcytosine)

SEQ ID NO: 230 - DNA, 141 bp, Homo sapiens; identical to SEQ ID NO: 224
  modified_base (89)..(89): Potential to be methylated (5-methylcytosine)

SEQ ID NO: 231 - DNA, 141 bp, Homo sapiens; identical to SEQ ID NO: 224
  modified_base (92)..(92): Potential to be methylated (5-methylcytosine)

SEQ ID NO: 232 - DNA, 141 bp, Homo sapiens; identical to SEQ ID NO: 224
  modified_base (98)..(98): Potential to be methylated (5-methylcytosine)

SEQ ID NO: 233 - DNA, 141 bp, Homo sapiens; identical to SEQ ID NO: 224
  modified_base (105)..(105): Potential to be methylated (5-methylcytosine)

SEQ ID NO: 234 - DNA, 141 bp, Homo sapiens; identical to SEQ ID NO: 224
  modified_base (113)..(113): Potential to be methylated (5-methylcytosine)

SEQ ID NO: 235 - DNA, 118 bp, Homo sapiens
  modified_base (33)..(33): Potential to be methylated (5-methylcytosine)
  modified_base (43)..(43): Potential to be methylated (5-methylcytosine)
  modified_base (64)..(64): Potential to be methylated (5-methylcytosine)
  modified_base (79)..(79): Potential to be methylated (5-methylcytosine)
  modified_base (81)..(81): Potential to be methylated (5-methylcytosine)
  modified_base (92)..(92): Potential to be methylated (5-methylcytosine)
tggttcgggtttctccgagttttgctaccagccgaggctgtgcgggcaactgggtcagcctcccgtcaggagagaagccgcgtctgtgggacgaagaccgggcacccgccagagaggg

SEQ ID NO: 236 - DNA, 118 bp, Homo sapiens; identical to SEQ ID NO: 235
  modified_base (33)..(33): Potential to be methylated (5-methylcytosine)

SEQ ID NO: 237 - DNA, 118 bp, Homo sapiens; identical to SEQ ID NO: 235
  modified_base (43)..(43): Potential to be methylated (5-methylcytosine)

SEQ ID NO: 238 - DNA, 118 bp, Homo sapiens; identical to SEQ ID NO: 235
  modified_base (64)..(64): Potential to be methylated (5-methylcytosine)

SEQ ID NO: 239 - DNA, 118 bp, Homo sapiens; identical to SEQ ID NO: 235
  modified_base (79)..(79): Potential to be methylated (5-methylcytosine)

SEQ ID NO: 240 - DNA, 118 bp, Homo sapiens; identical to SEQ ID NO: 235
  modified_base (81)..(81): Potential to be methylated (5-methylcytosine)

SEQ ID NO: 241 - DNA, 118 bp, Homo sapiens; identical to SEQ ID NO: 235
  modified_base (92)..(92): Potential to be methylated (5-methylcytosine)

SEQ ID NO: 242 - DNA, 155 bp, Homo sapiens
  modified_base (28)..(28): Potential to be methylated (5-methylcytosine)
  modified_base (31)..(31): Potential to be methylated (5-methylcytosine)
  modified_base (34)..(34): Potential to be methylated (5-methylcytosine)
  modified_base (36)..(36): Potential to be methylated (5-methylcytosine)
  modified_base (69)..(69): Potential to be methylated (5-methylcytosine)
  modified_base (110)..(110): Potential to be methylated (5-methylcytosine)
  modified_base (112)..(112): Potential to be methylated (5-methylcytosine)
  modified_base (121)..(121): Potential to be methylated (5-methylcytosine)
  modified_base (127)..(127): Potential to be methylated (5-methylcytosine)
ctcatctcagagcgcaggaagcaaacccgccgccgcgacctctccccaggctggggtgggctggcaggcggaggtgggcagtaaacagtcctattgtacaaatatatagcgcgggctgggcgggggcggtcaaccccggttccctggcacgggga

SEQ ID NO: 243 - DNA, 155 bp, Homo sapiens; identical to SEQ ID NO: 242
  modified_base (28)..(28): Potential to be methylated (5-methylcytosine)

SEQ ID NO: 244 - DNA, 155 bp, Homo sapiens; identical to SEQ ID NO: 242
  modified_base (31)..(31): Potential to be methylated (5-methylcytosine)

SEQ ID NO: 245 - DNA, 155 bp, Homo sapiens; identical to SEQ ID NO: 242
  modified_base (34)..(34): Potential to be methylated (5-methylcytosine)

SEQ ID NO: 246 - DNA, 155 bp, Homo sapiens; identical to SEQ ID NO: 242
  modified_base (36)..(36): Potential to be methylated (5-methylcytosine)

SEQ ID NO: 247 - DNA, 155 bp, Homo sapiens; identical to SEQ ID NO: 242
  modified_base (69)..(69): Potential to be methylated (5-methylcytosine)

SEQ ID NO: 248 - DNA, 155 bp, Homo sapiens; identical to SEQ ID NO: 242
  modified_base (110)..(110): Potential to be methylated (5-methylcytosine)

SEQ ID NO: 249 - DNA, 155 bp, Homo sapiens; identical to SEQ ID NO: 242
  modified_base (112)..(112): Potential to be methylated (5-methylcytosine)

SEQ ID NO: 250 - DNA, 155 bp, Homo sapiens; identical to SEQ ID NO: 242
  modified_base (121)..(121): Potential to be methylated (5-methylcytosine)

SEQ ID NO: 251 - DNA, 155 bp, Homo sapiens; identical to SEQ ID NO: 242
  modified_base (127)..(127): Potential to be methylated (5-methylcytosine)

SEQ ID NO: 252 - DNA, 102 bp, Homo sapiens
  modified_base (25)..(25): Potential to be methylated (5-methylcytosine)
  modified_base (43)..(43): Potential to be methylated (5-methylcytosine)
  modified_base (48)..(48): Potential to be methylated (5-methylcytosine)
  modified_base (56)..(56): Potential to be methylated (5-methylcytosine)
  modified_base (65)..(65): Potential to be methylated (5-methylcytosine)
  modified_base (71)..(71): Potential to be methylated (5-methylcytosine)
  modified_base (74)..(74): Potential to be methylated (5-methylcytosine)
gcccctccttgcgaccccgcaggccgccacatctgggaccagcggatcgcttggtcgctggagccgatcccgccggggccctagatatagttggacccagcg

SEQ ID NO: 253 - DNA, 102 bp, Homo sapiens; identical to SEQ ID NO: 252
  modified_base (25)..(25): Potential to be methylated (5-methylcytosine)

SEQ ID NO: 254 - DNA, 102 bp, Homo sapiens; identical to SEQ ID NO: 252
  modified_base (43)..(43): Potential to be methylated (5-methylcytosine)

SEQ ID NO: 255 - DNA, 102 bp, Homo sapiens; identical to SEQ ID NO: 252
  modified_base (48)..(48): Potential to be methylated (5-methylcytosine)

SEQ ID NO: 256 - DNA, 102 bp, Homo sapiens; identical to SEQ ID NO: 252
  modified_base (56)..(56): Potential to be methylated (5-methylcytosine)

SEQ ID NO: 257 - DNA, 102 bp, Homo sapiens; identical to SEQ ID NO: 252
  modified_base (65)..(65): Potential to be methylated (5-methylcytosine)

SEQ ID NO: 258 - DNA, 102 bp, Homo sapiens; identical to SEQ ID NO: 252
  modified_base (71)..(71): Potential to be methylated (5-methylcytosine)

SEQ ID NO: 259 - DNA, 102 bp, Homo sapiens; identical to SEQ ID NO: 252
  modified_base (74)..(74): Potential to be methylated (5-methylcytosine)

SEQ ID NO: 260 - DNA, 85 bp, Homo sapiens
  modified_base (24)..(24): Potential to be methylated (5-methylcytosine)
  modified_base (29)..(29): Potential to be methylated (5-methylcytosine)
  modified_base (37)..(37): Potential to be methylated (5-methylcytosine)
  modified_base (46)..(46): Potential to be methylated (5-methylcytosine)
  modified_base (52)..(52): Potential to be methylated (5-methylcytosine)
  modified_base (55)..(55): Potential to be methylated (5-methylcytosine)
caggccgccacatctgggaccagcggatcgcttggtcgctggagccgatcccgccggggccctagatatagttggacccagcgcg

SEQ ID NO: 261 - DNA, 85 bp, Homo sapiens; identical to SEQ ID NO: 260
  modified_base (24)..(24): Potential to be methylated (5-methylcytosine)

SEQ ID NO: 262 - DNA, 85 bp, Homo sapiens; identical to SEQ ID NO: 260
  modified_base (29)..(29): Potential to be methylated (5-methylcytosine)

SEQ ID NO: 263 - DNA, 85 bp, Homo sapiens; identical to SEQ ID NO: 260
  modified_base (37)..(37): Potential to be methylated (5-methylcytosine)

SEQ ID NO: 264 - DNA, 85 bp, Homo sapiens; identical to SEQ ID NO: 260
  modified_base (46)..(46): Potential to be methylated (5-methylcytosine)

SEQ ID NO: 265 - DNA, 85 bp, Homo sapiens; identical to SEQ ID NO: 260
  modified_base (52)..(52): Potential to be methylated (5-methylcytosine)

SEQ ID NO: 266 - DNA, 85 bp, Homo sapiens; identical to SEQ ID NO: 260
  modified_base (55)..(55): Potential to be methylated (5-methylcytosine)

SEQ ID NO: 267 - DNA, 155 bp, Homo sapiens
  modified_base (27)..(27): Potential to be methylated (5-methylcytosine)
  modified_base (29)..(29): Potential to be methylated (5-methylcytosine)
  modified_base (33)..(33): Potential to be methylated (5-methylcytosine)
  modified_base (50)..(50): Potential to be methylated (5-methylcytosine)
  modified_base (64)..(64): Potential to be methylated (5-methylcytosine)
  modified_base (104)..(104): Potential to be methylated (5-methylcytosine)
  modified_base (106)..(106): Potential to be methylated (5-methylcytosine)
  modified_base (119)..(119): Potential to be methylated (5-methylcytosine)
  modified_base (124)..(124): Potential to be methylated (5-methylcytosine)
  modified_base (127)..(127): Potential to be methylated (5-methylcytosine)
ccggtacaggtgcggctgcaggacctcgcgcacgttctggaggaactggcgggtgatcagcagcgtggccagcatctgggggcaggaaggggaaggagagaggcgcgtggggggcaagcggggcgccgggatcgggggactcaccctccctgggc

SEQ ID NO: 268 - DNA, 155 bp, Homo sapiens; identical to SEQ ID NO: 267
  modified_base (27)..(27): Potential to be methylated (5-methylcytosine)

SEQ ID NO: 269 - DNA, 155 bp, Homo sapiens; identical to SEQ ID NO: 267
  modified_base (29)..(29): Potential to be methylated (5-methylcytosine)

SEQ ID NO: 270 - DNA, 155 bp, Homo sapiens; identical to SEQ ID NO: 267
  modified_base (33)..(33): Potential to be methylated (5-methylcytosine)

SEQ ID NO: 271 - DNA, 155 bp, Homo sapiens; identical to SEQ ID NO: 267
  modified_base (50)..(50): Potential to be methylated (5-methylcytosine)

SEQ ID NO: 272 - DNA, 155 bp, Homo sapiens; identical to SEQ ID NO: 267
  modified_base (64)..(64): Potential to be methylated (5-methylcytosine)

SEQ ID NO: 273 - DNA, 155 bp, Homo sapiens; identical to SEQ ID NO: 267
  modified_base (104)..(104): Potential to be methylated (5-methylcytosine)

SEQ ID NO: 274 - DNA, 155 bp, Homo sapiens; identical to SEQ ID NO: 267
  modified_base (106)..(106): Potential to be methylated (5-methylcytosine)

SEQ ID NO: 275 - DNA, 155 bp, Homo sapiens; identical to SEQ ID NO: 267
  modified_base (119)..(119): Potential to be methylated (5-methylcytosine)

SEQ ID NO: 276 - DNA, 155 bp, Homo sapiens; identical to SEQ ID NO: 267
  modified_base (124)..(124): Potential to be methylated (5-methylcytosine)

SEQ ID NO: 277 - DNA, 155 bp, Homo sapiens; identical to SEQ ID NO: 267
  modified_base (127)..(127): Potential to be methylated (5-methylcytosine)

SEQ ID NO: 278 - DNA, 102 bp, Homo sapiens
  modified_base (48)..(48): Potential to be methylated (5-methylcytosine)
  modified_base (50)..(50): Potential to be methylated (5-methylcytosine)
  modified_base (63)..(63): Potential to be methylated (5-methylcytosine)
  modified_base (68)..(68): Potential to be methylated (5-methylcytosine)
  modified_base (71)..(71): Potential to be methylated (5-methylcytosine)
  modified_base (77)..(77): Potential to be methylated (5-methylcytosine)
tcagcagcgtggccagcatctgggggcaggaaggggaaggagagaggcgcgtggggggcaagcggggcgccgggatcgggggactcaccctccctgggcgcc

SEQ ID NO: 279 - DNA, 102 bp, Homo sapiens; identical to SEQ ID NO: 278
  modified_base (48)..(48): Potential to be methylated (5-methylcytosine)

SEQ ID NO: 280 - DNA, 102 bp, Homo sapiens; identical to SEQ ID NO: 278
  modified_base (50)..(50): Potential to be methylated (5-methylcytosine)

SEQ ID NO: 281 - DNA, 102 bp, Homo sapiens; identical to SEQ ID NO: 278
  modified_base (63)..(63): Potential to be methylated (5-methylcytosine)

SEQ ID NO: 282 - DNA, 102 bp, Homo sapiens; identical to SEQ ID NO: 278
  modified_base (68)..(68): Potential to be methylated (5-methylcytosine)

SEQ ID NO: 283 - DNA, 102 bp, Homo sapiens; identical to SEQ ID NO: 278
  modified_base (71)..(71): Potential to be methylated (5-methylcytosine)

SEQ ID NO: 284 - DNA, 102 bp, Homo sapiens; identical to SEQ ID NO: 278
  modified_base (77)..(77): Potential to be methylated (5-methylcytosine)

SEQ ID NO: 285 - DNA, 153 bp, Homo sapiens
  modified_base (27)..(27): Potential to be methylated (5-methylcytosine)
  modified_base (29)..(29): Potential to be methylated (5-methylcytosine)
  modified_base (49)..(49): Potential to be methylated (5-methylcytosine)
  modified_base (54)..(54): Potential to be methylated (5-methylcytosine)
  modified_base (101)..(101): Potential to be methylated (5-methylcytosine)
  modified_base (119)..(119): Potential to be methylated (5-methylcytosine)
acttcccggtcgagctcgaccagggccgcggtggcattatcctcctctcgaaacgctttgcctagcactgtaaagtgtcccataggcctcagggcagcctcgagggactcttggaattcggcatcatcacagtcctccgggatgcccaggatg

SEQ ID NO: 286 - DNA, 153 bp, Homo sapiens; identical to SEQ ID NO: 285
  modified_base (27)..(27): Potential to be methylated (5-methylcytosine)

SEQ ID NO: 287 - DNA, 153 bp, Homo sapiens; identical to SEQ ID NO: 285
  modified_base (29)..(29): Potential to be methylated (5-methylcytosine)

SEQ ID NO: 288 - DNA, 153 bp, Homo sapiens; identical to SEQ ID NO: 285
  modified_base (49)..(49): Potential to be methylated (5-methylcytosine)

SEQ ID NO: 289 - DNA, 153 bp, Homo sapiens; identical to SEQ ID NO: 285
  modified_base (54)..(54): Potential to be methylated (5-methylcytosine)

SEQ ID NO: 290 - DNA, 153 bp, Homo sapiens; identical to SEQ ID NO: 285
  modified_base (101)..(101): Potential to be methylated (5-methylcytosine)

SEQ ID NO: 291 - DNA, 153 bp, Homo sapiens; identical to SEQ ID NO: 285
  modified_base (119)..(119): Potential to be methylated (5-methylcytosine)

SEQ ID NO: 292 - DNA, 127 bp, Homo sapiens
  modified_base (29)..(29): Potential to be methylated (5-methylcytosine)
  modified_base (34)..(34): Potential to be methylated (5-methylcytosine)
  modified_base (81)..(81): Potential to be methylated (5-methylcytosine)
  modified_base (99)..(99): Potential to be methylated (5-methylcytosine)
cagggccgcggtggcattatcctcctctcgaaacgctttgcctagcactgtaaagtgtcccataggcctcagggcagcctcgagggactcttggaattcggcatcatcacagtcctccgggatgccc

SEQ ID NO: 293 - DNA, 127 bp, Homo sapiens; identical to SEQ ID NO: 292
  modified_base (29)..(29): Potential to be methylated (5-methylcytosine)

SEQ ID NO: 294 - DNA, 127 bp, Homo sapiens; identical to SEQ ID NO: 292
  modified_base (34)..(34): Potential to be methylated (5-methylcytosine)

SEQ ID NO: 295 - DNA, 127 bp, Homo sapiens; identical to SEQ ID NO: 292
  modified_base (81)..(81): Potential to be methylated (5-methylcytosine)

SEQ ID NO: 296 - DNA, 127 bp, Homo sapiens; identical to SEQ ID NO: 292
  modified_base (99)..(99): Potential to be methylated (5-methylcytosine)

SEQ ID NO: 297 - DNA, 20 bp, Artificial Sequence
  source: /note=“Description of Artificial Sequence Synthetic primer”
ggggtgyggggaggttgaga

SEQ ID NO: 298 - DNA, 20 bp, Artificial Sequence
  source: /note=“Description of Artificial Sequence Synthetic primer”
tccccractcccccaacctc

SEQ ID NO: 299 - DNA, 27 bp, Artificial Sequence
  source: /note=“Description of Artificial Sequence Synthetic primer”
ggygttgaagttggagaggttattttg

SEQ ID NO: 300 - DNA, 27 bp, Artificial Sequence
  source: /note=“Description of Artificial Sequence Synthetic primer”
ccraaactcttctccttaaaacaaaac

SEQ ID NO: 301 - DNA, 26 bp, Artificial Sequence
  source: /note=“Description of Artificial Sequence Synthetic primer”
ggttttttttagttttagygttttga

SEQ ID NO: 302 - DNA, 33 bp, Artificial Sequence
  source: /note=“Description of Artificial Sequence Synthetic primer”
tacaacaaaaaaacttataatccaattatcatc

SEQ ID NO: 303 - DNA, 24 bp, Artificial Sequence
  source: /note=“Description of Artificial Sequence Synthetic primer”
ygtgaggttggtgggtaggtttag

SEQ ID NO: 304 - DNA, 33 bp, Artificial Sequence
  source: /note=“Description of Artificial Sequence Synthetic primer”
ttcccctatccrccaacttacaaatatatcttc

SEQ ID NO: 305 - DNA, 26 bp, Artificial Sequence
  source: /note=“Description of Artificial Sequence Synthetic primer”
gttygttagtttgtaagtgtgttttt

SEQ ID NO: 306 - DNA, 26 bp, Artificial Sequence
  source: /note=“Description of Artificial Sequence Synthetic primer”
cracccattccraaaaacaaaatata

SEQ ID NO: 307 - DNA, 32 bp, Artificial Sequence
  source: /note=“Description of Artificial Sequence Synthetic primer”
gaataatagataagggtggttggtagtaagta

SEQ ID NO: 308 - DNA, 27 bp, Artificial Sequence
  source: /note=“Description of Artificial Sequence Synthetic primer”
tcacctaaaacaaacattccaaaaacc

SEQ ID NO: 309 - DNA, 29 bp, Artificial Sequence
  source: /note=“Description of Artificial Sequence Synthetic primer”
gtttatttggggtaggtattttagaagtt

SEQ ID NO: 310 - DNA, 35 bp, Artificial Sequence
  source: /note=“Description of Artificial Sequence Synthetic primer”
aaaaaacaacaaataaaaataactaacaataaaca

SEQ ID NO: 311 - DNA, 25 bp, Artificial Sequence
  source: /note=“Description of Artificial Sequence Synthetic primer”
tygggatagtattttgggagttggg

SEQ ID NO: 312 - DNA, 25 bp, Artificial Sequence
  source: /note=“Description of Artificial Sequence Synthetic primer”
accaaacrcccatccaatttaccta

SEQ ID NO: 313 - DNA, 34 bp, Artificial Sequence
  source: /note=“Description of Artificial Sequence Synthetic primer”
ttttgtattttttttagtagagaygggtttttat

SEQ ID NO: 314 - DNA, 26 bp, Artificial Sequence
  source: /note=“Description of Artificial Sequence Synthetic primer”
ttaaaaacccctctctcttccraata

SEQ ID NO: 315 - DNA, 31 bp, Artificial Sequence
  source: /note=“Description of Artificial Sequence Synthetic primer”
ttttygttttygtaggtattyggttattttg

SEQ ID NO: 316 - DNA, 22 bp, Artificial Sequence
  source: /note=“Description of Artificial Sequence Synthetic primer”
rcctcctcacraaaaaacaact

SEQ ID NO: 317 - DNA, 24 bp, Artificial Sequence
  source: /note=“Description of Artificial Sequence Synthetic primer”
tgtgtaaagtyggtgaggtgttga

SEQ ID NO: 318 - DNA, 35 bp, Artificial Sequence
  source: /note=“Description of Artificial Sequence Synthetic primer”
aaatccaaaataaaaatttaaaatcaaatcccttt

SEQ ID NO: 319 - DNA, 36 bp, Artificial Sequence
  source: /note=“Description of Artificial Sequence Synthetic primer”
ggtagagtgaagtataagtaataattttgtattatt

SEQ ID NO: 320 - DNA, 28 bp, Artificial Sequence
  source: /note=“Description of Artificial Sequence Synthetic primer”
aaataaactctaaactcraacaaaaaac

SEQ ID NO: 321 - DNA, 26 bp, Artificial Sequence
  source: /note=“Description of Artificial Sequence Synthetic primer”
aattygagtaggaggtyggttttttt

SEQ ID NO: 322 - DNA, 35 bp, Artificial Sequence
  source: /note=“Description of Artificial Sequence Synthetic primer”
acaaaataaaacacaaacaataatcctatattatt

SEQ ID NO: 323 - DNA, 35 bp, Artificial Sequence
  source: /note=“Description of Artificial Sequence Synthetic primer”
attttttttttttgtattttttgtagttyggtttt

SEQ ID NO: 324 - DNA, 28 bp, Artificial Sequence
  source: /note=“Description of Artificial Sequence Synthetic primer”
caaaaaccaacctaaacatctcaaataa

SEQ ID NO: 325 - DNA, 24 bp, Artificial Sequence
  source: /note=“Description of Artificial Sequence Synthetic primer”
ggtygggttataaggattygggaa

SEQ ID NO: 326 - DNA, 27 bp, Artificial Sequence
  source: /note=“Description of Artificial Sequence Synthetic primer”
ccaaccccaaatcttacraacaattcc

SEQ ID NO: 327 - DNA, 28 bp, Artificial Sequence
  source: /note=“Description of Artificial Sequence Synthetic primer”
gttttaygagttttygttygttttggtt

SEQ ID NO: 328 - DNA, 24 bp, Artificial Sequence
  source: /note=“Description of Artificial Sequence Synthetic primer”
aatacccratcttcrtcccacaaa

SEQ ID NO: 329 - DNA, 30 bp, Artificial Sequence
  source: /note=“Description of Artificial Sequence Synthetic primer”
yggyggttagaygtttatttaatttgtttg

SEQ ID NO: 330 - DNA, 33 bp, Artificial Sequence
  source: /note=“Description of Artificial Sequence Synthetic primer”
ctacaaatccraaacaacaccttaaaaactaaa

SEQ ID NO: 331 - DNA, 31 bp, Artificial Sequence
  source: /note=“Description of Artificial Sequence Synthetic primer”
gtgtttttttaaggtaagtatagatgagggg

SEQ ID NO: 332 - DNA, 27 bp, Artificial Sequence
  source: /note=“Description of Artificial Sequence Synthetic primer”
cccrcaataaaaataccttaaatctcc

SEQ ID NO: 333 - DNA, 28 bp, Artificial Sequence
  source: /note=“Description of Artificial Sequence Synthetic primer”
gatgttttaagtttttyggttttgggga

SEQ ID NO: 334 - DNA, 35 bp, Artificial Sequence
  source: /note=“Description of Artificial Sequence Synthetic primer”
ctcaatatcctcctaaaacaaacacaaataaaaaa

SEQ ID NO: 335 - DNA, 36 bp, Artificial Sequence
  source: /note=“Description of Artificial Sequence Synthetic primer”
gtttttggtagaggttagtttgggtattttaggtgg

SEQ ID NO: 336 - DNA, 24 bp, Artificial Sequence
  source: /note=“Description of Artificial Sequence Synthetic primer”
cctcctcctacacctcctacaacc

SEQ ID NO: 337 - DNA, 29 bp, Artificial Sequence
  source: /note=“Description of Artificial Sequence Synthetic primer”
tagttttygaagaaaagatgggtttgggg

SEQ ID NO: 338 - DNA, 27 bp, Artificial Sequence
  source: /note=“Description of Artificial Sequence Synthetic primer”
aacrccraatcaactcaacraaatctc

SEQ ID NO: 339 - DNA, 32 bp, Artificial Sequence
  source: /note=“Description of Artificial Sequence Synthetic primer”
ggagtygttatggaygttgagttttttagttt

SEQ ID NO: 340 - DNA, 23 bp, Artificial Sequence
  source: /note=“Description of Artificial Sequence Synthetic primer”
taaacrcraaccccaaaacccta

SEQ ID NO: 341 - DNA, 27 bp, Artificial Sequence
  source: /note=“Description of Artificial Sequence Synthetic primer”
attttagggttttaygtyggggttttg

SEQ ID NO: 342 - DNA, 32 bp, Artificial Sequence
  source: /note=“Description of Artificial Sequence Synthetic primer”
craaacttctatatcrcttccatcaaaaaaac

SEQ ID NO: 343 - DNA, 25 bp, Artificial Sequence
  source: /note=“Description of Artificial Sequence Synthetic primer”
ttttttttggygggtgttyggtttt

SEQ ID NO: 344 - DNA, 32 bp, Artificial Sequence
  source: /note=“Description of Artificial Sequence Synthetic primer”
taattcraatttctccraattttactaccaac

SEQ ID NO: 345 - DNA, 27 bp, Artificial Sequence
  source: /note=“Description of Artificial Sequence Synthetic primer”
ttttygtgttagggaatyggggttgat

SEQ ID NO: 346 - DNA, 27 bp, Artificial Sequence
  source: /note=“Description of Artificial Sequence Synthetic primer”
ctcatctcaaaacrcaaaaaacaaacc

SEQ ID NO: 347 - DNA, 24 bp, Artificial Sequence
  source: /note=“Description of Artificial Sequence Synthetic primer”
gtttttttttgygatttygtaggt

SEQ ID NO: 348 - DNA, 27 bp, Artificial Sequence
  source: /note=“Description of Artificial Sequence Synthetic primer”
crctaaatccaactatatctaaaaccc

SEQ ID NO: 349 - DNA, 29 bp, Artificial Sequence
  source: /note=“Description of Artificial Sequence Synthetic primer”
ygygttgggtttaattatatttagggttt

SEQ ID NO: 350 - DNA, 23 bp, Artificial Sequence
  source: /note=“Description of Artificial Sequence Synthetic primer”
caaaccrccacatctaaaaccaa

SEQ ID NO: 351 - DNA, 26 bp, Artificial Sequence
  source: /note=“Description of Artificial Sequence Synthetic primer”
tyggtataggtgyggttgtaggattt

SEQ ID NO: 352 - DNA, 27 bp, Artificial Sequence
  source: /note=“Description of Artificial Sequence Synthetic primer”
gcccaaaaaaaataaatcccccratcc

SEQ ID NO: 353 - DNA, 24 bp, Artificial Sequence
  source: /note=“Description of Artificial Sequence Synthetic primer”
ggygtttagggagggtgagttttt

SEQ ID NO: 354 - DNA, 30 bp, Artificial Sequence
  source: /note=“Description of Artificial Sequence Synthetic primer”
tcaacaacrtaaccaacatctaaaaacaaa

SEQ ID NO: 355 - DNA, 26 bp, Artificial Sequence
  source: /note=“Description of Artificial Sequence Synthetic primer”
atttttyggtygagttygattagggt

SEQ ID NO: 356 - DNA, 33 bp, Artificial Sequence
  source: /note=“Description of Artificial Sequence Synthetic primer”
catcctaaacatcccraaaaactataataatac

SEQ ID NO: 357 - DNA, 27 bp, Artificial Sequence
  source: /note=“Description of Artificial Sequence Synthetic primer”
gggtatttyggaggattgtgatgatgt

SEQ ID NO: 358 - DNA, 28 bp, Artificial Sequence
  source: /note=“Description of Artificial Sequence Synthetic primer”
caaaaccrcrataacattatcctcctct

(19) United States
(12) Patent Application Publication
(10) Pub. No.: US 2021/0062268 A1
(43) Pub. Date: Mar. 4, 2021

(54) Method of Identifying Metastatic Breast Cancer by Differentially Methylated Regions

(71) Applicants: UCL Business LTD, London (GB); Eurofins Genomics Europe Sequencing GmbH, Konstanz (DE); Genedata AG, Basel (CH)

(72) Inventors: Martin Widschwendter, Wildermieming (AT); Allison Jones, London (GB); Iona Evans, London (GB); Harri Lemppiainen, Binningen (CH); Johannes Eichner, Lorrach (DE); Tamas Rujan, Bottmingen (CH); Timo Wittenberger, Konstanz (DE); Tobias Paprotka, Konstanz (DE); Benjamin Lindner, Laupheim (DE)

(21) Appl. No.: 16/631,782

(22) PCT Filed: Jul. 20, 2018
(86) PCT No.: PCT/GB2018/052059

§ 371 Date: Jan. 16, 2020

(30) Foreign Application Priority Data
Jul. 21, 2017 (GB) 1711782.1

(51) Int. Cl. C12Q 1/6886

(52) U.S. Cl. CPC C12Q 1/6886; C12Q 2600/154; C12Q 2600/118

(57) ABSTRACT

The present invention relates to methods of identifying the presence of DNA from one or more metastatic breast cancer (mBC) cells in a sample from an individual. The invention also relates to methods of diagnosing metastatic breast cancer (mBC) by identifying the presence of mBC cell DNA in a sample from an individual. The invention also relates to methods of identifying a breast cancer patient as having a poor disease prognosis by identifying the presence of DNA from one or more mBC cells in a sample from an individual. The invention additionally relates to methods of identifying in DNA from an individual the presence of a methylation signature associated with mBC by identifying the presence of DNA from one or more mBC cells in a sample from an individual. The invention also relates to oligonucleotide primers for amplifying differentially methylated regions (DMRs) and/or methylation variable positions (MVPs), detection probes for detecting amplicons comprising DMRs and MVPs and kits comprising oligonucleotide primers, detection probes and reagents for use in the methods of the invention.

### FIELD OF THE INVENTION

The present invention relates to methods of identifying the presence of DNA from one or more metastatic breast cancer (mBC) cells in a sample from an individual. The invention also relates to methods of diagnosing metastatic breast cancer (mBC) by identifying the presence of mBC cell DNA in a sample from an individual. The invention also relates to methods of identifying a breast cancer patient as having a poor disease prognosis by identifying the presence of DNA from one or more mBC cells in a sample from an individual. The invention additionally relates to methods of identifying in DNA from an individual the presence of a methylation signature associated with mBC by identifying the presence of DNA from one or more mBC cells in a sample from an individual.

### BACKGROUND TO THE INVENTION

Breast cancer (BC) is by far the most frequent cancer among women. Every year 522,000 women die from BC [1].

Mammography is used as a screening tool for early diagnosis but has its limitations due to over-diagnosis and low specificity, leading to a modest impact on mortality [2]. In addition, there is clear evidence that women diagnosed with BCs that are not detected during a screening programme, so called “interval BCs”, have a much worse prognosis [3]. This is consistent with the recent evidence demonstrating that dissemination might occur during the early stages of tumor evolution [4].

Adjuvant systemic treatment is one of the main contributing factors leading to a substantial reduction in BC mortality over the last two to three decades [5]. The current strategy guiding administration of adjuvant systemic treatment is reliant upon primary tumor characteristics such as size, regional lymph node involvement and molecular characteristics. However, systemic relapse and subsequent death are caused by disseminated tumor cells whose biological properties may be very different to those comprising the primary tumor and lymph nodes [6].

Numerous studies have demonstrated that patients with disseminated tumor cells in the bone marrow [7-9], or circulating tumor cells (CTCs) [10-14], have an inferior prognosis. The immunocytochemical detection of CTCs is reliant upon the isolation of intact cells. This approach does not take into account necrotic tumor cell deposits, tumor derived exosomes, or cellular fragments that are released into the bloodstream.

Recently, markers based on DNA shed from tumor cells have shown great promise in monitoring treatment response and predicting prognosis [15-19]. However, efforts to characterise the cancer genome have shown that only a few genes are frequently mutated in cancer, and the site of mutation per gene differs across tumors. Hence the detection of somatic mutations is currently limited to patients who harbour such predefined mutations [20]. The necessity of prior knowledge regarding the specific genomic composition of tumor tissue is one of the limiting factors when using these ‘liquid biopsy’ approaches for early detection or monitoring response to treatment. A further limitation is that current technology only allows for the detection of a mutant allele fraction of 0.1% [15, 21].

Over the last decade, efforts to validate the involvement of epigenetic changes in cancer have been fast paced. DNA methylation (DNAme) has been shown to be a hallmark of cancer [22] and occurs very early in BC development [23]. DNAme has been demonstrated to effect distinct changes in cellular function. For instance, methylation of promotor regions has been shown to be associated with compacted chromatin structure and gene silencing. As such, there is significant interest in the study of DNAme to identify CpG biomarkers which associate and/or correlate with cancer. Of significant interest are the identification of CpG methylation loci linked to cancer disease etiology, so as to provide diagnostic and prognostic biomarkers, or to provide predictive biomarkers for risk associations.

DNAme is centred around specific regions (CpG islands) [22]. Analyses of the content, levels and patterns of CpG methylation have been greatly facilitated by technical advances such as bisulphite modification of DNA, which allows for the retrospective detection of a methylated CpG locus notwithstanding the loss of the methyl group following downstream processing of the initial sample of DNA. The ability of methylated CpG loci to provide readily-tractable and functionally-relevant biological markers in this way has led to rapid advances in the understanding of the role of methylation in physiology and disease, particularly in cancer.

Furthermore, DNAme is chemically and biologically stable. This enables the development of early detection tools and personalised treatment, based upon the analysis of cell-free DNA contained within serum or plasma [24-29].

However, two major challenges have to be overcome: (1) the very low abundance of cancer-DNA in the blood and (2) the high level of “background DNA” shed from white blood cells (WBC) [30] in banked samples (from population cohorts and clinical trials with long-term follow up) used for the validation of potential screening/predictive markers.

Thus there remains a need for improved methods for the detection of tumor-derived DNA in patient samples, particularly in liquid samples such as blood and serum (so-called “liquid biopsies”). In particular, there remains a need for improved methods for the detection in patient samples of DNA, e.g. cell-free DNA, derived from disseminated (metastatic) breast cancer (mBC) cells.

### SUMMARY OF THE INVENTION

The invention relates to a method of identifying the presence of metastatic breast cancer (mBC) cell DNA in a sample from an individual, the method comprising:
i. providing DNA from a sample from the individual, the sample DNA comprising a plurality of DNA molecules each having a defined differentially methylated region (DMR);
ii. determining the methylation status of four or more methylation variable positions (MVPs) within DMRs, wherein the MVPs are selected from a group of linked MVPs within the DMR;
iii. selecting a pre-defined DMR methylation pattern for the four or more MVPs within the DMR, wherein each one of the four or more MVPs is scored as methylated or unmethylated;
iv. determining a pattern frequency for the DMR methylation pattern; and
v. identifying mBC DNA within the sample DNA when the pattern frequency equals or exceeds a threshold value.

The invention also relates to a method of diagnosing metastatic breast cancer (mBC) by identifying the presence of mBC cell DNA in a sample from an individual, the method comprising:
i. providing DNA from a sample from the individual, the sample DNA comprising a plurality of DNA molecules each having a defined differentially methylated region (DMR);
ii. determining the methylation status of four or more linked methylation variable positions (MVPs) within DMRs, wherein the MVPs are selected from a group of linked MVPs within the DMR;
iii. selecting a pre-defined DMR methylation pattern for the four or more MVPs within the DMR, wherein each one of the four or more MVPs is scored as methylated or unmethylated;
iv. determining a pattern frequency for the DMR methylation pattern;
v. identifying mBC DNA within the sample DNA when the pattern frequency equals or exceeds a threshold value; and
vi. diagnosing metastatic breast cancer when mBC DNA is identified within the sample DNA in accordance with step (v).

The invention further relates to method of providing a disease prognosis to a breast cancer patient by identifying the presence of metastatic breast cancer (mBC) cell DNA in a sample from an individual, the method comprising:
i. providing DNA from a sample from the individual, the sample DNA comprising a plurality of DNA molecules each having a defined differentially methylated region (DMR);
ii. determining the methylation status of four or more linked methylation variable positions (MVPs) within DMRs, wherein the MVPs are selected from a group of linked MVPs within the DMR;
iii. selecting a pre-defined DMR methylation pattern for the four or more MVPs within the DMR, wherein each one of the four or more MVPs is scored as methylated or unmethylated;
iv. determining a pattern frequency for the DMR methylation pattern;
v. identifying mBC DNA within the sample DNA when the pattern frequency equals or exceeds a threshold value; and
vi. providing the breast cancer patient with a disease prognosis when mBC DNA is identified within the sample DNA in accordance with step (v).
   In such a method the disease prognosis may be provided as a hazard ratio for death score (HR).

The invention further relates to a method of identifying in DNA from an individual the presence of a methylation signature correlated with metastatic breast cancer (mBC) by identifying the presence of mBC DNA in a sample from an individual, the method comprising:
i. providing DNA from a sample from the individual, the sample DNA comprising a plurality of DNA molecules each having a defined differentially methylated region (DMR);
ii. determining the methylation status of four or more linked methylation variable positions (MVPs) within DMRs, wherein the MVPs are selected from a group of linked MVPs within the DMR;
iii. selecting a pre-defined DMR methylation pattern for the four or more MVPs within the DMR, wherein each one of the four or more MVPs is scored as methylated or unmethylated;
iv. determining a pattern frequency for the DMR methylation pattern;
v. identifying mBC DNA within the sample DNA when the pattern frequency equals or exceeds a threshold value; and
vi. identifying the methylation signature when mBC DNA is identified within the sample DNA in accordance with step (v).

### BRIEF DESCRIPTION OF THE FIGURES

FIG. 1 shows the study design used to identify Breast Cancer (BC)-specific differentially methylated regions (DMRs).

Using Reduced Representation Bisulfite Sequencing (RRBS), 31 human tissue samples were analysed to identify a total of 18 regions which underwent thorough technical validation. Six regions were selected whose methylation status was analysed in two sets consisting of 110 serum samples. One marker (EFC #93) was validated in two independent settings: (1) In SUCCESS study serum samples from BC patients before and after chemotherapy; and (2) in UKCTOCS serum samples from women prior to BC diagnosis (within 3 years) or who remained healthy for 5 years.

FIG. 2 shows the principles of methylation pattern discovery in tissue (A, B) and analyses in serum (C).

Reduced Representation Bisulfite Sequencing (RRBS) was used in tissue samples in order to identify CpG methylation patterns that are able to discriminate breast cancer from white blood cells (which were deemed to be the most abundant source of cell-free DNA). “0” represent an unmethylated CpG and “1” represents a methylated CpG. An example of region EFC #93 is provided which is a 136 base-pair long region containing 5 linked CpGs. The cancer pattern consists of reads in which all linked CpGs are methylated, indicated by “11111” (A). RRBS data were processed through a bioinformatic pipeline in order to identify the most promising markers (B). The principles of the serum DNA methylation assay are demonstrated in panel C.

FIG. 3 shows that serum positivity for DNA methylation marker EFC #93 is associated with metastatic BC and is a strong marker of poor prognosis for both relapse-free and overall survival.

EFC #93 serum DNA methylation analysis in breast cancer samples from prospective and SUCCESS trial, and in combination with circulating tumor cells.

Pattern frequency of EFC #93 serum DNAme in two prospectively independently collected cohorts. Panel A represents Set 1 and B is Set 2. A cut-off threshold of 0.0008 was set when Sets1 and 2 data were combined (C). Panels D to G are data generated from SUCCESS trial samples prior to chemotherapy. Kaplan-Meier analysis for relapse-free survival (D) and overall survival (E) according to the presence (EFC #93 pattern frequency ≥0.00008) or absence (EFC #93 pattern frequency <0.00008) of marker EFC #93 before chemotherapy. Kaplan-Meier analysis for relapse-free survival (F) and overall survival (G) according to the presence/absence of EFC #93 and CTCs. P values from a Mann-Whitney-U-test or two-sided log-rank test. HB, Healthy/Benign; BC, breast cancer; CTC-ve, no CTC present; CTC+ve, at least one CTC present.

FIG. 4 shows the pattern frequency of EFC #93 in women from the UK Collaborative Ovarian Cancer Screening Study (UKCTOCS).

EFC #93 pattern frequency in samples with low (A) or high (B) amount of DNA in the serum sample (cut-off threshold 0.00008). Performance of EFC #93 serum DNA methylation marker depending on time to diagnosis and whether or not women died subsequently (C). Data separated based on amount of DNA in the serum sample (95% Confidence Intervals in brackets). P values in A and B are from a Mann-Whitney-U-test and are relative to the control group. Control, no cancer developed; BC-D, Breast Cancer which eventually led to Death; BC-ND, Breast Cancer which did Not lead to Death; mo, months; yr, years.

FIG. 5 shows pipeline for assessment of samples from the SUCCESS trial analysed within this study.

FIG. 6 shows samples from the UKCTOCS cohort analysed within this study.

FIG. 7 shows amounts of DNA collected in serum samples.

DNA amount per mL serum in the prospectively collected serum (Set 1 and 2), SUCCESS cohort, and UKCTOCS cohort. P values are based on a Mann-Whitney-U-test.

FIG. 8 shows pattern frequency for EFC #93 in pre- and post-chemotherapy settings.

Pattern frequency for EFC #93 measured in SUCCESS serum set samples from women with no, 1-4 or ≥5 CTCs in the matched blood sample before (A) or after (B) chemotherapy. P values for a Mann-Whitney-U-test.

FIG. 9 shows relapse-free survival and overall survival percentages in CTC positive and negative samples.

Impact of the presence (+ve, ≥1 cancer cell in blood sample) or absence (−ve) of CTCs on patient outcome. Two-sided log-rank test.

FIG. 10 shows the impact of the presence (+ve, EFC #93 pattern frequency ≥0.00008) or absence (−ve) of serum cancer DNA methylation in CTC+ve (≥1 cancer cell in pre-chemo blood sample) or absence CTC-ve patients.

Two-sided log-rank test.

FIG. 11 shows that neither serum marker EFC #93 nor CTCs were predictive of the outcome in samples collected after chemotherapy.

Relapse-Free survival (A) and Overall survival (B) of samples taken after chemotherapy. Impact of the presence (+ve, EFC #93 pattern frequency ≥0.00008; ≥1 CTC) or absence (−ve) of EFC #93 methylation and/or CTC on patient survival. Two-sided log-rank test.

FIG. 12 shows that the average DNA amount extracted correlates with average UK temperature.

Boxplot of DNA amount extracted from UKCTOCS sample set, collected at certain months of the year. Blue line represents average monthly UK temperatures (average UK data from 1981-2010 data set; metoffice.gov.uk).

FIG. 13 shows that the average DNA fragment size of the DNA extracted correlates with average UK temperature.

Boxplot of DNA fragment size of the DNA extracted from UKCTOCS sample set, collected at certain months of the year. Blue line represents average monthly UK temperatures (average UK data from 1981-2010 data set; metoffice.gov.uk).

FIG. 14 shows the correlation of DNA fragment size and DNA amount. Scatter-plot of DNA fragment size and DNA amount extracted from UKCTOCS sample set.

FIG. 15 shows how the algorithm used in this study determines methylation pattern frequencies.

### DETAILED DESCRIPTION OF THE INVENTION

Monitoring treatment and early detection of fatal breast cancer (BC) remains a major unmet need. Aberrant circulating DNA methylation (DNAme) patterns are likely to provide a highly specific cancer signal.

The present inventors have used reduced representation bisulfite sequencing (RRBS) of 31 tissues and established serum assays based on ultra-high coverage bisulfite sequencing in two independent prospective serum sets (n=110).

18 BC specific DNAme methylation patterns were discovered in tissue, of which 6 were tested further in serum.

One particular candidate, EFC #93, was validated for clinical use in both predicting prognosis and monitoring treatment. EFC #93, was validated in 419 patients from the SUCCESS trial (pre and post adjuvant chemotherapy samples).

EFC #93 was identified as an independent poor prognostic marker in pre-chemotherapy samples [Hazard ratio (HR) for death 7.689] and superior to circulating tumour cells (CTCs) (HR for death 5.681). More than 70% of patients with both CTCs and EFC #93 serum DNAme positivity in their pre-chemotherapy samples relapsed within five years.

The inventors have determined that DNAme markers from samples from patients can diagnose fatal BCs up to one year in advance of diagnosis and could enable individualised BC treatment.

Detection of DNAme patterns in patient samples such as serum, and in particular detection of EFC #93 DNAme patterns in patient samples such as serum, offers a new tool for early diagnosis of high-risk cancers and management of adjuvant systemic treatment.

The present invention is concerned with methods of identifying the presence of DNA from one or more metastatic breast cancer (mBC) cells in a sample from an individual. The methods involve determining the methylation status of certain linked MVPs within a genomic region from a DNA sample, selecting a methylation pattern for the MVPs wherein in the pattern certain MVPs are scored as methylated or unmethylated, determining a pattern frequency for the methylation pattern within the sample DNA, and identifying mBC DNA within the sample DNA when the pattern frequency equals or exceeds a threshold value. The methods are defined in more detail herein.

The invention also relates to methods of identifying a breast cancer patient as having a poor disease prognosis by identifying the presence of DNA from one or more mBC cells in a sample from an individual, as described in more detail herein.

The invention additionally relates to methods of identifying in DNA from an individual the presence of a methylation signature associated with mBC by identifying the presence of DNA from one or more mBC cells in a sample from an individual, as described in more detail herein.

### Methylation Variable Positions (MVPs)

All methods described herein require a step of determining the methylation status of certain numbers of specific linked methylation variable positions (MVPs) within DMRs, as defined herein.

Methylation of DNA is a recognised form of epigenetic modification which has the capability of altering the expression of genes and other elements such as microRNAs [31]. In cancer development and progression, methylation may have the effect of e.g. silencing tumor suppressor genes and/or increasing the expression of oncogenes. Other forms of dysregulation may occur as a result of methylation.

Methylation of DNA occurs at discrete loci which are predominately dinucleotides consisting of a CpG motif, but may also occur at CHH motifs (where H is A, C, or T). In the methods described herein methylation preferably occurs at CpG dinucleotides. During methylation, a methyl group is added to the fifth carbon of cytosine bases to create methylcytosine.

Methylation can occur throughout the genome and is not limited to regions associated with an expressed sequence such as a gene. However, methylation typically, but not always, occurs in a promoter or in other regulatory regions of an expressed sequence such as enhancer elements. Most typically, methylation is clustered to CpG “islands” comprising multiple adjacent CpGs, for example CpG islands present in the regulatory regions of genes, especially in their promoter regions. DMRs may contain multiple adjacent CpGs and CpG islands, as explained further below.

For the purposes of this specification the term methylation variable position (MVP) is used interchangeably with CpG as a methylation site. A CpG which has the potential to be methylated within a DMR in sample DNA prior to bisulphite conversion of DNA is an MVP according to this invention. The term MVP is also used herein to refer to sites within DNA after bisulphite conversion. In bisulphite converted DNA, the MVP may be represented by the sequence CpG if the cytosine was methylated in sample DNA prior to bisulphite conversion. If the cytosine was unmethylated in sample DNA prior to bisulphite conversion, bisulphite treatment will convert the cytosine to uracil, in which case the MVP in bisulphite converted DNA may be represented by the sequence UpG. Following amplification of bisulphite converted DNA, e.g. via PCR, the sequence UpG in an MVP may be altered to ApG or TpG and may be detected accordingly.

Identifying mBC DNA within a Sample DNA

The methods described herein all require steps of: (i) providing DNA from a sample from an individual the sample DNA comprising a plurality of DNA molecules each having a defined differentially methylated region (DMR); (ii) determining the methylation status of specific linked MVPs within DMRs; (iii) selecting a DMR methylation pattern for the specific MVPs; (iv) determining a pattern frequency for the DMR methylation pattern within the sample DNA; and (v) identifying metastatic breast cancer (mBC) DNA within the sample DNA when the pattern frequency equals or exceeds a threshold value. The methods may thus be used to identify metastatic breast cancer (mBC) DNA within the sample DNA. The methods may also be used to diagnose metastatic breast cancer (mBC) in an individual and optionally to provide a therapeutic treatment for breast cancer. The methods may additionally be used to identify in DNA from an individual a methylation signature associated with metastatic breast cancer (mBC). The methods may further be used to provide a disease prognosis to a breast cancer patent. These various aspects of the invention are defined in more detail herein.

Providing DNA from a Sample from an Individual

All methods described herein require a step of providing DNA from a sample from the individual. The sample from the individual may be referred to as a biological sample. The DNA from a sample from the individual may be referred to herein as sample DNA.

In any of the methods described herein, the method may or may not encompass the step of obtaining from the individual the sample comprising the sample DNA.

Thus, any of the assays and methods described herein may involve obtaining a sample from the individual as the source of the individual's DNA for methylation analysis.

In methods which do not encompass the step of obtaining the sample from the individual, a sample which has previously been obtained from the individual is provided as the source of DNA for methylation analysis. Thus, any of the assays and methods described herein may involve providing a sample from the individual as the source of sample DNA for methylation analysis.

Any of the assays and methods described herein may involve providing sample DNA from a biological sample which biological sample has previously been obtained from the individual.

The sample from the individual may be any suitable sample which may contain, may be capable of containing and/or may be suspected of containing metastatic breast cancer (mBC) cells, and/or DNA derived from mBC cells (mBC DNA).

Samples of biological material may include biopsy samples, solid tissue samples, aspirates, samples of biological fluids, blood, serum/plasma, peripheral blood cells, cerebrospinal fluid, urine, synovial fluid, fine needle aspirate, saliva, sputum, breast or other hormone dependent tissue, breast milk, bone marrow, skin, epithelia (including buccal, cervical or vaginal epithelia) or other tissue derived from the ectoderm, vaginal fluid etc. Tissue scrapes may include biological material from e.g. buccal, oesophageal, bladder, vaginal, urethral or cervical scrapes. Biopsy or other samples may be taken from any organ or tissue where mBC cells and/or DNA may be present. For example, biopsy or other samples may be taken from the buccal cavity, nasal cavity, salivary gland, larynx, pharynx, trachea, lung, oesophagus, stomach, small intestine, large intestine, colon, rectum, kidney, liver, bladder, heart, pancreas, gall bladder, bile duct, spleen, thymus, lymph node, thyroid gland, pituitary gland, bone, brain, breast, ovary, uterus, endometrium, cervix, vagina or vulva.

Preferably, the sample from the individual comprising sample DNA is serum/plasma.

Procedures for obtaining a biological sample from the individual may be non-invasive, such as collecting cells from urine. Alternatively, invasive procedures such as biopsy may be used.

The sample may be provided directly from the individual for analysis or may be derived from stored material, e.g. refrigerated, frozen, preserved, fixed or cryopreserved material.

Methods for the isolation of biological sample material from an individual are well known to those skilled in the art. Any suitable methods may be used.

The methods described herein can be applied to sample DNA which is shed directly into the biological sample material within the individual. For example, the methods described herein can be applied to circulating cell-free DNA originally derived from whole cells and subsequently shed into plasma. In such cases sample DNA may be harvested directly from the biological sample material from the individual, such as from serum, without the need for cell collection, cell lysis, extraction of DNA from cell lysates and subsequent processing.

Alternatively, the methods described herein can be applied equally to sample DNA which is contained within whole cells within the biological sample material from the individual. For example, the methods described herein can be applied to DNA within circulating whole cells within plasma. In such cases sample DNA may be harvested from the cells within the biological sample material from the individual, such as from serum, by collection of cells, lysis of cells, extraction of DNA from cell lysates and subsequent processing.

The methods described herein can be applied to sample DNA which is a mixture of sample DNA extracted from whole cells as described above and sample DNA which was circulating cell-free DNA shed into the biological sample material as described above.

Preferably, the methods described herein are applied to sample DNA which was circulating cell-free DNA shed into the biological sample material as described above. Thus, preferably, sample DNA is cell-free DNA obtained directly from the sample and not from a cellular fraction of the sample. Preferably, sample DNA is circulating cell-free DNA obtained from a liquid fraction of serum following removal of cells from serum/plasma.

Methods for the isolation of cell-free DNA from a biological sample such as serum/plasma, as described above, are well known to those skilled in the art. Any suitable methods may be used. Methods for the extraction and isolation of sample DNA from whole cells contained within a biological sample are well known to those skilled in the art. Any suitable methods may be used. In addition, methods for the preparation of sample DNA for the purposes of assessing methylation status of DNA are well known to those skilled in the art. Any suitable methods may be used.

### Differentially Methylated Regions (DMRs)

All methods described herein require a step of providing DNA from a sample from the individual wherein the sample DNA comprises a plurality of DNA molecules each having a defined differentially methylated region (DMR).

A DMR is a region of a genome comprising multiple adjacent methylation sites that exhibit different methylation statuses amongst multiple samples.

Sample DNA from an individual will comprise a plurality of DNA molecules, and a proportion of such DNA molecules will each carry the same DMR. For example, sample DNA from an individual will comprise DNA molecules derived from genomes from many different cells from that individual. For instance, sample DNA from an individual's serum will comprise DNA molecules derived from many different non-cancerous (normal) cells from many different cell types, such as hematopoietic cells, white blood cells and nucleated red blood cells. DNA is routinely shed into plasma from such cells in healthy individuals and such DNA can be detected by routine means. Small quantities of DNA molecules derived from mBC cells may additionally be present in serum from individuals having breast cancer. Such circulating DNA derived from normal and mBC cells may comprise a singular intact defined DMR which can be detected and analysed.

Methylation sites (MVPs) which are linked within a DMR may exhibit different methylation statuses amongst multiple DNA molecules within samples. For example, in a DMR comprising ten linked MVPs, each MVP might be unmethylated in normal cells whereas each MVP might be methylated in cancer cells. In such an example situation, the identification of DMRs in sample DNA wherein each of the ten MVPs is methylated may correlate with cancer and may allow the detection in sample DNA of DNA derived from cancer cells. Intermediate patterns of methylation may exist which may correlate with normal cells or cancer cells. Thus, the identification of cancer-specific MVP methylation patterns within DMRs and scoring of the frequency at which such patterns are detected within populations of separate DNA molecules within sample DNA may form the basis of methods by which specific methylation signatures can be used for cancer cell detection.

In the present case the Inventors have identified 18 DMRs which are capable of providing detection signatures specific to metastatic breast cancer (mBC) cells. The identification of specific methylation patterns and specific pattern frequencies associated with such DMRs provides the basis for the mBC DNA detection methods described herein.

The 18 DMRs identified herein by the Inventors have been sequenced and characterised. Nucleic acid sequences corresponding with each genomic DMR are presented in the forward direction (5′ to 3′) in Table 1 below denoted by specific SEQ ID NOS. For each DMR, each MVP methylation site is identified in square brackets, i.e. [CG]. Table 1 additionally separately lists nucleic acid sequences of each MVPs methylation site within each genomic DMR.

All methods described herein require the step (step (i)) of providing sample DNA from an individual, wherein the sample DNA comprises a plurality of DNA molecules each having a defined DMR. For example, if DMR #1 is to be analysed, the sample DNA will be processed such that a plurality of DNA molecules each having DMR #1 will be detected and analysed. Each DMR identified herein comprises a group of MVP methylation sites of defined number. As noted above, these are identified in square brackets, i.e. [CG], as shown in Table 1.

Each DMR comprises nucleic acid sequences which flank the group of MVPs, i.e. sequences upstream and downstream of the CpG group, as can clearly be seen in Table 1. It will be appreciated that for step (i) of any method it will typically not be crucial to provide the entirety of the flanking sequences set out in Table 1 for a given DMR. In addition, it will be appreciated that minor sequence differences may exist within DMRs derived from different genomes from different cells within the sample from the individual as a result of random mutations and the like. For the purposes of performing the methods described herein, it is sufficient that the DMR is identified, such that the methylation status of the relevant CpG sites of the MVPs within the DMR can be assessed.

Determining the Methylation Status of Specific Linked CpGs within DMRs

All methods described herein require a step of determining the methylation status of certain numbers of specific linked MVPs within DMRs, as defined herein.

Typically, an assessment of DNA methylation status involves analysing the presence or absence of methyl groups in DNA, for example methyl groups on the 5 position of one or more cytosine nucleotides. In the present methods, the methylation status of one or more cytosine nucleotides present as a CpG dinucleotide (where C stands for cytosine, G for guanine and p for the phosphate group linking the two) is assessed.

A variety of techniques are available for the identification and assessment of MVP methylation status, as will be outlined briefly below. The methods described herein encompass any suitable technique for the determination of MVP methylation status. However, it is to be appreciated that the methods described herein involve the determination of the methylation status of multiple adjacent MVPs within a differentially methylated region (DMR). Thus, such multiple MVPs are linked on the same singular DNA molecule present within the sample DNA. This singular DNA molecule was ultimately derived from a single chromosome from a single genome within a single cell. As such, for the purposes of the methods described herein, the determination of the methylation status of a given MVP must be performed in a manner that preserves the linkage of the multiple adjacent MVPs under analysis within the given DMR.

Methyl groups are lost from a starting DNA molecule during conventional in vitro handling steps such as PCR and sequencing. To avoid this, techniques for the detection of methyl groups commonly involve the preliminary treatment of DNA prior to subsequent processing, in a way that preserves the methylation status information of the original DNA molecule. Such preliminary techniques involve three main categories of processing, i.e. bisulphite modification, restriction enzyme digestion and affinity-based analysis. Products of these techniques can then be coupled with sequencing or array-based platforms for subsequent identification or qualitative assessment of MVP methylation status.

Techniques involving bisulphite modification of DNA have become the most common methods for detection and assessment of methylation status of CpG dinucleotides. Treatment of DNA with bisulphite, e.g. sodium bisulphite, converts cytosine bases to uracil bases, but has no effect on 5-methylcytosines. Thus, the presence of a cytosine at an MVP in bisulphite-treated DNA is indicative of the presence of a cytosine base which was previously methylated at that MVP in the starting DNA molecule. The presence of a uracil at an MVP in bisulphite-treated DNA is indicative of the presence of a cytosine base which was previously unmethylated at that MVP in the starting DNA molecule. The uracil base may be altered to adenine or thymine following further treatment of bisulphite converted DNA, such as PCR amplification.

For the purposes of this specification MVPs/CpGs in bisulphite converted DNA may be referred to as methylated or unmethylated for ease of reference. It will be appreciated however that in this context the terms methylated or unmethylated mean that the relevant base corresponds with a cytosine at the same position in DNA prior to bisulphite conversion, wherein the cytosine was either methylated or unmethylated. Thus references to MVPs in bisulphite converted DNA as being methylated or unmethylated do not mean that the base is actually methylated or unmethylated following bisulphite conversion, but that the base corresponds with a cytosine that was methylated or unmethylated prior to bisulphite conversion.

The identity of bases at MVPs can be assessed by a variety of techniques.

For example, primers specific for unmethylated versus methylated DNA can be generated and used for PCR-based identification of methylated CpG dinucleotides. DNA is preferably amplified after bisulphite conversion. A separation/capture step may be performed, e.g. using binding molecules such as complementary oligonucleotide sequences. Standard and next-generation DNA sequencing protocols can also be used. Adaptor sequences and barcode sequences may be appended to DNA molecules to facilitate sequencing and subsequent analysis. All such methods are well known in the art.

Affinity-based techniques exploit binding interactions to capture fragments of methylated DNA for the purposes of enrichment. Binding molecules such as anti-5-methylcytosine antibodies may be employed prior to subsequent processing steps such as PCR and sequencing.

Olkhov-Mitsel and Bapat (2012) [31] provide a comprehensive review of techniques available for the identification and assessment of biomarkers involving methylcytosine.

For the purposes of assessing the methylation status of the MVP-based biomarkers characterised and described herein, any suitable method can be employed, provided that the linkage between adjacent MVPs to be analysed within a given DMR is preserved, as discussed above.

Particularly preferred methods for the analysis of MVPs within DMRs involve bisulphite treatment of DNA, amplification of the DMR comprising the relevant MVP loci, or amplification of a region of the DMR comprising the relevant MVP loci, followed by sequencing to determine the methylation status of relevant MVPs within the DMR or region.

Amplification of DMRs comprising relevant MVP loci can be achieved by a variety of approaches. Preferably, MVP loci are amplified using PCR. A variety of PCR-based approaches may be used.

A preferred method involves bisulphite converting sample DNA and then simply amplifying the entire DMR itself, or a sub-region of the DMR, using primers which flank adjacent MVPs to be analysed. Example primer sequences for amplifying the 18 DMRs described herein are presented in Table 34. Adaptor sequences may be added during the amplification step to facilitate DNA sequencing. Preferably, sample specific index sequences (barcode sequences) may additionally be introduced at the step of amplification. Such barcode sequences allow pooling of amplicons derived from different sample amplification reactions for the purposes of simultaneous pooled sequencing which reduces sample processing and handling steps during sequencing, and hence reduces costs.

Any suitable sequencing techniques may be employed to determine the methylation status of MVPs within DMRs. In the methods of the present invention the use of high-throughput, so-called “second generation”, “third generation” and “next generation” techniques to sequence bisulphite-treated DNA can be used.

In second generation techniques, large numbers of DNA molecules are sequenced in parallel. Typically, tens of thousands of molecules are anchored to a given location at high density and sequences are determined in a process dependent upon DNA synthesis. Reactions generally consist of successive reagent delivery and washing steps, e.g. to allow the incorporation of reversible labelled terminator bases, and scanning steps to determine the order of base incorporation. Array-based systems of this type are available commercially e.g. from Illumina, Inc. (San Diego, Calif.; http://www.illumina.com/).

Third generation techniques are typically defined by the absence of a requirement to halt the sequencing process between detection steps and can therefore be viewed as real-time systems. For example, the base-specific release of hydrogen ions, which occurs during the incorporation process, can be detected in the context of microwell systems (e.g. see the Ion Torrent system available from Life Technologies; http://www.lifetechnologies.com/). Similarly, in pyrosequencing the base-specific release of pyrophosphate (PPi) is detected and analysed. In nanopore technologies, DNA molecules are passed through or positioned next to nanopores, and the identities of individual bases are determined following movement of the DNA molecule relative to the nanopore. Systems of this type are available commercially e.g. from Oxford Nanopore Technologies (https://www.nanoporetech.com/). In an alternative method, a DNA polymerase enzyme is confined in a “zero-mode waveguide” and the identity of incorporated bases are determined with florescence detection of gamma-labeled phosphonucleotides (see e.g. Pacific Biosciences; http://www.pacificbiosciences.com/).

In the methods described above, sequences corresponding to DMR loci may also be subjected to an enrichment process if desired. DNA containing DMRs of interest may be captured by binding molecules such as oligonucleotide probes complementary to target sequence of interest. Sequences corresponding to DMR loci may be captured before or after bisulphite conversion or before or after amplification. Probes may be designed to be complementary to bisulphite converted DNA. Captured DNA may then be subjected to further processing steps to determine the status of MVPs, such as DNA sequencing steps.

Capture/separation steps may be custom designed. Alternatively a variety of such techniques are available commercially, e.g. the SureSelect target enrichment system available from Agilent Technologies (http://www.agilent.com/home). In this system biotinylated “bait” or “probe” sequences (e.g. RNA) complementary to the DNA containing MVP sequences of interest are hybridized to sample nucleic acids. Streptavidin-coated magnetic beads are then used to capture sequences of interest hybridized to bait sequences. Unbound fractions are discarded. Bait sequences are then removed (e.g. by digestion of RNA) thus providing an enriched pool of MVP target sequences separated from non-MVP sequences. Template DNA may be subjected to bisulphite conversion and target DMR loci amplified by PCR, e.g. using primers which are independent of the methylation status of the MVP. Following amplification, samples may be subjected to a capture step to enrich for PCR products containing the target MVP, e.g. captured and purified using magnetic beads, as described above. Following capture, a standard PCR reaction is carried out to incorporate DNA sequencing adaptors and optionally barcode sequences into MVP-containing amplicons. PCR products are again purified and then subjected to DNA sequencing and analysis to determine the presence or absence of a methylcytosine at the target genomic MVP [32].

Alternative means for amplifying bisulphite converted DMRs or sub-regions of DMRs are envisaged. For example, methylation-specific primers and probes may be hybridized to DNA containing the MVPs or to a portion of sequence within a DMR comprising relevant MVPs to be analysed. The primers and probes may be designed to provide amplification product only when certain methylation pattern criteria are met. Various techniques of this type are known in the art and may be used in the methods described herein, such as techniques referred to as HeavyMethyl [33] and MethyLight [34], as discussed in more detail below.

In the methods of the invention the step of determining the DMR methylation pattern for MVPs may performed by a single process comprising the steps of amplifying, preferably by PCR, bisulphite converted sample DNA to form methylation pattern amplicons comprising DMRs or sub-regions of DMRs and simultaneously determining the methylation status of MVPs and the DMR methylation pattern within DMRs or within sub-regions of DMRs by detecting the formation of methylation pattern amplicons.

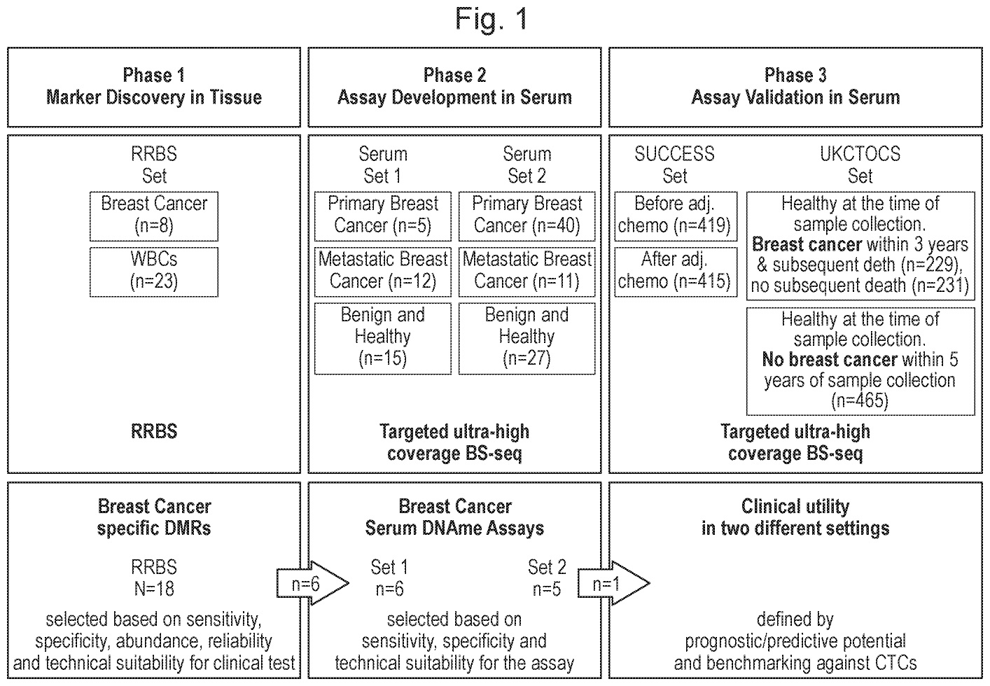

The amplification step may comprise the use of forward and reverse primers which are designed to anneal to sites which flank regions of MVPs to be analysed within DMRs or within sub-regions of DMRs. The formation of methylation pattern amplicons may be detected using one or more detection probes, wherein the one or more detection probes are designed to anneal to sites comprising MVPs to be analysed, wherein annealing is dependent upon the methylation status of MVPs, and wherein sequence-dependent annealing of the one or more detection probes is detected during or after the amplification step. Such a method is a variation of the method described in Eads et al. [34] (see FIG. 1 of Eads et al., application B). Such a method may further comprise the use of forward blocker oligonucleotides and/or reverse blocker oligonucleotides, wherein blocker oligonucleotides are designed to anneal to sites comprising MVPs to be analysed, provided that blocker oligonucleotides are designed not to anneal to a site comprising a sequence which prior to bisulphite conversion comprised MVPs whose methylation status matched the status of MVPs in a selected pre-defined DMR methylation pattern, wherein the annealing site for a forward blocker oligonucleotide and the annealing site for a reverse blocker oligonucleotide overlaps with the annealing site for forward and reverse primers respectively, and wherein annealing of a blocker oligonucleotide prevents annealing of a respective primer whereupon amplification is prevented. Such a method is a variation of the method described in Cottrell et al. [33] (see Cottrell et al, FIG. 1.). Such methods thus use a pool of different blockers, each designed to suppress the generation of amplicons if the methylation status of MVPs is not a perfect match with MVPs in a selected DMR methylation pattern. The forward and reverse primer binding sites are designed to overlap with blocker oligonucleotide binding sites. Alternatively, such a method may further comprise the use of a forward blocker oligonucleotide and/or a reverse blocker oligonucleotide, wherein blocker oligonucleotides are designed to anneal to sites comprising MVPs to be analysed and to anneal only when each MVP within the site was unmethylated prior to bisulphite conversion, wherein the annealing site for a forward blocker oligonucleotide and the annealing site for a reverse blocker oligonucleotide overlaps with the annealing site for forward and reverse primers respectively, and wherein annealing of a blocker oligonucleotide prevents annealing of a respective primer whereupon amplification prevented. In these methods, a single species of blocker is used, designed to suppress the generation of amplicons from DMRs which were completely unmethylated prior to bisulphite treatment (Cottrell et al., 2004 FIG. 1).

The methods may comprise amplifying using forward and reverse primers which are designed to anneal to sites comprising MVPs to be analysed, wherein annealing is dependent upon the methylation status of MVPs, and wherein the formation of methylation pattern amplicons is detected using one or more detection probes, wherein the one or more detection probes are designed to anneal to sites between MVPs to be analysed, and wherein sequence-dependent annealing of the one or more detection probes is detected during or after the amplification step. Such a method is a variation of the method described in Eads et al. [34] (see FIG. 1 of Eads et al., application C). The methods may alternatively comprise amplifying using forward and reverse primers which are designed to anneal to sites comprising MVPs to be analysed, wherein annealing is dependent upon the methylation status of MVPs, and wherein the formation of methylation pattern amplicons is detected using one or more detection probes, wherein the one or more detection probes are designed to anneal to sites comprising MVPs to be analysed, wherein annealing is dependent upon the methylation status of MVPs, and wherein sequence-dependent annealing of the one or more detection probes is detected during or after the amplification step. Such a method is a variation of the method described in Eads et al. [34] (see FIG. 1 of Eads et al., application D). These methods may further comprise the use of forward blocker oligonucleotides and/or reverse blocker oligonucleotides, wherein forward and reverse blocker oligonucleotides are designed to anneal to sites comprising MVPs to be analysed, and wherein the MVPs to be analysed are the same MVPs comprised respectively within forward and reverse primer binding sites, provided that a blocker oligonucleotide is designed not to anneal to a site wherein prior to bisulphite conversion the methylation status of MVPs within the site matched the status of MVPs within a selected pre-defined DMR methylation pattern, and wherein annealing of a blocker oligonucleotide prevents annealing of a respective primer whereupon amplification is prevented. Such methods thus use a pool of different blockers, each designed to suppress the generation of amplicons if the methylation status of MVPs is not a perfect match with MVPs in a selected DMR methylation pattern. Blocker binding sites are designed to be same or substantially the same as binding sites for forward and reverse primers (i.e. a modification of the method depicted in Cottrell et al., FIG. 1). Alternatively still, these methods may further comprise the use of forward blocker oligonucleotides and/or reverse blocker oligonucleotides, wherein forward and reverse blocker oligonucleotides are designed to anneal to sites comprising MVPs to be analysed, and wherein the MVPs to be analysed are the same MVPs comprised respectively within forward and reverse primer binding sites, provided that a blocker oligonucleotide is designed to anneal only when each MVP within the site was unmethylated prior to bisulphite conversion, and wherein annealing of a blocker oligonucleotide prevents annealing of a respective primer whereupon amplification is prevented. Thus such methods use a pool of different blockers, each designed to suppress the generation of amplicons which are not perfect matches with a selected DMR methylation pattern.

In any of the amplification-based analysis methods, the one or more detection probes may be an oligonucleotide comprising a fluorophore and a quencher and wherein quenching occurs by fluorescence resonance energy transfer (FRET) or by static/contact quenching. The detection probe may be designed such that when annealed, fluorescence from the fluorophore is quenched. Quenching of fluorescence may disrupted by the exonuclease action of DNA polymerase during the step of amplification, such as in TaqMan probes. Alternatively, the detection probe may be designed such that when annealed quenching of fluorescence is disrupted, such as in Molecular Beacon probes.

In other techniques, PCR primers may anneal to the CpG sequence of interest independently of the methylation status, and further processing steps may be used to determine the status of the CpG. Assays are designed so that the CpG site(s) are located between primer annealing sites. This method scheme is used in techniques such as bisulphite genomic sequencing [35], COBRA [36] and Ms-SNuPE [37]. In such methods, DNA can be bisulphite converted before or after amplification.

Methylation specific PCR (MSP) techniques [38] may be applied and used.

Following amplification of DMRs, or sub-regions of DMRs, amplified PCR products may be coupled to subsequent analytical platforms in order to determine the methylation status of the MVPs of interest. For example, the PCR products may be directly sequenced to determine the presence or absence of a methylcytosine at the target MVP or analysed by array-based techniques.

### Selecting a DMR Methylation Pattern for Specific Linked MVPs

All methods described herein require a step of selecting a DMR methylation pattern for specific MVPs within a DMR.

A specific DMR methylation pattern indicates which MVPs in a given DMR are methylated or unmethylated.

A DMR methylation pattern for a given DMR may, by way of illustration only, provide an indication of whether every MVP in the DMR is methylated or unmethylated. Thus, by way of illustration, a DMR methylation pattern for a DMR consisting of ten MVPs may provide that all ten MVPs of that DMR are methylated.

Alternatively, a DMR methylation pattern for a given DMR may, by way of illustration, provide an indication of whether each MVP of a subgroup of MVPs in the DMR is methylated or unmethylated. Thus, for example, a DMR methylation pattern for a DMR consisting of ten MVPs may provide that the first five MVPs of that DMR (in the 5′ to 3′ direction) are methylated, whereas the remaining five MVPs are unmethylated.

Intermediate DMR methylation patterns are envisaged. For example, a DMR methylation pattern for a DMR consisting of ten MVPs may provide that within a subgroup of five specific MVPs of that DMR any four of those five MVPs are methylated. Thus the remaining MVPs of that subgroup, and the remaining MVPs of the DMR outside of that MVP subgroup, may be methylated or unmethylated.

Since MVP methylation sites within a DMR are linked, a DMR methylation pattern is a pattern of MVP site-specific methylation at a specific DMR, i.e. at a specific location in the genome. The analysis of a specific DMR thus represents the analysis of a specific locus from a specific chromosome from a specific genome derived from a specific cell. Thus the analysis of a plurality of DNA molecules each having a defined DMR represents the interrogation of a specific genomic locus in a population of DNA molecules which may be derived from many different cells from the individual, including from mBC cells.

In all methods described herein, the positive identification of a given methylation pattern is intended to correlate with the presence of mBC DNA in the starting sample when the specific methylation pattern frequency exceeds a threshold value. The methylation pattern frequency is described in more detail herein. Specific methylation patterns are described further herein.

Determining a Pattern Frequency for the DMR Methylation Pattern and Identifying mBC DNA within Sample DNA when the Frequency Equals or Exceeds a Threshold Value

All methods described herein require a step of determining a pattern frequency for the DMR methylation pattern within the sample DNA.

A DMR methylation pattern frequency equates to the number of DNA molecules within a population of DNA molecules analysed which exhibit the specific DMR methylation pattern, wherein the population of DNA molecules analysed all have the defined DMR. Thus for example, if out of 10,000 DNA molecules analysed, all having a defined DMR, 8 DNA molecules possess the specific DMR methylation pattern then the pattern frequency for the DMR methylation pattern within the sample DNA is scored as 0.0008.

Typically, the methylation status of MVPs within a given DMR within a given DNA molecule is determined by bisulphite converting the DNA, amplifying DMRs or regions of DMRs followed by detection and/or sequencing of amplicons. Illustrative methods are described above and in the Examples herein. Thus each DMR sequence in each DNA molecule analysed can be interrogated for the presence or absence of a specific methylation pattern. Populations of individual DNA molecules can be interrogated to determine the pattern frequency of a specific methylation pattern. A computer algorithm can readily be employed to undertake such data processing. Illustrative methods are described in the Examples herein.

All methods described herein require a step of identifying metastatic breast cancer (mBC) DNA within the sample DNA when the DMR methylation pattern frequency equals or exceeds a threshold value. The Inventors have determined threshold values for identifying mBC DNA based on the analysis of sample cohorts.

In any of the methods described herein the DMR methylation pattern frequency threshold value may be 0.0001, or 0.0002, or 0.0003, or 0.0004, or 0.0005, or 0.0006, or 0.0007, or 0.0008, or 0.0009, or 0.001. Preferably the DMR methylation pattern frequency threshold value may be between 0.0001 to 0.001, preferably the DMR methylation pattern frequency threshold value may be 0.0008.

### Bioinformatic Tools and Statistical Metrics for MVP Analysis

Software programs which aid in the in silico analysis of bisulphite converted DNA sequences and in primer design for the purposes of methylation-specific analyses are generally available and have been described previously [39, 40, 41].

### Receiver Operating Characteristics

Sensitivity and specificity metrics for mBC DNA detection based on the MVP methylation status assays described herein may be defined using standard receiver operating characteristic (ROC) statistical analysis [42]. In ROC analysis 100% sensitivity corresponds to a finding of no false negatives, and 100% specificity corresponds to a finding of no false positives.

An assay to detect mBC DNA in accordance with the invention described herein can achieve a ROC sensitivity of 50% or greater, 51% or greater, 52% or greater, 53% or greater, 54% or greater, 55% or greater, 56% or greater, 57% or greater, 58% or greater, 59% or greater, 60% or greater, 61% or greater, 62% or greater, 63% or greater, 64% or greater, 65% or greater, 66% or greater, 67% or greater, 68% or greater, 69% or greater, 70% or greater, 71% or greater, 72% or greater, 73% or greater, 74% or greater, 75% or greater, 76% or greater, 77% or greater, 78% or greater, 79% or greater, 80% or greater, 81% or greater, 82% or greater, 83% or greater, 84% or greater, 85% or greater, 86% or greater, 87% or greater, 88% or greater, 89% or greater, 90% or greater, 91% or greater, 92% or greater, 93% or greater, 94% or greater, 95% or greater, 96% or greater, 97% or greater, 98% or greater, 99% or greater. The ROC sensitivity may be 100%.

An assay to detect mBC DNA in accordance with the invention can achieve a ROC specificity of 50% or greater, 51% or greater, 52% or greater, 53% or greater, 54% or greater, 55% or greater, 56% or greater, 57% or greater, 58% or greater, 59% or greater, 60% or greater, 61% or greater, 62% or greater, 63% or greater, 64% or greater, 65% or greater, 66% or greater, 67% or greater, 68% or greater, 69% or greater, 70% or greater, 71% or greater, 72% or greater, 73% or greater, 74% or greater, 75% or greater, 76% or greater, 77% or greater, 78% or greater, 79% or greater, 80% or greater, 81% or greater, 82% or greater, 83% or greater, 84% or greater, 85% or greater, 86% or greater, 87% or greater, 88% or greater, 89% or greater, 90% or greater, 91% or greater, 92% or greater, 93% or greater, 94% or greater, 95% or greater, 96% or greater, 97% or greater, 98% or greater, 99% or greater. The ROC specificity may be 100%.

An assay to detect mBC DNA in accordance with the invention may have an associated combination of ROC sensitivity and ROC specificity values wherein the combination is any one of the above-listed sensitivity values and any one of the above-listed specificity values, provided that the sensitivity value is equal to or less than the specificity value.

The ROC sensitivity may be 50% or greater, and the ROC specificity may be 50% or greater, 55% or greater, 60% or greater, 65% or greater, 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or 100%.

The ROC sensitivity may be 55% or greater, and the ROC specificity may be 55% or greater, 60% or greater, 65% or greater, 70% or greater, 75% or greater, 80% or greater, 85% or greater, 86% or greater, 87% or greater, 88% or greater, 89% or greater, 90% or greater, 91% or greater, 92% or greater, 93% or greater, 94% or greater, 95% or greater, 96% or greater, 97% or greater, 98% or greater, 99% or 100%.

The ROC sensitivity may be 60% or greater, and the ROC specificity may be 60% or greater, 65% or greater, 70% or greater, 75% or greater, 80% or greater, 85% or greater, 86% or greater, 87% or greater, 88% or greater, 89% or greater, 90% or greater, 91% or greater, 92% or greater, 93% or greater, 94% or greater, 95% or greater, 96% or greater, 97% or greater, 98% or greater, 99% or 100%.

The ROC sensitivity may be 65% or greater, and the ROC specificity may be 65% or greater, 70% or greater, 75% or greater, 80% or greater, 85% or greater, 86% or greater, 87% or greater, 88% or greater, 89% or greater, 90% or greater, 91% or greater, 92% or greater, 93% or greater, 94% or greater, 95% or greater, 96% or greater, 97% or greater, 98% or greater, 99% or 100%.

The ROC sensitivity may be 70% or greater, and the ROC specificity may be 70% or greater, 75% or greater, 80% or greater, 85% or greater, 86% or greater, 87% or greater, 88% or greater, 89% or greater, 90% or greater, 91% or greater, 92% or greater, 93% or greater, 94% or greater, 95% or greater, 96% or greater, 97% or greater, 98% or greater, 99% or 100%.

The ROC sensitivity may be 75% or greater, and the ROC specificity may be 75% or greater, 80% or greater, 85% or greater, 86% or greater, 87% or greater, 88% or greater, 89% or greater, 90% or greater, 91% or greater, 92% or greater, 93% or greater, 94% or greater, 95% or greater, 96% or greater, 97% or greater, 98% or greater, 99% or 100%.

The ROC sensitivity may be 80% or greater, and the ROC specificity may be 80% or greater, 85% or greater, 86% or greater, 87% or greater, 88% or greater, 89% or greater, 90% or greater, 91% or greater, 92% or greater, 93% or greater, 94% or greater, 95% or greater, 96% or greater, 97% or greater, 98% or greater, 99% or 100%.

The ROC sensitivity may be 85% or greater, and the ROC specificity may be 85% or greater, 86% or greater, 87% or greater, 88% or greater, 89% or greater, 90% or greater, 91% or greater, 92% or greater, 93% or greater, 94% or greater, 95% or greater, 96% or greater, 97% or greater, 98% or greater, 99% or 100%.

The ROC sensitivity may be 86% or greater, and the ROC specificity may be 86% or greater, 87% or greater, 88% or greater, 89% or greater, 90% or greater, 91% or greater, 92% or greater, 93% or greater, 94% or greater, 95% or greater, 96% or greater, 97% or greater, 98% or greater, 99% or 100%.

The ROC sensitivity may be 87% or greater, and the ROC specificity may be 87% or greater, 88% or greater, 89% or greater, 90% or greater, 91% or greater, 92% or greater, 93% or greater, 94% or greater, 95% or greater, 96% or greater, 97% or greater, 98% or greater, 99% or 100%.

The ROC sensitivity may be 88% or greater, and the ROC specificity may be 88% or greater, 89% or greater, 90% or greater, 91% or greater, 92% or greater, 93% or greater, 94% or greater, 95% or greater, 96% or greater, 97% or greater, 98% or greater, 99% or 100%.

The ROC sensitivity may be 89% or greater, and the ROC specificity may be 89% or greater, 90% or greater, 91% or greater, 92% or greater, 93% or greater, 94% or greater, 95% or greater, 96% or greater, 97% or greater, 98% or greater, 99% or 100%.

The ROC sensitivity may be 90% or greater, and the ROC specificity may be 90% or greater, 91% or greater, 92% or greater, 93% or greater, 94% or greater, 95% or greater, 96% or greater, 97% or greater, 98% or greater, 99% or 100%.

The ROC sensitivity may be 91% or greater, and the ROC specificity may be 91% or greater, 92% or greater, 93% or greater, 94% or greater, 95% or greater, 96% or greater, 97% or greater, 98% or greater, 99% or 100%.

The ROC sensitivity may be 92% or greater, and the ROC specificity may be 92% or greater, 93% or greater, 94% or greater, 95% or greater, 96% or greater, 97% or greater, 98% or greater, 99% or 100%.

The ROC sensitivity may be 93% or greater, and the ROC specificity may be 93% or greater, 94% or greater, 95% or greater, 96% or greater, 97% or greater, 98% or greater, 99% or 100%.

The ROC sensitivity may be 94% or greater, and the ROC specificity may be 94% or greater, 95% or greater, 96% or greater, 97% or greater, 98% or greater, 99% or 100%.

The ROC sensitivity may be 95% or greater, and the ROC specificity may be 95% or greater, 96% or greater, 97% or greater, 98% or greater, 99% or 100%.

The ROC sensitivity may be 96% or greater, and the ROC specificity may be 96% or greater, 97% or greater, 98% or greater, 99% or 100%.

The ROC sensitivity may be 97% or greater, and the ROC specificity may be 97% or greater, 98% or greater, 99% or 100%.

The ROC sensitivity may be 98% or greater, and the ROC specificity may be 98%, 99% or 100%.

The ROC sensitivity may be 99%, and the ROC specificity may be 99% or 100%.

The ROC sensitivity may be 100%, and the ROC specificity may be 100%.

Preferably, any of the methods herein may achieve a ROC sensitivity of at least 60% or greater and a ROC specificity of at least 90% or greater, more preferably the method may achieve a ROC sensitivity of at least 60.9% or greater and a ROC specificity of at least 92% or greater. Yet more preferably, any of the methods herein may achieve a ROC sensitivity of 95% or greater and a ROC specificity of 90% or greater, preferably a ROC sensitivity of 96% and a ROC specificity of 97%.

### Hazard Ratio for Death

The present invention also relates to a method of providing a disease prognosis to a breast cancer patient by identifying the presence of metastatic breast cancer (mBC) cell DNA in a sample from an individual using any of the methods described herein. In such prognostic methods the disease prognosis may be provided as a hazard ratio for death score (HR). HR is a commonly used parameter in the statistical assessment of survival metrics. HR is the ratio of the hazard rates corresponding to the conditions described by two levels of an explanatory variable.

In the context of the present invention, a patient found to have metastatic breast cancer (mBC) cell DNA in a sample due to the scoring of a positive pattern frequency for a DMR methylation pattern in sample DNA will have an increased risk of dying from the disease compared to a patient without detectable metastatic breast cancer (mBC) cell DNA. A risk ratio is provided referred to as the hazard ratio for death score (HR).

A patient who scores positive for the detection of metastatic breast cancer (mBC) cell DNA in a sample using any of the methods described herein may have a hazard ratio for death score (HR) of 6 or greater. Thus the patient will have a 7 fold or greater increased risk to die from the disease compared to a patient without detectable metastatic breast cancer (mBC) cell DNA. The HR may be 6.0 or greater, 6.1 or greater, 6.2 or greater, 6.3 or greater, 6.4 or greater, 6.5 or greater, 6.6 or greater, 6.7 or greater, 6.8 or greater, 6.9 or greater, 7.0 or greater, 7.1 or greater, 7.2 or greater, 7.3 or greater, 7.4 or greater, 7.5 or greater, 7.6 or greater, 7.7 or greater, 7.8 or greater, 7.9 or greater, 8.0 or greater, 8.1 or greater, 8.2 or greater, 8.3 or greater, 8.4 or greater, 8.5 or greater, 8.6 or greater, 8.7 or greater, 8.8 or greater, 8.9 or greater, 9.0 or greater, 9.1 or greater, 9.2 or greater, 9.3 or greater, 9.4 or greater, 9.5 or greater, 9.6 or greater, 9.7 or greater, 9.8 or greater, 9.9 or greater, 10.0 or greater.

Preferably, the hazard ratio for death score noted above is assessed on the basis of the detection of metastatic breast cancer (mBC) cell DNA in a sample before the patient has undertaken a therapeutic treatment. More preferably, the hazard ratio for death score noted above is assessed on the basis of the detection of metastatic breast cancer (mBC) cell DNA in a sample before the patient has undertaken chemotherapy.

Preferably, the hazard ratio for death score is 7.5 or greater, more preferably 7.689.

The hazard ratio for death score may be determined at a specific confidence interval. The 95% confidence interval of the hazard ratio for death score may be between about 3.0 to 17.0, preferably between 3.518 to 16.804.

The hazard ratio for death score may be 7.689 and the 95% confidence interval may be between 3.518 to 16.804.

Methods of Treating a Patient Having Metastatic Breast Cancer (mBC).

The present invention also relates to methods of treating a patient having metastatic breast cancer (mBC) comprising identifying mBC DNA within a sample from the individual by performing any of the methods described herein, and providing one or more cancer treatments to the patient.

The one or more cancer treatments may comprise one or more surgical procedures, one or more chemotherapeutic agents, one or more cytotoxic chemotherapeutic agents one or more radiotherapeutic agents, one or more immunotherapeutic agents or any combination of the above following a positive identification of mBC.

Cancer therapeutic agents are administered to a subject already suffering from a disorder or condition, in an amount sufficient to cure, alleviate or partially arrest the condition or one or more of its symptoms. Such therapeutic treatment may result in a decrease in severity of disease symptoms, or an increase in frequency or duration of symptom-free periods. An amount adequate to accomplish this is defined as “therapeutically effective amount”. Effective amounts for a given purpose will depend on the severity of the disease as well as the weight and general state of the subject. As used herein, the term “subject” includes any human.

The therapeutic agent may be directly attached, for example by chemical conjugation, to an antibody. Methods of conjugating agents or labels to an antibody are known in the art. For example, carbodiimide conjugation [43] may be used to conjugate a variety of agents, including doxorubicin, to antibodies or peptides. The water-soluble carbodiimide, 1-ethyl-3-(3-dimethylaminopropyl) carbodiimide (EDC) is particularly useful for conjugating a functional moiety to a binding moiety. Other methods for conjugating a moiety to antibodies can also be used. For example, sodium periodate oxidation followed by reductive alkylation of appropriate reactants can be used, as can glutaraldehyde cross-linking. However, it is recognised that, regardless of which method of producing a conjugate of the invention is selected, a determination must be made that the antibody maintains its targeting ability and that the functional moiety maintains its relevant function.

A cytotoxic moiety may be directly and/or indirectly cytotoxic. By “directly cytotoxic” it is meant that the moiety is one which on its own is cytotoxic. By “indirectly cytotoxic” it is meant that the moiety is one which, although is not itself cytotoxic, can induce cytotoxicity, for example by its action on a further molecule or by further action on it. The cytotoxic moiety may be cytotoxic only when intracellular and is preferably not cytotoxic when extracellular.

Cytotoxic chemotherapeutic agents are well known in the art. Cytotoxic chemotherapeutic agents, such as anticancer agents, include: alkylating agents including nitrogen mustards such as mechlorethamine (HN2), cyclophosphamide, ifosfamide, melphalan (L-sarcolysin) and chlorambucil; ethylenimines and methylmelamines such as hexamethylmelamine, thiotepa; alkyl sulphonates such as busulfan; nitrosoureas such as carmustine (BCNU), lomustine (CCNU), semustine (methyl-CCNU) and streptozocin (streptozotocin); and triazenes such as decarbazine (DTIC; dimethyltriazenoimidazole-carboxamide); Antimetabolites including folic acid analogues such as methotrexate (amethopterin); pyrimidine analogues such as fluorouracil (5-fluorouracil; 5-FU), floxuridine (fluorodeoxyuridine; FUdR) and cytarabine (cytosine arabinoside); and purine analogues and related inhibitors such as mercaptopurine (6-mercaptopurine; 6-MP), thioguanine (6-thioguanine; TG) and pentostatin (2′-deoxycoformycin). Natural Products including vinca alkaloids such as vinblastine (VLB) and vincristine; epipodophyllotoxins such as etoposide and teniposide; antibiotics such as dactinomycin (actinomycin D), daunorubicin (daunomycin; rubidomycin), doxorubicin, bleomycin, plicamycin (mithramycin) and mitomycin (mitomycin C); enzymes such as L-asparaginase; and biological response modifiers such as interferon alphenomes. Miscellaneous agents including platinum coordination complexes such as cisplatin (cis-DDP) and carboplatin; anthracenedione such as mitoxantrone and anthracycline; substituted urea such as hydroxyurea; methyl hydrazine derivative such as procarbazine (N-methylhydrazine, MIH); and adrenocortical suppressant such as mitotane (o,p′-DDD) and aminoglutethimide; taxol and analogues/derivatives; and hormone agonists/antagonists such as flutamide and tamoxifen.

A cytotoxic chemotherapeutic agent may be a cytotoxic peptide or polypeptide moiety which leads to cell death. Cytotoxic peptide and polypeptide moieties are well known in the art and include, for example, ricin, abrin, Pseudomonas exotoxin, tissue factor and the like. Methods for linking them to targeting moieties such as antibodies are also known in the art. Other ribosome inactivating proteins are described as cytotoxic agents in WO 96/06641. Pseudomonas exotoxin may also be used as the cytotoxic polypeptide. Certain cytokines, such as TNFα and IL-2, may also be useful as cytotoxic agents.

Certain radioactive atoms may also be cytotoxic if delivered in sufficient doses. Radiotherapeutic agents may comprise a radioactive atom which, in use, delivers a sufficient quantity of radioactivity to the target site so as to be cytotoxic. Suitable radioactive atoms include phosphorus-32, iodine-125, iodine-131, indium-111, rhenium-186, rhenium-188 or yttrium-90, or any other isotope which emits enough energy to destroy neighbouring cells, organelles or nucleic acid. Preferably, the isotopes and density of radioactive atoms in the agents of the invention are such that a dose of more than 4000 cGy (preferably at least 6000, 8000 or 10000 cGy) is delivered to the target site and, preferably, to the cells at the target site and their organelles, particularly the nucleus.

The radioactive atom may be attached to an antibody, antigen-binding fragment, variant, fusion or derivative thereof in known ways. For example, EDTA or another chelating agent may be attached to the binding moiety and used to attach 111In or 90Y. Tyrosine residues may be directly labelled with 125I or 131I.

A cytotoxic chemotherapeutic agent may be a suitable indirectly-cytotoxic polypeptide. In a particularly preferred embodiment, the indirectly cytotoxic polypeptide is a polypeptide which has enzymatic activity and can convert a non-toxic and/or relatively non-toxic prodrug into a cytotoxic drug. With antibodies, this type of system is often referred to as ADEPT (Antibody-Directed Enzyme Prodrug Therapy). The system requires that the antibody locates the enzymatic portion to the desired site in the body of the patient and after allowing time for the enzyme to localise at the site, administering a prodrug which is a substrate for the enzyme, the end product of the catalysis being a cytotoxic compound. The object of the approach is to maximise the concentration of drug at the desired site and to minimise the concentration of drug in normal tissues. In a preferred embodiment, the cytotoxic moiety is capable of converting a non-cytotoxic prodrug into a cytotoxic drug.

Breast cancer therapeutics further include hormone blocking therapeutics. Hormone receptor antagonists, including estrogen receptor antagonists such as tamoxifen, may be used. Estrogen blocking agents, including aromatase inhibitors such as anastrozole or letrozole, may be used.

Breast cancer therapeutics further include antibodies, including monoclonal antibodies, directed to cell surface proteins expressed on breast cancer cells. Antibodies directed to the HER2 cell surface receptor, such as trastuzumab/Herceptin, may be used.

The following Examples are provided to illustrate the invention but not to limit the invention.

### EXAMPLES

### Materials and Methods

### Patients and Sample Collection:

The Inventors used a total of 31 tissues and 1869 serum samples in five sets (FIG. 1). For serum sets 1 and 2, women attending hospitals in London, Munich and Prague were invited and consented. Blood samples (20-40 mL) were obtained (in VACUETTE® Z Serum Sep Clot Activator tubes), centrifuged at 3,000 rpm for 10 minutes and serum collected and stored at −80° C. The Inventors used serum samples from 419 patients obtained in the SUCCESS trial 11 where bloods were taken before and after chemotherapy and (within 96 hours) sent to the laboratory for CTC assessment and serum samples stored (FIG. S1). From the UK Collaborative Trial of Ovarian Cancer Screening (UKCTOCS) 31 the Inventors used serum samples from: (1) 229 women who were diagnosed with BC within the first three years after serum sample donation and subsequently died during follow-up (2) 231 matched women who developed BC within three years after sample donation and were alive at the end of follow-up, and (3) 465 women who did not develop BC within five years after sample donation (FIG. 6). Blood samples from all UKCTOCS volunteers were spun down for serum separation after having been transported at room temperature from trial centres to the central laboratory. The median time between sample collection and centrifugation was 22.1 hours. Only 1 mL of serum per UKCTOCS volunteer was available. All patients provided written informed consent. The study was approved by the Biobank Ethical Review Committee at UCL/UCLH (Reference Number: NC09.13). The study was also approved by the Charles University Ethics Committee of the General University Hospital, Prague and by the Ludwig-Maximilians-University Ethics Committee.

Isolation and bisulfite modification of DNA: DNA was isolated from tissue and serum samples at GATC Biotech (Konstanz, Germany). Tissue DNA was quantified using NanoDrop™ and Qubit™, and the size was assessed by agarose gel electrophoresis. Serum DNA was quantified using the Agilent Fragment Analyzer and the High Sensitivity Large Fragment Analysis Kit (AATI, USA). DNA was bisulfite converted at GATC Biotech.

### DNAme Analysis in Tissue:

Genome wide methylation analysis was performed by Reduced Representation Bisulfite Sequencing (RRBS) at GATC Biotech. DNA was digested with MspI followed by size selection of the library, providing enhanced coverage for the CpG-rich regions [44, 45]. The digested DNA was adapter ligated, bisulfite modified and PCR amplified. The libraries were sequenced on Illumina's HiSeq 2500 with 50 base pairs (bp) or 100 bp paired-end mode. Using Genedata Expressionist® for Genomic Profiling v9.1, the Inventors established a bioinformatics pipeline for the detection of cancer specific differentially methylated regions (DMRs). The most promising DMRs were taken forward for the development and validation of serum based clinical assays.

### Targeted Ultra-High Coverage Bisulfite Sequencing of Serum DNA:

Targeted bisulfite sequencing libraries were prepared at GATC Biotech. Bisulfite modification was performed with 1 mL serum equivalent. A two-step PCR approach was used to test up to three different markers per modified DNA sample. The first PCR amplifies the target region and adds linker sequences which are used in the second PCR to add barcodes for multiplexing and sequences needed for sequencing. Ultra-high coverage sequencing was performed on Illumina's Mi Seq or HiSeq 2500 with 75 bp or 125 bp paired-end mode.

### Data Analyses:

Genedata Expressionist® for Genomic Profiling was used to map reads to human genome version hg19, identify regions with tumor specific methylation patterns, quantify the occurrence of those patterns, and calculate relative pattern frequencies per sample. Pattern frequencies were calculated as number of reads containing the pattern divided by total reads covering the pattern region. The 95% CI intervals for sensitivity and specificity have been calculated according to the efficient-score method [46]. The endpoints were defined according to the STEEP criteria, with relapse-free survival and overall survival as the primary endpoints. The product-limit method according to Kaplan-Meier was used to estimate survival. The survival estimates in different groups were compared using the log-rank test. The Cox proportional hazards regression model was used for the analyses taking into account all variables simultaneously.

### Subjects and Sample Collection:

The Inventors analysed a total of 5 sets as detailed in FIG. 1:

### RRBS-Set:

Eight prospectively collected invasive ductal breast cancer samples (2/8 triple negative; mean age=56.6 years), and twenty three white blood cell samples (mean age=57.8) were assessed by RRBS. All samples were collected prospectively at the University College London Hospital in London (University College London Hospital, 235 Euston Rd, Fitzrovia, London NW1 2BU) and at the Charles University Hospital in Prague (Gynecological Oncology Center, Department of Obstetrics and Gynecology, Charles University in Prague, First Faculty of Medicine and General University Hospital, Prague, Apolinarska 18128 00 Prague 2, Czech Republic) and at the Department of Gynaecology and Obstetrics, Klinikum Innenstadt, Ludwig-Maximilians-Universitaet Muenchen, Maistr. 11, 80337 Munich, Germany. The study was approved by the local research ethics committees: UCL/UCLH Biobank for Studying Health & Disease NC09.13), the ethics committee of the General University Hospital, Prague and by the ethical committee of the Ludwig-Maximilians-University Munich. All patients provided written informed consent.

### Prospectively Collected Serum Sets

### Set 1:

Serum samples from the following volunteers have been collected (at the time of diagnosis, prior to treatment):

Healthy/Benign volunteers (n=15, mean age 40.2 years).

Patients with primary breast cancer (n=5, mean age 51.4 years).

Patients with metastatic (distant metastases) breast cancer (n=12, mean age 60.12 years).

### Set 2:

Serum samples from the following volunteers have been collected (at the time of diagnosis, prior to treatment):

Healthy/Benign volunteers (n=27, mean age 42.4 years).

Patients with primary breast cancer (n=40, mean age 59.6 years).

Patients with metastatic (distant metastases) breast cancer (n=11, mean age 60.2 years).

All samples were collected prospectively at the University College London Hospital in London and at the Charles University Hospital in Prague and the Department of Gynaecology and Obstetrics, Klinikum Innenstadt, Ludwig-Maximilians-Universitaet Muenchen, Maistr. 11, 80337 Munich, Germany. The study was approved by the local research ethics committees: UCL/UCLH Biobank for Studying Health & Disease NC09.13) and the ethics committee of the General University Hospital, Prague approval No.: 22/13 GRANT—7. RP—EPI-FEM-CARE as well as by the ethical committee of the Ludwig-Maximilians-University Munich. All patients provided written informed consent.

### SUCCESS Set:

SUCCESS was a prospective, randomized adjuvant study comparing three cycles of fluorouracil-epirubicin-cyclophosphamide (FEC; 500/100/500 mg/m2) followed by 3 cycles of docetaxel (100 mg/m2) every 3 weeks vs three cycles of FEC followed by 3 cycles of gemcitabine (1000 mg/m2 d1,8)-docetaxel (75 mg/m2) every 3 weeks. After the completion of chemotherapy, the patients were further randomized to receive either 2 or 5 years of zoledronate. Hormone receptor—positive women received adequate endocrine treatment. The research questions associated with CTC analysis, the blood sampling time points, and the methodology were prospectively designed, and the prognostic value of the CTCs was defined as a scientific objective of the study protocol. The study was approved by 37 German ethical boards (lead ethical board: Ludwig-Maximilians-University Munich) and conducted in accordance with the Declaration of Helsinki.

Blood samples for CTC enumeration as well as storage of serum were collected from patients after complete resection of the primary tumour and before adjuvant chemotherapy after written informed consent was obtained. The samples were collected within a time interval of less than 96 hours between the blood collection and sample preparation. A follow-up evaluation after chemotherapy and before the start of endocrine or bisphosphonate treatment was available for a subgroup. A total of 419 women had blood samples taken at both times points (i.e. before and after chemotherapy), had their CTCs enumerated at both time points, had sufficient serum available at both time points (Web FIG. 1). For further details see Rack et al (1).

### UKCTOCS Set:

From the UK Collaborative Trial of Ovarian Cancer Screening (UKCTOCS) (2) all 229 women (among the 202,628 women recruited between 2001-2005) who developed BC in the first three years after serum sample donation and died subsequent to this at cancer/death registry follow-up by 25 Mar. 2015 and 231 matched women who developed BC within three years after sample donation and were alive at the end of follow-up and 465 women who did not develop BC within five years after sample donation were analysed (appendix p 4, 8). Blood samples from all UKCTOCS volunteers were spun down for serum separation after having been transported at room temperature from trial centres to the central laboratory. The median time between sample collection and centrifugation of the sample set was 22.1 hours (IQR 19.7-24.3). Only 1 mL of serum per UKCTOCS volunteer was available. The study was approved by the local research ethics committees (UCL/UCLH Biobank for Studying Health & Disease N C09.13) and was approved as part of trial approval by the UK North West Multicentre Research Ethics Committees (North West MREC 00/8/34). All patients provided written informed consent. For further details see Jacobs, Menon et al [47].

### DNA Methylation Analyses in Tissue Samples:

DNA was isolated from tissue samples using the Qiagen DNeasy Blood and Tissue Kit (Qiagen Ltd, UK, 69506) and 600 ng was bisulfite converted using the Zymo methylation Kits (Zymo Research Inc, USA, D5004/8).

### Reduced Representation Bisulfite Sequencing (RRBS):

RRBS libraries were prepared by GATC Biotech using INVIEW RRBS-Seq according to proprietary SOPs. In brief, DNA was digested with the restriction endonuclease MspI that is specific for the CpG containing motif CCGG; later a size selection provides enhanced coverage for the CpG-rich regions including CpG islands, promoters and enhancer elements (3;4). The digested DNA is then adapter ligated, bisulfite modified and PCR-amplified. The libraries were sequenced on Illumina's HiSeq 2500 with 50 bp or 100 bp paired-end mode.

After sequencing raw data was trimmed using Trimmomatic (0.32) to remove adapter sequences and low quality bases at the beginning and end of reads. Subsequently, reads were trimmed with TrimGalore (0.3.3) to remove cytosines derived from library preparation which must not be included in the methylation analysis. Read pairs were mapped to the human genome (hg19) in Genedata Expressionist® for Genomic Profiling 8.0 applying Bisulfite Mapper based on BOWTIE v2.1.0 (5) with the settings --no-discordant--reorder-p 8--end-to-end--no-mixed-D 50-k 2--fr--norc-X 400-I 0--phred33. Further analysis was done using Genedata Expressionist® for Genomic Profiling 9.1.

### Computation of Methylation Pattern Frequencies

In order to allow the sensitive detection of low-abundant methylation patterns, the read data available for each sample type (i.e. breast cancer and white blood cells) was pooled across patients and sequencing runs. Candidate genomic regions for methylation pattern analysis were defined based on bundles of at least 10 paired-end reads covering at least consecutive 4 CpG sites which are located within a genomic range of at most 150 bp. As illustrated in FIG. 15, the algorithm first determines sets of consecutive CpG sites of maximum size, from which multiple potentially overlapping subsets are derived, which still meet the selection criteria. CpG sites located in the gap between the mate reads are ignored. For each derived set of CpG sites, the absolute and relative frequencies of all methylation patterns observed in the corresponding reads are determined. The methylation patterns are represented in terms of binary strings in which the methylation state of each CpG site is denoted by 1 if methylated or 0 if unmethylated. The algorithm for selecting candidate regions and calculating methylation pattern frequencies was implemented in the Inventors' software platform Genedata Expressionist® for Genomic Profiling.

### Procedure for the Selection of Tumour-Specific Patterns

In order to ensure that the pattern exclusively occurs in tumour samples, all patterns present in white blood cells were excluded. A score for assessing the relevance of each pattern was determined by integrating multiple subordinate scores which quantitatively capture desired properties of candidate biomarker patterns. First, for each pattern a Tumour Specificity Score S_{P}=DL·TP·TE·AF was calculated, which consists of the four components Dilution Factor DL, Tumour Prevalence TP, Tumour Enrichment Factor TE and Avoiding Factor AF. The formal definitions of the score components are given in the following:

$$D {L}_{W B C} = \frac{\# total reads}{\# r eads with pattern} * \frac{1}{1 {0}^{3}} \\ T {P}_{t u m o r} = \frac{\# reads with pattern in tumor}{\# total reads in tumor} * 10 \\ T {E}_{t u m o r} = \frac{\# observed reads with pattern in tumor}{\# expected reads with pattern in tumor} \\ A {F}_{W B C} = \frac{\# expected reads with pattern in WBC}{\# observed reads with pattern in WBC}$$

The Dilution Factor DL and Tumour Prevalence TP favour patterns which are supported by a high proportion of reads in tumour and low proportion of reads in WBC, respectively. A pattern observed in 1 out of 10 reads in tumour and in 1 out of 1000 reads in WBC scores 1 for both factors. The Tumour Enrichment Factor TE and Avoiding Factor AF were included to assess the overrepresentation of the pattern in tumour samples and its underrepresentation in WBC samples, respectively, relative to an expected number of pattern reads which is based on the observed overall methylation level in those tissues. In order to estimate the number of expected reads supporting the pattern, the methylation frequencies are calculated for each CpG site individually. Next, the number of expected reads with a specific pattern is calculated as the product of the relative frequencies of the tumour specific methylation states observed for each CpG site in the pattern times the number of reads stretching across the pattern. A TE>1 indicates that a pattern is more frequent in tumour than expected when randomly distributing the observed methylation levels across reads. Besides favouring tumour specificity the scoring procedure was also designed to make patterns with high variance of the highest priority (i.e. patterns for which a high number of transitions in the methylation state is observed between consecutive CpG sites). Such patterns may be a product of the epigenetic reprogramming of tumour cells and in order to account for the potentially increased biological relevance of these patterns another score component was introduced. The normalized variance V_{P} of a pattern p is defined as the pattern variance divided by the maximum variance, i.e. the pattern length minus 1. The scores for the tumour specificity S_{P} and pattern variance V_{P} were combined in the tumour-specific variance score SV_{P}=V_{P}·log(S_{P}). In order to facilitate the ranking of each candidate genomic region r based on the relevance of patterns p₁, . . . , p_{N} observed in the region the aggregation score ASᵣ was calculated based on the following formula:

$$A {S}_{r} = {\sum }_{i = 1}^{n} \frac{1}{i} S {V}_{P i}$$

The aggregation score ASᵣ corresponds to a weighted sum of the tumour-specific variance scores of the observed patterns. The weighting was included since an ordinary sum would introduce a bias towards regions, in which a high number of patterns have been observed due to a high read coverage and/or high CpG site density. All of the presented statistics for assessing the relevance of methylation patterns and genomic regions were implemented in Genedata Expressionist® for Genomic Profiling and R, respectively.

### DNA Methylation Analyses in Serum Samples:

### Serum Separation:

For Serum Sets 1-3 and the NACT Serum Set, women attending the hospitals in London and Prague have been invited, consented and 20-40 mL blood has been obtained (VACUETTE® Z Serum Sep Clot Activator tubes, Cat 455071, Greiner Bio One International GmbH), centrifuged at 3,000 rpm for 10 minutes and serum collected and stored at −80° C. The Inventors have applied non-stringent measures (i.e. allowed for up to 12 hours between blood draw and centrifugation) purposely in order to mimic the situation of UKCTOCS samples which have been sent from the recruiting centre to UCL within 24-48 hours before centrifugation.

### Serum DNA Isolation and Bisulfite Modification:

DNA was isolated at GATC Biotech (Konstanz, Germany). Serum DNA was quantified using the Fragment Analyzer and the High Sensitivity Large Fragment Analysis Kit (AATI, USA). DNA was bisulfite converted at GATC Biotech.

### Targeted Ultra-High Coverage Bisulfite Sequencing:

Targeted bisulfite sequencing was performed at GATC Biotech. To this end, a two-step PCR approach was used similar to the recently published BisPCR2 [48]. Bisulfite modification was performed with 1 mL serum equivalent. For each batch of samples positive and non-template controls were processed in parallel. Bisulfite converted DNA was used to test up to three different markers using automated workflows. After bisulfite modification the target regions were amplified using primers carrying the target specific sequence and a linker sequence. Amplicons were purified and quantified. All amplicons of the same sample were pooled equimolarly. In a second PCR, primers specific to the linker region were used to add sequences necessary for the sequencing and multiplexing of samples. Libraries were purified and quality controlled. Sequencing was performed on Illumina's MiSeq or HiSeq 2500 with 75 bp or 125 bp paired-end mode. Trimming of adapter sequences and low quality bases was performed with Trimmomatic as described for the RRBS data.

### Assessment of Pattern Frequency in Serum DNA:

After sequencing, raw data was trimmed using Trimmomatic (0.32) to remove adapter sequences and low quality bases at the beginning and end of reads. Subsequently, reads were trimmed with TrimGalore (0.3.3) to remove cytosines derived from library preparation which must not be included in the methylation analysis. Further analysis was done using Genedata Expressionist® for Genomic Profiling 9.1. Read pairs were mapped to the human genome (hg19) applying Bisulfite Mapper based on BOWTIE v2.2.5 (5) with the settings--no-discordant-p 8--norc--reorder-D 50--fr--end-to-end-X 500-I 0--phred33-k 2--no-mixed. Coverage was calculated per sample and target region using Numeric Data Feature Quantification activity by calculating the arithmetic mean of the coverage in each region. As part of the data quality control, efficiency of the bisulfite conversion was estimated in each sample by quantifying the methylation levels of CpHpG and CpHpH sites (where H is Any Nucleotide Except G), with minimum coverage of 10, within the target regions. Methylation pattern frequencies in serum samples for target regions were determined as described above. Relative pattern frequencies were calculated by dividing the number of reads containing the pattern by the total number of reads covering the pattern region.

### Example 1

### Identification of BC-Specific Methylation Patterns

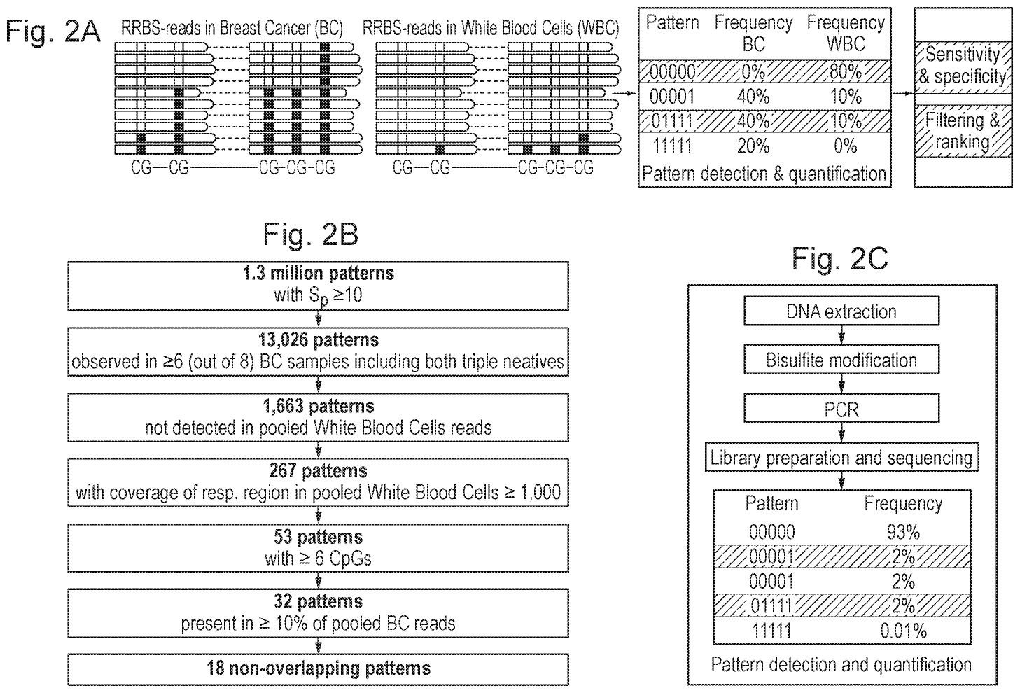

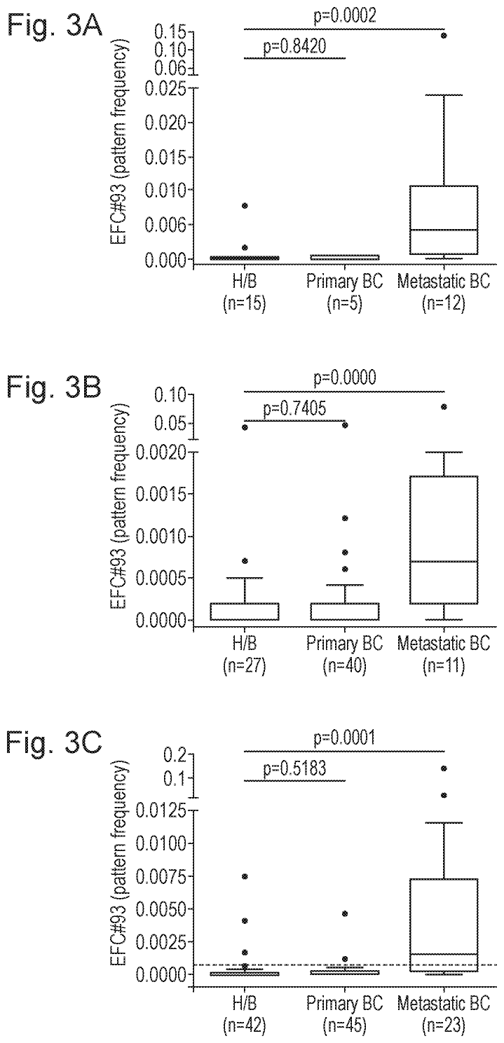

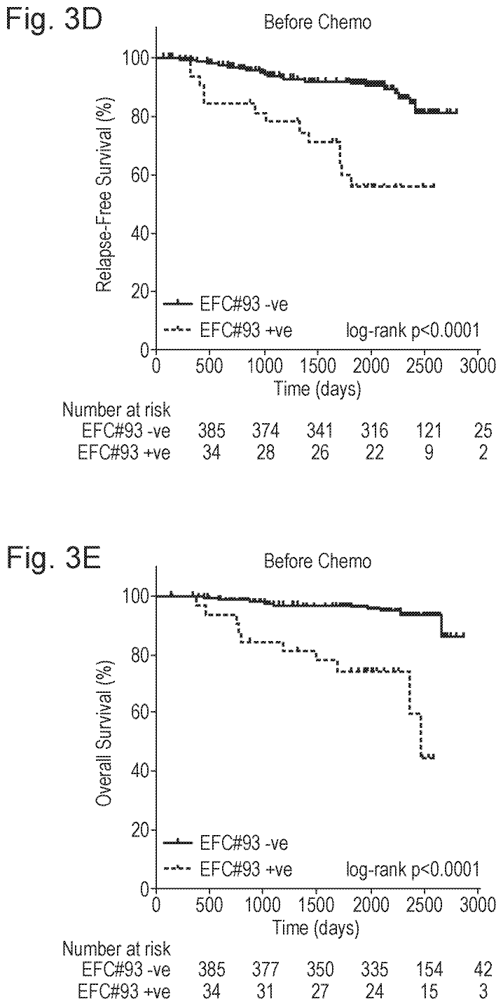

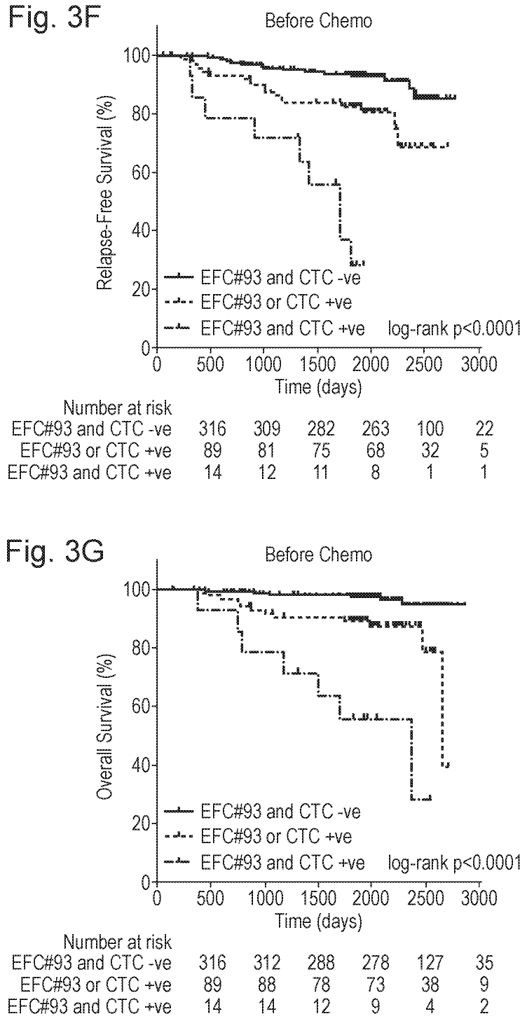

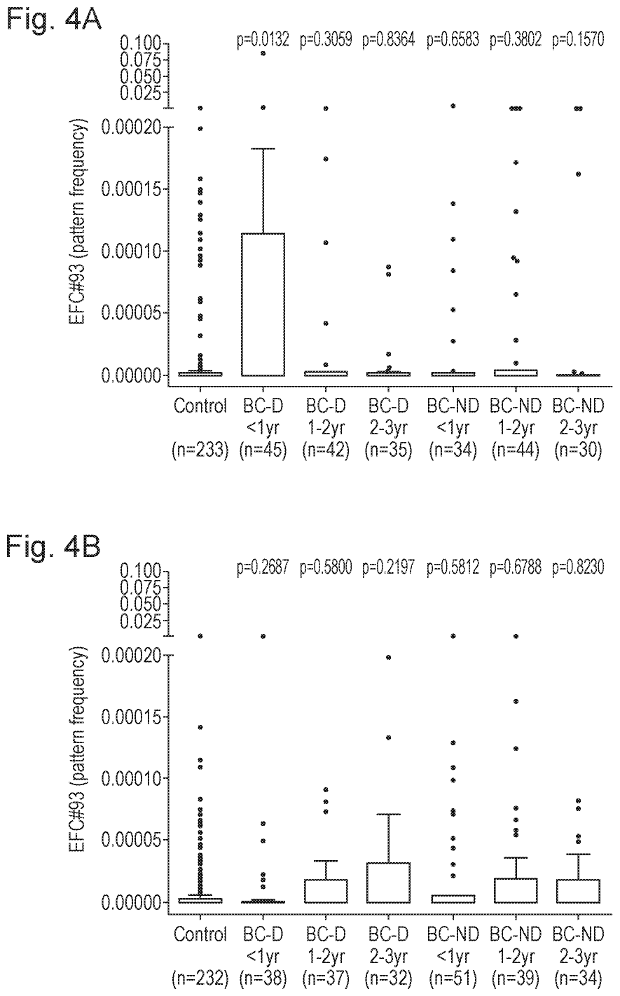

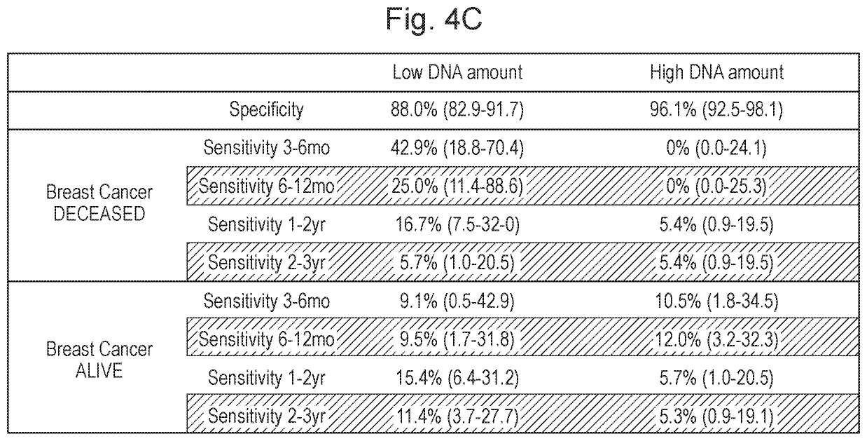

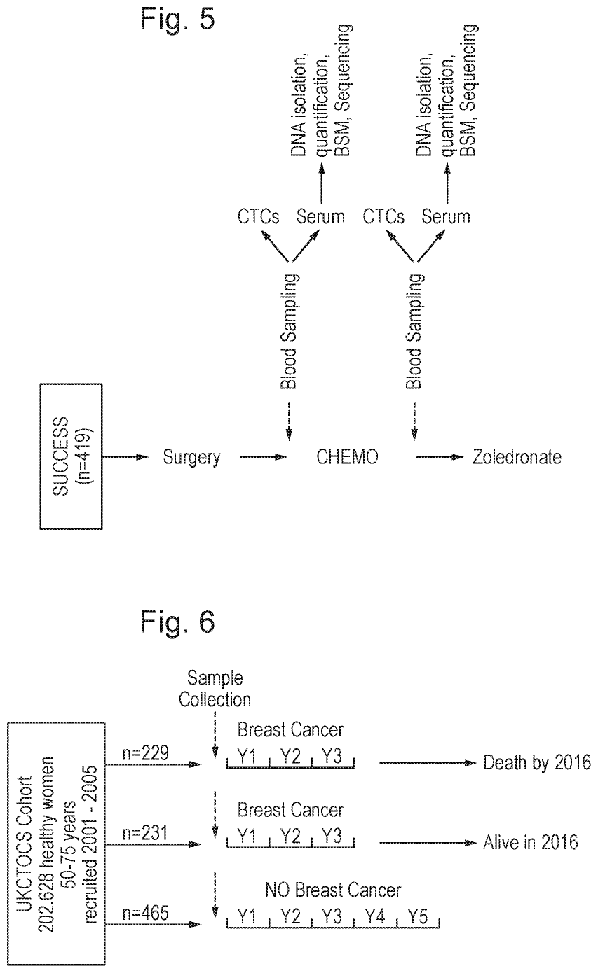

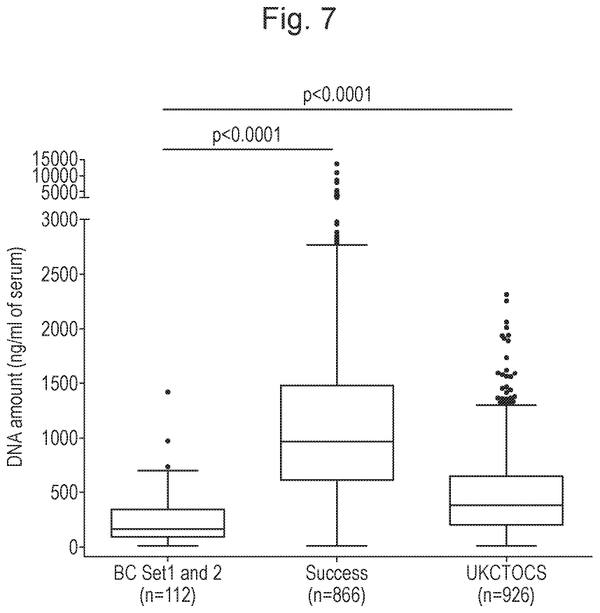

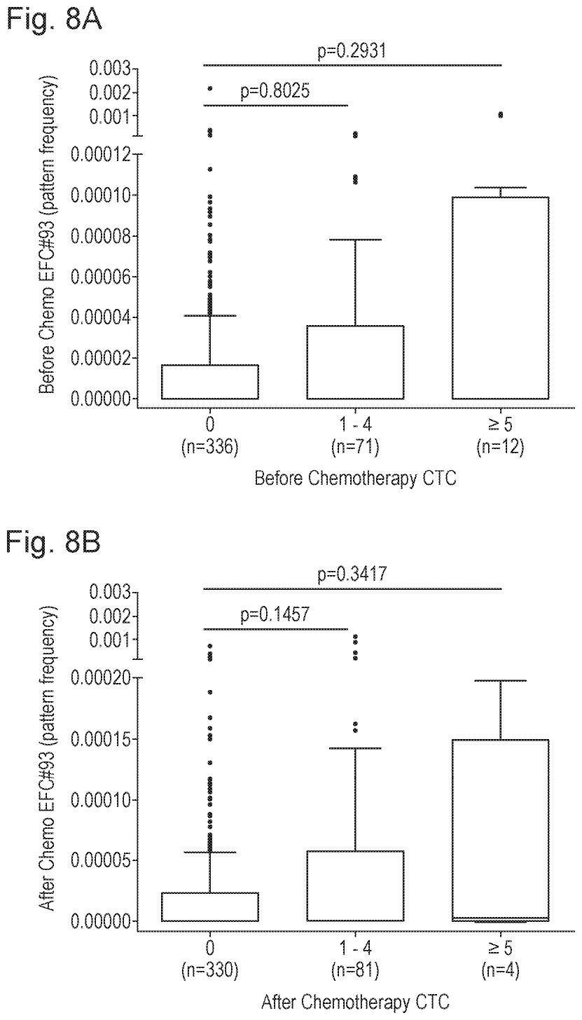

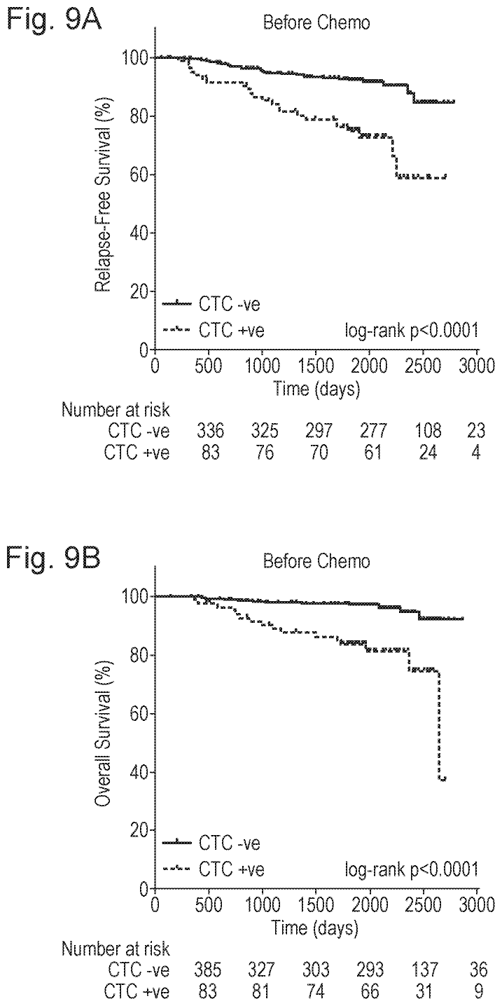

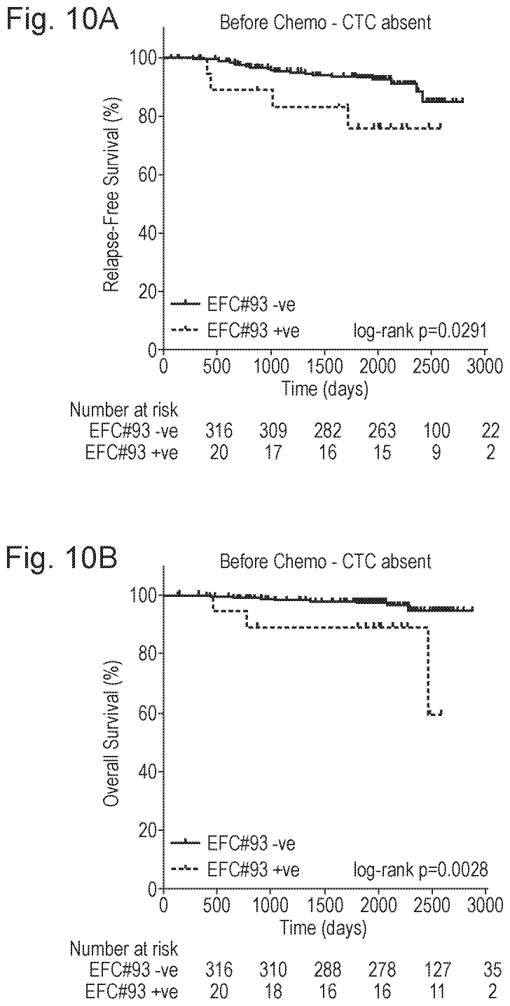

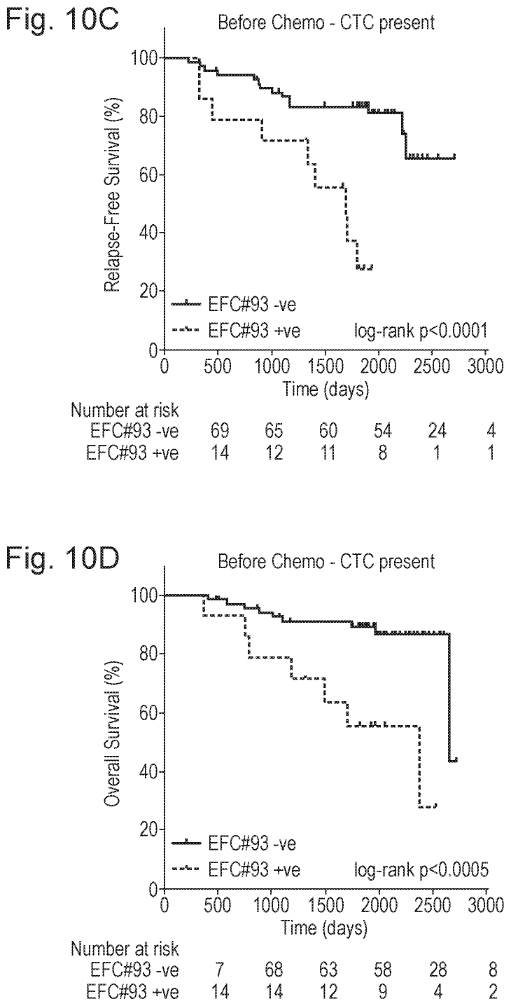

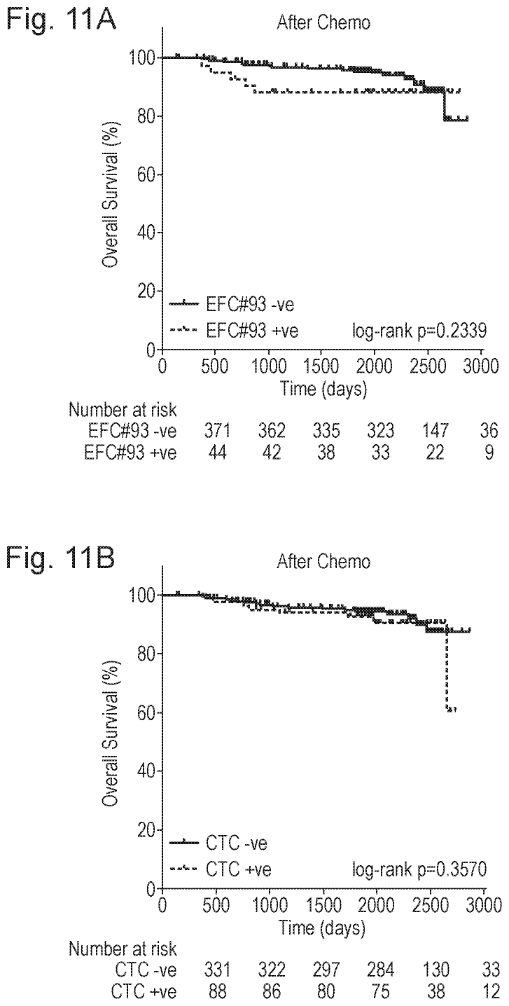

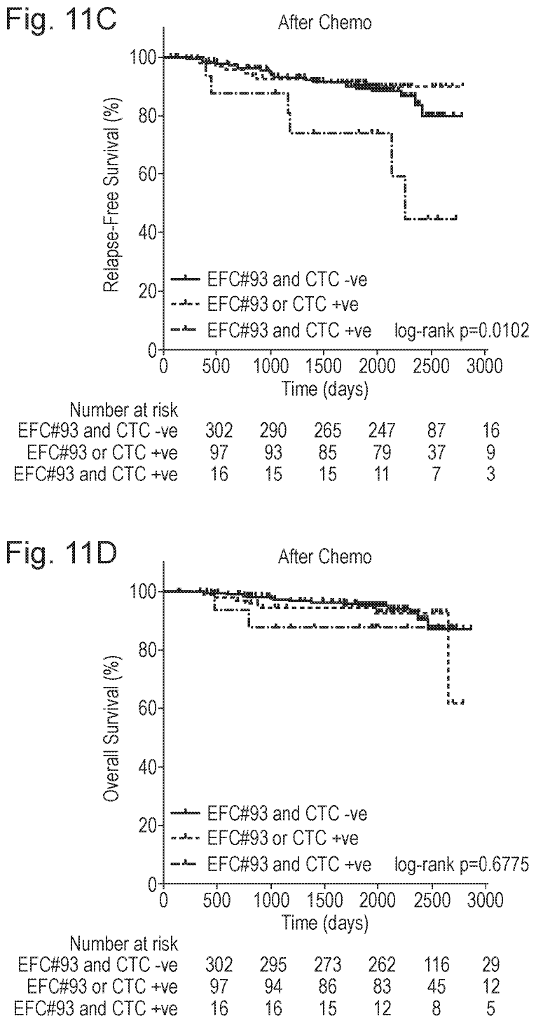

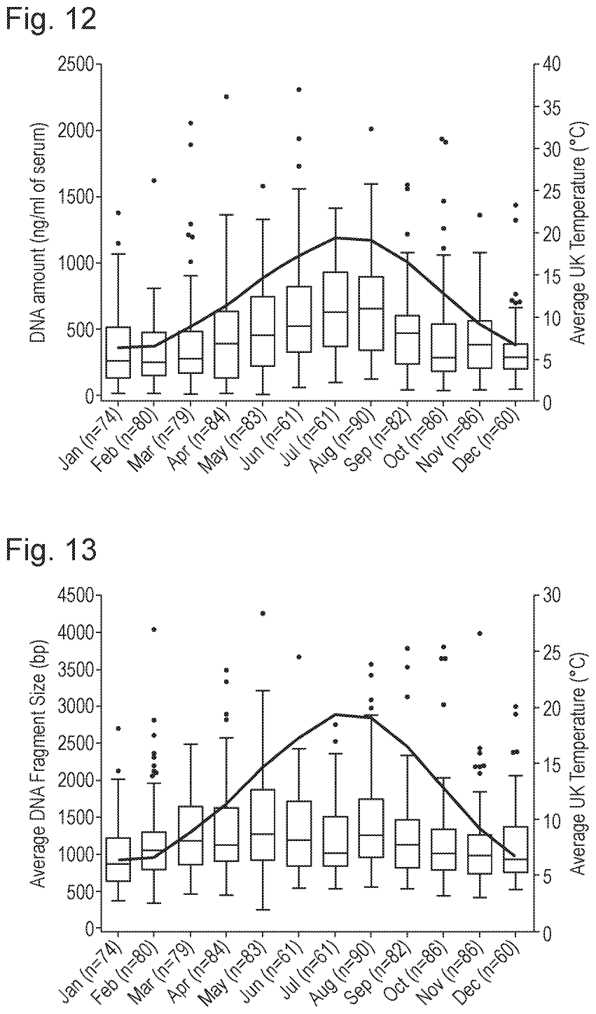

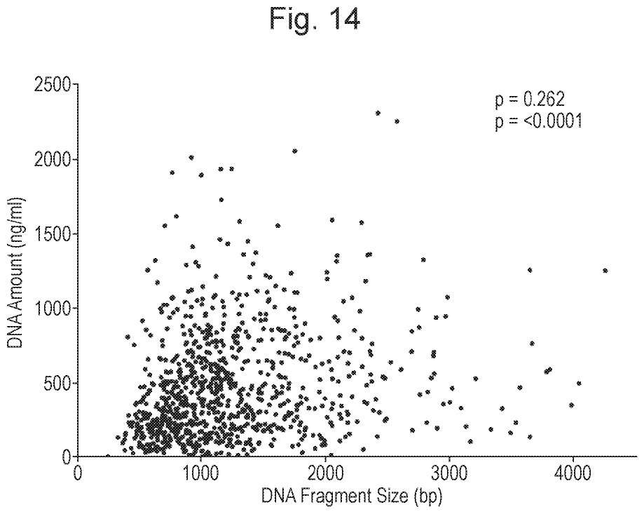

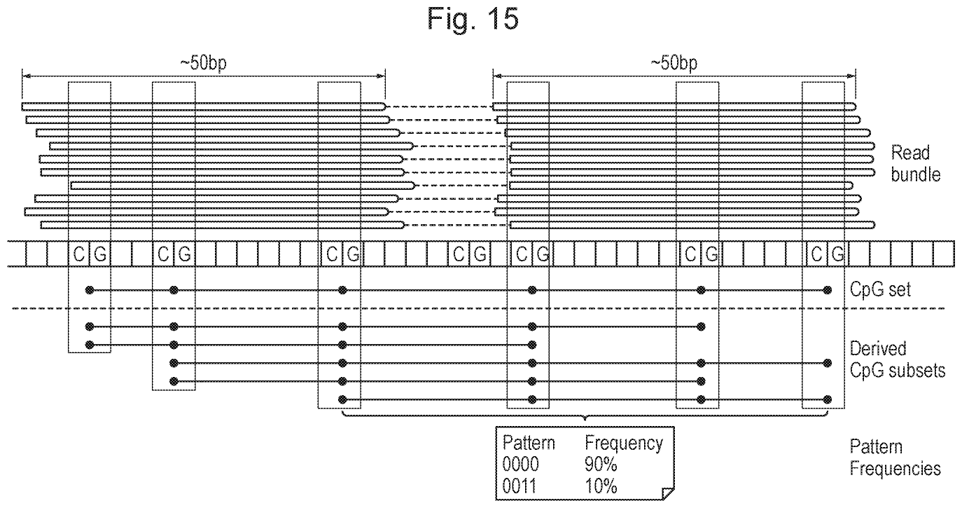

The samples, techniques and purpose of the three phases used in this study—marker discovery, assay development and assay validation—are summarized in FIG. 1. The inventors first identified DMRs based on their methylation patterns and frequencies in relevant genomic regions, within a BC tissue panel. Methylation patterns are represented in terms of a binary string, where the methylation state of each CpG site is denoted by, ‘1’ if methylated, or ‘0’ if unmethylated. The algorithm that the Inventors have developed scans the whole genome and identifies regions that contain at least 10 aligned paired-end reads. These read bundles are split into smaller regions of interest which contain at least 4 CpGs in a stretch of less than 150 bp. For each region and tissue/sample, the absolute frequency (number of supporting reads) for all observed methylation patterns was determined (FIG. 2A). This led to the discovery of tens of millions of patterns per tissue/sample. The patterns were filtered in a multi-step procedure to identify the methylation patterns which specifically occur in tumor samples. To increase the sensitivity and specificity of the pattern discovery procedure, the Inventors pooled reads from different tumor or WBC samples, and scored patterns based on over-representation within tumor tissue. The results were summarized in a specificity score, Sp, which reflects the cancer specificity of the patterns. After applying a cut-off of Sp≥10, 1.3 million patterns for BC remained, and were further filtered according to the various criteria demonstrated in FIG. 2B (Further details in Supplementary Appendix).

### Example 2

### Filtering and Validation of BC-Specific Candidates

The top 18 BC specific patterns identified by RRBS, were further validated using bisulfite sequencing. 31 bisulfite sequencing primer pairs (1-3 per region) were designed and technically validated (Table 21). The best 6 reactions were taken into Phase 2, for further testing and assay development, in prospectively collected serum sets. The inventors used ultra-deep bisulfite sequencing to develop assays for these candidate regions in 32 serum samples from Serum Set 1 (FIG. 1 and FIG. 2C). Based on sensitivity and specificity, and in particular their capacity to discriminate between metastatic and primary BC, five markers were selected for further validation in Serum Set 2 (n=78). DNA methylation marker EFC #93, which was identified in RRBS as a region of 10 linked CpGs methylated in BC, was optimized to a pattern of 5 linked CpGs, showed the best sensitivity and specificity, independently in the Set 1 and 2 (FIGS. 3A and B). A statistically higher pattern frequency, for the optimized marker EFC #93, was observed in the metastatic BC groups compared to the healthy/benign lesions or primary BC groups, in both Sets 1 and 2. This translates to an area under the curve (AUC) of a Receiver Operating Characteristics (ROC) curve of 0.850 (95% CI 0.745-0.955, P=0.000004) and 0.845 (95% CI 0.739-0.952, P=0.000004) to discriminate healthy/benign lesions or primary BC from metastatic BC in Set 1 and Set 2, respectively. When Set 1 and 2 data were combined, the pattern frequency threshold was set to 0.0008 (i.e. 8 in 10,000 reads demonstrated methylation at all CpGs in the EFC #93 region); which led to a sensitivity of 60.9% and a specificity of 92.0% to identify metastatic BC (FIG. 3C).

### Example 3

### Use of EFC #93 as a Prognostic and Predictive BC Marker

EFC #93 was then validated for use as a prognostic and predictive BC marker in clinical trial samples (FIG. 1). As expected, due to delayed sample processing within these trials, serum samples from both SUCCESS and UKCTOCS contained high levels of contaminating WBC DNA, which would lead to dilution of the cancer signal (FIG. 7 and supplementary appendix). In order to adjust for this, the inventors made an a priori decision to reduce the threshold for EFC #93 pattern frequency by a factor of 10 to 0.00008 (i.e. 8 in 100,000 reads demonstrated methylation at all 5 linked CpGs within the EFC #93 region). Table 1 shows SUCCESS patient characteristics, correlated with EFC #93 positivity/negativity, before and after chemotherapy. There was a substantial overlap of samples that were CTC and EFC #93 positive in both the pre- and post-chemotherapy setting (Table 1), although this was not statistically significant when comparing EFC #93 pattern frequencies (FIG. 8). Patients who underwent breast conserving therapy were more likely EFC #93 negative compared to patients who underwent a mastectomy; this is most likely explained by the fact that patients which presented with larger tumors tended to be EFC #93 positive and would not have been eligible for breast conserving surgery. None of the other clinic-pathological features correlated with cell-free DNA methylation of EFC #93 (Table 1). EFC #93 serum positivity before chemotherapy was a very strong marker of poor prognosis, for both relapse-free and overall survival (Table 2 and FIGS. 3D and E). This was independent of the prognostic capability of CTCs (FIGS. 9 and 10). Hazard ratios (95% CI) for overall survival in the multivariable model were 5.973 (2.634-13.542) and 3.623 (1.681-7.812) for EFC #93 and CTCs, respectively (Table 2). Patients who were CTC and EFC #93 positive had an extremely poor outcome, with >70% of these patients relapsing within 5 years (FIGS. 3F and G). Neither serum marker EFC #93 nor CTCs were predictive of the outcome in samples collected after chemotherapy (FIG. 11).

### Example 4

### Detection of EFC #93 Serum DNAme as a Tool to Diagnose Poor Prognosis BC.

To assess whether EFC #93 serum DNAme is able to diagnose women with poor prognostic BC earlier, the inventors analysed serum samples from 925 women from their UKCTOCS cohort. As expected, the amount of the DNA as well as the fragment length was dramatically higher than expected and correlated with the average UK temperature (FIGS. 12 and 13) and there was a good correlation between DNA amount and fragment length (FIG. 14) indicating a massive leak of blood cell DNA into the serum during the blood transport. Within this nested case/control setting, the women with BC (cases) had provided serum samples up to three years prior to diagnosis. Again, the inventors a priori hypothesised that the high background levels of DNA from lysed blood cells would impact on assay sensitivity—particularly in a pre-clinical setting where only traces of cancer DNA were expected in the circulation. The inventors therefore split all samples into two groups: (1) Low serum DNA amount, and (2) High serum DNA amount. In the “low DNA” group, the Inventors observed a significantly higher EFC #93 serum DNAme pattern frequency in the women who developed BC within one year after sample donation and subsequently died (FIG. 4A; cut-off threshold of 0.00008). Due to the high levels of background DNA, no significant findings were observed in the “high DNA” sample groups (FIG. 4B). In the “low DNA” group, EFC #93 DNAme was able to identify 43% of women 3-6 months prior, and 25% of women 6-12 months prior to the diagnosis of a BC which eventually led to death, with a specificity of 88% (FIG. 4C). The sensitivity of serum EFC #93 methylation to detect fatal BCs up to one year in advance of diagnosis was ˜4-fold higher compared to non-fatal BCs (33.9% compared to 9.3%). In fact, the sensitivity for non-fatal BCs was within the false positive range of the healthy samples, indicating that non-fatal BCs are not detected with this marker.

### Example 5

### Discussion

The inventors have demonstrated that serum DNA methylation marker EFC #93 can be detected up to one year in advance of BC diagnosis and is a marker for poor prognosis in the adjuvant primary treatment setting. Moreover, EFC #93 is able to diagnose poor prognostic BCs independently of conventional prognostic markers such as CTCs and in combination with CTCs indicates particularly poor prognostic cancers.

The use of tumor-specific methylated DNA in serum using targeted ultra-high bisulfite sequencing has the following advantages compared to alternative strategies: (1) Patient plasma/serum DNA can be amplified to increase assay sensitivity; (2) Abnormal DNAme is a stable tumor-specific marker occurring early in carcinogenesis and is conserved throughout disease progression [22]; (3) Selection of CpG island hypermethylation simplifies assay design; (4) DNAme over several linked CpGs constitutes a positively detectable signal with a higher specificity (due to alleviated sensitivity to sequencing errors).

A key limitation of any current large scale population-based cell-free DNA study, such as this one, is the lack of high quality samples. This was evident in both the SUCCESS and UKCTOCS samples, where the blood samples were not processed until 24-96 hours after the blood was drawn. In addition, the lack of blood stabilizers within the collection tubes was particularly evident during the summer months, for the UKCTOCS samples. In addition to the increased DNA amount, the average DNA fragment size was also dramatically higher than that previously documented. In healthy individuals, cell-free DNA is normally present at concentrations between 0 and 100 ng/mL and an average of 30 ng/mL [35]. DNA derived from tumor cells is also shorter than that from non-malignant cells in the plasma of cancer patients and typically 166 base-pairs long [36]. Blood tubes which stabilize cell-free DNA and prevent leakage of WBC DNA are now available [37]. These would be beneficial for any prospective or future blood collection, but still not solve the problem with existing banked samples.

The leaked DNA in these serum samples will no doubt have led to a preferential amplification of non-cancer DNA. Despite these complicating factors, EFC #93 serum DNAme, prior to treatment, was a strong prognostic factor, and was complementary to CTCs. Some previous studies on CTCs used a cut-off value of >5 cells/mL; this might certainly be valid and useful for metastatic BC patients. In the SUCCESS setting of primary BC patients, only 8 of the 419 patients (1.9%) had >5 CTCs/mL. Had the Inventors taken this CTC cut-off, the relapse-free survival HR would have been 4.8 with relatively wide 95% Confidence Intervals of 1.5-15.5; (P=0.009). Hence, the chosen threshold that the Inventors had pre-specified in previous work [12] (i.e. CTCs detectable or not) is well justified in this primary cancer setting.

For the current genetic cell-free DNA markers the detection limit is in the range of 0.1% allele frequency (i.e. 1 mutated in the background of 1000 non-mutated alleles can be detected^{15,21}). Ultra-high coverage bisulfite-sequencing however, allows for much more sensitive testing. Mammography screening in women aged 50-75 yrs has a sensitivity of 82-86% and a specificity of 88-92% for detecting any BC; however the majority of these cancers are not fatal [38]. EFC #93 serum DNAme has a sensitivity of 43% in identifying fatal breast cancer, up to 6 months in advance of current diagnosis at a similar specificity (88%) to mammography, supporting the rationale for incorporating serum DNAme markers in future cancer-screening trials.

Overall and for the first time, this study provides evidence that serum DNAme markers can diagnose fatal BCs up to one year in advance of diagnosis and enable individualised BC treatment. The recent advance of purposed blood tubes which stabilize circulating DNA and prevent leakage of DNA from blood cells will facilitate clinical implementation of DNAme pattern detection of cell free DNA as a clinical tool in cancer medicine.

TABLE 1
Table 1. SUCCESS Patient characteristics before and after chemotherapy for EFC#93
serum DNAme. EFC#93 serum DNAme was deemed positive (+ve) at or above a
pattern frequency of 0.00008.

| | | Before Chemotherapy | | | After Chemotherapy | | |
|---|---|---|---|---|---|---|---|
| Characteristic | | EFC#93 −ve (%) | EFC#93 +ve (%) | p value* | EFC#93 −ve (%) | EFC#93 +ve (%) | p value* |
| Number of patients | | 385 (91.9) | 34 (8.1%) | | 371 (89.4) | 44 (10.6) | |
| Age (mean +/− SD) | | 53.7 +/− 10.3 | 55.2 +/− 10.1 | 0.380 | 53.5 +/− 10.4 | 56.2 +/− 9.3 | 0.097 |
| Menopausal | premenopausal | 165 (42.9) | 15 (44.1) | 1.000 | 165 (44.5) | 15 (34.1) | 0.202 |
| Status | postmenopausal | 220 (57.1) | 19 (55.9) | | 206 (55.5) | 29 (65.9) | |
| Stage (T) | T1 | 158 (41.0) | 9 (26.5) | 0.110 | 157 (42.3) | 10 (22.7) | 0.014 |
| | T2-4 | 227 (59.0) | 25 (73.5) | | 214 (57.7) | 34 (77.3) | |
| Nodes (N) | NO | 130 (33.9) | 7 (20.6) | 0.130 | 124 (33.4) | 13 (30.2) | 0.735 |
| | N1-3 | 254 (66.1) | 27 (79.4) | | 247 (66.6) | 30 (69.8) | |
| Histology | invasive ductal | 310 (80.5) | 25 (73.5) | 0.370 | 296 (79.8) | 36 (81.8) | 0.844 |
| | others | 75 (19.5) | 9 (26.5) | | 75 (20.2) | 8 (18.2) | |
| Grading | grade 1/2 | 15 (3.9) | 1 (2.9) | 0.721 | 190 (51.2) | 23 (52.3) | 1.000 |
| | grade 3 | 184 (47.8) | 15 (44.1) | | 181 (48.8) | 21 (47.7) | |
| Estrogen | ER −ve | 128 (33.2) | 10 (29.4) | 0.708 | 128 (34.5) | 10 (22.7) | 0.130 |
| Receptor | ER +ve | 257 (66.8) | 24 (70.6) | | 243 (65.5) | 34 (77.3) | |
| Progesterone | PR −ve | 155 (40.4) | 11 (32.4) | 0.465 | 150 (40.5) | 16 (36.4) | 0.629 |
| Receptor | PR +ve | 229 (59.6) | 23 (67.6) | | 220 (59.5) | 28 (63.6) | |
| HER2 Status | HER2 −ve | 294 (77.0) | 24 (70.6) | 0.403 | 276 (75.0) | 38 (86.4) | 0.132 |
| | HER2 +ve | 88 (23.0) | 10 (29.4) | | 92 (25.0) | 6 (13.6) | |
| Surgery | breast conserving | 273 (70.9) | 16 (47.1) | 0.006 | 264 (71.2) | 23 (52.3) | 0.015 |
| | mastectomy | 112 (29.1) | 18 (52.9) | | 107 (28.8) | 21 (47.7) | |
| Chemotherapy | FEC-D | 193 (50.1) | 18 (52.9) | 0.858 | 186 (50.1) | 22 (50.0) | 1.000 |
| | FEC-DG | 192 (49.9) | 16 (47.1) | | 185 (49.9) | 22 (50.0) | |
| Bisphosponates | Zometa 2 yrs | 193 (50.1) | 17 (50.0) | 1.000 | 185 (49.9) | 23 (52.3) | 0.874 |
| | Zometa 5 yrs | 192 (49.9) | 17 (50.0) | | 186 (50.1) | 21 (47.7) | |
| Circulating | before chemo −ve | 316 (82.1) | 20 (58.8) | 0.003 | 303 (81.7) | 32 (72.3) | 0.160 |
| Tumour Cells | before chemo +ve | 69 (17.9) | 14 (41.2) | | 68 (18.3) | 12 (27.7) | |
| | after chemo −ve | 304 (79.0) | 27 (79.4) | 1.000 | 302 (81.4) | 28 (63.6) | 0.009 |
| | after chemo +ve | 81 (21.0) | 7 (20.6) | | 69 (18.6) | 16 (36.4) | |

FEC-D = fluorouracil-epirubicin-cyclophosphamide (500/100/500 mg/m2, FEC) followed by docetaxel (100 mg/mg2); FEC-DG = fluorouracil-epirubicin-cyclophosphamide (500/100/500 mg/m2, FEC) followed by gemcitabine (1,000 mg/m2 d1,8)-docetaxel (75 mg/m2); SD = standard deviation.
*Two sided t-test (in case of age) or chi square test (for all other parameters).

TABLE 2
Table 2. Univariate and multivariable proportional hazards model for relapse-free and
overall survival for SUCCESS serum samples. Cox proportional hazards models.
All statistical tests were two-sided.

| | Univariate analyses | | | |
|---|---|---|---|---|
| | Relapse-free survival | | Overall survival | |
| Characteristic | HR (96% CI) | p-value | HR (96% CI) | p-value |
| Menopausal status, pre vs post | 1.323 (0.750-2.333) | 0.335 | 2.872 (1.164-7.086) | 0.022 |
| Tumour size, T1 vs T2-4 | 2.268 (1.187-4.332) | 0.013 | 3.881 (1.343-11.218) | 0.012 |
| Lymph node involvement, N0 vs N1-3 | 1.645 (0.861-3.142) | 0.132 | 3.012 (1.045-8.683) | 0.041 |
| Estrogen receptor status, +ve vs −ve | 1.316 (0.999-1.734) | 0.051 | 1.333 (0.918-1.934) | 0.131 |
| Progesterone receptor status, +ve vs −ve | 1.180 (0.897-1.554) | 0.237 | 1.219 (0.839-1.772) | 0.298 |
| HER2 status, −ve vs +ve | 1.907 (0.858-4.241) | 0.113 | 1.789 (0.618-5.178) | 0.283 |
| Grading, G1/2 vs G3 | 1.079 (0.623-1.868) | 0.786 | 1.129 (0.535-2.384) | 0.75 |
| CTCs before chemo, −ve vs +ve | 3.666 (2.110-6.368) | <0.0001 | 5.681 (2.686-12.014) | <0.0001 |
| CTCs after chemo, −ve vs +ve | 1.401 (0.757-2.592) | 0.283 | 1.467 (0.646-3.331) | 0.36 |
| EFC#93 before chemo, −ve vs +ve | 4.912 (2.613-9.233) | <0.0001 | 7.689 (3.518-16.804) | <0.0001 |
| EFC#93 after chemo, −ve vs +ve | 1.913 (0.927-3.949) | 0.079 | 1.807 (0.673-4.853) | 0.24 |
| | Multivariable analyses | | | |
| | Relapse-free survival | | Overall survival | |
| | HR (96% CI) | p-value | HR (96% CI) | p-value |
| Menopausal status, pre vs post | 1.294 (0.728-2.302) | 0.379 | 2.688 (1.070-6.750) | 0.035 |
| Tumour size, T1 vs T2-4 | 1.763 (0.914-3.401) | 0.091 | 2.945 (1.009-8.597) | 0.048 |
| Lymph node involvement, N0 vs N1-3 | 1.442 (0.750-2.775) | 0.273 | 2.242 (0.765-6.566) | 0.141 |
| CTCs before chemo, −ve vs +ve | 2.847 (1.613-5.024) | 0.0003 | 3.623 (1.681-7.812) | 0.001 |
| EFC#93 before chemo, −ve vs +ve | 3.782 (1.965-7.281) | <0.0001 | 5.973 (2.634-13.542) | <0.0001 |

CI = confidence interval; CTC = circulating tumor cell; HR = hazard ratio

TABLE 3
Table 3 below lists a nucleic acid sequence (SEQ ID NO: 1) comprising DMR EFC#93
(genome version - hg19, chromosome - chr3, coordinates with primers -
chr3:194118853-194118957). Each MVP within the DMR is identified as [CG]
with the cytosine being the site of potential methylation.
Also listed are nucleic acid sequences (SEQ ID NOS: 2 to 12) each comprising
the same nucleic acid sequence as presented in SEQ ID NO: 1 but wherein each
MVP is individually and separately identified as [CG].

| Position of | | SEQ |
|---|---|---|
| marker | | ID |
| CpGs | Full genomic sequence with CpG highlighted | NO. |
| | CGTGAGGTTGGTGGGCAGGCCTAG[CG][CG]GAGATG[CG][CG]CCA[CG]T[CG]CCCCC[CG]AGCACTG | 1 |
| | [CG][CG]G[CG]TCC[CG]GAAGACACACTTGCAAGCTGGCGGACAGGGGAA | |
| 194118923 | CGTGAGGTTGGTGGGCAGGCCTAGCGCGGAGATGCGCGCCACGTCGCCCCCCGAGCACTGCGCGGCGTCC[CG] | 2 |
| | GAAGACACACTTGCAAGCTGGCGGACAGGGGAA | |
| 194118918− | CGTGAGGTTGGTGGGCAGGCCTAGCGCGGAGATGCGCGCCACGTCGCCCCCCGAGCACTGCGCGG[CG]TCC | 3 |
| | CGGAAGACACACTTGCAAGCTGGCGGACAGGGGAA | |
| 194118915− | CGTGAGGTTGGTGGGCAGGCCTAGCGCGGAGATGCGCGCCACGTCGCCCCCCGAGCACTGCG[CG]GCGTCC | 4 |
| | CGGAAGACACACTTGCAAGCTGGCGGACAGGGGAA | |
| 194118913− | CGTGAGGTTGGTGGGCAGGCCTAGCGCGGAGATGCGCGCCACGTCGCCCCCCGAGCACTG[CG]CGGCGTCC | 5 |
| | CGGAAGACACACTTGCAAGCTGGCGGACAGGGGAA | |
| 194118904− | CGTGAGGTTGGTGGGCAGGCCTAGCGCGGAGATGCGCGCCACGTCGCCCCC[CG]AGCACTGCGCGGCGTCC | 6 |
| | CGGAAGACACACTTGCAAGCTGGCGGACAGGGGAA | |
| 194118897− | CGTGAGGTTGGTGGGCAGGCCTAGCGCGGAGATGCGCGCCACGT[CG]CCCCCCGAGCACTGCGCGGCGTCC | 7 |
| | CGGAAGACACACTTGCAAGCTGGCGGACAGGGGAA | |
| 194118894− | CGTGAGGTTGGTGGGCAGGCCTAGCGCGGAGATGCGCGCCA[CG]TCGCCCCCCGAGCACTGCGCGGCGTCC | 8 |
| | CGGAAGACACACTTGCAAGCTGGCGGACAGGGGAA | |
| 194118889- | CGTGAGGTTGGTGGGCAGGCCTAGCGCGGAGATGCG[CG]CCACGTCGCCCCCCGAGCACTGCGCGGCGTCC | 9 |
| | CGGAAGACACACTTGCAAGCTGGCGGACAGGGGAA | |
| 194118887− | CGTGAGGTTGGTGGGCAGGCCTAGCGCGGAGATG[CG]CGCCACGTCGCCCCCCGAGCACTGCGCGGCGTCC | 10 |
| | CGGAAGACACACTTGCAAGCTGGCGGACAGGGGAA | |
| 194118879- | CGTGAGGTTGGTGGGCAGGCCTAGCG[CG]GAGATGCGCGCCACGTCGCCCCCCGAGCACTGCGCGGCGTCC | 11 |
| | CGGAAGACACACTTGCAAGCTGGCGGACAGGGGAA | |
| 19411887 | CGTGAGGTTGGTGGGCAGGCCTAG[CG]CGGAGATGCGCGCCACGTCGCCCCCCGAGCACTGCGCGGCGTCC | 12 |
| | CGGAAGACACACTTGCAAGCTGGCGGACAGGGGAA | |

TABLE 4
Table 4 below lists a nucleic acid sequence (SEQ ID NO: 13) comprising DMR EFC#89.
Each MVP withinthe DMR is identified as [CG] with the cytosine being the site of
potential methylation. Also listed are nucleic acid sequences (SEQ ID NOS: 14 to 24)
each comprising the same nucleic acid sequence as presented in SEQ ID NO: 13 but
wherein each MVP is individually and separately identified as [CG].

| | | | Position of | | SEQ |
|---|---|---|---|---|---|
| DMR | Position with | | marker | | ID |
| # | primers | Strand | CpGs | Full genomic sequence with CpG highlighted | NO. |
| 89 | chr1:3157511-3157650 | + | | GGGGTGCGGGGAGGTTGAGAG[CG][CG]G[CG]GC[CG] | 13 |
| | | | | CTGCCAGCAAT[CG]AGGAGCCAG[CG]G[CG][CG]TGTGCTGA | |
| | | | | GGGCCCAGCTAGCAAAATAAAGAGGGTTTTCAG[CG]GAG | |
| | | | | [CG]G[CG]GCTCAGGCGAGGCTGGGGGAGCCGGGGA | |
| 89 | chr1:3157511-3157650 | + | 3157532 | GGGGTGCGGGGAGGTTGAGAG[CG]CGGCGGCCGCTGCCAGC | 14 |
| | | | | AATCGAGGAGCCAGCGGCGCGTGTGCTGAGGGCCCAGCTAGCA | |
| | | | | AAATAAAGAGGGTTTTCAGCGGAGCGGCGGCTCAGGCGAGGCTGGGGGAGCCGGGGA | |
| 89 | chr1:3157511-3157650 | + | 3157534 | GGGGTGCGGGGAGGTTGAGAGCG[CG]GCGGCCGCTGCCAGC | 15 |
| | | | | AATCGAGGAGCCAGCGGCGCGTGTGCTGAGGGCCCAGCTAGCA | |
| | | | | AAATAAAGAGGGTTTTCAGCGGAGCGGCGGCTCAGGCGAGGCTGGGGGAGCCGGGGA | |
| 89 | chr1:3157511-3157650 | + | 3157537 | GGGGTGCGGGGAGGTTGAGAGCGCGG[CG]GCCGCTGCCAGC | 16 |
| | | | | AATCGAGGAGCCAGCGGCGCGTGTGCTGAGGGCCCAGCTAGCA | |
| | | | | AAATAAAGAGGGTTTTCAGCGGAGCGGCGGCTCAGGCGAGGCTGGGGGAGCCGGGGA | |
| 89 | chr1:3157511-3157650 | + | 3157541 | GGGGTGCGGGGAGGTTGAGAGCGCGGCGGC[CG]CTGCCAGC | 17 |
| | | | | AATCGAGGAGCCAGCGGCGCGTGTGCTGAGGGCCCAGCTAGCA | |
| | | | | AAATAAAGAGGGTTTTCAGCGGAGCGGCGGCTCAGGCGAGGCTGGGGGAGCCGGGGA | |
| 89 | chr1:3157511-3157650 | + | 3157554 | GGGGTGCGGGGAGGTTGAGAGCGCGGCGGCCGCTGCCAGCAAT[CG] | 18 |
| | | | | AGGAGCCAGCGGCGCGTGTGCTGAGGGCCCAGCTAGCA | |
| | | | | AAATAAAGAGGGTTTTCAGCGGAGCGGCGGCTCAGGCGAGGCTGGGGGAGCCGGGGA | |
| 89 | chr1:3157511-3157650 | + | 3157565 | GGGGTGCGGGGAGGTTGAGAGCGCGGCGGCCGCTGCCAGCAAT | 19 |
| | | | | CGAGGAGCCAG[CG]GCGCGTGTGCTGAGGGCCCAGCTAGCA | |
| | | | | AAATAAAGAGGGTTTTCAGCGGAGCGGCGGCTCAGGCGAGGCTGGGGGAGCCGGGGA | |
| 89 | chr1:3157511-3157650 | + | 3157568 | GGGGTGCGGGGAGGTTGAGAGCGCGGCGGCCGCTGCCAGCAAT | 20 |
| | | | | CGAGGAGCCAGCGG[CG]CGTGTGCTGAGGGCCCAGCTAGCA | |
| | | | | AAATAAAGAGGGTTTTCAGCGGAGCGGCGGCTCAGGCGAGGCTGGGGGAGCCGGGGA | |
| 89 | chr1:3157511-3157650 | + | 3157570 | GGGGTGCGGGGAGGTTGAGAGCGCGGCGGCCGCTGCCAGCAAT | 21 |
| | | | | CGAGGAGCCAGCGGCG[CG]TGTGCTGAGGGCCCAGCTAGCA | |
| | | | | AAATAAAGAGGGTTTTCAGCGGAGCGGCGGCTCAGGCGAGGCTGGGGGAGCCGGGGA | |
| 89 | chr1:3157511-3157650 | + | 3157613 | GGGGTGCGGGGAGGTTGAGAGCGCGGCGGCCGCTGCCAGCAAT | 22 |
| | | | | CGAGGAGCCAGCGGCGCGTGTGCTGAGGGCCCAGCTAGCAAA | |
| | | | | ATAAAGAGGGTTTTCAG[CG]GAGCGGCGGCTCAGGCGAGGCTGGGGGAGCCGGGGA | |
| 89 | chr1:3157511-3157650 | + | 3157618 | GGGGTGCGGGGAGGTTGAGAGCGCGGCGGCCGCTGCCAGCAAT | 23 |
| | | | | CGAGGAGCCAGCGGCGCGTGTGCTGAGGGCCCAGCTAGCAAA | |
| | | | | ATAAAGAGGGTTTTCAGCGGAG[CG]GCGGCTCAGGCGAGGCTGGGGGAGCCGGGGA | |
| 89 | chr1:3157511-3157650 | + | 3157621 | GGGGTGCGGGGAGGTTGAGAGCGCGGCGGCCGCTGCCAGCAAT | 24 |
| | | | | CGAGGAGCCAGCGGCGCGTGTGCTGAGGGCCCAGCTAGCAAA | |
| | | | | ATAAAGAGGGTTTTCAGCGGAGCGG[CG]GCTCAGGCGAGGCTGGGGGAGCCGGGGA | |

TABLE 5
Table 5 below lists a nucleic acid sequence (SEQ ID NO: 25) comprising DMR EFC#91.
Each MVP within the DMR is identified as [CG] with the cytosine being the site of
potential methylation. Also listed are nucleic acid sequences (SEQ ID NOS: 26 to 36)
each comprising the same nucleic acid sequence as presented in SEQ ID NO: 25
but wherein each MVP is individually and separately identified as [CG].

| | | | Position | | SEQ |
|---|---|---|---|---|---|
| DMR | Position with | | of marker | | ID |
| # | primers | Strand | CpGs | Full genomic sequence with CpG highlighted | NO. |
| 91 | chr2:19550330-19550456 | + | | GGCGCTGAAGCTGGAGAGGCCATCCTG[CG]CTTGGGAAAGGC | 25 |
| | | | | [CG][CG]GG[CG]CCAC[CG]CCTG[CG][CG]GTCC[CG] | |
| | | | | [CG]GTCAGGG[CG]CTGGAGCTGGGGGGAGCCC[CG]CCTTGC | |
| | | | | CCCAAGGAGAAGAGCCCCGG | |
| 91 | chr2:19550330-19550456 | + | 19550357 | GGCGCTGAAGCTGGAGAGGCCATCCTG[CG]CTTGGGAAAG | 26 |
| | | | | GCCGCGGGCGCCACCGCCTGCGCGGTCCCGCGGTCAGGGCGCTG | |
| | | | | GAGCTGGGGGGAGCCCCGCCTTGCCCCAAGGAGAAGAGCCCCGG | |
| 91 | chr2:19550330-19550456 | + | 19550371 | GGCGCTGAAGCTGGAGAGGCCATCCTGCGCTTGGGAAAGGC[CG] | 27 |
| | | | | CGGGCGCCACCGCCTGCGCGGTCCCGCGGTCAGGGCGCTG | |
| | | | | GAGCTGGGGGGAGCCCCGCCTTGCCCCAAGGAGAAGAGCCCCGG | |
| 91 | chr2:19550330-19550456 | + | 19550373 | GGCGCTGAAGCTGGAGAGGCCATCCTGCGCTTGGGAAAGG | 28 |
| | | | | CCG[CG]GGCGCCACCGCCTGCGCGGTCCCGCGGTCAGGGCGCTG | |
| | | | | GAGCTGGGGGGAGCCCCGCCTTGCCCCAAGGAGAAGAGCCCCGG | |
| 91 | chr2:19550330-19550456 | + | 19550377 | GGCGCTGAAGCTGGAGAGGCCATCCTGCGCTTGGGAAAGGC | 29 |
| | | | | CGCGGG[CG]CCACCGCCTGCGCGGTCCCGCGGTCAGGGCGCTG | |
| | | | | GAGCTGGGGGGAGCCCCGCCTTGCCCCAAGGAGAAGAGCCCCGG | |
| 91 | chr2:19550330-19550456 | + | 19550383 | GGCGCTGAAGCTGGAGAGGCCATCCTGCGCTTGGGAAAGGC | 30 |
| | | | | CGCGGGCGCCAC[CG]CCTGCGCGGTCCCGCGGTCAGGGCGCTG | |
| | | | | GAGCTGGGGGGAGCCCCGCCTTGCCCCAAGGAGAAGAGCCCCGG | |
| 91 | chr2:19550330-19550456 | + | 19550389 | GGCGCTGAAGCTGGAGAGGCCATCCTGCGCTTGGGAAAGGCCGC | 31 |
| | | | | GGGCGCCACCGCCTG[CG]CGGTCCCGCGGTCAGGGCGCTG | |
| | | | | GAGCTGGGGGGAGCCCCGCCTTGCCCCAAGGAGAAGAGCCCCGG | |
| 91 | chr2:19550330-19550456 | + | 19550391 | GGCGCTGAAGCTGGAGAGGCCATCCTGCGCTTGGGAAAGGCCGC | 32 |
| | | | | GGGCGCCACCGCCTGCG[CG]GTCCCGCGGTCAGGGCGCTG | |
| | | | | GAGCTGGGGGGAGCCCCGCCTTGCCCCAAGGAGAAGAGCCCCGG | |
| 91 | chr2:19550330-19550456 | + | 19550397 | GGCGCTGAAGCTGGAGAGGCCATCCTGCGCTTGGGAAAGGCCGC | 33 |
| | | | | GGGCGCCACCGCCTGCGCGGTCC[CG]CGGTCAGGGCGCTG | |
| | | | | GAGCTGGGGGGAGCCCCGCCTTGCCCCAAGGAGAAGAGCCCCGG | |
| 91 | chr2:19550330-19550456 | + | 19550399 | GGCGCTGAAGCTGGAGAGGCCATCCTGCGCTTGGGAAAGGCCGC | 34 |
| | | | | GGGCGCCACCGCCTGCGCGGTCCCG[CG]GTCAGGGCGCTG | |
| | | | | GAGCTGGGGGGAGCCCCGCCTTGCCCCAAGGAGAAGAGCCCCGG | |
| 91 | chr2:19550330-19550456 | + | 19550408 | GGCGCTGAAGCTGGAGAGGCCATCCTGCGCTTGGGAAAGGCCGC | 35 |
| | | | | GGGCGCCACCGCCTGCGCGGTCCCGCGGTCAGGG[CG]CTG | |
| | | | | GAGCTGGGGGGAGCCCCGCCTTGCCCCAAGGAGAAGAGCCCCGG | |
| 91 | chr2:19550330-19550456 | + | 19550429 | GGCGCTGAAGCTGGAGAGGCCATCCTGCGCTTGGGAAAGGCCGC | 36 |
| | | | | GGGCGCCACCGCCTGCGCGGTCCCGCGGTCAGGGCGCTGGA | |
| | | | | GCTGGGGGGAGCCC[CG]CCTTGCCCCAAGGAGAAGAGCCCCGG | |

TABLE 6
Table 6 below lists a nucleic acid sequence (SEQ ID NO: 37) comprising DMR EFC#92.
Each MVP withinthe DMR is identified as [CG] with the cytosine being the site
of potential methylation. Also listed are nucleic acid sequences (SEQ ID NOS: 38 to 53)
each comprising the same nucleic acid sequence as presented in SEQ ID NO: 37 but
wherein each MVP is individually and separately identified as [CG].

| | | | Position of | | SEQ |
|---|---|---|---|---|---|
| DMR | Position with | | marker | | ID |
| # | primers | Strand | CpGs | Full genomic sequence with CpG highlighted | NO. |
| 92 | chr2:19550279-19550427 | − | | TGCAGCAGGGAAGCTTATAGTCCAGTTGTCATC[CG][CG]GC[CG] | 37 |
| | | | | C[CG][CG]CTC[CG]GG[CG]CTGAAGCTGGAGAGGCCATCCTG[CG] | |
| | | | | CTTGGGAAAGGC[CG][CG]GG[CG]CCAC[CG]CCTG[CG][CG]GTCC | |
| | | | | [CG][CG]GTCAGGGCGCTGGAGCTGGGGGGAGCC | |
| 92 | chr2:19550279-19550427 | − | 19550312 | TGCAGCAGGGAAGCTTATAGTCCAGTTGTCATC[CG] | 38 |
| | | | | CGGCCGCCGCGCTCCGGGCGCTGAAGCTGGAGAGGCCATCCTGCGCTT | |
| | | | | GGGAAAGGCCGCGGGCGCCACCGCCTGCGCGGTCCCGCGGTCAGGGCG | |
| | | | | CTGGAGCTGGGGGGAGCC | |
| 92 | chr2:19550279-19550427 | − | 19550314 | TGCAGCAGGGAAGCTTATAGTCCAGTTGTCATCCG[CG] | 39 |
| | | | | GCCGCCGCGCTCCGGGCGCTGAAGCTGGAGAGGCCATCCTGCGCTT | |
| | | | | GGGAAAGGCCGCGGGCGCCACCGCCTGCGCGGTCCCGCGGTCAGGG | |
| | | | | CGCTGGAGCTGGGGGGAGCC | |
| 92 | chr2:19550279-19550427 | − | 19550318 | TGCAGCAGGGAAGCTTATAGTCCAGTTGTCATCCGCGGC[CG] | 40 |
| | | | | CCGCGCTCCGGGCGCTGAAGCTGGAGAGGCCATCCTGCGCTT | |
| | | | | GGGAAAGGCCGCGGGCGCCACCGCCTGCGCGGTCCCGCGGTCAGGG | |
| | | | | CGCTGGAGCTGGGGGGAGCC | |
| 92 | chr2:19550279-19550427 | − | 19550321 | TGCAGCAGGGAAGCTTATAGTCCAGTTGTCATCCGCGGCCGC[CG] | 41 |
| | | | | CGCTCCGGGCGCTGAAGCTGGAGAGGCCATCCTGCGCTT | |
| | | | | GGGAAAGGCCGCGGGCGCCACCGCCTGCGCGGTCCCGCGGTCAGGG | |
| | | | | CGCTGGAGCTGGGGGGAGCC | |
| 92 | chr2:19550279-19550427 | − | 19550323 | TGCAGCAGGGAAGCTTATAGTCCAGTTGTCATCCGCGGCCGCCG[CG] | 42 |
| | | | | CTCCGGGCGCTGAAGCTGGAGAGGCCATCCTGCGCTT | |
| | | | | GGGAAAGGCCGCGGGCGCCACCGCCTGCGCGGTCCCGCGGTCAGGGC | |
| | | | | GCTGGAGCTGGGGGGAGCC | |
| 92 | chr2:19550279-19550427 | − | 19550328 | TGCAGCAGGGAAGCTTATAGTCCAGTTGTCATCCGCGGCCGCCGCGCTC | 43 |
| | | | | [CG]GGCGCTGAAGCTGGAGAGGCCATCCTGCGCTT | |
| | | | | GGGAAAGGCCGCGGGCGCCACCGCCTGCGCGGTCCCGCGGTCAGGGCGC | |
| | | | | TGGAGCTGGGGGGAGCC | |
| 92 | chr2:19550279-19550427 | − | 19550332 | TGCAGCAGGGAAGCTTATAGTCCAGTTGTCATCCGCGGCCGCCGCGCTC | 44 |
| | | | | CGGG[CG]CTGAAGCTGGAGAGGCCATCCTGCGCTT | |
| | | | | GGGAAAGGCCGCGGGCGCCACCGCCTGCGCGGTCCCGCGGTCAGGGCGC | |
| | | | | TGGAGCTGGGGGGAGCC | |
| 92 | chr2:19550279-19550427 | − | 19550357 | TGCAGCAGGGAAGCTTATAGTCCAGTTGTCATCCGCGGCCGCCGCGCTC | 45 |
| | | | | CGGGCGCTGAAGCTGGAGAGGCCATCCTG[CG]CTT | |
| | | | | GGGAAAGGCCGCGGGCGCCACCGCCTGCGCGGTCCCGCGGTCAGGGCGC | |
| | | | | TGGAGCTGGGGGGAGCC | |
| 92 | chr2:19550279-19550427 | − | 19550371 | TGCAGCAGGGAAGCTTATAGTCCAGTTGTCATCCGCGGCCGCCGCGCTC | 46 |
| | | | | CGGGCGCTGAAGCTGGAGAGGCCATCCTGCGCTTGG | |
| | | | | GAAAGGC[CG]CGGGCGCCACCGCCTGCGCGGTCCCGCGGTCAGGGCGC | |
| | | | | TGGAGCTGGGGGGAGCC | |
| 92 | chr2:19550279-19550427 | − | 19550373 | TGCAGCAGGGAAGCTTATAGTCCAGTTGTCATCCGCGGCCGCCGCGCTC | 47 |
| | | | | CGGGCGCTGAAGCTGGAGAGGCCATCCTGCGCTTGG | |
| | | | | GAAAGGCCG[CG]GGCGCCACCGCCTGCGCGGTCCCGCGGTCAGGGCGC | |
| | | | | TGGAGCTGGGGGGAGCC | |
| 92 | chr2:19550279-19550427 | − | 19550377 | TGCAGCAGGGAAGCTTATAGTCCAGTTGTCATCCGCGGCCGCCGCGCTC | 48 |
| | | | | CGGGCGCTGAAGCTGGAGAGGCCATCCTGCGCTTGG | |
| | | | | GAAAGGCCGCGGG[CG]CCACCGCCTGCGCGGTCCCGCGGTCAGGGCGC | |
| | | | | TGGAGCTGGGGGGAGCC | |
| 92 | chr2:19550279-19550427 | − | 19550383 | TGCAGCAGGGAAGCTTATAGTCCAGTTGTCATCCGCGGCCGCCGCGCTC | 49 |
| | | | | CGGGCGCTGAAGCTGGAGAGGCCATCCTGCGCTTGG | |
| | | | | GAAAGGCCGCGGGCGCCAC[CG]CCTGCGCGGTCCCGCGGTCAGGGCGC | |
| | | | | TGGAGCTGGGGGGAGCC | |
| 92 | chr2:19550279-19550427 | − | 19550389 | TGCAGCAGGGAAGCTTATAGTCCAGTTGTCATCCGCGGCCGCCGCGCTC | 50 |
| | | | | CGGGCGCTGAAGCTGGAGAGGCCATCCTGCGCTTGG | |
| | | | | GAAAGGCCGCGGGCGCCACCGCCTG[CG]CGGTCCCGCGGTCAGGGCGC | |
| | | | | TGGAGCTGGGGGGAGCC | |
| 92 | chr2:19550279-19550427 | − | 19550391 | TGCAGCAGGGAAGCTTATAGTCCAGTTGTCATCCGCGGCCGCCGCGCTC | 51 |
| | | | | CGGGCGCTGAAGCTGGAGAGGCCATCCTGCGCTTGG | |
| | | | | GAAAGGCCGCGGGCGCCACCGCCTGCG[CG]GTCCCGCGGTCAGGGCGC | |
| | | | | TGGAGCTGGGGGGAGCC | |
| 92 | chr2:19550279-19550427 | − | 19550397 | TGCAGCAGGGAAGCTTATAGTCCAGTTGTCATCCGCGGCCGCCGCGCTC | 52 |
| | | | | CGGGCGCTGAAGCTGGAGAGGCCATCCTGCGCTTGG | |
| | | | | GAAAGGCCGCGGGCGCCACCGCCTGCGCGGTCC[CG]CGGTCAGGGCGC | |
| | | | | TGGAGCTGGGGGGAGCC | |
| 92 | chr2:19550279-19550427 | − | 19550399 | TGCAGCAGGGAAGCTTATAGTCCAGTTGTCATCCGCGGCCGCCGCGCTC | 53 |
| | | | | CGGGCGCTGAAGCTGGAGAGGCCATCCTGCGCTTGG | |
| | | | | GAAAGGCCGCGGGCGCCACCGCCTGCGCGGTCCCG[CG]GTCAGGGCGC | |
| | | | | TGGAGCTGGGGGGAGCC | |

TABLE 7
Table 7 below lists a nucleic acid sequence (SEQ ID NO: 54) comprising DMR EFC#94.
Each MVP within the DMR is identified as [CG] with the cytosine being the site
of potential methylation. Also listed are nucleic acid sequences (SEQ ID NOS: 55 to 66)
each comprising the same nucleic acid sequence as presented in SEQ ID NO: 54
but wherein each MVP is individually and separately identified as [CG].

| | | | Position | | SEQ |
|---|---|---|---|---|---|
| DMR | Position with | | of marker | | ID |
| # | primers | Strand | CpGs | Full genomic sequence with CpG highlighted | NO. |
| 94 | chr3:194118827-194118950 | − | | CGGCCCATTCCGAAGAGCAGGATGTG[CG]TGAGGTTGGTGGGCAGG | 54 |
| | | | | CCTAG[CG][CG]GAGATG[CG][CG]CCA[CG]T[CG]CCCCC[CG] | |
| | | | | AGCACTG[CG][CG]G[CG]TCC[CG]GAAGACACACTTGCAAGCTG | |
| | | | | GCGGAC | |
| 94 | chr3:194118827-194118950 | − | 194118853 | CGGCCCATTCCGAAGAGCAGGATGTG[CG]TGAGGTTG | 55 |
| | | | | GTGGGCAGGCCTAGCGCGGAGATGCGCGCCACGTCGCCCCCCGAGCA | |
| | | | | CTGCGCGGCGTCCCGGAAGACACACTTGCAAGCTGGCGGAC | |
| 94 | chr3:194118827-194118950 | − | 194118877 | CGGCCCATTCCGAAGAGCAGGATGTGCGTGAGGTTGGTGGGCA | 56 |
| | | | | GGCCTAG[CG]CGGAGATGCGCGCCACGTCGCCCCCCGAGCA | |
| | | | | CTGCGCGGCGTCCCGGAAGACACACTTGCAAGCTGGCGGAC | |
| 94 | chr3:194118827-194118950 | − | 194118879 | CGGCCCATTCCGAAGAGCAGGATGTGCGTGAGGTTGGTGGGCA | 57 |
| | | | | GGCCTAGCG[CG]GAGATGCGCGCCACGTCGCCCCCCGAGCA | |
| | | | | CTGCGCGGCGTCCCGGAAGACACACTTGCAAGCTGGCGGAC | |
| 94 | chr3:194118827-194118950 | − | 194118887 | CGGCCCATTCCGAAGAGCAGGATGTGCGTGAGGTTGGTGGGCA | 58 |
| | | | | GGCCTAGCGCGGAGATG[CG]CGCCACGTCGCCCCCCGAGCA | |
| | | | | CTGCGCGGCGTCCCGGAAGACACACTTGCAAGCTGGCGGAC | |
| 94 | chr3:194118827-194118950 | − | 194118889 | CGGCCCATTCCGAAGAGCAGGATGTGCGTGAGGTTGGTG | 59 |
| | | | | GGCAGGCCTAGCGCGGAGATGCG[CG]CCACGTCGCCCCCCGAGCA | |
| | | | | CTGCGCGGCGTCCCGGAAGACACACTTGCAAGCTGGCGGAC | |
| 94 | chr3:194118827-194118950 | − | 194118894 | CGGCCCATTCCGAAGAGCAGGATGTGCGTGAGGTTGGTG | 60 |
| | | | | GGCAGGCCTAGCGCGGAGATGCGCGCCA[CG]TCGCCCCCCGAGCA | |
| | | | | CTGCGCGGCGTCCCGGAAGACACACTTGCAAGCTGGCGGAC | |
| 94 | chr3:194118827-194118950 | − | 194118897 | CGGCCCATTCCGAAGAGCAGGATGTGCGTGAGGTTGGTG | 61 |
| | | | | GGCAGGCCTAGCGCGGAGATGCGCGCCACGT[CG]CCCCCCGAGCA | |
| | | | | CTGCGCGGCGTCCCGGAAGACACACTTGCAAGCTGGCGGAC | |
| 94 | chr3:194118827-194118950 | − | 194118904 | CGGCCCATTCCGAAGAGCAGGATGTGCGTGAGGTTGGTG | 62 |
| | | | | GGCAGGCCTAGCGCGGAGATGCGCGCCACGTCGCCCCC[CG]AGCA | |
| | | | | CTGCGCGGCGTCCCGGAAGACACACTTGCAAGCTGGCGGAC | |
| 94 | chr3:194118827-194118950 | − | 194118913 | CGGCCCATTCCGAAGAGCAGGATGTGCGTGAGGTTGGTG | 63 |
| | | | | GGCAGGCCTAGCGCGGAGATGCGCGCCACGTCGCCCCCCGAGCACT | |
| | | | | G[CG]CGGCGTCCCGGAAGACACACTTGCAAGCTGGCGGAC | |
| 94 | chr3:194118827-194118950 | − | 194118915 | CGGCCCATTCCGAAGAGCAGGATGTGCGTGAGGTTGGTG | 64 |
| | | | | GGCAGGCCTAGCGCGGAGATGCGCGCCACGTCGCCCCCCGAGCACT | |
| | | | | GCG[CG]GCGTCCCGGAAGACACACTTGCAAGCTGGCGGAC | |
| 94 | chr3:194118827-194118950 | − | 194118918 | CGGCCCATTCCGAAGAGCAGGATGTGCGTGAGGTTGGTG | 65 |
| | | | | GGCAGGCCTAGCGCGGAGATGCGCGCCACGTCGCCCCCCGAGCACT | |
| | | | | GCGCGG[CG]TCCCGGAAGACACACTTGCAAGCTGGCGGAC | |
| 94 | chr3:194118827-194118950 | − | 194118923 | CGGCCCATTCCGAAGAGCAGGATGTGCGTGAGGTTGGTG | 66 |
| | | | | GGCAGGCCTAGCGCGGAGATGCGCGCCACGTCGCCCCCCGAGCACT | |
| | | | | GCGCGGCGTCC[CG]GAAGACACACTTGCAAGCTGGCGGAC | |

TABLE 8
Table 8 below lists a nucleic acid sequence (SEQ ID NO: 67) comprising DMR EFC#95.
Each MVP within the DMR is identified as [CG] with the cytosine being the site
of potential methylation. Also listed are nucleic acid sequences (SEQ ID NOS: 68 to 74)
each comprising the same nucleic acid sequence as presented in SEQ ID NO: 67
but wherein each MVP is individually and separately identified as [CG].

| | | | Position | | SEQ |
|---|---|---|---|---|---|
| DMR | Position with | | of marker | | ID |
| # | primers | Strand | CpGs | Full genomic sequence with CpG highlighted | NO. |
| 95 | chr3:128712373-128712480 | + | | GAACAACAGATAAGGGTGGCTGGCAGTAAGCA[CG]A[CG] | 67 |
| | | | | A[CG]AGCAACCC[CG]TTTCCTT[CG]CCTAACCAGGAG | |
| | | | | TCAGT[CG]C[CG]GGCTTCTGGAATGCCTGCCCCAGGTGA | |
| 95 | chr3:128712373-128712480 | + | 128712405 | GAACAACAGATAAGGGTGGCTGGCAGTAAGCA[CG] | 68 |
| | | | | ACGACGAGCAACCCCGTTTCCTTCGCCTAACCAGGAGTCA | |
| | | | | GTCGCCGGGCTTCTGGAATGCCTGCCCCAGGTGA | |
| 95 | chr3:128712373-128712480 | + | 128712408 | GAACAACAGATAAGGGTGGCTGGCAGTAAGCACGA[CG] | 69 |
| | | | | ACGAGCAACCCCGTTTCCTTCGCCTAACCAGGAGTCAGT | |
| | | | | CGCCGGGCTTCTGGAATGCCTGCCCCAGGTGA | |
| 95 | chr3:128712373-128712480 | + | 128712411 | GAACAACAGATAAGGGTGGCTGGCAGTAAGCACGACGA[CG] | 70 |
| | | | | AGCAACCCCGTTTCCTTCGCCTAACCAGGAGTCAGTCGCCGGG | |
| | | | | CTTCTGGAATGCCTGCCCCAGGTGA | |
| 95 | chr3:128712373-128712480 | + | 128712421 | GAACAACAGATAAGGGTGGCTGGCAGTAAGCACGACGACGAG | 71 |
| | | | | CAACCC[CG]TTTCCTTCGCCTAACCAGGAGTCAGTCGCCGGG | |
| | | | | CTTCTGGAATGCCTGCCCCAGGTGA | |
| 95 | chr3:128712373-128712480 | + | 128712430 | GAACAACAGATAAGGGTGGCTGGCAGTAAGCACGACGACGAG | 72 |
| | | | | CAACCCCGTTTCCTT[CG]CCTAACCAGGAGTCAGTCGCCGGG | |
| | | | | CTTCTGGAATGCCTGCCCCAGGTGA | |
| 95 | chr3:128712373-128712480 | + | 128712449 | GAACAACAGATAAGGGTGGCTGGCAGTAAGCACGACGACGAG | 73 |
| | | | | CAACCCCGTTTCCTTCGCCTAACCAGGAGTCAGT[CG]CCGGG | |
| | | | | CTTCTGGAATGCCTGCCCCAGGTGA | |
| 95 | chr3:128712373-128712480 | + | 128712452 | GAACAACAGATAAGGGTGGCTGGCAGTAAGCACGACGACGAG | 74 |
| | | | | CAACCCCGTTTCCTTCGCCTAACCAGGAGTCAGTCGC[CG]GG | |
| | | | | CTTCTGGAATGCCTGCCCCAGGTGA | |

TABLE 9
Table 9 below lists a nucleic acid sequence
(SEQ ID NO: 75) comprising DMR EFC#96.
Each MVP within the DMR is identified
as [GC] with the cytosine being the
site of potential methylation.
Also listed are nucleic acid sequences
(SEQ ID NOS: 76 to 82) each comprising
the same nucleic acid sequence as
presented in SEQ ID NO: 75 but wherein
each MVP is individually and separately
identified as [GC].

| | | | Position | Full genomic | |
|---|---|---|---|---|---|
| | Position | | of | sequence | SEQ |
| DMR | with | | marker | with CpG | ID |
| # | primers | Strand | CpGs | highlighted | NO. |
| 96 | chr3: | − | | AAGGAACAACAGATAAG | 75 |
| | 128712370- | | | GGTGGCTGGCAGTAAGC | |
| | 128712482 | | | A[GC]A[GC]A[GC]AG | |
| | | | | CAACCC[GC]TTTCCTT | |
| | | | | [GC]CCTAACCAGGAGT | |
| | | | | CAGT[GC]C[GC]GGCT | |
| | | | | TCTGGAATGCCTGCCCC | |
| | | | | AGGTGAGC | |
| 96 | chr3: | − | 128712405 | AAGGAACAACAGATAAG | 76 |
| | 128712370- | | | GGTGGCTGGCAGTAAGC | |
| | 128712482 | | | A[GC]ACGACGAGCAAC | |
| | | | | CCCGTTTCCTTCGCCTA | |
| | | | | ACCAGGAGTCAGTCGCC | |
| | | | | GGGCTTCTGGAATGCCT | |
| | | | | GCCCCAGGTGAGC | |
| 96 | chr3: | − | 128712408 | AAGGAACAACAGATAAG | 77 |
| | 128712370- | | | GGTGGCTGGCAGTAAGC | |
| | 128712482 | | | ACGA[GC]ACGAGCAAC | |
| | | | | CCCGTTTCCTTCGCCTA | |
| | | | | ACCAGGAGTCAGTCGCC | |
| | | | | GGGCTTCTGGAATGCCT | |
| | | | | GCCCCAGGTGAGC | |
| 96 | chr3: | − | 128712411 | AAGGAACAACAGATAAG | 78 |
| | 128712370- | | | GGTGGCTGGCAGTAAGC | |
| | 128712482 | | | ACGACGA[GC]AGCAAC | |
| | | | | CCCGTTTCCTTCGCCTA | |
| | | | | ACCAGGAGTCAGTCGCC | |
| | | | | GGGCTTCTGGAATGCCT | |
| | | | | GCCCCAGGTGAGC | |
| 96 | chr3: | − | 128712421 | AAGGAACAACAGATAAG | 79 |
| | 128712370- | | | GGTGGCTGGCAGTAAGC | |
| | 128712482 | | | ACGACGACGAGCAACCC | |
| | | | | [GC]TTTCCTTCGCCTA | |
| | | | | ACCAGGAGTCAGTCGCC | |
| | | | | GGGCTTCTGGAATGCCT | |
| | | | | GCCCCAGGTGAGC | |
| 96 | chr3: | − | 128712430 | AAGGAACAACAGATAAG | 80 |
| | 128712370- | | | GGTGGCTGGCAGTAAGC | |
| | 128712482 | | | ACGACGACGAGCAACCC | |
| | | | | CGTTTCCTT[GC]CCTA | |
| | | | | ACCAGGAGTCAGTCGCC | |
| | | | | GGGCTTCTGGAATGCCT | |
| | | | | GCCCCAGGTGAGC | |
| 96 | chr3: | − | 128712449 | AAGGAACAACAGATAAG | 81 |
| | 128712370- | | | GGTGGCTGGCAGTAAGC | |
| | 128712482 | | | ACGACGACGAGCAACCC | |
| | | | | CGTTTCCTTCGCCTAAC | |
| | | | | CAGGAGTCAGT[GC]CC | |
| | | | | GGGCTTCTGGAATGCCT | |
| | | | | GCCCCAGGTGAGC | |
| 96 | chr: | − | 128712452 | AAGGAACAACAGATAAG | 82 |
| | 128712370- | | | GGTGGCTGGCAGTAAGC | |
| | 128712482 | | | ACGACGACGAGCAACCC | |
| | | | | CGTTTCCTTCGCCTAAC | |
| | | | | CAGGAGTCAGTCGC[GC] | |
| | | | | GGCTTCTGGAATGCCTG | |
| | | | | CCCCAGGTGAGC | |

TABLE 10
Table 10 below lists a nucleic acid sequence
(SEQ ID NO: 83) comprising DMR EFC#97.
Each MVP within the DMR is identified as
[CG] with the cytosine being the site of
potential methylation. Also listed are
nucleic acid sequences (SEQ ID NOS: 84 to 88)
each comprising the same nucleic acid
sequence as presented in SEQ ID NO:
83 but wherein each MVP is individually
and separately identified as [CG].

| | | | Position | Full genomic | |
|---|---|---|---|---|---|
| | Position | | of | sequence | SEQ |
| DMR | with | | marker | with CpG | ID |
| # | primers | Strand | CpGs | highlighted | NO. |
| 97 | chr4: | + | | CCGGGACAGCACCTTG | 83 |
| | 139483017- | | | GGAGCTGGG[CG]GAG | |
| | 139483134 | | | A[CG]CTTAAATCCCA | |
| | | | | A[CG]CTTCCAGAAAG | |
| | | | | AAGTTTGTGAAGAAAA | |
| | | | | GGTGAAGAG[CG]AGT | |
| | | | | TCC[CG]CAGGCAAAT | |
| | | | | TGGATGGGCGTCTGGC | |
| 97 | chr4: | + | 139483042 | CCGGGACAGCACCTTG | 84 |
| | 139483017- | | | GGAGCTGGG[CG]GAG | |
| | 139483134 | | | ACGCTTAAATCCCAAC | |
| | | | | GCTTCCAGAAAGAAGT | |
| | | | | TTGTGAAGAAAAGGTG | |
| | | | | AAGAGCGAGTTCCCGC | |
| | | | | AGGCAAATTGGATGGG | |
| | | | | CGTCTGGC | |
| 97 | chr4: | + | 139483048 | CCGGGACAGCACCTTG | 85 |
| | 139483017- | | | GGAGCTGGGCGGAGA | |
| | 139483134 | | | [CG]CTTAAATCCCAA | |
| | | | | CGCTTCCAGAAAGAAG | |
| | | | | TTTGTGAAGAAAAGGT | |
| | | | | GAAGAGCGAGTTCCCG | |
| | | | | CAGGCAAATTGGATGG | |
| | | | | GCGTCTGGC | |
| 97 | chr4: | + | 139483062 | CCGGGACAGCACCTTG | 86 |
| | 139483017- | | | GGAGCTGGGCGGAGAC | |
| | 139483134 | | | GCTTAAATCCCAA | |
| | | | | [CG]CTTCCAGAAAGA | |
| | | | | AGTTTGTGAAGAAAAG | |
| | | | | GTGAAGAGCGAGTTCC | |
| | | | | CGCAGGCAAATTGGAT | |
| | | | | GGGCGTCTGGC | |
| 97 | chr4: | + | 139483100 | CCGGGACAGCACCTTG | 87 |
| | 139483017- | | | GGAGCTGGGCGGAGAC | |
| | 139483134 | | | GCTTAAATCCCAACGC | |
| | | | | TTCCAGAAAGAAGTTT | |
| | | | | GTGAAGAAAAGGTGAA | |
| | | | | GAG[CG]AGTTCCCGC | |
| | | | | AGGCAAATTGGATGGG | |
| | | | | CGTCTGGC | |
| 97 | chr4: | + | 139483108 | CCGGGACAGCACCTTG | 88 |
| | 139483017- | | | GAGGCTGGGCGGAGAC | |
| | 139483134 | | | GCTTAAATCCCAACGC | |
| | | | | TTCCAGAAAGAAGTTT | |
| | | | | GTGAAGAAAAGGTGAA | |
| | | | | GAGCGAGTTCC[CG]C | |
| | | | | AGGCAAATTGGATGGG | |
| | | | | CGTCTGGC | |

TABLE 11
Table 11 below lists a nucleic acid sequence
(SEQ ID NO: 89) comprising DMR EFC#99.
Each MVP within the DMR is identified as
[CG] with the cytosine being the site
of potential methylation. Also listed are
nucleic acid sequences (SEQ ID NOS: 90 to 96)
each comprising the same nucleic acid
sequence as presented in SEQ ID NO:
89 but wherein each MVP is individually
and separately identified as [CG].

| | | | Position | Full genomic | |
|---|---|---|---|---|---|
| | Position | | of | sequence | SEQ |
| DMR | with | | marker | with CpG | ID |
| # | primers | Strand | CpGs | highlighted | NO. |
| 99 | chr8: | − | | TTAAAAACCCCT | 89 |
| | 103629512- | | | CTCTCTTCCGGG | |
| | 103629661 | | | TG[CG]GTGGCT | |
| | | | | CA[CG]CCTGTA | |
| | | | | ATCCCAGCACTT | |
| | | | | TGGGAGGC[CG] | |
| | | | | AGG[CG]GGTGG | |
| | | | | ATCA[CG]AGGT | |
| | | | | CAGGAGAT[CG] | |
| | | | | AGACCATCCTGG | |
| | | | | TTAACA[CG]AT | |
| | | | | GAAAACCCGTCT | |
| | | | | CTACTAAAAAAA | |
| | | | | ATACAAAA | |
| 99 | chr8: | − | 103629538 | TTAAAAACCCCT | 90 |
| | 103629512- | | | CTCTCTTCCGGG | |
| | 103629661 | | | TG[CG]GTGGCT | |
| | | | | CACGCCTGTAAT | |
| | | | | CCCAGCACTTTG | |
| | | | | GGAGGCCGAGGC | |
| | | | | GGGTGGATCACG | |
| | | | | AGGTCAGGAGAT | |
| | | | | CGAGACCATCCT | |
| | | | | GGTTAACACGAT | |
| | | | | GAAAACCCGTCT | |
| | | | | CTACTAAAAAAA | |
| | | | | ATACAAAA | |
| 99 | chr8: | − | 103629548 | TTAAAAACCCCT | 91 |
| | 103629512- | | | CTCTCTTCCGGG | |
| | 103629661 | | | TGCGGTGGCTCA | |
| | | | | [CG]CCTGTAAT | |
| | | | | CCCAGCACTTTG | |
| | | | | GGAGGCCGAGGC | |
| | | | | GGGTGGATCACG | |
| | | | | AGGTCAGGAGAT | |
| | | | | CGAGACCATCCT | |
| | | | | GGTTAACACGAT | |
| | | | | GAAAACCCGTCT | |
| | | | | CTACTAAAAAAA | |
| | | | | ATACAAAA | |
| 99 | chr8: | − | 103629576 | TTAAAAACCCCT | 92 |
| | 103629512- | | | CTCTCTTCCGGG | |
| | 103629661 | | | TGCGGTGGCTCA | |
| | | | | CGCCTGTAATCC | |
| | | | | CAGCACTTTGGG | |
| | | | | AGGC[CG]AGGC | |
| | | | | GGGTGGATCACG | |
| | | | | AGGTCAGGAGAT | |
| | | | | CGAGACCATCCT | |
| | | | | GGTTAACACGAT | |
| | | | | GAAAACCCGTCT | |
| | | | | CTACTAAAAAAA | |
| | | | | ATACAAAA | |
| 99 | chr8: | − | 103629581 | TTAAAAACCCCT | 93 |
| | 103629512- | | | CTCTCTTCCGGG | |
| | 103629661 | | | TGCGGTGGCTCA | |
| | | | | CGCCTGTAATCC | |
| | | | | CAGCACTTTGGG | |
| | | | | AGGCCGAGG[CG] | |
| | | | | GGTGGATCACGA | |
| | | | | GGTCAGGAGATC | |
| | | | | GAGACCATCCTG | |
| | | | | GTTAACACGATG | |
| | | | | AAAACCCGTCTC | |
| | | | | TACTAAAAAAAA | |
| | | | | TACAAAA | |
| 99 | chr8: | − | 103629592 | TTAAAAACCCCT | 94 |
| | 103629512- | | | CTCTCTTCCGGG | |
| | 103629661 | | | TGCGGTGGCTCA | |
| | | | | CGCCTGTAATCC | |
| | | | | CAGCACTTTGGG | |
| | | | | AGGCCGAGGCGG | |
| | | | | GTGGATCA[CG] | |
| | | | | AGGTCAGGAGAT | |
| | | | | CGAGACCATCCT | |
| | | | | GGTTAACACGAT | |
| | | | | GAAAACCCGTCT | |
| | | | | CTACTAAAAAAA | |
| | | | | ATACAAAA | |
| 99 | chr8: | − | 103629606 | TTAAAAACCCCT | 95 |
| | 103629512- | | | CTCTCTTCCGGG | |
| | 103629661 | | | TGCGGTGGCTCA | |
| | | | | CGCCTGTAATCC | |
| | | | | CAGCACTTTGGG | |
| | | | | AGGCCGAGGCGG | |
| | | | | GTGGATCACGAG | |
| | | | | GTCAGGAGAT | |
| | | | | [CG]AGACCATC | |
| | | | | CTGGTTAACACG | |
| | | | | ATGAAAACCCGT | |
| | | | | CTCTACTAAAAA | |
| | | | | AAATACAAAA | |
| 99 | chr8: | − | 103629626 | TTAAAAACCCCT | 96 |
| | 103629512- | | | CTCTCTTCCGGG | |
| | 103629661 | | | TGCGGTGGCTCA | |
| | | | | CGCCTGTAATCC | |
| | | | | CAGCACTTTGGG | |
| | | | | AGGCCGAGGCGG | |
| | | | | GTGGATCACGAG | |
| | | | | GTCAGGAGATCG | |
| | | | | AGACCATCCTGG | |
| | | | | TTAACA[CG]AT | |
| | | | | GAAAACCCGTCT | |
| | | | | CTACTAAAAAAA | |
| | | | | ATACAAAA | |

TABLE 12
Table 12 below lists a nucleic acid sequence
(SEQ ID NO: 97) comprising DMR EFC#101.
Each MVP within the DMR is identified as
[CG] with the cytosine being the site
of potential methylation. Also listed are
nucleic acid sequences (SEQ ID NOS 98 to 111)
each comprising the same nucleic acid
sequence as presented in SEQ ID NO: 97
but wherein each MVP is individually
and separately identified as [CG]

| | | | Position | Full genomic | |
|---|---|---|---|---|---|
| | Position | | of | sequence | SEQ |
| DMR | with | | marker | with CpG | ID |
| # | primers | Strand | CpGs | highlighted | NO. |
| 101 | chr8: | − | | GCCTCCTCACG | 97 |
| | 145106870- | | | AAAGAGCAGCT | |
| | 145106994 | | | [CG][CG]GGT | |
| | | | | GA[CG]C[CG] | |
| | | | | T[CG]C[CG] | |
| | | | | CCT[CG]GAAG | |
| | | | | [CG]GCCTCTG | |
| | | | | CCCCC[CG]AG | |
| | | | | CCCCC[CG]C | |
| | | | | [CG]CAGCT | |
| | | | | [CG]AAG[CG] | |
| | | | | G[CG]CAGGAT | |
| | | | | GACCGGGTACC | |
| | | | | TGCGAGGGCGA | |
| | | | | GGA | |
| 101 | chr8: | − | 145106892 | GCCTCCTCACG | 98 |
| | 145106870- | | | AAAGAGCAGCT | |
| | 145106994 | | | [CG]CGGGTGA | |
| | | | | CGCCGTCGCCG | |
| | | | | CCTCGGAAGCG | |
| | | | | GCCTCTGCCCC | |
| | | | | CCGAGCCCCCC | |
| | | | | GCCGCAGCTCG | |
| | | | | AAGCGGCGCAG | |
| | | | | GATGACCGGGT | |
| | | | | ACCTGCGAGGG | |
| | | | | CGAGGA | |
| 101 | chr8: | − | 145106894 | GCCTCCTCACG | 99 |
| | 145106870- | | | AAAGAGCAGCT | |
| | 145106994 | | | CG[CG]GGTGA | |
| | | | | CGCCGTCGCCG | |
| | | | | CCTCGGAAGCG | |
| | | | | GCCTCTGCCCC | |
| | | | | CCGAGCCCCCC | |
| | | | | GCCGCAGCTCG | |
| | | | | AAGCGGCGCAG | |
| | | | | GATGACCGGGT | |
| | | | | ACCTGCGAGGG | |
| | | | | CGAGGA | |
| 101 | chr8: | − | 145106901 | GCCTCCTCACG | 100 |
| | 145106870- | | | AAAGAGCAGCT | |
| | 145106994 | | | CGCGGGTGA | |
| | | | | [CG]CCGTCGC | |
| | | | | CGCCTCGGAAG | |
| | | | | CGGCCTCTGCC | |
| | | | | CCCCGAGCCCC | |
| | | | | CCGCCGCAGCT | |
| | | | | CGAAGCGGCGC | |
| | | | | AGGATGACCGG | |
| | | | | GTACCTGCGAG | |
| | | | | GGCGAGGA | |
| 101 | chr8: | − | 145106904 | GCCTCCTCACG | 101 |
| | 145106870- | | | AAAGAGCAGCT | |
| | 145106994 | | | CGCGGGTGACG | |
| | | | | C[CG]TCGCCG | |
| | | | | CCTCGGAAGCG | |
| | | | | GCCTCTGCCCC | |
| | | | | CCGAGCCCCCC | |
| | | | | GCCGCAGCTCG | |
| | | | | AAGCGGCGCAG | |
| | | | | GATGACCGGGT | |
| | | | | ACCTGCGAGGG | |
| | | | | CGAGGA | |
| 101 | chr8: | − | 145106907 | GCCTCCTCACG | 102 |
| | 145106870- | | | AAAGAGCAGCT | |
| | 145106994 | | | CGCGGGTGACG | |
| | | | | CCGT[CG]CCG | |
| | | | | CCTCGGAAGCG | |
| | | | | GCCTCTGCCCC | |
| | | | | CCGAGCCCCCC | |
| | | | | GCCGCAGCTCG | |
| | | | | AAGCGGCGCAG | |
| | | | | GATGACCGGGT | |
| | | | | ACCTGCGAGGG | |
| | | | | CGAGGA | |
| 101 | chr8: | − | 145106910 | GCCTCCTCACG | 103 |
| | 145106870- | | | AAAGAGCAGCT | |
| | 145106994 | | | CGCGGGTGACG | |
| | | | | CCGTCGC[CG] | |
| | | | | CCTCGGAAGCG | |
| | | | | GCCTCTGCCCC | |
| | | | | CCGAGCCCCCC | |
| | | | | GCCGCAGCTCG | |
| | | | | AAGCGGCGCAG | |
| | | | | GATGACCGGGT | |
| | | | | ACCTGCGAGGG | |
| | | | | CGAGGA | |
| 101 | chr8: | − | 145106915 | GCCTCCTCACG | 104 |
| | 145106870- | | | AAAGAGCAGCT | |
| | 145106994 | | | CGCGGGTGACG | |
| | | | | CCGTCGCCGCC | |
| | | | | T[CG]GAAGCG | |
| | | | | GCCTCTGCCCC | |
| | | | | CCGAGCCCCCC | |
| | | | | GCCGCAGCTCG | |
| | | | | AAGCGGCGCAG | |
| | | | | GATGACCGGGT | |
| | | | | ACCTGCGAGGG | |
| | | | | CGAGGA | |
| 101 | chr8: | − | 145106921 | GCCTCCTCACG | 105 |
| | 145106870- | | | AAAGAGCAGCT | |
| | 145106994 | | | CGCGGGTGACG | |
| | | | | CCGTCGCCGCC | |
| | | | | TCGGAAG[CG] | |
| | | | | GCCTCTGCCCC | |
| | | | | CCGAGCCCCCC | |
| | | | | GCCGCAGCTCG | |
| | | | | AAGCGGCGCAG | |
| | | | | GATGACCGGGT | |
| | | | | ACCTGCGAGGG | |
| | | | | CGAGGA | |
| 101 | chr8: | − | 145106935 | GCCTCCTCACG | 106 |
| | 145106870- | | | AAAGAGCAGCT | |
| | 145106994 | | | CGCGGGTGACG | |
| | | | | CCGTCGCCGCC | |
| | | | | TCGGAAGCGGC | |
| | | | | CTCTGCCCCC | |
| | | | | [CG]AGCCCCC | |
| | | | | CGCCGCAGCTC | |
| | | | | GAAGCGGCGCA | |
| | | | | GGATGACCGGG | |
| | | | | TACCTGCGAGG | |
| | | | | GCGAGGA | |
| 101 | chr8: | − | 145106944 | GCCTCCTCACG | 107 |
| | 145106870- | | | AAAGAGCAGCT | |
| | 145106994 | | | CGCGGGTGACG | |
| | | | | CCGTCGCCGCC | |
| | | | | TCGGAAGCGGC | |
| | | | | CTCTGCCCCCC | |
| | | | | GAGCCCCC[CG] | |
| | | | | CCGCAGCTCGA | |
| | | | | AGCGGCGCAGG | |
| | | | | ATGACCGGGTA | |
| | | | | CCTGCGAGGGC | |
| | | | | GAGGA | |
| 101 | chr8: | − | 145106947 | GCCTCCTCACGA | 108 |
| | 145106870- | | | AAGAGCAGCTCG | |
| | 145106994 | | | CGGGTGACGCCG | |
| | | | | TCGCCGCCTCGG | |
| | | | | AAGCGGCCTCTG | |
| | | | | CCCCCCGAGCCC | |
| | | | | CCCGC[CG]CAG | |
| | | | | CTCGAAGCGGCG | |
| | | | | CAGGATGACCGG | |
| | | | | GTACCTGCGAGG | |
| | | | | GCGAGGA | |
| 101 | chr8: | − | 145106954 | GCCTCCTCACGA | 109 |
| | 145106870- | | | AAGAGCAGCTCG | |
| | 145106994 | | | CGGGTGACGCCG | |
| | | | | TCGCCGCCTCGG | |
| | | | | AAGCGGCCTCTG | |
| | | | | CCCCCCGAGCCC | |
| | | | | CCCGCCGCAGCT | |
| | | | | [CG]AAGCGGCG | |
| | | | | CAGGATGACCGG | |
| | | | | GTACCTGCGAGG | |
| | | | | GCGAGGA | |
| 101 | chr8: | − | 145106959 | GCCTCCTCACGA | 110 |
| | 145106870- | | | AAGAGCAGCTCG | |
| | 145106994 | | | CGGGTGACGCCG | |
| | | | | TCGCCGCCTCGG | |
| | | | | AAGCGGCCTCTG | |
| | | | | CCCCCCGAGCCC | |
| | | | | CCCGCCGCAGCT | |
| | | | | CGAAG[CG]GCG | |
| | | | | CAGGATGACCGG | |
| | | | | GTACCTGCGAGG | |
| | | | | GCGAGGA | |
| 101 | chr8: | − | 145106962 | GCCTCCTCACGA | 111 |
| | 145106870- | | | AAGAGCAGCTCG | |
| | 145106994 | | | CGGGTGACGCCG | |
| | | | | TCGCCGCCTCGG | |
| | | | | AAGCGGCCTCTG | |
| | | | | CCCCCCGAGCCC | |
| | | | | CCCGCCGCAGCT | |
| | | | | CGAAGCGG[CG] | |
| | | | | CAGGATGACCGG | |
| | | | | GTACCTGCGAGG | |
| | | | | GCGAGGA | |

TABLE 13
Table 13 below lists a nucleic acid sequence
(SEQ ID NO: 112) comprising DMR EFC#105.
Each MVP within the DMR is identified as
[CG] with the cytosine being the site of
potential methylation. Also listed are
nucleic acid sequences (SEQ ID NOS: 113
to 119) each comprising the same nucleic
acid sequence as presented in SEQ ID NO
112 but wherein each MVP is individually
and separately identified as [CG].

| | | | Position | Full genomic | |
|---|---|---|---|---|---|
| | Position | | of | sequence | SEQ |
| DMR | with | | marker | with CpG | ID |
| # | primers | Strand | CpGs | highlighted | NO. |
| 105 | chr8: | − | | GGATCCAGGG | 112 |
| | 145103775- | | | TGGGGATTTG | |
| | 145103893 | | | AGATCAGGTC | |
| | | | | CCTTT[CG]G | |
| | | | | GTTTTCTTTT | |
| | | | | TGAAG[CG]C | |
| | | | | CCCTCTGCCT | |
| | | | | C[CG]CC[CG] | |
| | | | | [CG]CCTC[CG] | |
| | | | | CCAGGCT[CG] | |
| | | | | CTGCGTCAGCA | |
| | | | | CCTCACCGGCT | |
| | | | | TTGCACA | |
| 105 | chr8: | − | 145103810 | GGATCCAGGGT | 113 |
| | 145103775- | | | GGGGATTTGAG | |
| | 145103893 | | | ATCAGGTCCCT | |
| | | | | TT[CG]GGTTT | |
| | | | | TCTTTTTGAAG | |
| | | | | CGCCCCTCTGC | |
| | | | | CTCCGCCCGCG | |
| | | | | CCTCCGCCAGG | |
| | | | | CTCGCTGCGTC | |
| | | | | AGCACCTCACC | |
| | | | | GGCTTTGCACA | |
| 105 | chr8: | − | 145103828 | GGATCCAGGGT | 114 |
| | 145103775- | | | GGGGATTTGAG | |
| | 145103893 | | | ATCAGGTCCCT | |
| | | | | TTCGGGTTTTC | |
| | | | | TTTTTGAAG[CG] | |
| | | | | CCCCTCTGCC | |
| | | | | TCCGCCCGCGC | |
| | | | | CTCCGCCAGGC | |
| | | | | TCGCTGCGTCA | |
| | | | | GCACCTCACCG | |
| | | | | GCTTTGCACA | |
| 105 | chr8: | − | 145103842 | GGATCCAGGGT | 115 |
| | 145103775- | | | GGGGATTTGAG | |
| | 145103893 | | | ATCAGGTCCCT | |
| | | | | TTCGGGTTTTC | |
| | | | | TTTTTGAAGCG | |
| | | | | CCCCTCTGCCT | |
| | | | | C[CG]CCCGCG | |
| | | | | CCTCCGCCAGG | |
| | | | | CTCGCTGCGTC | |
| | | | | AGCACCTCACC | |
| | | | | GGCTTTGCACA | |
| 105 | chr8: | − | 145103846 | GGATCCAGGGT | 116 |
| | 145103775- | | | GGGGATTTGAG | |
| | 145103893 | | | ATCAGGTCCCT | |
| | | | | TTCGGGTTTTC | |
| | | | | TTTTTGAAGCG | |
| | | | | CCCCTCTGCCT | |
| | | | | CCGCC[CG]CG | |
| | | | | CCTCCGCCAGG | |
| | | | | CTCGCTGCGTC | |
| | | | | AGCACCTCACC | |
| | | | | GGCTTTGCACA | |
| 105 | chr8: | − | 145103848 | GGATCCAGGGT | 117 |
| | 145103775- | | | GGGGATTTGAG | |
| | 145103893 | | | ATCAGGTCCCT | |
| | | | | TTCGGGTTTTC | |
| | | | | TTTTTGAAGCG | |
| | | | | CCCCTCTGCCT | |
| | | | | CCGCCCG[CG] | |
| | | | | CCTCCGCCAGG | |
| | | | | CTCGCTGCGTC | |
| | | | | AGCACCTCACC | |
| | | | | GGCTTTGCACA | |
| 105 | chr8: | − | 145103854 | GGATCCAGGGT | 118 |
| | 145103775- | | | GGGGATTTGAG | |
| | 145103893 | | | ATCAGGTCCCT | |
| | | | | TTCGGGTTTTC | |
| | | | | TTTTTGAAGCG | |
| | | | | CCCCTCTGCCT | |
| | | | | CCGCCCGCGCC | |
| | | | | TC[CG]CCAGG | |
| | | | | CTCGCTGCGTC | |
| | | | | AGCACCTCACC | |
| | | | | GGCTTTGCACA | |
| 105 | chr8: | − | 145103863 | GGATCCAGGGT | 119 |
| | 145103775- | | | GGGGATTTGAG | |
| | 145103893 | | | ATCAGGTCCCT | |
| | | | | TTCGGGTTTTC | |
| | | | | TTTTTGAAGCG | |
| | | | | CCCCTCTGCCT | |
| | | | | CCGCCCGCGCC | |
| | | | | TCCGCCAGGCT | |
| | | | | [CG]CTGCGTC | |
| | | | | AGCACCTCACC | |
| | | | | GGCTTTGCACA | |

TABLE 14
Table 14 below lists a nucleic acid sequence
(SEQ ID NO: 120) comprising DMR EFC#106.
Each MVP within the DMR is identified as
[CG] with the cytosine being the site of
potential methylation. Also listed are
nucleic acid sequences (SEQ ID NOS: 121
to 128) each comprising the same nucleic
acid sequence as presented in SEQ ID NO
120 but wherein each MVP is individually
and separately identified as [CG].

| | | | Position | Full genomic | |
|---|---|---|---|---|---|
| | Position | | of | sequence | SEQ |
| DMR | with | | marker | with CpG | ID |
| # | primers | Strand | CpGs | highlighted | NO. |
| 106 | chr9: | + | | GGCAGAGTGAAG | 120 |
| | 100069971- | | | CACAAGCAATAA | |
| | 100070085 | | | TCCTGTATTATT | |
| | | | | [CG][CG]TTCC | |
| | | | | CAGAGTCCCTT | |
| | | | | [CG]GATTTG | |
| | | | | [CG]CCATG | |
| | | | | [CG][CG]G | |
| | | | | [CG]GGGAGAAC | |
| | | | | [CG]GCCTCCTG | |
| | | | | CTCGAGTTCAGA | |
| | | | | GCTCATCT | |
| 106 | chr9: | + | 100070007 | GGCAGAGTGAAG | 121 |
| | 100069971- | | | CACAAGCAATAA | |
| | 100070085 | | | TCCTGTATTATT | |
| | | | | [CG]CGTTCCCA | |
| | | | | GAGTCCCTTCGG | |
| | | | | ATTTGCGCCATG | |
| | | | | CGCGGCGGGGAG | |
| | | | | AACCGGCCTCCT | |
| | | | | GCTCGAGTTCAG | |
| | | | | AGCTCATCT | |
| 106 | chr9: | + | 100070009 | GGCAGAGTGAAG | 122 |
| | 100069971- | | | CACAAGCAATAA | |
| | 100070085 | | | TCCTGTATTATT | |
| | | | | CG[CG]TTCCCA | |
| | | | | GAGTCCCTTCGG | |
| | | | | ATTTGCGCCATG | |
| | | | | CGCGGCGGGGAG | |
| | | | | AACCGGCCTCCT | |
| | | | | GCTCGAGTTCAG | |
| | | | | AGCTCATCT | |
| 106 | chr9: | + | 100070026 | GGCAGAGTGAAG | 123 |
| | 100069971- | | | CACAAGCAATAA | |
| | 100070085 | | | TCCTGTATTATT | |
| | | | | CGCGTTCCCAGA | |
| | | | | GTCCCTT[CG]G | |
| | | | | ATTTGCGCCATG | |
| | | | | CGCGGCGGGGAG | |
| | | | | AACCGGCCTCCT | |
| | | | | GCTCGAGTTCAG | |
| | | | | AGCTCATCT | |
| 106 | chr9: | + | 100070034 | GGCAGAGTGAAG | 124 |
| | 100069971- | | | CACAAGCAATAA | |
| | 100070085 | | | TCCTGTATTATT | |
| | | | | CGCGTTCCCAGA | |
| | | | | GTCCCTTCGGAT | |
| | | | | TTG[CG]CCATG | |
| | | | | CGCGGCGGGGAG | |
| | | | | AACCGGCCTCCT | |
| | | | | GCTCGAGTTCAG | |
| | | | | AGCTCATCT | |
| 106 | Chr9: | + | 100070041 | GGCAGAGTGA | 125 |
| | 100069971- | | | AGCACAAGCA | |
| | 100070085 | | | ATAATCCTGT | |
| | | | | ATTATTCGCG | |
| | | | | TTCCCAGAGT | |
| | | | | CCCTTCGGAT | |
| | | | | TTGCGCCATG | |
| | | | | [CG]CGGCGG | |
| | | | | GGAGAACCGG | |
| | | | | CCTCCTGCTC | |
| | | | | GAGTTCAGAG | |
| | | | | CTCATCT | |
| 106 | Chr9: | + | 100070043 | GGCAGAGTGA | 126 |
| | 100069971- | | | AGCACAAGCA | |
| | 100070085 | | | ATAATCCTGT | |
| | | | | ATTATTCGCG | |
| | | | | TTCCCAGAGT | |
| | | | | CCCTTCGGAT | |
| | | | | TTGCGCCATG | |
| | | | | CG[CG]GCGG | |
| | | | | GGAGAACCGG | |
| | | | | CCTCCTGCTC | |
| | | | | GAGTTCAGAG | |
| | | | | CTCATCT | |
| 106 | Chr9: | + | 100070046 | GGCAGAGTGA | 127 |
| | 100069971- | | | AGCACAAGCA | |
| | 100070085 | | | ATAATCCTGT | |
| | | | | ATTATTCGCG | |
| | | | | TTCCCAGAGT | |
| | | | | CCCTTCGGAT | |
| | | | | TTGCGCCATG | |
| | | | | CGCGG[CG]G | |
| | | | | GGAGAACCGG | |
| | | | | CCTCCTGCTC | |
| | | | | GAGTTCAGAG | |
| | | | | CTCATCT | |
| 106 | Chr9: | + | 100070056 | GGCAGAGTGA | 128 |
| | 100069971- | | | AGCACAAGCA | |
| | 100070085 | | | ATAATCCTGT | |
| | | | | ATTATTCGCG | |
| | | | | TTCCCAGAGT | |
| | | | | CCCTTCGGAT | |
| | | | | TTGCGCCATG | |
| | | | | CGCGGCGGGG | |
| | | | | AGAAC[CG]G | |
| | | | | CCTCCTGCTC | |
| | | | | GAGTTCAGAG | |
| | | | | CTCATCT | |

```
Table 15
Table 15 below lists a nucleic acid sequence
  (SEQ ID NO: 129) comprising DMR EFC#107.
Each MVP within the DMR is identified as
  [CG] with the cytosine being the site of
potential methylation. Also listed are
nucleic acid sequences (SEQ ID NOS: 130
to 136) each comprising the same nucleic
acid sequence as presented in SEQ ID
NO: 129 but wherein each MVP is
individually and separately
  identified as [CG].
```

| | | | Position | Full genomic | |
|---|---|---|---|---|---|
| | Position | | of | sequence | SEQ |
| DMR | with | | marker | with CpG | ID |
| # | primers | Strand | CpGs | highlighted | NO. |
| 107 | chr9: | − | | GCAGAGTGAAG | 129 |
| | 100069972- | | | CACAAGCAATA | |
| | 100070073 | | | ATCCTGTATTA | |
| | | | | TT[CG][CG]T | |
| | | | | TCCCAGAGTCC | |
| | | | | CTT[CG]GATT | |
| | | | | TG[CG]CCATG | |
| | | | | [CG][CG]G | |
| | | | | [CG]GGGAGAA | |
| | | | | CCGGCCTCCTG | |
| | | | | CTCGAGTT | |
| 107 | chr9: | − | 100070007 | GCAGAGTGAAG | 130 |
| | 100069972- | | | CACAAGCAATA | |
| | 100070073 | | | ATCCTGTATTA | |
| | | | | TT[CG]CGTTC | |
| | | | | CCAGAGTCCCT | |
| | | | | TCGGATTTGCG | |
| | | | | CCATGCGCGGC | |
| | | | | GGGGAGAACCG | |
| | | | | GCCTCCTGCTC | |
| | | | | GAGTT | |
| 107 | chr9: | − | 100070009 | GCAGAGTGAAG | 131 |
| | 100069972- | | | CACAAGCAATA | |
| | 100070073 | | | ATCCTGTATTA | |
| | | | | TTCG[CG]TTC | |
| | | | | CCAGAGTCCCT | |
| | | | | TCGGATTTGCG | |
| | | | | CCATGCGCGGC | |
| | | | | GGGGAGAACCG | |
| | | | | GCCTCCTGCTC | |
| | | | | GAGTT | |
| 107 | chr9: | − | 100070026 | GCAGAGTGAAG | 132 |
| | 100069972- | | | CACAAGCAATA | |
| | 100070073 | | | ATCCTGTATTA | |
| | | | | TTCGCGTTCCC | |
| | | | | AGAGTCCCTT | |
| | | | | [CG]GATTTGC | |
| | | | | GCCATGCGCGG | |
| | | | | CGGGGAGAACC | |
| | | | | GGCCTCCTGCT | |
| | | | | CGAGTT | |
| 107 | chr9: | − | 100070034 | GCAGAGTGAAG | 133 |
| | 100069972- | | | CACAAGCAATA | |
| | 100070073 | | | ATCCTGTATTA | |
| | | | | TTCGCGTTCCC | |
| | | | | AGAGTCCCTTC | |
| | | | | GGATTTG[CG] | |
| | | | | CCATGCGCGGC | |
| | | | | GGGGAGAACCG | |
| | | | | GCCTCCTGCTC | |
| | | | | GAGTT | |
| 107 | chr9: | − | 100070041 | GCAGAGTGAAG | 134 |
| | 100069972- | | | CACAAGCAATA | |
| | 100070073 | | | ATCCTGTATTA | |
| | | | | TTCGCGTTCCC | |
| | | | | AGAGTCCCTTC | |
| | | | | GGATTTGCGCC | |
| | | | | ATG[CG]CGGC | |
| | | | | GGGGAGAACCG | |
| | | | | GCCTCCTGCT | |
| | | | | CGAGTT | |
| 107 | chr9: | − | 100070043 | GCAGAGTGAAG | 135 |
| | 100069972- | | | CACAAGCAATA | |
| | 100070073 | | | ATCCTGTATTA | |
| | | | | TTCGCGTTCCC | |
| | | | | AGAGTCCCTTC | |
| | | | | GGATTTGCGCC | |
| | | | | ATGCG[CG]GC | |
| | | | | GGGGAGAACCG | |
| | | | | GCCTCCTGCTC | |
| | | | | GAGTT | |
| 107 | chr9: | − | 100070046 | GCAGAGTGAAG | 136 |
| | 100069972- | | | CACAAGCAATA | |
| | 100070073 | | | ATCCTGTATTA | |
| | | | | TTCGCGTTCCC | |
| | | | | AGAGTCCCTTC | |
| | | | | GGATTTGCGCC | |
| | | | | ATGCGCGG | |
| | | | | [CG]GGGAGAA | |
| | | | | CCGGCCTCCTG | |
| | | | | CTCGAGTT | |

TABLE 16
Table 16 below lists a nucleic acid sequence
(SEQ ID NO: 137) comprising DMR EFC#108.
Each MVP within the DMR is identified as
[CG] with the cytosine being the site of
potential methylation. Also listed are
nucleic acid sequences (SEQ ID NOS: 138
to 143) each comprising the same nucleic
acid sequence as presented in SEQ ID
NO: 137 but wherein each MVP is
individually and separately
identified as [CG].

| | | | Position | Full genomic | |
|---|---|---|---|---|---|
| | Position | | of | sequence | SEQ |
| DMR | with | | marker | with CpG | ID |
| # | primers | Strand | CpGs | highlighted | NO. |
| 108 | chr9: | + | | ACTCCCTCCTC | 137 |
| | 139553849- | | | CTGCACCTCCT | |
| | 139553943 | | | GCAGCCCGGCT | |
| | | | | CC[CG][CG]G | |
| | | | | C[CG][CG]CC | |
| | | | | TGGTGCCCCTC | |
| | | | | TGTCT[CG] | |
| | | | | [CG]CCACCT | |
| | | | | GAGATGCCCAG | |
| | | | | GCTGGCCTCTG | |
| 108 | Chr9: | + | 139553884 | ACTCCCTCCTC | 138 |
| | 139553849- | | | CTGCACCTCCT | |
| | 139553943 | | | GCAGCCCGGCT | |
| | | | | CC[CG]CGGCC | |
| | | | | GCGCCTGGTGC | |
| | | | | CCCTCTGTCTC | |
| | | | | GCGCCACCTGA | |
| | | | | GATGCCCAGGC | |
| | | | | TGGCCTCTG | |
| 108 | chr9: | + | 139553886 | ACTCCCTCCTC | 139 |
| | 139553849- | | | CTGCACCTCCT | |
| | 139553943 | | | GCAGCCCGGCT | |
| | | | | CCCG[CG]GCC | |
| | | | | GCGCCTGGTGC | |
| | | | | CCCTCTGTCTC | |
| | | | | GCGCCACCTGA | |
| | | | | GATGCCCAGGC | |
| | | | | TGGCCTCTG | |
| 108 | chr9: | + | 139553890 | ACTCCCTCCTC | 140 |
| | 139553849- | | | CTGCACCTCCT | |
| | 139553943 | | | GCAGCCCGGCT | |
| | | | | CCCGCGGC | |
| | | | | [CG]CGCCTGG | |
| | | | | TGCCCCTCTGT | |
| | | | | CTCGCGCCACC | |
| | | | | TGAGATGCCCA | |
| | | | | GGCTGGCCTCT | |
| | | | | G | |
| 108 | chr9: | + | 139553892 | ACTCCCTCCTC | 141 |
| | 139553849- | | | CTGCACCTCCT | |
| | 139553943 | | | GCAGCCCGGCT | |
| | | | | CCCGCGGCCG | |
| | | | | [CG]CCTGGTG | |
| | | | | CCCCTCTGTCT | |
| | | | | CGCGCCACCTG | |
| | | | | AGATGCCCAGG | |
| | | | | CTGGCCTCTG | |
| 108 | chr9: | + | 139553912 | ACTCCCTCCTC | 141 |
| | 139553849- | | | CTGCACCTCCT | |
| | 139553943 | | | GCAGCCCGGCT | |
| | | | | CCCGCGGCCGC | |
| | | | | GCCTGGTGCCC | |
| | | | | CTCTGTCT[CG] | |
| | | | | CGCCACCTGAG | |
| | | | | ATGCCCAGGCT | |
| | | | | GGCCTCTG | |
| 108 | chr9: | + | 139553914 | ACTCCCTCCTC | 142 |
| | 139553849- | | | CTGCACCTCCT | |
| | 139553943 | | | GCAGCCCGGCT | |
| | | | | CCCGCGGCCGC | |
| | | | | GCCTGGTGCCC | |
| | | | | CTCTGTCTCG | |
| | | | | [CG]CCACCTG | |
| | | | | AGATGCCCAGG | |
| | | | | CTGGCCTCTG | |

TABLE 17
Table 17 below lists a nucleic acid sequence
(SEQ ID NO: 144) comprising DMR EFC#111.
Each MVP within the DMR is identified as
[CG] with the cytosine being the site of
potential methylation. Also listed are
nucleic acid sequences (SEQ ID NOS: 145
to 153) each comprising the same nucleic
acid sequence as presented in SEQ ID
NO: 144 but wherein each MVP is
individually and separately
identified as [CG].

| | | | Position | Full genomic | |
|---|---|---|---|---|---|
| | Position | | of | sequence | SEQ |
| DMR | with | | marker | with CpG | ID |
| # | primers | Strand | CpGs | highlighted | NO. |
| 111 | chr11: | − | | CCAGCCCCAAG | 144 |
| | 62693550- | | | TCTTGCGGGCA | |
| | 62693659 | | | GTTCC[CG]AA | |
| | | | | GAAAAGATGGG | |
| | | | | TTTGGGG[CG] | |
| | | | | GT[CG][CG]A | |
| | | | | AAG[CG]G | |
| | | | | [CG]CCT[CG] | |
| | | | | [CG]TGTTTTC | |
| | | | | CTGC[CG]TTC | |
| | | | | CCGGGTCCTTA | |
| | | | | TAGCCCGGCC | |
| 111 | chr11: | − | 62693577 | CCAGCCCCAAG | 145 |
| | 62693550- | | | TCTTGCGGGCA | |
| | 62693659 | | | GTTCC[CG]AA | |
| | | | | GAAAAGATGGG | |
| | | | | TTTGGGGCGGT | |
| | | | | CGCGAAAGCGG | |
| | | | | CGCCTCGCGTG | |
| | | | | TTTTCCTGCCG | |
| | | | | TTCCCGGGTCC | |
| | | | | TTATAGCCCGG | |
| | | | | CC | |
| 111 | chr11: | − | 62693599 | CCAGCCCCAAG | 146 |
| | 62693550- | | | TCTTGCGGGCA | |
| | 62693659 | | | GTTCCCGAAGA | |
| | | | | AAAGATGGGTT | |
| | | | | TGGGG[CG]GT | |
| | | | | CGCGAAAGCGG | |
| | | | | CGCCTCGCGTG | |
| | | | | TTTTCCTGCCG | |
| | | | | TTCCCGGGTCC | |
| | | | | TTATAGCCCGG | |
| | | | | CC | |
| 111 | chr11: | − | 62693603 | CCAGCCCCAAG | 147 |
| | 62693550- | | | TCTTGCGGGCA | |
| | 62693659 | | | GTTCCCGAAGA | |
| | | | | AAAGATGGGTT | |
| | | | | TGGGGCGGT | |
| | | | | [CG]CGAAAGC | |
| | | | | GGCGCCTCGCG | |
| | | | | TGTTTTCCTGC | |
| | | | | CGTTCCCGGGT | |
| | | | | CCTTATAGCCC | |
| | | | | GGCC | |
| 111 | chr11: | − | 62693605 | CCAGCCCCAAG | 148 |
| | 62693550- | | | TCTTGCGGGCA | |
| | 62693659 | | | GTTCCCGAAGA | |
| | | | | AAAGATGGGTT | |
| | | | | TGGGGCGGTCG | |
| | | | | [CG]AAAGCGG | |
| | | | | CGCCTCGCGTG | |
| | | | | TTTTCCTGCCG | |
| | | | | TTCCCGGGTCC | |
| | | | | TTATAGCCCGG | |
| | | | | CC | |
| 111 | chr11: | − | 62693611 | CCAGCCCCAAG | 149 |
| | 62693550- | | | TCTTGCGGGCA | |
| | 62693659 | | | GTTCCCGAAGA | |
| | | | | AAAGATGGGTT | |
| | | | | TGGGGCGGTCG | |
| | | | | CGAAAG[CG]G | |
| | | | | CGCCTCGCGTG | |
| | | | | TTTTCCTGCCG | |
| | | | | TTCCCGGGTCC | |
| | | | | TTATAGCCCGG | |
| | | | | CC | |
| 111 | chr11: | − | 62693614 | CCAGCCCCAAG | 150 |
| | 62693550- | | | TCTTGCGGGCA | |
| | 62693659 | | | GTTCCCGAAGA | |
| | | | | AAAGATGGGTT | |
| | | | | TGGGGCGGTCG | |
| | | | | CGAAAGCGG | |
| | | | | [CG]CCTCGCG | |
| | | | | TGTTTTCCTGC | |
| | | | | CGTTCCCGGGT | |
| | | | | CCTTATAGCCC | |
| | | | | GGCC | |
| 111 | chr11: | − | 62693619 | CCAGCCCCAAG | 151 |
| | 62693550- | | | TCTTGCGGGCA | |
| | 62693659 | | | GTTCCCGAAGA | |
| | | | | AAAGATGGGTT | |
| | | | | TGGGGCGGTCG | |
| | | | | CGAAAGCGGCG | |
| | | | | CCT[CG]CGTG | |
| | | | | TTTTCCTGCCG | |
| | | | | TTCCCGGGTCC | |
| | | | | TTATAGCCCGG | |
| | | | | CC | |
| 111 | chr11: | − | 62693621 | CCAGCCCCAAG | 152 |
| | 62693550- | | | TCTTGCGGGCA | |
| | 62693659 | | | GTTCCCGAAGA | |
| | | | | AAAGATGGGTT | |
| | | | | TGGGGCGGTCG | |
| | | | | CGAAAGCGGCG | |
| | | | | CCTCG[CG]TG | |
| | | | | TTTTCCTGCCG | |
| | | | | TTCCCGGGTCC | |
| | | | | TTATAGCCCGG | |
| | | | | CC | |
| 111 | chr11: | − | 62693634 | CCAGCCCCAAG | 153 |
| | 62693550- | | | TCTTGCGGGCA | |
| | 62693659 | | | GTTCCCGAAGA | |
| | | | | AAAGATGGGTT | |
| | | | | TGGGGCGGTCG | |
| | | | | CGAAAGCGGCG | |
| | | | | CCTCGCGTGTT | |
| | | | | TTCCTGC[CG] | |
| | | | | TTCCCGGGTCC | |
| | | | | TTATAGCCCGG | |
| | | | | CC | |

TABLE 18
Table 18 below lists a nucleic acid sequence
(SEQ ID NO: 154) comprising DMR EFC#114.
Each MVP within the DMR is identified as
[CG] with the cytosine being the site
of potential methylation. Also listed are
nucleic acid sequences (SEQ ID NOS: 155
to 161) each comprising the same nucleic
acid sequence as presented in SEQ ID
NO: 154 but wherein each MVP is
individually and separately
identified as [CG].

| | | | Position | Full genomic | |
|---|---|---|---|---|---|
| | Position | | of | sequence | SEQ |
| DMR | with | | marker | with CpG | ID |
| # | primers | Strand | CpGs | highlighted | NO. |
| 114 | chr16: | + | | GCCCCACGAGCC | 154 |
| | 1271174- | | | TCCGTCCGTTCT | |
| | 1271302 | | | GGTT[CG]GGTT | |
| | | | | TCTC[CG]AGTT | |
| | | | | TTGCTACCAGC | |
| | | | | [CG]AGGCTGTG | |
| | | | | [CG]GGCAACTG | |
| | | | | GGTCAGCCTCC | |
| | | | | [CG]TCAGGAGA | |
| | | | | GAAGC[CG] | |
| | | | | [CG]TCTGTGGG | |
| | | | | ACGAAGACCGGG | |
| | | | | CACC | |
| 114 | chr16: | + | 1271202 | GCCCCACGAGCC | 155 |
| | l271l74- | | | TCCGTCCGTTCT | |
| | 1271302 | | | GGTT[CG]GGTT | |
| | | | | TCTCCGAGTTTT | |
| | | | | GCTACCAGCCGA | |
| | | | | GGCTGTGCGGGC | |
| | | | | AACTGGGTCAGC | |
| | | | | CTCCCGTCAGGA | |
| | | | | GAGAAGCCGCGT | |
| | | | | CTGTGGGACGAA | |
| | | | | GACCGGGCACC | |
| 114 | chr16: | + | 1271212 | GCCCCACGAGCC | 156 |
| | 1271174- | | | TCCGTCCGTTCT | |
| | 1271302 | | | GGTTCGGGTTTC | |
| | | | | TC[CG]AGTTTT | |
| | | | | GCTACCAGCCGA | |
| | | | | GGCTGTGCGGGC | |
| | | | | AACTGGGTCAGC | |
| | | | | CTCCCGTCAGGA | |
| | | | | GAGAAGCCGCGT | |
| | | | | CTGTGGGACGAA | |
| | | | | GACCGGGCACC | |
| 114 | chr16: | + | 1271229 | GCCCCACGAGCC | 157 |
| | 1271174- | | | TCCGTCCGTTCT | |
| | 1271302 | | | GGTTCGGGTTTC | |
| | | | | TCCGAGTTTTGC | |
| | | | | TACCAGC[CG]A | |
| | | | | GGCTGTGCGGGC | |
| | | | | AACTGGGTCAGC | |
| | | | | CTCCCGTCAGGA | |
| | | | | GAGAAGCCGCGT | |
| | | | | CTGTGGGACGAA | |
| | | | | GACCGGGCACC | |
| 114 | chr16: | + | 1271239 | GCCCCACGAGCC | 158 |
| | 1271174- | | | TCCGTCCGTTCT | |
| | 1271302 | | | GGTTCGGGTTTC | |
| | | | | TCCGAGTTTTGC | |
| | | | | TACCAGCCGAGG | |
| | | | | CTGTG[CG]GGC | |
| | | | | AACTGGGTCAGC | |
| | | | | CTCCCGTCAGGA | |
| | | | | GAGAAGCCGCGT | |
| | | | | CTGTGGGACGAA | |
| | | | | GACCGGGCACC | |
| 114 | chr16: | + | 1271260 | GCCCCACGAGC | 159 |
| | 1271174- | | | CTCCGTCCGTT | |
| | 1271302 | | | CTGGTTCGGGT | |
| | | | | TTCTCCGAGTT | |
| | | | | TTGCTACCAGC | |
| | | | | CGAGGCTGTGC | |
| | | | | GGGCAACTGGG | |
| | | | | TCAGCCTCC | |
| | | | | [CG]TCAGGAG | |
| | | | | AGAAGCCGCGT | |
| | | | | CTGTGGGACGA | |
| | | | | AGACCGGGCAC | |
| | | | | C | |
| 114 | chr16: | + | 1271275 | GCCCCACGAGC | 160 |
| | 1271174- | | | CTCCGTCCGTT | |
| | 1271302 | | | CTGGTTCGGGT | |
| | | | | TTCTCCGAGTT | |
| | | | | TTGCTACCAGC | |
| | | | | CGAGGCTGTGC | |
| | | | | GGGCAACTGGG | |
| | | | | TCAGCCTCCCG | |
| | | | | TCAGGAGAGAA | |
| | | | | GC[CG]CGTCT | |
| | | | | GTGGGACGAAG | |
| | | | | ACCGGGCACC | |
| 114 | chr16: | + | 1271277 | GCCCCACGAGC | 161 |
| | 1271174- | | | CTCCGTCCGTT | |
| | 1271302 | | | CTGGTTCGGGT | |
| | | | | TTCTCCGAGTT | |
| | | | | TTGCTACCAGC | |
| | | | | CGAGGCTGTGC | |
| | | | | GGGCAACTGGG | |
| | | | | TCAGCCTCCCG | |
| | | | | TCAGGAGAGAA | |
| | | | | GCCG[CG]TCT | |
| | | | | GTGGGACGAAG | |
| | | | | ACCGGGCACC | |

Table 19
Table 19 below lists a nucleic acid sequence
(SEQ ID NO: 162) comprising DMR EFC#98.
Each MVP within the DMR is identified as
[CG] with the cytosine being the site of
potential methylation. Also listed are
nucleic acid sequences (SEQ ID NOS: 163
to 167) each comprising the same nucleic
acid sequence as presented in SEQ ID
NO: 162 but wherein each MVP is
individually and separately
identified as [CG].

| | | | Position | Full genomic | |
|---|---|---|---|---|---|
| | Position | | of | sequence | SEQ |
| DMR | with | | marker | with CpG | ID |
| # | primers | Strand | CpGs | highlighted | NO. |
| 98 | chr4: | − | | CTACAGGTCCG | 162 |
| | 139483009- | | | GGACAGCACCT | |
| | 139483139 | | | TGGGAGCTGGG | |
| | | | | [CG]GAGA[CG] | |
| | | | | CTTAAATCCCA | |
| | | | | A[CG]CTTCCA | |
| | | | | GAAAGAAGTTT | |
| | | | | GTGAAGAAAAG | |
| | | | | GTGAAGAG[CG] | |
| | | | | AGTTCC[CG]C | |
| | | | | AGGCAAATTGG | |
| | | | | ATGGGCGTCTG | |
| | | | | GCCGCCG | |
| 98 | chr4: | − | 139483042 | CTACAGGTCCG | 163 |
| | 139483009- | | | GGACAGCACCT | |
| | 139483139 | | | TGGGAGCTGGG | |
| | | | | [CG]GAGACGC | |
| | | | | TTAAATCCCAA | |
| | | | | CGCTTCCAGAA | |
| | | | | AGAAGTTTGTG | |
| | | | | AAGAAAAGGTG | |
| | | | | AAGAGCGAGTT | |
| | | | | CCCGCAGGCAA | |
| | | | | ATTGGATGGGC | |
| | | | | GTCTGGCCGCC | |
| | | | | G | |
| 98 | chr4: | − | 139483048 | CTACAGGTCCG | 164 |
| | 139483009- | | | GGACAGCACCT | |
| | 139483139 | | | TGGGAGCTGGG | |
| | | | | CGGAGA[CG]C | |
| | | | | TTAAATCCCAA | |
| | | | | CGCTTCCAGAA | |
| | | | | AGAAGTTTGTG | |
| | | | | AAGAAAAGGTG | |
| | | | | AAGAGCGAGTT | |
| | | | | CCCGCAGGCAA | |
| | | | | ATTGGATGGGC | |
| | | | | GTCTGGCCGCC | |
| | | | | G | |
| 98 | chr4: | − | 139483062 | CTACAGGTCCG | 165 |
| | 139483009- | | | GGACAGCACCT | |
| | 139483139 | | | TGGGAGCTGGG | |
| | | | | CGGAGACGCTT | |
| | | | | AAATCCCAA | |
| | | | | [CG]CTTCCAG | |
| | | | | AAAGAAGTTTG | |
| | | | | TGAAGAAAAGG | |
| | | | | TGAAGAGCGAG | |
| | | | | TTCCCGCAGGC | |
| | | | | AAATTGGATGG | |
| | | | | GCGTCTGGCCG | |
| | | | | CCG | |
| 98 | chr4: | − | 139483100 | CTACAGGTCCG | 166 |
| | 139483009- | | | GGACAGCACCT | |
| | 139483139 | | | TGGGAGCTGGG | |
| | | | | CGGAGACGCTT | |
| | | | | AAATCCCAACG | |
| | | | | CTTCCAGAAAG | |
| | | | | AAGTTTGTGAA | |
| | | | | GAAAAGGTGAA | |
| | | | | GAG[CG]AGTT | |
| | | | | CCCGCAGGCAA | |
| | | | | ATTGGATGGGC | |
| | | | | GTCTGGCCGCC | |
| | | | | G | |
| 98 | chr4: | − | 139483108 | CTACAGGTCCG | 167 |
| | 139483009- | | | GGACAGCACCT | |
| | 139483139 | | | TGGGAGCTGGG | |
| | | | | CGGAGACGCTT | |
| | | | | AAATCCCAACG | |
| | | | | CTTCCAGAAAG | |
| | | | | AAGTTTGTGAA | |
| | | | | GAAAAGGT | |
| | | | | GAAGAGCGAGT | |
| | | | | TCC[CG]CAGG | |
| | | | | CAAATTGGATG | |
| | | | | GGCGTCTGGCC | |
| | | | | GCCG | |

TABLE 20
Table 20 below lists a nucleic acid sequence
(SEQ ID NO: 168) comprising DMR EFC#102.
Each MVP within the DMR is identified as
[CG] with the cytosine being the site
of potential methylation. Also listed are
nucleic acid sequences (SEQ ID NOS: 169
to 180) each comprising the same nucleic
acid sequence as presented in SEQ ID
NO: 168 but wherein each MVP is
individually and separately
identified as [CG].

| | | | Position | Full genomic | |
|---|---|---|---|---|---|
| | Position | | of | sequence | SEQ |
| DMR | with | | marker | with CpG | ID |
| # | primers | Strand | CpGs | highlighted | NO. |
| 102 | chr8: | + | | GTGTCCTCCTA | 168 |
| | 145106096- | | | AGGCAAGCACA | |
| | 145106222 | | | GATGAGGGG | |
| | | | | [CG][CG] | |
| | | | | [CG]GCTGG | |
| | | | | CG+[CG]CACA | |
| | | | | GACA[CG]ACT | |
| | | | | [CG]GAGCA | |
| | | | | [CG]AACTAG | |
| | | | | G[CG]C[CG] | |
| | | | | TAGCTG[CG] | |
| | | | | TCCCCAGAA | |
| | | | | C[CG]GGAGA | |
| | | | | CTTAAGGCAT | |
| | | | | CTTTATTGCG | |
| | | | | GG | |
| 102 | Chr8: | + | 145106127 | GTGTCCTCCTA | 169 |
| | 145106096- | | | AGGCAAGCACA | |
| | 145106222 | | | GATGAGGGG | |
| | | | | [CG]CGCGGCT | |
| | | | | GGCGCGCACAG | |
| | | | | ACACGACTCGG | |
| | | | | AGCACGAACTA | |
| | | | | GGCGCCGTAGC | |
| | | | | TGCGTCCCCAG | |
| | | | | AACCGGGAGAC | |
| | | | | TTAAGGCATCT | |
| | | | | TTATTGCGGG | |
| 102 | chr8: | + | 145106129 | GTGTCCTCCTA | 170 |
| | 145106096- | | | AGGCAAGCACA | |
| | 145106222 | | | GATGAGGGGCG | |
| | | | | [CG]CGGCTGG | |
| | | | | CGCGCACAGAC | |
| | | | | ACGACTCGGAG | |
| | | | | CACGAACTAGG | |
| | | | | CGCCGTAGCTG | |
| | | | | CGTCCCCAGAA | |
| | | | | CCGGGAGACTT | |
| | | | | AAGGCATCTTT | |
| | | | | ATTGCGGG | |
| 102 | chr8: | + | 145106131 | GTGTCCTCCTA | 171 |
| | 145106096- | | | AGGCAAGCACA | |
| | 145106222 | | | GATGAGGGGCG | |
| | | | | CG[CG]GCTGG | |
| | | | | CGCGCACAGAC | |
| | | | | ACGACTCGGAG | |
| | | | | CACGAACTAGG | |
| | | | | CGCCGTAGCTG | |
| | | | | CGTCCCCAGAA | |
| | | | | CCGGGAGACTT | |
| | | | | AAGGCATCTTT | |
| | | | | ATTGCGGG | |
| 102 | chr8: | + | 145106138 | GTGTCCTCCTA | |
| | 145106096- | | | AGGCAAGCACA | 172 |
| | 145106222 | | | GATGAGGGGCG | |
| | | | | CGCGGCTGG | |
| | | | | [CG]CGCACAG | |
| | | | | ACACGACTCGG | |
| | | | | AGCACGAACTA | |
| | | | | GGCGCCGTAGC | |
| | | | | TGCGTCCCCAG | |
| | | | | AACCGGGAGAC | |
| | | | | TTAAGGCATCT | |
| | | | | TTATTGCGGG | |
| 102 | chr8: | + | 145106140 | GTGTCCTCCTA | 173 |
| | 145106096- | | | AGGCAAGCACA | |
| | 145106222 | | | GATGAGGGGCG | |
| | | | | CGCGGCTGGCG | |
| | | | | [CG]CACAGAC | |
| | | | | ACGACTCGGAG | |
| | | | | CACGAACTAGG | |
| | | | | CGCCGTAGCTG | |
| | | | | CGTCCCCAGAA | |
| | | | | CCGGGAGACTT | |
| | | | | AAGGCATCTTT | |
| | | | | ATTGCGGG | |
| 102 | chr8: | + | 145106150 | GTGTCCTCCTA | 174 |
| | 145106096- | | | AGGCAAGCACA | |
| | 145106222 | | | GATGAGGGGCG | |
| | | | | CGCGGCTGGCG | |
| | | | | CGCACAGACA | |
| | | | | [CG]ACTCGGA | |
| | | | | GCACGAACTAG | |
| | | | | GCGCCGTAGCT | |
| | | | | GCGTCCCCAGA | |
| | | | | ACCGGGAGACT | |
| | | | | TAAGGCATCTT | |
| | | | | TATTGCGGG | |
| 102 | chr8: | + | 145106155 | GTGTCCTCCTA | 175 |
| | 145106096- | | | AGGCAAGCACA | |
| | 145106222 | | | GATGAGGGGCG | |
| | | | | CGCGGCTGGCG | |
| | | | | CGCACAGACAC | |
| | | | | GACT[CG]GAG | |
| | | | | CACGAACTAGG | |
| | | | | CGCCGTAGCTG | |
| | | | | CGTCCCCAGAA | |
| | | | | CCGGGAGACTT | |
| | | | | AAGGCATCTTT | |
| | | | | ATTGCGGG | |
| 102 | chr8: | + | 145106162 | GTGTCCTCCTA | 176 |
| | 145106096- | | | AGGCAAGCACA | |
| | 145106222 | | | GATGAGGGGCG | |
| | | | | CGCGGCTGGCG | |
| | | | | CGCACAGACAC | |
| | | | | GACTCGGAGCA | |
| | | | | [CG]AACTAGG | |
| | | | | CGCCGTAGCTG | |
| | | | | CGTCCCCAGAA | |
| | | | | CCGGGAGACTT | |
| | | | | AAGGCATCTTT | |
| | | | | ATTGCGGG | |
| 102 | chr8: | + | 145106171 | GTGTCCTCCTA | 177 |
| | 145106096- | | | AGGCAAGCACA | |
| | 145106222 | | | GATGAGGGGCG | |
| | | | | CGCGGCTGGCG | |
| | | | | CGCACAGACAC | |
| | | | | GACTCGGAGCA | |
| | | | | CGAACTAGG | |
| | | | | [CG]CCGTAG | |
| | | | | CTGCGTCCCCA | |
| | | | | GAACCGGGAGA | |
| | | | | CTTAAGGCATC | |
| | | | | TTTATTGCGGG | |
| 102 | chr8: | + | 145106174 | GTGTCCTCCTA | 178 |
| | 145106096- | | | AGGCAAGCACA | |
| | 145106222 | | | GATGAGGGGCG | |
| | | | | CGCGGCTGGCG | |
| | | | | CGCACAGACAC | |
| | | | | GACTCGGAGCA | |
| | | | | CGAACTAGGCG | |
| | | | | C[CG]TAGCTG | |
| | | | | CGTCCCCAGAA | |
| | | | | CCGGGAGACTT | |
| | | | | AAGGCATCTTT | |
| | | | | ATTGCGGG | |
| 102 | chr8: | + | 145106182 | GTGTCCTCCTA | 179 |
| | 145106096- | | | AGGCAAGCACA | |
| | 145106222 | | | GATGAGGGGCG | |
| | | | | CGCGGCTGGCG | |
| | | | | CGCACAGACAC | |
| | | | | GACTCGGAGCA | |
| | | | | CGAACTAGGCG | |
| | | | | CCGTAGCTG | |
| | | | | [CG]TCCCCAG | |
| | | | | AACCGGGAGAC | |
| | | | | TTAAGGCATCT | |
| | | | | TTATTGCGGG | |
| 102 | chr8: | + | 145106194 | GTGTCCTCCTA | 180 |
| | 145106096- | | | AGGCAAGCACA | |
| | 145106222 | | | GATGAGGGGCG | |
| | | | | CGCGGCTGGCG | |
| | | | | CGCACAGACAC | |
| | | | | GACTCGGAGCA | |
| | | | | CGAACTAGGCG | |
| | | | | CCGTAGCTGCG | |
| | | | | TCCCCAGAAC | |
| | | | | [CG]GGAGACT | |
| | | | | TAAGGCATCTT | |
| | | | | TATTGCGGG | |

TABLE 21
Table 21 below lists a nucleic acid sequence
(SEQ ID NO: 181) comprising DMR EFC#103.
Each MVP within the DMR is identified as
[CG] with the cytosine being the site
of potential methylation. Also listed are
nucleic acid sequences (SEQ ID NOS: 182
to 192) each comprising the same nucleic
acid sequence as presented in SEQ ID
NO: 181 but wherein each MVP is
individually and separately
identified as [CG].

| | | | Position | Full genomic | |
|---|---|---|---|---|---|
| | Position | | of | sequence | SEQ |
| DMR | with | | marker | with CpG | ID |
| # | primers | Strand | CpGs | highlighted | NO. |
| 103 | chr8: | - | | CTCAGTGTCCT | 181 |
| | 145106092- | | | CCTAAGGCAAG | |
| | 145106211 | | | CACAGATGAGG | |
| | | | | GG[CG][CG] | |
| | | | | [CG]GCTGG | |
| | | | | [CG][CG]CAC | |
| | | | | AGACA[CG]AC | |
| | | | | T[CG]GAGCA | |
| | | | | [CG]AACTAGG | |
| | | | | [CG]C[CG]TA | |
| | | | | GCTG[CG]TCC | |
| | | | | CCAGAACCGGG | |
| | | | | AGACTTAAGGC | |
| | | | | ATC | |
| 103 | chr8: | - | 145106127 | CTCAGTGTCCTC | 182 |
| | 145106092- | | | CTAAGGCAAGCA | |
| | 145106211 | | | CAGATGAGGGG | |
| | | | | [CG]CGCGGCTG | |
| | | | | GCGCGCACAGAC | |
| | | | | ACGACTCGGAGC | |
| | | | | ACGAACTAGGCG | |
| | | | | CCGTAGCTGCGT | |
| | | | | CCCCAGAACCGG | |
| | | | | GAGACTTAAGGC | |
| | | | | ATC | |
| 103 | chr8: | - | 145106129 | CTCAGTGTCCTC | 183 |
| | 145106092- | | | CTAAGGCAAGCA | |
| | 145106211 | | | CAGATGAGGGGC | |
| | | | | G[CG]CGGCTGG | |
| | | | | CGCGCACAGACA | |
| | | | | CGACTCGGAGCA | |
| | | | | CGAACTAGGCGC | |
| | | | | CGTAGCTGCGTC | |
| | | | | CCCAGAACCGGG | |
| | | | | AGACTTAAGGCA | |
| | | | | TC | |
| 103 | chr8: | - | 145106131 | CTCAGTGTCCTC | 184 |
| | 145106092- | | | CTAAGGCAAGCA | |
| | 145106211 | | | CAGATGAGGGGC | |
| | | | | GCG[CG]GCTGG | |
| | | | | CGCGCACAGACA | |
| | | | | CGACTCGGAGCA | |
| | | | | CGAACTAGGCGC | |
| | | | | CGTAGCTGCGTC | |
| | | | | CCCAGAACCGGG | |
| | | | | AGACTTAAGGCA | |
| | | | | TC | |
| 103 | chr8: | - | 145106138 | CTCAGTGTCCTC | 185 |
| | 145106092- | | | CTAAGGCAAGCA | |
| | 145106211 | | | CAGATGAGGGGC | |
| | | | | GCGCGGCTGG | |
| | | | | [CG]CGCACAGA | |
| | | | | CACGACTCGGAG | |
| | | | | CACGAACTAGGC | |
| | | | | GCCGTAGCTGCG | |
| | | | | TCCCCAGAACCG | |
| | | | | GGAGACTTAAGG | |
| | | | | CATC | |
| 103 | chr8: | - | 145106140 | CTCAGTGTCCT | 186 |
| | 145106092- | | | CCTAAGGCAAG | |
| | 145106211 | | | CACAGATGAGG | |
| | | | | GGCGCGCGGCT | |
| | | | | GGCG[CG]CAC | |
| | | | | AGACACGACTC | |
| | | | | GGAGCACGAAC | |
| | | | | TAGGCGCCGTA | |
| | | | | GCTGCGTCCCC | |
| | | | | AGAACCGGGAG | |
| | | | | ACTTAAGGCAT | |
| | | | | C | |
| 103 | chr8: | - | 145106150 | CTCAGTGTCCT | 187 |
| | 145106092- | | | CCTAAGGCAAG | |
| | 145106211 | | | CACAGATGAGG | |
| | | | | GGCGCGCGGCT | |
| | | | | GGCGCGCACAG | |
| | | | | ACA[CG]ACTC | |
| | | | | GGAGCACGAAC | |
| | | | | TAGGCGCCGTA | |
| | | | | GCTGCGTCCCC | |
| | | | | AGAACCGGGAG | |
| | | | | ACTTAAGGCAT | |
| | | | | C | |
| 103 | chr8: | - | 145106155 | CTCAGTGTCCT | 188 |
| | 145106092- | | | CCTAAGGCAAG | |
| | 145106211 | | | CACAGATGAGG | |
| | | | | GGCGCGCGGCT | |
| | | | | GGCGCGCACAG | |
| | | | | ACACGACT | |
| | | | | [CG]GAGCACG | |
| | | | | AACTAGGCGCC | |
| | | | | GTAGCTGCGTC | |
| | | | | CCCAGAACCGG | |
| | | | | GAGACTTAAGG | |
| | | | | CATC | |
| 103 | chr8: | - | 145106162 | CTCAGTGTCCT | 189 |
| | 145106092- | | | CCTAAGGCAAG | |
| | 145106211 | | | CACAGATGAGG | |
| | | | | GGCGCGCGGCT | |
| | | | | GGCGCGCACAG | |
| | | | | ACACGACTCGG | |
| | | | | AGCA[CG]AAC | |
| | | | | TAGGCGCCGTA | |
| | | | | GCTGCGTCCCC | |
| | | | | AGAACCGGGAG | |
| | | | | ACTTAAGGCAT | |
| | | | | C | |
| 103 | chr8: | - | 145106171 | CTCAGTGTCCT | 190 |
| | 145106092- | | | CCTAAGGCAAG | |
| | 145106211 | | | CACAGATGAGG | |
| | | | | GGCGCGCGGCT | |
| | | | | GGCGCGCACAG | |
| | | | | ACACGACTCGG | |
| | | | | AGCACGAACTA | |
| | | | | GG[CG]CCGTA | |
| | | | | GCTGCGTCCCC | |
| | | | | AGAACCGGGAG | |
| | | | | ACTTAAGGCAT | |
| | | | | C | |
| 103 | chr8: | - | 145106174 | CTCAGTGTCCT | 191 |
| | 145106092- | | | CCTAAGGCAAG | |
| | 145106211 | | | CACAGATGAGG | |
| | | | | GGCGCGCGGCT | |
| | | | | GGCGCGCACAG | |
| | | | | ACACGACTCGG | |
| | | | | AGCACGAACTA | |
| | | | | GGCGC[CG]TA | |
| | | | | GCTGCGTCCCC | |
| | | | | AGAACCGGGAG | |
| | | | | ACTTAAGGCAT | |
| | | | | C | |
| 103 | chr8: | - | 145106182 | CTCAGTGTCCT | 192 |
| | 145106092- | | | CCTAAGGCAAG | |
| | 145106211 | | | CACAGATGAGG | |
| | | | | GGCGCGCGGCT | |
| | | | | GGCGCGCACAG | |
| | | | | ACACGACTCGG | |
| | | | | AGCACGAACTA | |
| | | | | GGCGCCGTAGC | |
| | | | | TG[CG]TCCCC | |
| | | | | AGAACCGGGAG | |
| | | | | ACTTAAGGCAT | |
| | | | | C | |

TABLE 22
Table 22 below lists a nucleic acid sequence (SEQ ID NO: 193)
comprising DMR EFC#109. Each MVP within the DMR is identified as [CG]
with the cytosine being the site of potential methylation.
Also listed are nucleic acid sequences (SEQ ID NOS: 194 to 200)
each comprising the same nucleic acid sequence as presented in
SEQ ID NO: 193 but wherein each MVP is individually and separately
identified as [CG].

| | Position | | Position | | SEQ |
|---|---|---|---|---|---|
| DMR | with | | of marker | Full genomic sequence | ID |
| # | primers | Strand | CpGs | with CpG highlighted | NO. |
| 109 | chr9: | - | | CCTCCTCCTGCACCTCCTGCAGCC[CG]GCT | 193 |
| | 139553853- | | | CC[CG][CG]GC[CG][CG]CCTGGTGCCCC | |
| | 139553951 | | | TCTGTCT[CG][CG]CCACCTGAGATGCCCA | |
| | | | | GGCTGGCCTCTGCCAGGGGC | |
| 109 | chr9: | - | 139553877 | CCTCCTCCTGCACCTCCTGCAGCC[CG]GCT | 194 |
| | 139553853- | | | CCCGCGGCCGCGCCTGGTGCCCCTCTGTCTC | |
| | 139553951 | | | GCGCCACCTGAGATGCCCAGGCTGGCCTCTG | |
| | | | | CCAGGGGC | |
| 109 | chr9: | - | 139553884 | CCTCCTCCTGCACCTCCTGCAGCCCGGCTCC | 195 |
| | 139553853- | | | [CG]CGGCCGCGCCTGGTGCCCCTCTGTCTC | |
| | 139553951 | | | GCGCCACCTGAGATGCCCAGGCTGGCCTCTG | |
| | | | | CCAGGGGC | |
| 109 | chr9: | - | 139553886 | CCTCCTCCTGCACCTCCTGCAGCCCGGCTCC | 196 |
| | 139553853- | | | CG[CG]GCCGCGCCTGGTGCCCCTCTGTCTC | |
| | 139553951 | | | GCGCCACCTGAGATGCCCAGGCTGGCCTCTG | |
| | | | | CCAGGGGC | |
| 109 | chr9: | - | 139553890 | CCTCCTCCTGCACCTCCTGCAGCCCGGCTCC | 197 |
| | 139553853- | | | CGCGGC[CG]CGCCTGGTGCCCCTCTGTCTC | |
| | 139553951 | | | GCGCCACCTGAGATGCCCAGGCTGGCCTCTG | |
| | | | | CCAGGGGC | |
| 109 | chr9: | - | 139553892 | CCTCCTCCTGCACCTCCTGCAGCCCGGCTCC | 198 |
| | 139553853- | | | CGCGGCCG[CG]CCTGGTGCCCCTCTGTCTC | |
| | 139553951 | | | GCGCCACCTGAGATGCCCAGGCTGGCCTCTG | |
| | | | | CCAGGGGC | |
| 109 | chr9: | - | 139553912 | CCTCCTCCTGCACCTCCTGCAGCCCGGCTCC | 199 |
| | 139553853- | | | CGCGGCCGCGCCTGGTGCCCCTCTGTCT | |
| | 139553951 | | | [CG]CGCCACCTGAGATGCCCAGGCTGGCCT | |
| | | | | CTGCCAGGGGC | |
| 109 | chr9: | - | 139553914 | CCTCCTCCTGCACCTCCTGCAGCCCGGCTCC | 200 |
| | 139553853- | | | CGCGGCCGCGCCTGGTGCCCCTCTGTCTCG | |
| | 139553951 | | | [CG]CCACCTGAGATGCCCAGGCTGGCCTCT | |
| | | | | GCCAGGGGC | |

TABLE 23
Table 23 below lists a nucleic acid sequence (SEQ ID NO: 201)
comprising DMR EFC#110. Each MVP within the DMR is identified as [CG]
with the cytosine being the site of potential methylation.
Also listed are nucleic acid sequences (SEQ ID NOS: 202 to 212)
each comprising the same nucleic acid sequence as presented in
SEQ ID NO: 201 but wherein each MVP is individually and separately
identified as [CG].

| | Position | | Position | | SEQ |
|---|---|---|---|---|---|
| DMR | with | | of marker | Full genomic sequence | ID |
| # | primers | Strand | CpGs | with CpG highlighted | NO. |
| 110 | chr11: | + | | CAGTTCCCGAAGAAAAGATGGGTTTGGGG | 201 |
| | 62693570- | | | [CG]GT[CG][CG]AAAG[CG]G[CG]CCT | |
| | 62693687 | | | [CG][CG]TGTTTTCCTGC[CG]TTCC[CG] | |
| | | | | GGTCCTTATAGCC[CG]GC[CG]GAGACTCC | |
| | | | | GCTGAGTTGACTCGGCGCC | |
| 110 | chr11: | + | 62693599 | CAGTTCCCGAAGAAAAGATGGGTTTGGGG | 202 |
| | 62693570- | | | [CG]GTCGCGAAAGCGGCGCCTCGCGTGTTT | |
| | 62693687 | | | TCCTGCCGTTCCCGGGTCCTTATAGCCCGGC | |
| | | | | CGGAGACTCCGCTGAGTTGACTCGGCGCC | |
| 110 | chr11: | + | 62693603 | CAGTTCCCGAAGAAAAGATGGGTTTGGGGCG | 203 |
| | 62693570- | | | GT[CG]CGAAAGCGGCGCCTCGCGTGTTTTC | |
| | 62693687 | | | CTGCCGTTCCCGGGTCCTTATAGCCCGGCCG | |
| | | | | GAGACTCCGCTGAGTTGACTCGGCGCC | |
| 110 | chr11: | + | 62693605 | CAGTTCCCGAAGAAAAGATGGGTTTGGGGCG | 204 |
| | 62693570- | | | GTCG[CG]AAAGCGGCGCCTCGCGTGTTTTC | |
| | 62693687 | | | CTGCCGTTCCCGGGTCCTTATAGCCCGGCCG | |
| | | | | GAGACTCCGCTGAGTTGACTCGGCGCC | |
| 110 | chr11: | + | 62693611 | CAGTTCCCGAAGAAAAGATGGGTTTGGGGCG | 205 |
| | 62693570- | | | GTCGCGAAAG[CG]GCGCCTCGCGTGTTTTC | |
| | 62693687 | | | CTGCCGTTCCCGGGTCCTTATAGCCCGGCCG | |
| | | | | GAGACTCCGCTGAGTTGACTCGGCGCC | |
| 110 | chr11: | + | 62693614 | CAGTTCCCGAAGAAAAGATGGGTTTGGGGCG | 206 |
| | 62693570- | | | GTCGCGAAAGCGG[CG]CCTCGCGTGTTTTC | |
| | 62693687 | | | CTGCCGTTCCCGGGTCCTTATAGCCCGGCCG | |
| | | | | GAGACTCCGCTGAGTTGACTCGGCGCC | |
| 110 | chr11: | + | 62693619 | CAGTTCCCGAAGAAAAGATGGGTTTGGGGCG | 207 |
| | 62693570- | | | GTCGCGAAAGCGGCGCCT[CG]CGTGTTTTC | |
| | 62693687 | | | CTGCCGTTCCCGGGTCCTTATAGCCCGGCCG | |
| | | | | GAGACTCCGCTGAGTTGACTCGGCGCC | |
| 110 | chr11: | + | 62693621 | CAGTTCCCGAAGAAAAGATGGGTTTGGGGCG | 208 |
| | 62693570- | | | GTCGCGAAAGCGGCGCCTCG[CG]TGTTTTC | |
| | 62693687 | | | CTGCCGTTCCCGGGTCCTTATAGCCCGGCCG | |
| | | | | GAGACTCCGCTGAGTTGACTCGGCGCC | |
| 110 | chr11: | + | 62693634 | CAGTTCCCGAAGAAAAGATGGGTTTGGGGCG | 209 |
| | 62693570- | | | GTCGCGAAAGCGGCGCCTCGCGTGTTTTCCT | |
| | 62693687 | | | GC[CG]TTCCCGGGTCCTTATAGCCCGGCCG | |
| | | | | GAGACTCCGCTGAGTTGACTCGGCGCC | |
| 110 | chr11: | + | 62693640 | CAGTTCCCGAAGAAAAGATGGGTTTGGGGCG | 210 |
| | 62693570- | | | GTCGCGAAAGCGGCGCCTCGCGTGTTTTCCT | |
| | 62693687 | | | GCCGTTCC[CG]GGTCCTTATAGCCCGGCCG | |
| | | | | GAGACTCCGCTGAGTTGACTCGGCGCC | |
| 110 | chr11: | + | 62693655 | CAGTTCCCGAAGAAAAGATGGGTTTGGGGCG | 211 |
| | 62693570- | | | GTCGCGAAAGCGGCGCCTCGCGTGTTTTCCT | |
| | 62693687 | | | GCCGTTCCCGGGTCCTTATAGCC[CG]GCCG | |
| | | | | GAGACTCCGCTGAGTTGACTCGGCGCC | |
| 110 | chr11: | + | 62693659 | CAGTTCCCGAAGAAAAGATGGGTTTGGGGCG | 212 |
| | 62693570- | | | GTCGCGAAAGCGGCGCCTCGCGTGTTTTCCT | |
| | 62693687 | | | GCCGTTCCCGGGTCCTTATAGCCCGGC[CG] | |
| | | | | GAGACTCCGCTGAGTTGACTCGGCGCC | |

TABLE 24
Table 24 below lists a nucleic acid sequence (SEQ ID NO: 213)
comprising DMR EFC#112. Each MVP within the DMR is identified as [CG]
with the cytosine being the site of potential methylation.
Also listed are nucleic acid sequences (SEQ ID NOS: 214 to 223)
each comprising the same nucleic acid sequence as presented in
SEQ ID NO: 213 but wherein each MVP is individually and separately
identified as [CG].

| | Position | | Position | | SEQ |
|---|---|---|---|---|---|
| DMR | with | | of marker | Full genomic sequence | ID |
| # | primers | Strand | CpGs | with CpG highlighted | NO. |
| 112 | chr12: | + | | GGAGCCGCTATGGACGCTGAGCTCCTCAGCT | 213 |
| | 49390739- | | | T[CG]TC[CG]TGTC[CG]AGTCAGGGGCTG | |
| | 49390861 | | | TGTGG[CG]G[CG]GATA[CG]GGACA[CG] | |
| | | | | GCTTCT[CG]CAGGGCCC[CG]G[CG]TAGG | |
| | | | | GCCCTGGGGTCCGCGCCCA | |
| 112 | chr12: | + | 49390771 | GGAGCCGCTATGGACGCTGAGCTCCTCAGCT | 214 |
| | 49390739- | | | T[CG]TCCGTGTCCGAGTCAGGGGCTGTGTG | |
| | 49390861 | | | GCGGCGGATACGGGACACGGCTTCTCGCAGG | |
| | | | | GCCCCGGCGTAGGGCCCTGGGGTCCGCGCCC | |
| | | | | A | |
| 112 | chr12: | + | 49390775 | GGAGCCGCTATGGACGCTGAGCTCCTCAGCT | 215 |
| | 49390739- | | | TCGTC[CG]TGTCCGAGTCAGGGGCTGTGTG | |
| | 49390861 | | | GCGGCGGATACGGGACACGGCTTCTCGCAGG | |
| | | | | GCCCCGGCGTAGGGCCCTGGGGTCCGCGCCC | |
| | | | | A | |
| 112 | chr12: | + | 49390781 | GGAGCCGCTATGGACGCTGAGCTCCTCAGCT | 216 |
| | 49390739- | | | TCGTCCGTGTC[CG]AGTCAGGGGCTGTGTG | |
| | 49390861 | | | GCGGCGGATACGGGACACGGCTTCTCGCAGG | |
| | | | | GCCCCGGCGTAGGGCCCTGGGGTCCGCGCCC | |
| | | | | A | |
| 112 | chr12: | + | 49390800 | GGAGCCGCTATGGACGCTGAGCTCCTCAGCT | 217 |
| | 49390739- | | | TCGTCCGTGTCCGAGTCAGGGGCTGTGTGG | |
| | 49390861 | | | [CG]GCGGATACGGGACACGGCTTCTCGCAG | |
| | | | | GGCCCCGGCGTAGGGCCCTGGGGTCCGCGCC | |
| | | | | CA | |
| 112 | chr12: | + | 49390803 | GGAGCCGCTATGGACGCTGAGCTCCTCAGCT | 218 |
| | 49390739- | | | TCGTCCGTGTCCGAGTCAGGGGCTGTGTGGC | |
| | 49390861 | | | GG[CG]GATACGGGACACGGCTTCTCGCAGG | |
| | | | | GCCCCGGCGTAGGGCCCTGGGGTCCGCGCCC | |
| | | | | A | |
| 112 | chr12: | + | 49390809 | GGAGCCGCTATGGACGCTGAGCTCCTCAGCT | 219 |
| | 49390739- | | | TCGTCCGTGTCCGAGTCAGGGGCTGTGTGGC | |
| | 49390861 | | | GGCGGATA[CG]GGACACGGCTTCTCGCAGG | |
| | | | | GCCCCGGCGTAGGGCCCTGGGGTCCGCGCCC | |
| | | | | A | |
| 112 | chr12: | + | 49390816 | GGAGCCGCTATGGACGCTGAGCTCCTCAGCT | 220 |
| | 49390739- | | | TCGTCCGTGTCCGAGTCAGGGGCTGTGTGGC | |
| | 49390861 | | | GGCGGATACGGGACA[CG]GCTTCTCGCAGG | |
| | | | | GCCCCGGCGTAGGGCCCTGGGGTCCGCGCCC | |
| | | | | A | |
| 112 | chr12: | + | 49390824 | GGAGCCGCTATGGACGCTGAGCTCCTCAGCT | 221 |
| | 49390739- | | | TCGTCCGTGTCCGAGTCAGGGGCTGTGTGGC | |
| | 49390861 | | | GGCGGATACGGGACACGGCTTCT[CG]CAGG | |
| | | | | GCCCCGGCGTAGGGCCCTGGGGTCCGCGCCC | |
| | | | | A | |
| 112 | chr12: | + | 49390834 | GGAGCCGCTATGGACGCTGAGCTCCTCAGCT | 222 |
| | 49390739- | | | TCGTCCGTGTCCGAGTCAGGGGCTGTGTGGC | |
| | 49390861 | | | GGCGGATACGGGACACGGCTTCTCGCAGGGC | |
| | | | | CC[CG]GCGTAGGGCCCTGGGGTCCGCGCCC | |
| | | | | A | |
| 112 | chr12: | + | 49390837 | GGAGCCGCTATGGACGCTGAGCTCCTCAGCT | 223 |
| | 49390739- | | | TCGTCCGTGTCCGAGTCAGGGGCTGTGTGGC | |
| | 49390861 | | | GGCGGATACGGGACACGGCTTCTCGCAGGGC | |
| | | | | CCCGG[CG]TAGGGCCCTGGGGTCCGCGCCC | |
| | | | | A | |

TABLE 25
Table 25 below lists a nucleic acid sequence (SEQ ID NO: 224)
comprising DMR EFC#113. Each MVP within the DMR is identified as [CG]
with the cytosine being the site of potential methylation.
Also listed are nucleic acid sequences (SEQ ID NOS: 225 to 234)
each comprising the same nucleic acid sequence as presented in
SEQ ID NO: 224 but wherein each MVP is individually and separately
identified as [CG].

| | Position | | Position | | SEQ |
|---|---|---|---|---|---|
| DMR | with | | of marker | Full genomic sequence | ID |
| # | primers | Strand | CpGs | with CpG highlighted | NO. |
| 113 | chr12: | - | | CGGGGCTTCTGTGTCGCTTCCATCAGAGGAG | 224 |
| | 49390712- | | | C[CG]CTATGGA[CG]CTGAGCTCCTCAGCT | |
| | 49390852 | | | T[CG]TC[CG]TGTC[CG]AGTCAGGGGCTG | |
| | | | | TGTGG[CG]G[CG]GATA[CG]GGACA[CG] | |
| | | | | GCTTCT[CG]CAGGGCCCCGGCGTAGGGCCC | |
| | | | | TGGGGT | |
| 113 | chr12: | - | 49390744 | CGGGGCTTCTGTGTCGCTTCCATCAGAGGAG | 225 |
| | 49390712- | | | C[CG]CTATGGACGCTGAGCTCCTCAGCTTC | |
| | 49390852 | | | GTCCGTGTCCGAGTCAGGGGCTGTGTGGCGG | |
| | | | | CGGATACGGGACACGGCTTCTCGCAGGGCCC | |
| | | | | CGGCGTAGGGCCCTGGGGT | |
| 113 | chr12: | - | 49390753 | CGGGGCTTCTGTGTCGCTTCCATCAGAGGAG | 226 |
| | 49390712- | | | CCGCTATGGA[CG]CTGAGCTCCTCAGCTTC | |
| | 49390852 | | | GTCCGTGTCCGAGTCAGGGGCTGTGTGGCGG | |
| | | | | CGGATACGGGACACGGCTTCTCGCAGGGCCC | |
| | | | | CGGCGTAGGGCCCTGGGGT | |
| 113 | chr12: | - | 49390771 | CGGGGCTTCTGTGTCGCTTCCATCAGAGGAG | 227 |
| | 49390712- | | | CCGCTATGGACGCTGAGCTCCTCAGCTT | |
| | 49390852 | | | [CG]TCCGTGTCCGAGTCAGGGGCTGTGTGG | |
| | | | | CGGCGGATACGGGACACGGCTTCTCGCAGGG | |
| | | | | CCCCGGCGTAGGGCCCTGGGGT | |
| 113 | chr12: | - | 49390775 | CGGGGCTTCTGTGTCGCTTCCATCAGAGGAG | 228 |
| | 49390712- | | | CCGCTATGGACGCTGAGCTCCTCAGCTTCGT | |
| | 49390852 | | | C[CG]TGTCCGAGTCAGGGGCTGTGTGGCGG | |
| | | | | CGGATACGGGACACGGCTTCTCGCAGGGCCC | |
| | | | | CGGCGTAGGGCCCTGGGGT | |
| 113 | chr12: | - | 49390781 | CGGGGCTTCTGTGTCGCTTCCATCAGAGGAG | 229 |
| | 49390712- | | | CCGCTATGGACGCTGAGCTCCTCAGCTTCGT | |
| | 49390852 | | | CCGTGTC[CG]AGTCAGGGGCTGTGTGGCGG | |
| | | | | CGGATACGGGACACGGCTTCTCGCAGGGCCC | |
| | | | | CGGCGTAGGGCCCTGGGGT | |
| 113 | chr12: | - | 49390800 | CGGGGCTTCTGTGTCGCTTCCATCAGAGGAG | 230 |
| | 49390712- | | | CCGCTATGGACGCTGAGCTCCTCAGCTTCGT | |
| | 49390852 | | | CCGTGTCCGAGTCAGGGGCTGTGTGG[CG]G | |
| | | | | CGGATACGGGACACGGCTTCTCGCAGGGCCC | |
| | | | | CGGCGTAGGGCCCTGGGGT | |
| 113 | chr12: | - | 49390803 | CGGGGCTTCTGTGTCGCTTCCATCAGAGGAG | 231 |
| | 49390712- | | | CCGCTATGGACGCTGAGCTCCTCAGCTTCGT | |
| | 49390852 | | | CCGTGTCCGAGTCAGGGGCTGTGTGGCGG | |
| | | | | [CG]GATACGGGACACGGCTTCTCGCAGGGC | |
| | | | | CCCGGCGTAGGGCCCTGGGGT | |
| 113 | chr12: | - | 49390809 | CGGGGCTTCTGTGTCGCTTCCATCAGAGGAG | 232 |
| | 49390712- | | | CCGCTATGGACGCTGAGCTCCTCAGCTTCGT | |
| | 49390852 | | | CCGTGTCCGAGTCAGGGGCTGTGTGGCGGCG | |
| | | | | GATA[CG]GGACACGGCTTCTCGCAGGGCCC | |
| | | | | CGGCGTAGGGCCCTGGGGT | |
| 113 | chr12: | - | 49390816 | CGGGGCTTCTGTGTCGCTTCCATCAGAGGAG | 233 |
| | 49390712- | | | CCGCTATGGACGCTGAGCTCCTCAGCTTCGT | |
| | 49390852 | | | CCGTGTCCGAGTCAGGGGCTGTGTGGCGGCG | |
| | | | | GATACGGGACA[CG]GCTTCTCGCAGGGCCC | |
| | | | | CGGCGTAGGGCCCTGGGGT | |
| 113 | chr12: | - | 49390824 | CGGGGCTTCTGTGTCGCTTCCATCAGAGGAG | 234 |
| | 49390712- | | | CCGCTATGGACGCTGAGCTCCTCAGCTTCGT | |
| | 49390852 | | | CCGTGTCCGAGTCAGGGGCTGTGTGGCGGCG | |
| | | | | GATACGGGACACGGCTTCT[CG]CAGGGCCC | |
| | | | | CGGCGTAGGGCCCTGGGGT | |

TABLE 26
Table 26 below lists a nucleic acid sequence (SEQ ID NO: 235)
comprising DMR EFC#115. Each MVP within the DMR is identified as [CG]
with the cytosine being the site of potential methylation.
Also listed are nucleic acid sequences (SEQ ID NOS: 236 to 241)
each comprising the same nucleic acid sequence as presented in
SEQ ID NO: 235 but wherein each MVP is individually and separately
identified as [CG].

| | Position | | Position | | SEQ |
|---|---|---|---|---|---|
| DMR | with | | of marker | Full genomic sequence | ID |
| # | primers | Strand | CpGs | with CpG highlighted | NO. |
| 115 | chr16: | - | | TGGTTCGGGTTTCTCCGAGTTTTGCTACCAG | 235 |
| | 1271197- | | | C[CG]AGGCTGTG[CG]GGCAACTGGGTCAG | |
| | 1271314 | | | CCTCC[CG]TCAGGAGAGAAGC[CG][CG]T | |
| | | | | CTGTGGGA[CG]AAGACCGGGCACCCGCCAG | |
| | | | | AGAGGG | |
| 115 | chr16: | - | 1271229 | TGGTTCGGGTTTCTCCGAGTTTTGCTACCAG | 236 |
| | 1271197- | | | C[CG]AGGCTGTGCGGGCAACTGGGTCAGCC | |
| | 1271314 | | | TCCCGTCAGGAGAGAAGCCGCGTCTGTGGGA | |
| | | | | CGAAGACCGGGCACCCGCCAGAGAGGG | |
| 115 | chr16: | - | 1271239 | TGGTTCGGGTTTCTCCGAGTTTTGCTACCAG | 237 |
| | 1271197- | | | CCGAGGCTGTG[CG]GGCAACTGGGTCAGCC | |
| | 1271314 | | | TCCCGTCAGGAGAGAAGCCGCGTCTGTGGGA | |
| | | | | CGAAGACCGGGCACCCGCCAGAGAGGG | |
| 115 | chr16: | - | 1271260 | TGGTTCGGGTTTCTCCGAGTTTTGCTACCAG | 238 |
| | 1271197- | | | CCGAGGCTGTGCGGGCAACTGGGTCAGCCTC | |
| | 1271314 | | | C[CG]TCAGGAGAGAAGCCGCGTCTGTGGGA | |
| | | | | CGAAGACCGGGCACCCGCCAGAGAGGG | |
| 115 | chr16: | - | 1271275 | TGGTTCGGGTTTCTCCGAGTTTTGCTACCAG | 239 |
| | 1271197- | | | CCGAGGCTGTGCGGGCAACTGGGTCAGCCTC | |
| | 1271314 | | | CCGTCAGGAGAGAAGC[CG]CGTCTGTGGGA | |
| | | | | CGAAGACCGGGCACCCGCCAGAGAGGG | |
| 115 | chr16: | - | 1271277 | TGGTTCGGGTTTCTCCGAGTTTTGCTACCAG | 240 |
| | 1271197- | | | CCGAGGCTGTGCGGGCAACTGGGTCAGCCTC | |
| | 1271314 | | | CCGTCAGGAGAGAAGCCG[CG]TCTGTGGGA | |
| | | | | CGAAGACCGGGCACCCGCCAGAGAGGG | |
| 115 | chr16: | - | 1271288 | TGGTTCGGGTTTCTCCGAGTTTTGCTACCAG | 241 |
| | 1271197- | | | CCGAGGCTGTGCGGGCAACTGGGTCAGCCTC | |
| | 1271314 | | | CCGTCAGGAGAGAAGCCGCGTCTGTGGGA | |
| | | | | [CG]AAGACCGGGCACCCGCCAGAGAGGG | |

TABLE 27
Table 27 below lists a nucleic acid sequence (SEQ ID NO: 242)
comprising DMR EFC#116. Each MVP within the DMR is identified as [CG]
with the cytosine being the site of potential methylation.
Also listed are nucleic acid sequences (SEQ ID NOS: 243 to 251)
each comprising the same nucleic acid sequence as presented in
SEQ ID NO: 242 but wherein each MVP is individually and separately
identified as [CG].

| | Position | | Position | | SEQ |
|---|---|---|---|---|---|
| DMR | with | | of marker | Full genomic sequence | ID |
| # | primers | Strand | CpGs | with CpG highlighted | NO. |
| 116 | chr17: | - | | CTCATCTCAGAGCGCAGGAAGCAAACC[CG] | 242 |
| | 43037200- | | | C[CG]C[CG][CG]ACCTCTCCCCAGGCTGG | |
| | 43037354 | | | GGTGGGCTGGCAGG[CG]GAGGTGGGCAGTA | |
| | | | | AACAGTCCTATTGTACAAATATATAG[CG] | |
| | | | | [CG]GGCTGGG[CG]GGGG[CG]GTCAACCC | |
| | | | | CGGTTCCCTGGCACGGGGA | |
| 116 | chr17: | - | 43037227 | CTCATCTCAGAGCGCAGGAAGCAAACC[CG] | 243 |
| | 43037200- | | | CCGCCGCGACCTCTCCCCAGGCTGGGGTGGG | |
| | 43037354 | | | CTGGCAGGCGGAGGTGGGCAGTAAACAGTCC | |
| | | | | TATTGTACAAATATATAGCGCGGGCTGGGCG | |
| | | | | GGGGCGGTCAACCCCGGTTCCCTGGCACGGG | |
| | | | | GA | |
| 116 | chr17: | - | 43037230 | CTCATCTCAGAGCGCAGGAAGCAAACCCGC | 244 |
| | 43037200- | | | [CG]CCGCGACCTCTCCCCAGGCTGGGGTGG | |
| | 43037354 | | | GCTGGCAGGCGGAGGTGGGCAGTAAACAGTC | |
| | | | | CTATTGTACAAATATATAGCGCGGGCTGGGC | |
| | | | | GGGGGCGGTCAACCCCGGTTCCCTGGCACGG | |
| | | | | GGA | |
| 116 | chr17: | - | 43037233 | CTCATCTCAGAGCGCAGGAAGCAAACCCGCC | 245 |
| | 43037200- | | | GC[CG]CGACCTCTCCCCAGGCTGGGGTGGG | |
| | 43037354 | | | CTGGCAGGCGGAGGTGGGCAGTAAACAGTCC | |
| | | | | TATTGTACAAATATATAGCGCGGGCTGGGCG | |
| | | | | GGGGCGGTCAACCCCGGTTCCCTGGCACGGG | |
| | | | | GA | |
| 116 | chr17: | - | 43037235 | CTCATCTCAGAGCGCAGGAAGCAAACCCGCC | 246 |
| | 43037200- | | | GCCG[CG]ACCTCTCCCCAGGCTGGGGTGGG | |
| | 43037354 | | | CTGGCAGGCGGAGGTGGGCAGTAAACAGTCC | |
| | | | | TATTGTACAAATATATAGCGCGGGCTGGGCG | |
| | | | | GGGGCGGTCAACCCCGGTTCCCTGGCACGGG | |
| | | | | GA | |
| 116 | chr17: | - | 43037268 | CTCATCTCAGAGCGCAGGAAGCAAACCCGCC | 247 |
| | 43037200- | | | GCCGCGACCTCTCCCCAGGCTGGGGTGGGCT | |
| | 43037354 | | | GGCAGG[CG]GAGGTGGGCAGTAAACAGTCC | |
| | | | | TATTGTACAAATATATAGCGCGGGCTGGGCG | |
| | | | | GGGGCGGTCAACCCCGGTTCCCTGGCACGGG | |
| | | | | GA | |
| 116 | chr17: | - | 43037309 | CTCATCTCAGAGCGCAGGAAGCAAACCCGCC | 248 |
| | 43037200- | | | GCCGCGACCTCTCCCCAGGCTGGGGTGGGCT | |
| | 43037354 | | | GGCAGGCGGAGGTGGGCAGTAAACAGTCCTA | |
| | | | | TTGTACAAATATATAG[CG]CGGGCTGGGCG | |
| | | | | GGGGCGGTCAACCCCGGTTCCCTGGCACGGG | |
| | | | | GA | |
| 116 | chr17: | - | 43037311 | CTCATCTCAGAGCGCAGGAAGCAAACCCGCC | 249 |
| | 43037200- | | | GCCGCGACCTCTCCCCAGGCTGGGGTGGGCT | |
| | 43037354 | | | GGCAGGCGGAGGTGGGCAGTAAACAGTCCTA | |
| | | | | TTGTACAAATATATAGCG[CG]GGCTGGGCG | |
| | | | | GGGGCGGTCAACCCCGGTTCCCTGGCACGGG | |
| | | | | GA | |
| 116 | chr17: | - | 43037320 | CTCATCTCAGAGCGCAGGAAGCAAACCCGCC | 250 |
| | 43037200- | | | GCCGCGACCTCTCCCCAGGCTGGGGTGGGCT | |
| | 43037354 | | | GGCAGGCGGAGGTGGGCAGTAAACAGTCCTA | |
| | | | | TTGTACAAATATATAGCGCGGGCTGGG[CG] | |
| | | | | GGGGCGGTCAACCCCGGTTCCCTGGCACGGG | |
| | | | | GA | |
| 116 | chr17: | - | 43037326 | CTCATCTCAGAGCGCAGGAAGCAAACCCGCC | 251 |
| | 43037200- | | | GCCGCGACCTCTCCCCAGGCTGGGGTGGGCT | |
| | 43037354 | | | GGCAGGCGGAGGTGGGCAGTAAACAGTCCTA | |
| | | | | TTGTACAAATATATAGCGCGGGCTGGGCGGG | |
| | | | | GG[CG]GTCAACCCCGGTTCCCTGGCACGGG | |
| | | | | GA | |

TABLE 28
Table 28 below lists a nucleic acid sequence (SEQ ID NO: 252)
comprising DMR EFC#117. Each MVP within the DMR is identified as [CG]
with the cytosine being the site of potential methylation.
Also listed are nucleic acid sequences (SEQ ID NOS: 253 to 259)
each comprising the same nucleic acid sequence as presented in
SEQ ID NO: 252 but wherein each MVP is individually and separately
identified as [CG].

| | Position | | Position | | SEQ |
|---|---|---|---|---|---|
| DMR | with | | of marker | Full genomic sequence | ID |
| # | primers | Strand | CpGs | with CpG highlighted | NO. |
| 117 | chr17: | + | | GCCCCTCCTTGCGACCCCGCAGGC[CG]CCA | 252 |
| | 59532206- | | | CATCTGGGACCAG[CG]GAT[CG]CTTGGT | |
| | 59532307 | | | [CG]CTGGAGC[CG]ATCC[CG]C[CG]GGG | |
| | | | | CCCTAGATATAGTTGGACCCAGCG | |
| 117 | chr17: | + | 59532230 | GCCCCTCCTTGCGACCCCGCAGGC[CG]CCA | 253 |
| | 59532206- | | | CATCTGGGACCAGCGGATCGCTTGGTCGCTG | |
| | 59532307 | | | GAGCCGATCCCGCCGGGGCCCTAGATATAGT | |
| | | | | TGGACCCAGCG | |
| 117 | chr17: | + | 59532248 | GCCCCTCCTTGCGACCCCGCAGGCCGCCACA | 254 |
| | 59532206- | | | TCTGGGACCAG[CG]GATCGCTTGGTCGCTG | |
| | 59532307 | | | GAGCCGATCCCGCCGGGGCCCTAGATATAGT | |
| | | | | TGGACCCAGCG | |
| 117 | chr17: | + | 59532253 | GCCCCTCCTTGCGACCCCGCAGGCCGCCACA | 255 |
| | 59532206- | | | TCTGGGACCAGCGGAT[CG]CTTGGTCGCTG | |
| | 59532307 | | | GAGCCGATCCCGCCGGGGCCCTAGATATAGT | |
| | | | | TGGACCCAGCG | |
| 117 | chr17: | + | 59532261 | GCCCCTCCTTGCGACCCCGCAGGCCGCCACA | 256 |
| | 59532206- | | | TCTGGGACCAGCGGATCGCTTGGT[CG]CTG | |
| | 59532307 | | | GAGCCGATCCCGCCGGGGCCCTAGATATAGT | |
| | | | | TGGACCCAGCG | |
| 117 | chr17: | + | 59532270 | GCCCCTCCTTGCGACCCCGCAGGCCGCCACA | 257 |
| | 59532206- | | | TCTGGGACCAGCGGATCGCTTGGTCGCTGGA | |
| | 59532307 | | | GC[CG]ATCCCGCCGGGGCCCTAGATATAGT | |
| | | | | TGGACCCAGCG | |
| 117 | chr17: | + | 59532276 | GCCCCTCCTTGCGACCCCGCAGGCCGCCACA | 258 |
| | 59532206- | | | TCTGGGACCAGCGGATCGCTTGGTCGCTGGA | |
| | 59532307 | | | GCCGATCC[CG]CCGGGGCCCTAGATATAGT | |
| | | | | TGGACCCAGCG | |
| 117 | chr17: | + | 59532279 | GCCCCTCCTTGCGACCCCGCAGGCCGCCACA | 259 |
| | 59532206- | | | TCTGGGACCAGCGGATCGCTTGGTCGCTGGA | |
| | 59532307 | | | GCCGATCCCGC[CG]GGGCCCTAGATATAGT | |
| | | | | TGGACCCAGCG | |

TABLE 29
Table 29 below lists a nucleic acid sequence (SEQ ID NO: 260)
comprising DMR EFC#118. Each MVP within the DMR is identified as [CG]
with the cytosine being the site of potential methylation.
Also listed are nucleic acid sequences (SEQ ID NOS: 261 to 266)
each comprising the same nucleic acid sequence as presented in
SEQ ID NO: 260 but wherein each MVP is individually and separately
identified as [CG].

| | Position | | Position | | SEQ |
|---|---|---|---|---|---|
| DMR | with | | of marker | Full genomic sequence | ID |
| # | primers | Strand | CpGs | with CpG highlighted | NO. |
| 118 | chr17: | - | | CAGGCCGCCACATCTGGGACCAG[CG]GAT | 260 |
| | 59532225- | | | [CG]CTTGGT[CG]CTGGAGC[CG]ATCC | |
| | 59532309 | | | [CG]C[CG]GGGCCCTAGATATAGTTGGACC | |
| | | | | CAGCGCG | |
| 118 | chr17: | - | 59532248 | CAGGCCGCCACATCTGGGACCAG[CG]GATC | 261 |
| | 59532225- | | | GCTTGGTCGCTGGAGCCGATCCCGCCGGGGC | |
| | 59532309 | | | CCTAGATATAGTTGGACCCAGCGCG | |
| 118 | chr17: | - | 59532253 | CAGGCCGCCACATCTGGGACCAGCGGAT | 262 |
| | 59532225- | | | [CG]CTTGGTCGCTGGAGCCGATCCCGCCGG | |
| | 59532309 | | | GGCCCTAGATATAGTTGGACCCAGCGCG | |
| 118 | chr17: | - | 59532261 | CAGGCCGCCACATCTGGGACCAGCGGATCGC | 263 |
| | 59532225- | | | TTGGT[CG]CTGGAGCCGATCCCGCCGGGGC | |
| | 59532309 | | | CCTAGATATAGTTGGACCCAGCGCG | |
| 118 | chr17: | - | 59532270 | CAGGCCGCCACATCTGGGACCAGCGGATCGC | 264 |
| | 59532225- | | | TTGGTCGCTGGAGC[CG]ATCCCGCCGGGGC | |
| | 59532309 | | | CCTAGATATAGTTGGACCCAGCGCG | |
| 118 | chr17: | - | 59532276 | CAGGCCGCCACATCTGGGACCAGCGGATCGC | 265 |
| | 59532225- | | | TTGGTCGCTGGAGCCGATCC[CG]CCGGGGC | |
| | 59532309 | | | CCTAGATATAGTTGGACCCAGCGCG | |
| 118 | chr17: | - | 59532279 | CAGGCCGCCACATCTGGGACCAGCGGATCGC | 266 |
| | 59532225- | | | TTGGTCGCTGGAGCCGATCCCGC[CG]GGGC | |
| | 59532309 | | | CCTAGATATAGTTGGACCCAGCGCG | |

TABLE 30
Table 30 below lists a nucleic acid sequence (SEQ ID NO: 267)
comprising DMR EFC#119. Each MVP within the DMR is identified as [CG]
with the cytosine being the site of potential methylation.
Also listed are nucleic acid sequences (SEQ ID NOS: 268 to 277)
each comprising the same nucleic acid sequence as presented in
SEQ ID NO: 267 but wherein each MVP is individually and separately
identified as [CG].

| | Position | | Position | | SEQ |
|---|---|---|---|---|---|
| DMR | with | | of marker | Full genomic sequence | ID |
| # | primers | Strand | CpGs | with CpG highlighted | NO. |
| 119 | chr19: | + | | CCGGTACAGGTGCGGCTGCAGGACCT[CG] | 267 |
| | 17439718- | | | [CG]CA[CG]TTCTGGAGGAACTGG[CG]GG | |
| | 17439872 | | | TGATCAGCAG[CG]TGGCCAGCATCTGGGGG | |
| | | | | CAGGAAGGGGAAGGAGAGAGG[CG][CG]TG | |
| | | | | GGGGGCAAG[CG]GGG[CG]C[CG]GGATCG | |
| | | | | GGGGACTCACCCTCCCTGGGC | |
| 119 | chr19: | + | 17439744 | CCGGTACAGGTGCGGCTGCAGGACCT[CG]C | 268 |
| | 17439718- | | | GCACGTTCTGGAGGAACTGGCGGGTGATCAG | |
| | 17439872 | | | CAGCGTGGCCAGCATCTGGGGGCAGGAAGGG | |
| | | | | GAAGGAGAGAGGCGCGTGGGGGGCAAGCGGG | |
| | | | | GCGCCGGGATCGGGGGACTCACCCTCCCTGG | |
| | | | | GC | |
| 119 | chr19: | + | 17439746 | CCGGTACAGGTGCGGCTGCAGGACCTCG | 269 |
| | 17439718- | | | [CG]CACGTTCTGGAGGAACTGGCGGGTGAT | |
| | 17439872 | | | CAGCAGCGTGGCCAGCATCTGGGGGCAGGAA | |
| | | | | GGGGAAGGAGAGAGGCGCGTGGGGGGCAAGC | |
| | | | | GGGGCGCCGGGATCGGGGGACTCACCCTCCC | |
| | | | | TGGGC | |
| 119 | chr19: | + | 17439750 | CCGGTACAGGTGCGGCTGCAGGACCTCGCGC | 270 |
| | 17439718- | | | A[CG]TTCTGGAGGAACTGGCGGGTGATCAG | |
| | 17439872 | | | CAGCGTGGCCAGCATCTGGGGGCAGGAAGGG | |
| | | | | GAAGGAGAGAGGCGCGTGGGGGGCAAGCGGG | |
| | | | | GCGCCGGGATCGGGGGACTCACCCTCCCTGG | |
| | | | | GC | |
| 119 | Chr19: | + | 17439767 | CCGGTACAGGTGCGGCTGCAGGACCTCGCGC | 271 |
| | 17439718- | | | ACGTTCTGGAGGAACTGG[CG]GGTGATCAG | |
| | 17439872 | | | CAGCGTGGCCAGCATCTGGGGGCAGGAAGGG | |
| | | | | GAAGGAGAGAGGCGCGTGGGGGGCAAGCGGG | |
| | | | | GCGCCGGGATCGGGGGACTCACCCTCCCTGG | |
| | | | | GC | |
| 119 | chr19: | + | 17439781 | CCGGTACAGGTGCGGCTGCAGGACCTCGCGC | 272 |
| | 17439718- | | | ACGTTCTGGAGGAACTGGCGGGTGATCAGCA | |
| | 17439872 | | | G[CG]TGGCCAGCATCTGGGGGCAGGAAGGG | |
| | | | | GAAGGAGAGAGGCGCGTGGGGGGCAAGCGGG | |
| | | | | GCGCCGGGATCGGGGGACTCACCCTCCCTGG | |
| | | | | GC | |
| 119 | chr19: | + | 17439821 | CCGGTACAGGTGCGGCTGCAGGACCTCGCGC | 273 |
| | 17439718- | | | ACGTTCTGGAGGAACTGGCGGGTGATCAGCA | |
| | 17439872 | | | GCGTGGCCAGCATCTGGGGGCAGGAAGGGGA | |
| | | | | AGGAGAGAGG[CG]CGTGGGGGGCAAGCGGG | |
| | | | | GCGCCGGGATCGGGGGACTCACCCTCCCTGG | |
| | | | | GC | |
| 119 | chr19: | + | 17439823 | CCGGTACAGGTGCGGCTGCAGGACCTCGCGC | 274 |
| | 17439718- | | | ACGTTCTGGAGGAACTGGCGGGTGATCAGCA | |
| | 17439872 | | | GCGTGGCCAGCATCTGGGGGCAGGAAGGGGA | |
| | | | | AGGAGAGAGGCG[CG]TGGGGGGCAAGCGGG | |
| | | | | GCGCCGGGATCGGGGGACTCACCCTCCCTGG | |
| | | | | GC | |
| 119 | chr19: | + | 17439836 | CCGGTACAGGTGCGGCTGCAGGACCTCGCGC | 275 |
| | 17439718- | | | ACGTTCTGGAGGAACTGGCGGGTGATCAGCA | |
| | 17439872 | | | GCGTGGCCAGCATCTGGGGGCAGGAAGGGGA | |
| | | | | AGGAGAGAGGCGCGTGGGGGGCAAG[CG]GG | |
| | | | | GCGCCGGGATCGGGGGACTCACCCTCCCTGG | |
| | | | | GC | |
| 119 | chr19: | + | 17439841 | CCGGTACAGGTGCGGCTGCAGGACCTCGCGC | 276 |
| | 17439718- | | | ACGTTCTGGAGGAACTGGCGGGTGATCAGCA | |
| | 17439872 | | | GCGTGGCCAGCATCTGGGGGCAGGAAGGGGA | |
| | | | | AGGAGAGAGGCGCGTGGGGGGCAAGCGGGG | |
| | | | | [CG]CCGGGATCGGGGGACTCACCCTCCCTG | |
| | | | | GGC | |
| 119 | chr19: | + | 17439844 | CCGGTACAGGTGCGGCTGCAGGACCTCGCGC | 277 |
| | 17439718- | | | ACGTTCTGGAGGAACTGGCGGGTGATCAGCA | |
| | 17439872 | | | GCGTGGCCAGCATCTGGGGGCAGGAAGGGGA | |
| | | | | AGGAGAGAGGCGCGTGGGGGGCAAGCGGGGC | |
| | | | | GC[CG]GGATCGGGGGACTCACCCTCCCTGG | |
| | | | | GC | |

TABLE 31
Table 31 below lists a nucleic acid sequence (SEQ ID NO: 278)
comprising DMR EFC#120. Each MVP within the DMR is identified as [CG]
with the cytosine being the site of potential methylation.
Also listed are nucleic acid sequences (SEQ ID NOS: 279 to 284)
each comprising the same nucleic acid sequence as presented in
SEQ ID NO: 278 but wherein each MVP is individually and separately
identified as [CG].

| | Position | | Position | | SEQ |
|---|---|---|---|---|---|
| DMR | with | | of marker | Full genomic sequence | ID |
| # | primers | Strand | CpGs | with CpG highlighted | NO. |
| 120 | chr19: | - | | TCAGCAGCGTGGCCAGCATCTGGGGGCAGGA | 278 |
| | 17439774- | | | AGGGGAAGGAGAGAGG[CG][CG]TGGGGGG | |
| | 17439875 | | | CAAG[CG]GGG[CG]C[CG]GGAT[CG]GGG | |
| | | | | GACTCACCCTCCCTGGGCGCC | |
| 120 | chr19: | - | 17439821 | TCAGCAGCGTGGCCAGCATCTGGGGGCAGGA | 279 |
| | 17439774- | | | AGGGGAAGGAGAGAGG[CG]CGTGGGGGGCA | |
| | 17439875 | | | AGCGGGGCGCCGGGATCGGGGGACTCACCCT | |
| | | | | CCCTGGGCGCC | |
| 120 | chr19: | - | 17439823 | TCAGCAGCGTGGCCAGCATCTGGGGGCAGGA | 280 |
| | 17439774- | | | AGGGGAAGGAGAGAGGCG[CG]TGGGGGGCA | |
| | 17439875 | | | AGCGGGGCGCCGGGATCGGGGGACTCACCCT | |
| | | | | CCCTGGGCGCC | |
| 120 | chr19: | - | 17439836 | TCAGCAGCGTGGCCAGCATCTGGGGGCAGGA | 281 |
| | 17439774- | | | AGGGGAAGGAGAGAGGCGCGTGGGGGGCAAG | |
| | 17439875 | | | [CG]GGGCGCCGGGATCGGGGGACTCACCCT | |
| | | | | CCCTGGGCGCC | |
| 120 | chr19: | - | 17439841 | TCAGCAGCGTGGCCAGCATCTGGGGGCAGGA | 282 |
| | 17439774- | | | AGGGGAAGGAGAGAGGCGCGTGGGGGGCAAG | |
| | 17439875 | | | CGGGG[CG]CCGGGATCGGGGGACTCACCCT | |
| | | | | CCCTGGGCGCC | |
| 120 | chr19: | - | 17439844 | TCAGCAGCGTGGCCAGCATCTGGGGGCAGGA | 283 |
| | 17439774- | | | AGGGGAAGGAGAGAGGCGCGTGGGGGGCAAG | |
| | 17439875 | | | CGGGGCGC[CG]GGATCGGGGGACTCACCCT | |
| | | | | CCCTGGGCGCC | |
| 120 | chr19: | - | 17439850 | TCAGCAGCGTGGCCAGCATCTGGGGGCAGGA | 284 |
| | 17439774- | | | AGGGGAAGGAGAGAGGCGCGTGGGGGGCAAG | |
| | 17439875 | | | CGGGGCGCCGGGAT[CG]GGGGACTCACCCT | |
| | | | | CCCTGGGCGCC | |

TABLE 32
Table 32 below lists a nucleic acid sequence (SEQ ID NO: 285)
comprising DMR EFC#121. Each MVP within the DMR is identified as [CG]
with the cytosine being the site of potential methylation.
Also listed are nucleic acid sequences (SEQ ID NOS: 286 to 291)
each comprising the same nucleic acid sequence as presented in
SEQ ID NO: 285 but wherein each MVP is individually and separately
identified as [CG].

| | Position | | Position | | SEQ |
|---|---|---|---|---|---|
| DMR | with | | of marker | Full genomic sequence | ID |
| # | primers | Strand | CpGs | with CpG highlighted | NO. |
| 121 | chrX: | + | | ACTTCCCGGTCGAGCTCGACCAGGGC[CG] | 285 |
| | 152245134- | | | [CG]GTGGCATTATCCTCCTCT[CG]AAA | |
| | 152245286 | | | [CG]CTTTGCCTAGCACTGTAAAGTGTCCCA | |
| | | | | TAGGCCTCAGGGCAGCCT[CG]AGGGACTCT | |
| | | | | TGGAATT[CG]GCATCATCACAGTCCTCCGG | |
| | | | | GATGCCCAGGATG | |
| 121 | chrX: | + | 152245160 | ACTTCCCGGTCGAGCTCGACCAGGGC[CG]C | 286 |
| | 152245134- | | | GGTGGCATTATCCTCCTCTCGAAACGCTTTG | |
| | 152245286 | | | CCTAGCACTGTAAAGTGTCCCATAGGCCTCA | |
| | | | | GGGCAGCCTCGAGGGACTCTTGGAATTCGGC | |
| | | | | ATCATCACAGTCCTCCGGGATGCCCAGGATG | |
| 121 | chrX: | + | 152245162 | ACTTCCCGGTCGAGCTCGACCAGGGCCG | 287 |
| | 152245134- | | | [CG]GTGGCATTATCCTCCTCTCGAAACGCT | |
| | 152245286 | | | TTGCCTAGCACTGTAAAGTGTCCCATAGGCC | |
| | | | | TCAGGGCAGCCTCGAGGGACTCTTGGAATTC | |
| | | | | GGCATCATCACAGTCCTCCGGGATGCCCAGG | |
| | | | | ATG | |
| 121 | chrX: | + | 152245182 | ACTTCCCGGTCGAGCTCGACCAGGGCCGCGG | 288 |
| | 152245134- | | | TGGCATTATCCTCCTCT[CG]AAACGCTTTG | |
| | 152245286 | | | CCTAGCACTGTAAAGTGTCCCATAGGCCTCA | |
| | | | | GGGCAGCCTCGAGGGACTCTTGGAATTCGGC | |
| | | | | ATCATCACAGTCCTCCGGGATGCCCAGGATG | |
| 121 | chrX: | + | 152245187 | ACTTCCCGGTCGAGCTCGACCAGGGCCGCGG | 289 |
| | 152245134- | | | TGGCATTATCCTCCTCTCGAAA[CG]CTTTG | |
| | 152245286 | | | CCTAGCACTGTAAAGTGTCCCATAGGCCTCA | |
| | | | | GGGCAGCCTCGAGGGACTCTTGGAATTCGGC | |
| | | | | ATCATCACAGTCCTCCGGGATGCCCAGGATG | |
| 121 | chrX: | + | 152245234 | ACTTCCCGGTCGAGCTCGACCAGGGCCGCGG | 290 |
| | 152245134- | | | TGGCATTATCCTCCTCTCGAAACGCTTTGCC | |
| | 152245286 | | | TAGCACTGTAAAGTGTCCCATAGGCCTCAGG | |
| | | | | GCAGCCT[CG]AGGGACTCTTGGAATTCGGC | |
| | | | | ATCATCACAGTCCTCCGGGATGCCCAGGATG | |
| 121 | chrX: | + | 152245252 | ACTTCCCGGTCGAGCTCGACCAGGGCCGCGG | 291 |
| | 152245134- | | | TGGCATTATCCTCCTCTCGAAACGCTTTGCC | |
| | 152245286 | | | TAGCACTGTAAAGTGTCCCATAGGCCTCAGG | |
| | | | | GCAGCCTCGAGGGACTCTTGGAATT[CG]GC | |
| | | | | ATCATCACAGTCCTCCGGGATGCCCAGGATG | |

TABLE 33
Table 33 below lists a nucleic acid sequence (SEQ ID NO: 292)
comprising DMR EFC#122. Each MVP within the DMR is identified as [CG]
with the cytosine being the site of potential methylation.
Also listed are nucleic acid sequences (SEQ ID NOS: 293 to 296)
each comprising the same nucleic acid sequence as presented in
SEQ ID NO: 292 but wherein each MVP is individually and separately
identified as [CG].

| | Position | | Position | | SEQ |
|---|---|---|---|---|---|
| DMR | with | | of marker | Full genomic sequence | ID |
| # | primers | Strand | CpGs | with CpG highlighted | NO. |
| 122 | chrX: | - | | CAGGGCCGCGGTGGCATTATCCTCCTCT | 292 |
| | 152245154- | | | [CG]AAA[CG]CTTTGCCTAGCACTGTAAAG | |
| | 152245280 | | | TGTCCCATAGGCCTCAGGGCAGCCT[CG]AG | |
| | | | | GGACTCTTGGAATT[CG]GCATCATCACAGT | |
| | | | | CCTCCGGGATGCCC | |
| 122 | chrX: | - | 152245182 | CAGGGCCGCGGTGGCATTATCCTCCTCT | 293 |
| | 152245154- | | | [CG]AAACGCTTTGCCTAGCACTGTAAAGTG | |
| | 152245280 | | | TCCCATAGGCCTCAGGGCAGCCTCGAGGGAC | |
| | | | | TCTTGGAATTCGGCATCATCACAGTCCTCCG | |
| | | | | GGATGCCC | |
| 122 | chrX: | - | 152245187 | CAGGGCCGCGGTGGCATTATCCTCCTCTCGA | 294 |
| | 152245154- | | | AA[CG]CTTTGCCTAGCACTGTAAAGTGTCC | |
| | 152245280 | | | CATAGGCCTCAGGGCAGCCTCGAGGGACTCT | |
| | | | | TGGAATTCGGCATCATCACAGTCCTCCGGGA | |
| | | | | TGCCC | |
| 122 | chrX: | - | 152245234 | CAGGGCCGCGGTGGCATTATCCTCCTCTCGA | 295 |
| | 152245154- | | | AACGCTTTGCCTAGCACTGTAAAGTGTCCCA | |
| | 152245280 | | | TAGGCCTCAGGGCAGCCT[CG]AGGGACTCT | |
| | | | | TGGAATTCGGCATCATCACAGTCCTCCGGGA | |
| | | | | TGCCC | |
| 122 | chrX: | - | 152245252 | CAGGGCCGCGGTGGCATTATCCTCCTCTCGA | 296 |
| | 152245154- | | | AACGCTTTGCCTAGCACTGTAAAGTGTCCCA | |
| | 152245280 | | | TAGGCCTCAGGGCAGCCTCGAGGGACTCTTG | |
| | | | | GAATT[CG]GCATCATCACAGTCCTCCGGGA | |
| | | | | TGCCC | |

TABLE 34
Table 34 below shows the coordinates and primers used to amplify the
identified target regions using bisulfite sequencing.

| | | | SEQ ID | | SEQ ID | |
|---|---|---|---|---|---|---|
| | | | NOs for | | NOs for | |
| | | | primer | | primer | |
| DMR | | | sequence | | sequence | Amplicon |
| # | Coordinates | Primer sequence 1 | 1 | Primer sequence 2 | 2 | size |
| 89 | chr1: | GGGGTGYGGGGAGGTTGA | 297 | TCCCCRACTCCCCCAACC | 298 | 140 |
| | 3157511-3157650 | GA | | TC | | |
| 91 | chr2: | GGYGTTGAAGTTGGAGAG | 299 | CCRAAACTCTTCTCCTTA | 300 | 127 |
| | 19550330-19550456 | GTTATTTTG | | AAACAAAAC | | |
| 92 | chr2: | GGTTTTTTTTAGTTTTAG | 301 | TACAACAAAAAAACTTAT | 302 | 149 |
| | 19550279-19550427 | YGTTTTGA | | AATCCAATTATCATC | | |
| 93 | chr3: | YGTGAGGTTGGTGGGTAG | 303 | TTCCCCTATCCRCCAACT | 304 | 105 |
| | 194118853-194118957 | GTTTAG | | TACAAATATATCTTC | | |
| 94 | chr3: | GTTYGTTAGTTTGTAAGT | 305 | CRACCCATTCCRAAAAAC | 306 | 124 |
| | 194118827-194118950 | GTGTTTTT | | AAAATATA | | |
| 95 | chr3: | GAATAATAGATAAGGGTG | 307 | TCACCTAAAACAAACATT | 308 | 108 |
| | 128712373-128712480 | GTTGGTAGTAAGTA | | CCAAAAACC | | |
| 96 | chr3: | GTTTATTTGGGGTAGGTA | 309 | AAAAAACAACAAATAAAA | 310 | 113 |
| | 128712370-128712482 | TTTTAGAAGTT | | ATAACTAACAATAAACA | | |
| 97 | chr4: | TYGGGATAGTATTTTGGG | 311 | ACCAAACRCCCATCCAAT | 312 | 118 |
| | 139483017-139483134 | AGTTGGG | | TTACCTA | | |
| 99 | chr8: | TTTTGTATTTTTTTTAGT | 313 | TTAAAAACCCCTCTCTCT | 314 | 150 |
| | 103629512-103629661 | AGAGAYGGGTTTTTAT | | TCCRAATA | | |
| 101 | chr8: | TTTTYGTTTTYGTAGGTA | 315 | RCCTCCTCACRAAAAAAC | 316 | 125 |
| | 145106870-145106994 | TTYGGTTATTTTG | | AACT | | |
| 105 | chr8: | TGTGTAAAGTYGGTGAGG | 317 | AAATCCAAAATAAAAATT | 318 | 119 |
| | 145103775-145103893 | TGTTGA | | TAAAATCAAATCCCTTT | | |
| 106 | chr9: | GGTAGAGTGAAGTATAAG | 319 | AAATAAACTCTAAACTCR | 320 | 11 [symbol] |
| | 100069971-100070085 | TAATAATTTTGTATTATT | | AACAAAAAAC | | |
| 107 | chr9: | AATTYGAGTAGGAGGTYG | 321 | ACAAAATAAAACACAAAC | 322 | 10 [symbol] |
| | 100069972-100070073 | GTTTTTTT | | AATAATCCTATATTATT | | |
| 108 | chr9: | ATTTTTTTTTTTTGTATT | 323 | CAAAAACCAACCTAAACA | 324 | 95 |
| | 139553849-139553943 | TTTTGTAGTTYGGTTTT | | TCTCAAATAA | | |
| 111 | chr11: | GGTYGGGTTATAAGGATT | 325 | CCAACCCCAAATCTTACR | 326 | 11 [symbol] |
| | 62693550-62693659 | YGGGAA | | AACAATTCC | | |
| 114 | chr16: | GTTTTAYGAGTTTTYGTT | 327 | AATACCCRATCTTCRTCC | 328 | 12 [symbol] |
| | 1271174-1271302 | YGTTTTGGTT | | CACAAA | | |
| 98 | chr4: | YGGYGGTTAGAYGTTTAT | 329 | CTACAAATCCRAAACAAC | 330 | 13 [symbol] |
| | 139483009-139483139 | TTAATTTGTTTG | | ACCTTAAAAACTAAA | | |
| 102 | chr8: | GTGTTTTTTTAAGGTAAG | 331 | CCCRCAATAAAAATACCT | 332 | 12 [symbol] |
| | 145106096-145106222 | TATAGATGAGGGG | | TAAATCTCC | | |
| 103 | chr8: | GATGTTTTAAGTTTTTYG | 333 | CTCAATATCCTCCTAAAA | 334 | 12 [symbol] |
| | 145106092-145106211 | GTTTTGGGGA | | CAAACACAAATAAAAAA | | |
| 109 | chr9: | GTTTTTGGTAGAGGTTAG | 335 | CCTCCTCCTACACCTCCT | 336 | 99 |
| | 139553853-139553951 | TTTGGGTATTTTAGGTGG | | ACAACC | | |
| 110 | chr11: | TAGTTTTYGAAGAAAAGA | 337 | AACRCCRAATCAACTCAA | 338 | 118 |
| | 62693570-62693687 | TGGGTTTGGGG | | CRAAATCTC | | |
| 112 | chr12: | GGAGTYGTTATGGAYGTT | 339 | TAAACRCRAACCCCAAAA | 340 | 123 |
| | 49390739-49390861 | GAGTTTTTTAGTTT | | CCCTA | | |
| 113 | chr12: | ATTTTAGGGTTTTAYGTY | 341 | CRAAACTTCTATATCRCT | 342 | 141 |
| | 49390712-49390852 | GGGGTTTTG | | TCCATCAAAAAAAC | | |
| 115 | chr16: | TTTTTTTTGGYGGGTGTT | 343 | TAATTCRAATTTCTCCRA | 344 | 118 |
| | 1271197-1271314 | YGGTTTT | | ATTTTACTACCAAC | | |
| 116 | chr17: | TTTTYGTGTTAGGGAATY | 345 | CTCATCTCAAAACRCAAA | 346 | 155 |
| | 43037200-43037354 | GGGGTTGAT | | AAACAAACC | | |
| 117 | chr17: | GTTTTTTTTTGYGATTTY | 347 | CRCTAAATCCAACTATAT | 348 | 102 |
| | 59532206-59532307 | GTAGGT | | CTAAAACCC | | |
| 118 | chr17: | YGYGTTGGGTTTAATTAT | 349 | CAAACCRCCACATCTAAA | 350 | 85 |
| | 59532225-59532309 | ATTTAGGGTTT | | ACCAA | | |
| 119 | chr19: | TYGGTATAGGTGYGGTTG | 351 | GCCCAAAAAAAATAAATC | 352 | 155 |
| | 17439718-17439872 | TAGGATTT | | CCCCRATCC | | |
| 120 | chr19: | GGYGTTTAGGGAGGGTGA | 353 | TCAACAACRTAACCAACA | 354 | 102 |
| | 17439774-17439875 | GTTTTT | | TCTAAAAACAAA | | |
| 121 | chrX: | ATTTTTYGGTYGAGTTYG | 355 | CATCCTAAACATCCCRAA | 356 | 153 |
| | 152245134-152245286 | ATTAGGGT | | AAACTATAATAATAC | | |
| 122 | chrX: | GGGTATTTYGGAGGATTG | 357 | CAAAACCRCRATAACATT | 358 | 127 |
| | 152245154-152245280 | TGATGATGT | | ATCCTCCTCT | | |

[symbol]

indicates data missing or illegible when filed

It is to be understood that different applications of the disclosed methods and products may be tailored to the specific needs in the art. It is also to be understood that the terminology used herein is for the purpose of describing particular embodiments of the invention only, and is not intended to be limiting.

As used in this specification and the appended claims, the singular forms “a”, “an”, and “the” include plural referents unless the content clearly dictates otherwise. Thus, for example, reference to “a ligation polynucleotide” includes two or more such polynucleotides, reference to “a scaffold polynucleotide” includes two or more such scaffold polynucleotides, and the like.

All publications, patents and patent applications cited herein are hereby incorporated by reference in their entirety.

### REFERENCES

1. Torre L A, Bray F, Siegel R L, et al: Global cancer statistics, 2012. CA Cancer J Clin 65:87-108, 2015.
2. Marmot M G, Altman D G, Cameron D A, et al: The benefits and harms of breast cancer screening: an independent review. Br. J Cancer 108:2205-2240, 2013.
3. Mook S, Van 't Veer L J, Rutgers E J, et al: Independent prognostic value of screen detection in invasive breast cancer. J Natl. Cancer Inst 103:585-597, 2011.
4. Harper K L, Sosa M S, Entenberg D, et al: Mechanism of early dissemination and metastasis in Her2+ mammary cancer. Nature, 2016.
5. Welch H G, Prorok P C, O'Malley A J, et al: Breast-Cancer Tumor Size, Overdiagnosis, and Mammography Screening Effectiveness. N. Engl. J Med 375:1438-1447, 2016.
6. Klein C A: Parallel progression of primary tumours and metastases. Nat. Rev. Cancer 9:302-312, 2009.
7. Braun S, Vogl F D, Naume B, et al: A pooled analysis of bone marrow micrometastasis in breast cancer. N. Engl. J. Med 353:793-802, 2005.
8. Mansi J L, Gogas H, Bliss J M, et al: Outcome of primary-breast-cancer patients with micrometastases: a long-term follow-up study. Lancet 354:197-202, 1999.
9. Klein C A, Blankenstein T J, Schmidt-Kittler O, et al: Genetic heterogeneity of single disseminated tumour cells in minimal residual cancer. Lancet 360:683-689, 2002.
10. Bidard F C, Peeters D J, Fehm T, et al: Clinical validity of circulating tumour cells in patients with metastatic breast cancer: a pooled analysis of individual patient data. Lancet Oncol 15:406-414, 2014.
11. Lucci A, Hall C S, Lodhi A K, et al: Circulating tumour cells in non-metastatic breast cancer: a prospective study. Lancet Oncol 13:688-695, 2012.
12. Rack B, Schindlbeck C, Juckstock J, et al: Circulating tumor cells predict survival in early average-to-high risk breast cancer patients. J Natl. Cancer Inst 106, 2014.
13. Cristofanilli M, Budd G T, Ellis M J, et al: Circulating tumor cells, disease progression, and survival in metastatic breast cancer. N. Engl. J Med 351:781-791, 2004.
14. Janni W J, Rack B, Terstappen L W, et al: Pooled Analysis of the Prognostic Relevance of Circulating Tumor Cells in Primary Breast Cancer. Clin. Cancer Res 22:2583-2593, 2016.
15. Dawson S J, Tsui D W, Murtaza M, et al: Analysis of circulating tumor DNA to monitor metastatic breast cancer. N. Engl. J Med 368:1199-1209, 2013.
16. Murtaza M, Dawson S J, Tsui D W, et al: Non-invasive analysis of acquired resistance to cancer therapy by sequencing of plasma DNA. Nature 497:108-112, 2013.
17. Wang Y, Springer S, Mulvey C L, et al: Detection of somatic mutations and HPV in the saliva and plasma of patients with head and neck squamous cell carcinomas. Sci. Transl Med 7:293ra104, 2015.
18. Siravegna G, Mussolin B, Buscarino M, et al: Clonal evolution and resistance to EGFR blockade in the blood of colorectal cancer patients. Nat. Med 21:827, 2015.
19. Bettegowda C, Sausen M, Leary R J, et al: Detection of circulating tumor DNA in early- and late-stage human malignancies. Sci. Transl Med 6:224ra24, 2014.
20. De Mattos-Arruda L, Caldas C: Cell-free circulating tumour DNA as a liquid biopsy in breast cancer. Mol. Oncol 10:464-474, 2016.
21. Lanman R B, Mortimer S A, Zill O A, et al: Analytical and Clinical Validation of a Digital Sequencing Panel for Quantitative, Highly Accurate Evaluation of Cell-Free Circulating Tumor DNA. PLoS. One 10:e0140712, 2015.
22. Baylin S B, Jones P A: A decade of exploring the cancer epigenome—biological and translational implications. Nat. Rev. Cancer 11:726-734, 2011.
23. Teschendorff A E, Gao Y, Jones A, et al: DNA methylation outliers in normal breast tissue identify field defects that are enriched in cancer. Nat. Commun 7:10478, 2016.
24. Fackler M J, Lopez B Z, Umbricht C, et al: Novel methylated biomarkers and a robust assay to detect circulating tumor DNA in metastatic breast cancer. Cancer Res 74:2160-2170, 2014.
25. Fiegl H, Millinger S, Mueller-Holzner E, et al: Circulating tumor-specific DNA: a marker for monitoring efficacy of adjuvant therapy in cancer patients. Cancer Res 65:1141-1145, 2005.
26. Muller H M, Widschwendter A, Fiegl H, et al: DNA methylation in serum of breast cancer patients: an independent prognostic marker. Cancer Res 63:7641-7645, 2003.
27. Muller H M, Fiegl H, Widschwendter A, et al: Prognostic DNA methylation marker in serum of cancer patients. Ann. N. Y. Acad. Sci 1022:44-49, 2004.
28. Warton K, Mahon K L, Samimi G: Methylated circulating tumor DNA in blood: power in cancer prognosis and response. Endocr. Relat Cancer 23:R157-R171, 2016.
29. Wittenberger T, Sleigh S, Reisel D, et al: DNA methylation markers for early detection of women's cancer: promise and challenges. Epigenomics 6:311-327, 2014.
30. Sun K, Jiang P, Chan K C, et al: Plasma DNA tissue mapping by genome-wide methylation sequencing for noninvasive prenatal, cancer, and transplantation assessments. Proc. Natl. Acad. Sci. U.S.A 112:E5503-E5512, 2015.
31. Olkhov-Mitsel, E and Bapat, B: Strategies for discovery and validation of methylated and hydroxymethylated DNA biomarkers. Cancer Medicine 2012, 1(2): 237-260.
32. Paul D S, Guilhamon P, Karpathakis A, Butcher L M, Thirlwell C, Feber A, Beck S: Assessment of RainDrop BS-seq as a method for large-scale, targeted bisulfite sequencing. Epigenetics 2014, 9.
33. Cottrell, S. E., Distler, J., Goodman, N. S., Mooney, S. H., Kluth, A., Olek, A., Schwope, I., Tetzner, R., Ziebarth, H. and Berlin, K. A real-time PCR assay for DNA-methylation using methylation-specific blockers. Nucleic Acids Research, 2004, 32(1) e10, pp 1-8.
34. Eads, C. A., Danenberg, K. D., Kawakami, K., Saltz, L. B., Blake, C., Shibata, D., Danenberg, P. V., Laird P. W. MethyLight: a high-throughput assay to measure DNA methylation. Nucleic Acids Research. 2000, 28(8): E32.
35. Frommer, M. et al.: A genomic sequencing protocol that yields a positive display of 5-methylcytosine residues in individual DNA strands. Proc. Natl Acad. Sci. USA 1992, 89: 1827-1831.
36. Xiong, Z. & Laird, P. W.: COBRA: a sensitive and quantitative DNA methylation assay. Nucleic Acids Res. 1997, 25: 2532-2534.
37. Gonzalgo, M. L. & Jones, P. A.: Rapid quantitation of methylation differences at specific sites using methylationsensitive single nucleotide primer extension (Ms-SNuPE). Nucleic Acids Res. 1997, 25: 2529-2531.
38. Herman, J. G., Graff, J. R., Myohanen, S., Nelkin, B. D. & Baylin, S. B.: Methylation-specific PCR: a novel PCR assay for methylation status of CpG islands. Proc. Natl Acad. Sci. USA 1996, 93: 9821-9826.
39. Singal, R. & Grimes, S. R.: Microsoft Word macro for analysis of cytosine methylation by the bisulfite deamination reaction. Biotechniques 2001, 30: 116-120.
40. Anbazhagan, R., Herman, J. G., Enika, K. & Gabrielson, E.: Spreadsheet-based program for the analysis of DNA methylation. Biotechniques 2001, 30: 110-114.
41. Li, L. C. & Dahiya, R.: MethPrimer: designing primers for methylation PCRs. Bioinformatics 2002, 18: 1427-1431.
42. Eng, J: Receiver Operating Characteristic Analysis: A Primer. Academic Radiology 2005, 12(7): 909-916.
43. Bauminger, S. & Wilchek, M. The use of carbodiimides in the preparation of immunizing conjugates. (1980) Methods Enzymol. 70, 151-159.
44. Gu H, Smith Z D, Bock C, et al: Preparation of reduced representation bisulfite sequencing libraries for genome-scale DNA methylation profiling. Nat. Protoc 6:468-481, 2011.
45. Lee Y K, Jin S, Duan S, et al: Improved reduced representation bisulfite sequencing for epigenomic profiling of clinical samples. Biol. Proced. Online 16:1, 2014.
46. Newcombe R G: Two-sided confidence intervals for the single proportion: comparison of seven methods. Stat. Med 17:857-872, 1998.
47. Jacobs I J, Menon U, Ryan A, et al: Ovarian cancer screening and mortality in the UK Collaborative Trial of Ovarian Cancer Screening (UKCTOCS): a randomised controlled trial. Lancet 387:945-956, 2016.
48. Bernstein, D. L., Kameswaran, V., John E Le Lay, J. E., Sheaffer, K. L., and Kaestner, K. H. The BisPCR2 method for targeted bisulfite sequencing. Epigenetics & Chromatin (2015) 8(27), pp 1-9.
49. Gormally E, Caboux E, Vineis P, et al: Circulating free DNA in plasma or serum as biomarker of carcinogenesis: practical aspects and biological significance. Mutat. Res 635:105-117, 2007.
50. Jiang P, Lo Y M: The Long and Short of Circulating Cell-Free DNA and the Ins and Outs of Molecular Diagnostics. Trends Genet 32:360-371, 2016.
51. Kang Q, Henry N L, Paoletti C, et al: Comparative analysis of circulating tumor DNA stability In KEDTA, Streck, and CellSave blood collection tubes. Clin. Biochem, 2016.
52. Fenton J J, Taplin S H, Carney P A, et al: Influence of computer-aided detection on performance of screening mammography. NJ. Engl. Med 356:1399-1409, 2007.

1. A method of identifying the presence of metastatic breast cancer (mBC) cell DNA in a sample from an individual, the method comprising:
i. providing DNA from a sample from the individual, the sample DNA comprising a plurality of DNA molecules each having a defined differentially methylated region (DMR);
ii. determining the methylation status of four or more methylation variable positions (MVPs) within DMRs, wherein the MVPs are selected from a group of linked MVPs within the DMR;
iii. selecting a pre-defined DMR methylation pattern for the four or more MVPs within the DMR, wherein each one of the four or more MVPs is scored as methylated or unmethylated;
iv. determining a pattern frequency for the DMR methylation pattern; and
v. identifying mBC DNA within the sample DNA when the pattern frequency equals or exceeds a threshold value.

2. A method of diagnosing metastatic breast cancer (mBC) by identifying the presence of mBC cell DNA in a sample from an individual, the method comprising:
i. providing DNA from a sample from the individual, the sample DNA comprising a plurality of DNA molecules each having a defined differentially methylated region (DMR);
ii. determining the methylation status of four or more linked methylation variable positions (MVPs) within DMRs, wherein the MVPs are selected from a group of linked MVPs within the DMR;
iii. selecting a pre-defined DMR methylation pattern for the four or more MVPs within the DMR, wherein each one of the four or more MVPs is scored as methylated or unmethylated;
iv. determining a pattern frequency for the DMR methylation pattern;
v. identifying mBC DNA within the sample DNA when the pattern frequency equals or exceeds a threshold value; and
vi. diagnosing metastatic breast cancer when mBC DNA is identified within the sample DNA in accordance with step (v).

3. A method of providing a disease prognosis to a breast cancer patient by identifying the presence of metastatic breast cancer (mBC) cell DNA in a sample from an individual, the method comprising:
i. providing DNA from a sample from the individual, the sample DNA comprising a plurality of DNA molecules each having a defined differentially methylated region (DMR);
ii. determining the methylation status of four or more linked methylation variable positions (MVPs) within DMRs, wherein the MVPs are selected from a group of linked MVPs within the DMR;
iii. selecting a pre-defined DMR methylation pattern for the four or more MVPs within the DMR, wherein each one of the four or more MVPs is scored as methylated or unmethylated;
iv. determining a pattern frequency for the DMR methylation pattern;
v. identifying mBC DNA within the sample DNA when the pattern frequency equals or exceeds a threshold value; and
vi. providing the breast cancer patient with a disease prognosis when mBC DNA is identified within the sample DNA in accordance with step (v).

4. A method according to claim 3, wherein the disease prognosis is provided as a hazard ratio for death score (HR).

5. A method according to claim 4, wherein the HR is 6 or more.

6. A method according to claim 4, wherein the HR is between about 6 and about 9, preferably 7.7.

7. A method according to claim 5 or claim 6, wherein the HR score 95% confidence interval is 2.5-17.5, preferably 3.5-16.8.

8. A method according to any one of claims 2 to 7, wherein the prognosis is provided before the patient has undertaken a therapeutic treatment, e.g. chemotherapy.

9. A method of identifying in DNA from an individual the presence of a methylation signature correlated with metastatic breast cancer (mBC) by identifying the presence of mBC DNA in a sample from an individual, the method comprising:
i. providing DNA from a sample from the individual, the sample DNA comprising a plurality of DNA molecules each having a defined differentially methylated region (DMR);
ii. determining the methylation status of four or more linked methylation variable positions (MVPs) within DMRs, wherein the MVPs are selected from a group of linked MVPs within the DMR;
iii. selecting a pre-defined DMR methylation pattern for the four or more MVPs within the DMR, wherein each one of the four or more MVPs is scored as methylated or unmethylated;
iv. determining a pattern frequency for the DMR methylation pattern;
v. identifying mBC DNA within the sample DNA when the pattern frequency equals or exceeds a threshold value; and
vi. identifying the methylation signature when mBC DNA is identified within the sample DNA in accordance with step (v).

10. A method according to any one of claims 1 to 9, wherein in step (iii) the DMR methylation pattern is defined to score at least three of the four or more MVPs as methylated, or wherein the DMR methylation pattern is defined to score at least four of the four or more MVPs as methylated.

11. A method according to any one of claims 1 to 9, wherein step (ii) comprises determining the methylation status of at least five or more linked MVPs within the DMR.

12. A method according to claim 11, wherein in step (iii) the DMR methylation pattern is defined to score at least all five of the five or more MVPs as methylated.

13. A method according to any one of the preceding claims, wherein in step (v) the DMR methylation pattern frequency threshold value is 0.0001, or 0.0002, or 0.0003, or 0.0004, or 0.0005, or 0.0006, or 0.0007, or 0.0008, or 0.0009, or 0.001, preferably 0.0008.

14. A method according to any one of the preceding claims, wherein the method achieves a ROC sensitivity of 60% or more.

15. A method according to any one of the preceding claims, wherein the method achieves a ROC specificity of 90% or more.

16. A method according to any one of the preceding claims, wherein the method achieves a ROC sensitivity of 60% or more and a ROC specificity of 90% or more, preferably wherein the method achieves a ROC sensitivity of 60.9% or more and a ROC specificity of 92% or more.

17. A method according to any one of claims 1 to 16, wherein in step (i) the DMR is comprised within the sequence set forth in SEQ ID NO: 1, and in step (ii) the group of linked MVPs are the 11 MVPs of SEQ ID NOS: 2 to 12 denoted by [CG].

18. A method according to claim 17, wherein step (ii) comprises determining the methylation status of at least four of the five MVPs of SEQ ID NOS: 2 to 6 denoted by [CG].

19. A method according to claim 18, wherein step (ii) comprises determining the methylation status of all five MVPs of SEQ ID NOS: 2 to 6 denoted by [CG].

20. A method according to any one of claims 17 to 19, wherein in step (iii) the methylation pattern is defined to score as methylated each MVP in a group of MVPs comprising the MVPs of SEQ ID NOS: 2 to 6 denoted by [CG].

21. A method according to any one of claims 17 to 19, wherein in step (iii) the methylation pattern is defined to score as methylated each MVP in a group of MVPs consisting of the MVPs of SEQ ID NOS: 2 to 6 denoted by [CG].

22. A method according to claim 17, wherein step (ii) comprises determining the methylation status of a group of MVPs comprising or consisting of all five MVPs of SEQ ID NOS: 2 to 6 denoted by [CG]; wherein in step (iii) the methylation pattern is defined to score as methylated all five MVPs of SEQ ID NOS: 2 to 6 denoted by [CG]; and wherein in step (v) the DMR methylation pattern frequency threshold value in the sample DNA is 0.0008.

23. A method according to claim 22, wherein the method achieves a ROC sensitivity of 60% or more and a ROC specificity of 90% or more, preferably wherein the method achieves a ROC sensitivity of 60.9% or more and a ROC specificity of 92.0% or more.

24. A method according to any one of claims 1 to 16, wherein in step (i) the DMR is comprised within the sequence set forth in SEQ ID NO: 13, and in step (ii) the group of linked MVPs are the 11 MVPs of SEQ ID NOS: 14 to 24 denoted by [CG].

25. A method according to claim 24, wherein step (ii) comprises determining the methylation status of at least four or at least five MVPs of SEQ ID NOS: 14 to 24 denoted by [CG]; or wherein step (ii) comprises determining the methylation status of all 11 MVPs of SEQ ID NOS: 14 to 24 denoted by [CG].

26. A method according to any one of claims 24 to 25, wherein in step (iii) the methylation pattern is defined to score as methylated at least four or at least five of the at least four or five MVPs whose methylation status is determined in step (ii); or wherein in step (iii) the methylation pattern is defined to score as methylated each MVP in a group of MVPs comprising the MVPs of SEQ ID NOS: 14 to 24 denoted by [CG]; or wherein in step (iii) the methylation pattern is defined to score as methylated each MVP in a group of MVPs consisting of the MVPs of SEQ ID NOS: 14 to 24 denoted by [CG].

27. A method according to claim 24, wherein step (ii) comprises determining the methylation status of a group of MVPs comprising or consisting of all 11 MVPs of SEQ ID NOS: 14 to 24 denoted by [CG]; wherein in step (iii) the methylation pattern is defined to score as methylated all 11 MVPs of SEQ ID NOS: 14 to 24 denoted by [CG]; and wherein in step (v) the DMR methylation pattern frequency threshold value in the sample DNA is 0.0005 or more, preferably 0.0008.

28. A method according to any one of claims 1 to 16, wherein in step (i) the DMR is comprised within the sequence set forth in SEQ ID NO: 25, and in step (ii) the group of linked MVPs are the 11 MVPs of SEQ ID NOS: 26 to 36 denoted by [CG].

29. A method according to claim 28, wherein step (ii) comprises determining the methylation status of at least four or at least five or at least seven MVPs of SEQ ID NOS: 26 to 36 denoted by [CG], optionally determining the methylation status of at least the seven MVPs of SEQ ID NOS:30 to 36 denoted by [CG].

30. A method according to claim 29, wherein step (ii) comprises determining the methylation status of all 11 MVPs of SEQ ID NOS: 26 to 36 denoted by [CG].

31. A method according to claim 29, wherein in step (iii) the methylation pattern is defined to score as methylated each MVP in a group of MVPs comprising the MVPs of SEQ ID NOS: 30 to 36 denoted by [CG]; or wherein in step (iii) the methylation pattern is defined to score as methylated each MVP in a group of MVPs consisting of the MVPs of SEQ ID NOS: 30 to 36 denoted by [CG]; or a method according to any one of claims 28 to 30, wherein in step (iii) the methylation pattern is defined to score as methylated each MVP in a group of MVPs comprising the MVPs of SEQ ID NOS: 26 to 36 denoted by [CG]; or wherein in step (iii) the methylation pattern is defined to score as methylated each MVP in a group of MVPs consisting of the MVPs of SEQ ID NOS: 26 to 36 denoted by [CG].

32. A method according to claim 28, wherein step (ii) comprises determining the methylation status of a group of MVPs comprising or consisting of all 11 MVPs of SEQ ID NOS: 26 to 36 denoted by [CG]; wherein in step (iii) the methylation pattern is defined to score as methylated all 11 MVPs of SEQ ID NOS: 26 to 36 denoted by [CG]; and wherein in step (v) the DMR methylation pattern frequency threshold value in the sample DNA is 0.0005 or more, preferably 0.0008.

33. A method according to any one of claims 1 to 16, wherein in step (i) the DMR is comprised within the sequence set forth in SEQ ID NO: 37, and in step (ii) the group of linked MVPs are the 16 MVPs of SEQ ID NOS: 38 to 53 denoted by [CG].

34. A method according to claim 33, wherein step (ii) comprises determining the methylation status of at least four or at least five MVPs of SEQ ID NOS: 38 to 53 denoted by [CG]; or wherein step (ii) comprises determining the methylation status of all 16 MVPs of SEQ ID NOS: 38 to 53 denoted by [CG].

35. A method according to any one of claims 33 to 34, wherein in step (iii) the methylation pattern is defined to score as methylated each MVP in a group of MVPs comprising the MVPs of SEQ ID NOS: 38 to 53 denoted by [CG]; or wherein in step (iii) the methylation pattern is defined to score as methylated each MVP in a group of MVPs consisting of the MVPs of SEQ ID NOS: 38 to 53 denoted by [CG].

36. A method according to claim 33, wherein step (ii) comprises determining the methylation status of a group of MVPs comprising or consisting of all 16 MVPs of SEQ ID NOS: 38 to 53 denoted by [CG]; wherein in step (iii) the methylation pattern is defined to score as methylated all 16 MVPs of SEQ ID NOS: 38 to 53 denoted by [CG]; and wherein in step (v) the DMR methylation pattern frequency threshold value in the sample DNA is 0.0005 or more, preferably 0.0008.

37. A method according to any one of claims 1 to 16, wherein in step (i) the DMR is comprised within the sequence set forth in SEQ ID NO: 54, and in step (ii) the group of linked MVPs are the 12 MVPs of SEQ ID NOS: 55 to 66 denoted by [CG].

38. A method according to claim 37, wherein step (ii) comprises determining the methylation status of at least four or at least five MVPs of SEQ ID NOS: 55 to 66 denoted by [CG]; or wherein step (ii) comprises determining the methylation status of all 12 MVPs of SEQ ID NOS: 55 to 66 denoted by [CG].

39. A method according to any one of claims 37 to 38, wherein in step (iii) the methylation pattern is defined to score as methylated each MVP in a group of MVPs comprising the MVPs of SEQ ID NOS: 55 to 66 denoted by [CG]; or wherein in step (iii) the methylation pattern is defined to score as methylated each MVP in a group of MVPs consisting of the MVPs of SEQ ID NOS: 55 to 66 denoted by [CG].

40. A method according to claim 37, wherein step (ii) comprises determining the methylation status of a group of MVPs comprising or consisting of all 12 MVPs of SEQ ID NOS: 55 to 66 denoted by [CG]; wherein in step (iii) the methylation pattern is defined to score as methylated all 12 MVPs of SEQ ID NOS: 55 to 66 denoted by [CG]; and wherein in step (v) the DMR methylation pattern frequency threshold value in the sample DNA is 0.0005 or more, preferably 0.0008.

41. A method according to any one of claims 1 to 16, wherein in step (i) the DMR is comprised within the sequence set forth in SEQ ID NO: 67, and in step (ii) the group of linked MVPs are the 7 MVPs of SEQ ID NOS: 68 to 74 denoted by [CG].

42. A method according to claim 41, wherein step (ii) comprises determining the methylation status of at least four or at least five MVPs of SEQ ID NOS: 68 to 74 denoted by [CG]; or wherein step (ii) comprises determining the methylation status of all 7 MVPs of SEQ ID NOS: 68 to 74 denoted by [CG].

43. A method according to any one of claims 41 to 42, wherein in step (iii) the methylation pattern is defined to score as methylated each MVP in a group of MVPs comprising the MVPs of SEQ ID NOS: 68 to 74 denoted by [CG]; or wherein in step (iii) the methylation pattern is defined to score as methylated each MVP in a group of MVPs consisting of the MVPs of SEQ ID NOS: 68 to 74 denoted by [CG].

44. A method according to claim 41, wherein step (ii) comprises determining the methylation status of a group of MVPs comprising or consisting of all 7 MVPs of SEQ ID NOS: 68 to 74 denoted by [CG]; wherein in step (iii) the methylation pattern is defined to score as methylated all 7 MVPs of SEQ ID NOS: 68 to 74 denoted by [CG]; and wherein in step (v) the DMR methylation pattern frequency threshold value in the sample DNA is 0.0005 or more, preferably 0.0008.

45. A method according to any one of claims 1 to 16, wherein in step (i) the DMR is comprised within the sequence set forth in SEQ ID NO: 75, and in step (ii) the group of linked MVPs are the 7 MVPs of SEQ ID NOS: 76 to 82 denoted by [CG].

46. A method according to claim 45, wherein step (ii) comprises determining the methylation status of at least four or at least five MVPs of SEQ ID NOS: 76 to 82 denoted by [CG]; or wherein step (ii) comprises determining the methylation status of all 7 MVPs of SEQ ID NOS: 76 to 82 denoted by [CG].

47. A method according to any one of claims 45 to 46, wherein in step (iii) the methylation pattern is defined to score as methylated each MVP in a group of MVPs comprising the MVPs of SEQ ID NOS: 76 to 82 denoted by [CG]; or wherein in step (iii) the methylation pattern is defined to score as methylated each MVP in a group of MVPs consisting of the MVPs of SEQ ID NOS: 76 to 82 denoted by [CG].

48. A method according to claim 45, wherein step (ii) comprises determining the methylation status of a group of MVPs comprising or consisting of all 7 MVPs of SEQ ID NOS: 76 to 82 denoted by [CG]; wherein in step (iii) the methylation pattern is defined to score as methylated all 7 MVPs of SEQ ID NOS: 76 to 82 denoted by [CG]; and wherein in step (v) the DMR methylation pattern frequency threshold value in the sample DNA is 0.0005 or more, preferably 0.0008.

49. A method according to any one of claims 1 to 16, wherein in step (i) the DMR is comprised within the sequence set forth in SEQ ID NO: 83, and in step (ii) the group of linked MVPs are the 5 MVPs of SEQ ID NOS: 84 to 88 denoted by [CG].

50. A method according to claim 49, wherein step (ii) comprises determining the methylation status of at least four or at least five MVPs of SEQ ID NOS: 84 to 88 denoted by [CG]; or wherein step (ii) comprises determining the methylation status of all 5 MVPs of SEQ ID NOS: 84 to 88 denoted by [CG].

51. A method according to any one of claims 49 to 50, wherein in step (iii) the methylation pattern is defined to score as methylated each MVP in a group of MVPs comprising the MVPs of SEQ ID NOS: 84 to 88 denoted by [CG]; or wherein in step (iii) the methylation pattern is defined to score as methylated each MVP in a group of MVPs consisting of the MVPs of SEQ ID NOS: 84 to 88 denoted by [CG].

52. A method according to claim 49, wherein step (ii) comprises determining the methylation status of a group of MVPs comprising or consisting of all 5 MVPs of SEQ ID NOS: 84 to 88 denoted by [CG]; wherein in step (iii) the methylation pattern is defined to score as methylated all 5 MVPs of SEQ ID NOS: 84 to 88 denoted by [CG]; and wherein in step (v) the DMR methylation pattern frequency threshold value in the sample DNA is 0.0005 or more, preferably 0.0008.

53. A method according to any one of claims 1 to 16, wherein in step (i) the DMR is comprised within the sequence set forth in SEQ ID NO: 89, and in step (ii) the group of linked MVPs are the 7 MVPs of SEQ ID NOS: 90 to 96 denoted by [CG].

54. A method according to claim 53, wherein step (ii) comprises determining the methylation status of at least four or at least five MVPs of SEQ ID NOS: 90 to 96 denoted by [CG]; or wherein step (ii) comprises determining the methylation status of all 7 MVPs of SEQ ID NOS: 90 to 96 denoted by [CG].

55. A method according to any one of claims 53 to 54, wherein in step (iii) the methylation pattern is defined to score as methylated each MVP in a group of MVPs comprising the MVPs of SEQ ID NOS: 90 to 96 denoted by [CG]; or wherein in step (iii) the methylation pattern is defined to score as methylated each MVP in a group of MVPs consisting of the MVPs of SEQ ID NOS: 90 to 96 denoted by [CG].

56. A method according to claim 53, wherein step (ii) comprises determining the methylation status of a group of MVPs comprising or consisting of all 7 MVPs of SEQ ID NOS: 90 to 96 denoted by [CG]; wherein in step (iii) the methylation pattern is defined to score as methylated all 7 MVPs of SEQ ID NOS: 90 to 96 denoted by [CG]; and wherein in step (v) the DMR methylation pattern frequency threshold value in the sample DNA is 0.0005 or more, preferably 0.0008.

57. A method according to any one of claims 1 to 16, wherein in step (i) the DMR is comprised within the sequence set forth in SEQ ID NO: 97, and in step (ii) the group of linked MVPs are the 14 MVPs of SEQ ID NOS: 98 to 111 denoted by [CG].

58. A method according to claim 57, wherein step (ii) comprises determining the methylation status of at least four or at least five MVPs of SEQ ID NOS: 98 to 111 denoted by [CG; or wherein step (ii) comprises determining the methylation status of all 14 MVPs of SEQ ID NOS: 98 to 111 denoted by [CG].

59. A method according to any one of claims 57 to 58, wherein in step (iii) the methylation pattern is defined to score as methylated each MVP in a group of MVPs comprising the MVPs of SEQ ID NOS: 98 to 111 denoted by [CG]; or wherein in step (iii) the methylation pattern is defined to score as methylated each MVP in a group of MVPs consisting of the MVPs of SEQ ID NOS: 98 to 111 denoted by [CG].

60. A method according to claim 57, wherein step (ii) comprises determining the methylation status of a group of MVPs comprising or consisting of all 14 MVPs of SEQ ID NOS: 98 to 111 denoted by [CG]; wherein in step (iii) the methylation pattern is defined to score as methylated all 14 MVPs of SEQ ID NOS: 98 to 111 denoted by [CG]; and wherein in step (v) the DMR methylation pattern frequency threshold value in the sample DNA is 0.0005 or more, preferably 0.0008.

61. A method according to any one of claims 1 to 16, wherein in step (i) the DMR is comprised within the sequence set forth in SEQ ID NO: 112, and in step (ii) the group of linked MVPs are the 7 MVPs of SEQ ID NOS: 113 to 119 denoted by [CG].

62. A method according to claim 61, wherein step (ii) comprises determining the methylation status of at least four or at least five MVPs of SEQ ID NOS: 113 to 119 denoted by [CG]; or wherein step (ii) comprises determining the methylation status of all 7 MVPs of SEQ ID NOS: 113 to 119 denoted by [CG].

63. A method according to any one of claims 61 to 62, wherein in step (iii) the methylation pattern is defined to score as methylated each MVP in a group of MVPs comprising the MVPs of SEQ ID NOS: 113 to 119 denoted by [CG]; or wherein in step (iii) the methylation pattern is defined to score as methylated each MVP in a group of MVPs consisting of the MVPs of SEQ ID NOS: 113 to 119 denoted by [CG].

64. A method according to claim 61, wherein step (ii) comprises determining the methylation status of a group of MVPs comprising or consisting of all 7 MVPs of SEQ ID NOS: 113 to 119 denoted by [CG]; wherein in step (iii) the methylation pattern is defined to score as methylated all 7 MVPs of SEQ ID NOS: 113 to 119 denoted by [CG]; and wherein in step (v) the DMR methylation pattern frequency threshold value in the sample DNA is 0.0005 or more, preferably 0.0008.

65. A method according to any one of claims 1 to 16, wherein in step (i) the DMR is comprised within the sequence set forth in SEQ ID NO: 120, and in step (ii) the group of linked MVPs are the 8 MVPs of SEQ ID NOS: 121 to 128 denoted by [CG].

66. A method according to claim 65, wherein step (ii) comprises determining the methylation status of at least four or at least five MVPs of SEQ ID NOS: 121 to 128 denoted by [CG]; or wherein step (ii) comprises determining the methylation status of all 8 MVPs of SEQ ID NOS: 121 to 128 denoted by [CG].

67. A method according to any one of claims 65 to 66, wherein in step (iii) the methylation pattern is defined to score as methylated each MVP in a group of MVPs comprising the MVPs of SEQ ID NOS: 121 to 128 denoted by [CG]; or wherein in step (iii) the methylation pattern is defined to score as methylated each MVP in a group of MVPs consisting of the MVPs of SEQ ID NOS: 121 to 128 denoted by [CG].

68. A method according to claim 65, wherein step (ii) comprises determining the methylation status of a group of MVPs comprising or consisting of all 8 MVPs of SEQ ID NOS: 121 to 128 denoted by [CG]; wherein in step (iii) the methylation pattern is defined to score as methylated all 8 MVPs of SEQ ID NOS: 121 to 128 denoted by [CG]; and wherein in step (v) the DMR methylation pattern frequency threshold value in the sample DNA is 0.0005 or more, preferably 0.0008.

69. A method according to any one of claims 1 to 16, wherein in step (i) the DMR is comprised within the sequence set forth in SEQ ID NO: 129, and in step (ii) the group of linked MVPs are the 7 MVPs of SEQ ID NOS: 130 to 136 denoted by [CG].

70. A method according to claim 67, wherein step (ii) comprises determining the methylation status of at least four or at least five MVPs of SEQ ID NOS: 130 to 136 denoted by [CG]; or wherein step (ii) comprises determining the methylation status of all 7 MVPs of SEQ ID NOS: 130 to 136 denoted by [CG].

71. A method according to any one of claims 67 to 68, wherein in step (iii) the methylation pattern is defined to score as methylated each MVP in a group of MVPs comprising the MVPs of SEQ ID NOS: 130 to 136 denoted by [CG]; or wherein in step (iii) the methylation pattern is defined to score as methylated each MVP in a group of MVPs consisting of the MVPs of SEQ ID NOS: 130 to 136 denoted by [CG].

72. A method according to claim 67, wherein step (ii) comprises determining the methylation status of a group of MVPs comprising or consisting of all 7 MVPs of SEQ ID NOS: 130 to 136 denoted by [CG]; wherein in step (iii) the methylation pattern is defined to score as methylated all 7 MVPs of SEQ ID NOS: 130 to 136 denoted by [CG]; and wherein in step (v) the DMR methylation pattern frequency threshold value in the sample DNA is 0.0005 or more, preferably 0.0008.

73. A method according to any one of claims 1 to 16, wherein in step (i) the DMR is comprised within the sequence set forth in SEQ ID NO: 137, and in step (ii) the group of linked MVPs are the 6 MVPs of SEQ ID NOS: 138 to 143 denoted by [CG].

74. A method according to claim 73, wherein step (ii) comprises determining the methylation status of at least four or at least five MVPs of SEQ ID NOS: 138 to 143 denoted by [CG]; or wherein step (ii) comprises determining the methylation status of all 6 MVPs of SEQ ID NOS: 138 to 143 denoted by [CG].

75. A method according to any one of claims 73 to 74, wherein in step (iii) the methylation pattern is defined to score as methylated each MVP in a group of MVPs comprising the MVPs of SEQ ID NOS: 138 to 143 denoted by [CG]; or wherein in step (iii) the methylation pattern is defined to score as methylated each MVP in a group of MVPs consisting of the MVPs of SEQ ID NOS: 138 to 143 denoted by [CG].

76. A method according to claim 73, wherein step (ii) comprises determining the methylation status of a group of MVPs comprising or consisting of all 6 MVPs of SEQ ID NOS: 138 to 143 denoted by [CG]; wherein in step (iii) the methylation pattern is defined to score as methylated all 6 MVPs of SEQ ID NOS: 138 to 143 denoted by [CG]; and wherein in step (v) the DMR methylation pattern frequency threshold value in the sample DNA is 0.0005 or more, preferably 0.0008.

77. A method according to any one of claims 1 to 16, wherein in step (i) the DMR is comprised within the sequence set forth in SEQ ID NO: 144, and in step (ii) the group of linked MVPs are the 9 MVPs of SEQ ID NOS: 145 to 153 denoted by [CG].

78. A method according to claim 77, wherein step (ii) comprises determining the methylation status of at least four or at least five MVPs of SEQ ID NOS: 145 to 153 denoted by [CG]; or wherein step (ii) comprises determining the methylation status of all 9 MVPs of SEQ ID NOS: 145 to 153 denoted by [CG].

79. A method according to any one of claims 77 to 78, wherein in step (iii) the methylation pattern is defined to score as methylated each MVP in a group of MVPs comprising the MVPs of SEQ ID NOS: 145 to 153 denoted by [CG]; or wherein in step (iii) the methylation pattern is defined to score as methylated each MVP in a group of MVPs consisting of the MVPs of SEQ ID NOS: 145 to 153 denoted by [CG].

80. A method according to claim 77, wherein step (ii) comprises determining the methylation status of a group of MVPs comprising or consisting of all 9 MVPs of SEQ ID NOS: 145 to 153 denoted by [CG]; wherein in step (iii) the methylation pattern is defined to score as methylated all 9 MVPs of SEQ ID NOS: 145 to 153 denoted by [CG]; and wherein in step (v) the DMR methylation pattern frequency threshold value in the sample DNA is 0.0005 or more, preferably 0.0008.

81. A method according to any one of claims 1 to 16, wherein in step (i) the DMR is comprised within the sequence set forth in SEQ ID NO: 154, and in step (ii) the group of linked MVPs are the 7 MVPs of SEQ ID NOS: 155 to 161 denoted by [CG].

82. A method according to claim 81, wherein step (ii) comprises determining the methylation status of at least four or at least five MVPs of SEQ ID NOS: 155 to 161 denoted by [CG]; or wherein step (ii) comprises determining the methylation status of all 7 MVPs of SEQ ID NOS: 155 to 161 denoted by [CG].

83. A method according to any one of claims 81 to 82, wherein in step (iii) the methylation pattern is defined to score as methylated each MVP in a group of MVPs comprising the MVPs of SEQ ID NOS: 155 to 161 denoted by [CG]; or wherein in step (iii) the methylation pattern is defined to score as methylated each MVP in a group of MVPs consisting of the MVPs of SEQ ID NOS: 155 to 161 denoted by [CG].

84. A method according to claim 81, wherein step (ii) comprises determining the methylation status of a group of MVPs comprising or consisting of all 7 MVPs of SEQ ID NOS: 155 to 161 denoted by [CG]; wherein in step (iii) the methylation pattern is defined to score as methylated all 7 MVPs of SEQ ID NOS: 155 to 161 denoted by [CG]; and wherein in step (v) the DMR methylation pattern frequency threshold value in the sample DNA is 0.0005 or more, preferably 0.0008.

85. A method according to any one of claims 1 to 16, wherein in step (i) the DMR is comprised within the sequence set forth in SEQ ID NO: 162, and in step (ii) the group of linked MVPs are the 5 MVPs of SEQ ID NOS: 163 to 167 denoted by [CG].

86. A method according to claim 85, wherein step (ii) comprises determining the methylation status of at least four or at least five MVPs of SEQ ID NOS: 163 to 167 denoted by [CG]; or wherein step (ii) comprises determining the methylation status of all 5 MVPs of SEQ ID NOS: 163 to 167 denoted by [CG].

87. A method according to any one of claims 85 to 86, wherein in step (iii) the methylation pattern is defined to score as methylated each MVP in a group of MVPs comprising the MVPs of SEQ ID NOS: 163 to 167 denoted by [CG]; or wherein in step (iii) the methylation pattern is defined to score as methylated each MVP in a group of MVPs consisting of the MVPs of SEQ ID NOS: 163 to 167 denoted by [CG].

88. A method according to claim 85, wherein step (ii) comprises determining the methylation status of a group of MVPs comprising or consisting of all 5 MVPs of SEQ ID NOS: 163 to 167 denoted by [CG]; wherein in step (iii) the methylation pattern is defined to score as methylated all 5 MVPs of SEQ ID NOS: 163 to 167 denoted by [CG]; and wherein in step (v) the DMR methylation pattern frequency threshold value in the sample DNA is 0.0005 or more, preferably 0.0008.

89. A method according to any one of claims 1 to 16, wherein in step (i) the DMR is comprised within the sequence set forth in SEQ ID NO: 168, and in step (ii) the group of linked MVPs are the 12 MVPs of SEQ ID NOS: 169 to 180 denoted by [CG].

90. A method according to claim 89, wherein step (ii) comprises determining the methylation status of at least four or at least five MVPs of SEQ ID NOS: 169 to 180 denoted by [CG]; or wherein step (ii) comprises determining the methylation status of all 12 MVPs of SEQ ID NOS: 169 to 180 denoted by [CG].

91. A method according to any one of claims 89 to 90, wherein in step (iii) the methylation pattern is defined to score as methylated each MVP in a group of MVPs comprising the MVPs of SEQ ID NOS: 169 to 180 denoted by [CG]; or wherein in step (iii) the methylation pattern is defined to score as methylated each MVP in a group of MVPs consisting of the MVPs of SEQ ID NOS: 169 to 180 denoted by [CG].

92. A method according to claim 89, wherein step (ii) comprises determining the methylation status of a group of MVPs comprising or consisting of all 12 MVPs of SEQ ID NOS: 169 to 180 denoted by [CG]; wherein in step (iii) the methylation pattern is defined to score as methylated all 12 MVPs of SEQ ID NOS: 169 to 180 denoted by [CG]; and wherein in step (v) the DMR methylation pattern frequency threshold value in the sample DNA is 0.0005 or more, preferably 0.0008.

93. A method according to any one of claims 1 to 16, wherein in step (i) the DMR is comprised within the sequence set forth in SEQ ID NO: 181, and in step (ii) the group of linked MVPs are the 11 MVPs of SEQ ID NOS: 182 to 192 denoted by [CG].

94. A method according to claim 93, wherein step (ii) comprises determining the methylation status of at least four or at least five MVPs of SEQ ID NOS: 182 to 192 denoted by [CG]; or wherein step (ii) comprises determining the methylation status of all 11 MVPs of SEQ ID NOS: 182 to 192 denoted by [CG].

95. A method according to any one of claims 93 to 94, wherein in step (iii) the methylation pattern is defined to score as methylated each MVP in a group of MVPs comprising the MVPs of SEQ ID NOS: 182 to 192 denoted by [CG]; or wherein in step (iii) the methylation pattern is defined to score as methylated each MVP in a group of MVPs consisting of the MVPs of SEQ ID NOS: 182 to 192 denoted by [CG].

96. A method according to claim 93, wherein step (ii) comprises determining the methylation status of a group of MVPs comprising or consisting of all 11 MVPs of SEQ ID NOS: 182 to 192 denoted by [CG]; wherein in step (iii) the methylation pattern is defined to score as methylated all 11 MVPs of SEQ ID NOS: 182 to 192 denoted by [CG]; and wherein in step (v) the DMR methylation pattern frequency threshold value in the sample DNA is 0.0005 or more, preferably 0.0008.

97. A method according to any one of claims 1 to 16, wherein in step (i) the DMR is comprised within the sequence set forth in SEQ ID NO: 193, and in step (ii) the group of linked MVPs are the 7 MVPs of SEQ ID NOS: 194 to 200 denoted by [CG].

98. A method according to claim 97, wherein step (ii) comprises determining the methylation status of at least four or at least five MVPs of SEQ ID NOS: 194 to 200 denoted by [CG]; or wherein step (ii) comprises determining the methylation status of all 7 MVPs of SEQ ID NOS: 194 to 200 denoted by [CG].

99. A method according to any one of claims 97 to 98, wherein in step (iii) the methylation pattern is defined to score as methylated each MVP in a group of MVPs comprising the MVPs of SEQ ID NOS: 194 to 200 denoted by [CG]; or wherein in step (iii) the methylation pattern is defined to score as methylated each MVP in a group of MVPs consisting of the MVPs of SEQ ID NOS: 194 to 200 denoted by [CG].

100. A method according to claim 97, wherein step (ii) comprises determining the methylation status of a group of MVPs comprising or consisting of all 7 MVPs of SEQ ID NOS: 194 to 200 denoted by [CG]; wherein in step (iii) the methylation pattern is defined to score as methylated all 7 MVPs of SEQ ID NOS: 194 to 200 denoted by [CG]; and wherein in step (v) the DMR methylation pattern frequency threshold value in the sample DNA is 0.0005 or more, preferably 0.0008.

101. A method according to any one of claims 1 to 16, wherein in step (i) the DMR is comprised within the sequence set forth in SEQ ID NO: 201, and in step (ii) the group of linked MVPs are the 11 MVPs of SEQ ID NOS: 202 to 212 denoted by [CG].

102. A method according to claim 101, wherein step (ii) comprises determining the methylation status of at least four or at least five MVPs of SEQ ID NOS: 202 to 212 denoted by [CG]; or wherein step (ii) comprises determining the methylation status of all 11 MVPs of SEQ ID NOS: 202 to 212 denoted by [CG].

103. A method according to any one of claims 101 to 102, wherein in step (iii) the methylation pattern is defined to score as methylated each MVP in a group of MVPs comprising the MVPs of SEQ ID NOS: 202 to 212 denoted by [CG]; or wherein in step (iii) the methylation pattern is defined to score as methylated each MVP in a group of MVPs consisting of the MVPs of SEQ ID NOS: 202 to 212 denoted by [CG].

104. A method according to claim 101, wherein step (ii) comprises determining the methylation status of a group of MVPs comprising or consisting of all 11 MVPs of SEQ ID NOS: 202 to 212 denoted by [CG]; wherein in step (iii) the methylation pattern is defined to score as methylated all 11 MVPs of SEQ ID NOS: 202 to 212 denoted by [CG]; and wherein in step (v) the DMR methylation pattern frequency threshold value in the sample DNA is 0.0005 or more, preferably 0.0008.

105. A method according to any one of claims 1 to 16, wherein in step (i) the DMR is comprised within the sequence set forth in SEQ ID NO: 213, and in step (ii) the group of linked MVPs are the 10 MVPs of SEQ ID NOS: 214 to 223 denoted by [CG].

106. A method according to claim 105, wherein step (ii) comprises determining the methylation status of at least four or at least five MVPs of SEQ ID NOS: 214 to 223 denoted by [CG]; or wherein step (ii) comprises determining the methylation status of all 10 MVPs of SEQ ID NOS: 214 to 223 denoted by [CG].

107. A method according to any one of claims 105 to 106, wherein in step (iii) the methylation pattern is defined to score as methylated each MVP in a group of MVPs comprising the MVPs of SEQ ID NOS: 214 to 223 denoted by [CG]; or wherein in step (iii) the methylation pattern is defined to score as methylated each MVP in a group of MVPs consisting of the MVPs of SEQ ID NOS: 214 to 223 denoted by [CG].

108. A method according to claim 105, wherein step (ii) comprises determining the methylation status of a group of MVPs comprising or consisting of all 10 MVPs of SEQ ID NOS: 214 to 223 denoted by [CG]; wherein in step (iii) the methylation pattern is defined to score as methylated all 10 MVPs of SEQ ID NOS: 214 to 223 denoted by [CG]; and wherein in step (v) the DMR methylation pattern frequency threshold value in the sample DNA is 0.0005 or more, preferably 0.0008.

109. A method according to any one of claims 1 to 16, wherein in step (i) the DMR is comprised within the sequence set forth in SEQ ID NO: 224, and in step (ii) the group of linked MVPs are the 10 MVPs of SEQ ID NOS: 225 to 234 denoted by [CG].

110. A method according to claim 109, wherein step (ii) comprises determining the methylation status of at least four or at least five MVPs of SEQ ID NOS: 225 to 234 denoted by [CG]; or wherein step (ii) comprises determining the methylation status of all 10 MVPs of SEQ ID NOS: 225 to 234 denoted by [CG].

111. A method according to any one of claims 109 to 110, wherein in step (iii) the methylation pattern is defined to score as methylated each MVP in a group of MVPs comprising the MVPs of SEQ ID NOS: 225 to 234 denoted by [CG]; or wherein in step (iii) the methylation pattern is defined to score as methylated each MVP in a group of MVPs consisting of the MVPs of SEQ ID NOS: 225 to 234 denoted by [CG].

112. A method according to claim 109, wherein step (ii) comprises determining the methylation status of a group of MVPs comprising or consisting of all 10 MVPs of SEQ ID NOS: 225 to 234 denoted by [CG]; wherein in step (iii) the methylation pattern is defined to score as methylated all 10 MVPs of SEQ ID NOS: 225 to 234 denoted by [CG]; and wherein in step (v) the DMR methylation pattern frequency threshold value in the sample DNA is 0.0005 or more, preferably 0.0008.

113. A method according to any one of claims 1 to 16, wherein in step (i) the DMR is comprised within the sequence set forth in SEQ ID NO: 235, and in step (ii) the group of linked MVPs are the 6 MVPs of SEQ ID NOS: 236 to 241 denoted by [CG].

114. A method according to claim 113, wherein step (ii) comprises determining the methylation status of at least four or at least five MVPs of SEQ ID NOS: 236 to 241 denoted by [CG]; or wherein step (ii) comprises determining the methylation status of all 6 MVPs of SEQ ID NOS: 236 to 241 denoted by [CG].

115. A method according to any one of claims 113 to 114, wherein in step (iii) the methylation pattern is defined to score as methylated each MVP in a group of MVPs comprising the MVPs of SEQ ID NOS: 236 to 241 denoted by [CG]; or wherein in step (iii) the methylation pattern is defined to score as methylated each MVP in a group of MVPs consisting of the MVPs of SEQ ID NOS: 236 to 241 denoted by [CG].

116. A method according to claim 113, wherein step (ii) comprises determining the methylation status of a group of MVPs comprising or consisting of all 6 MVPs of SEQ ID NOS: 236 to 241 denoted by [CG]; wherein in step (iii) the methylation pattern is defined to score as methylated all 6 MVPs of SEQ ID NOS: 236 to 241 denoted by [CG]; and wherein in step (v) the DMR methylation pattern frequency threshold value in the sample DNA is 0.0005 or more, preferably 0.0008.

117. A method according to any one of claims 1 to 16, wherein in step (i) the DMR is comprised within the sequence set forth in SEQ ID NO: 242, and in step (ii) the group of linked MVPs are the 9 MVPs of SEQ ID NOS: 243 to 251 denoted by [CG].

118. A method according to claim 117, wherein step (ii) comprises determining the methylation status of at least four or at least five MVPs of SEQ ID NOS: 243 to 251 denoted by [CG]; or wherein step (ii) comprises determining the methylation status of all 9 MVPs of SEQ ID NOS: 243 to 251 denoted by [CG].

119. A method according to any one of claims 117 to 118, wherein in step (iii) the methylation pattern is defined to score as methylated each MVP in a group of MVPs comprising the MVPs of SEQ ID NOS: 243 to 251 denoted by [CG]; or wherein in step (iii) the methylation pattern is defined to score as methylated each MVP in a group of MVPs consisting of the MVPs of SEQ ID NOS: 243 to 251 denoted by [CG].

120. A method according to claim 117, wherein step (ii) comprises determining the methylation status of a group of MVPs comprising or consisting of all 9 MVPs of SEQ ID NOS: 243 to 251 denoted by [CG]; wherein in step (iii) the methylation pattern is defined to score as methylated all 9 MVPs of SEQ ID NOS: 243 to 251 denoted by [CG]; and wherein in step (v) the DMR methylation pattern frequency threshold value in the sample DNA is 0.0005 or more, preferably 0.0008.

121. A method according to any one of claims 1 to 16, wherein in step (i) the DMR is comprised within the sequence set forth in SEQ ID NO: 252, and in step (ii) the group of linked MVPs are the 7 MVPs of SEQ ID NOS: 253 to 259 denoted by [CG].

122. A method according to claim 121, wherein step (ii) comprises determining the methylation status of at least four or at least five MVPs of SEQ ID NOS: 253 to 259 denoted by [CG]; or wherein step (ii) comprises determining the methylation status of all 7 MVPs of SEQ ID NOS: 253 to 259 denoted by [CG].

123. A method according to any one of claims 121 to 123, wherein in step (iii) the methylation pattern is defined to score as methylated each MVP in a group of MVPs comprising the MVPs of SEQ ID NOS: 253 to 259 denoted by [CG]; or wherein in step (iii) the methylation pattern is defined to score as methylated each MVP in a group of MVPs consisting of the MVPs of SEQ ID NOS: 253 to 259 denoted by [CG].

124. A method according to claim 121, wherein step (ii) comprises determining the methylation status of a group of MVPs comprising or consisting of all 7 MVPs of SEQ ID NOS: 253 to 259 denoted by [CG]; wherein in step (iii) the methylation pattern is defined to score as methylated all 7 MVPs of SEQ ID NOS: 253 to 259 denoted by [CG]; and wherein in step (v) the DMR methylation pattern frequency threshold value in the sample DNA is 0.0005 or more, preferably 0.0008.

125. A method according to any one of claims 1 to 16, wherein in step (i) the DMR is comprised within the sequence set forth in SEQ ID NO: 260, and in step (ii) the group of linked MVPs are the 6 MVPs of SEQ ID NOS: 261 to 266 denoted by [CG].

126. A method according to claim 125, wherein step (ii) comprises determining the methylation status of at least four or at least five MVPs of SEQ ID NOS: 261 to 266 denoted by [CG]; or wherein step (ii) comprises determining the methylation status of all 6 MVPs of SEQ ID NOS: 261 to 266 denoted by [CG].

127. A method according to any one of claims 125 to 126, wherein in step (iii) the methylation pattern is defined to score as methylated each MVP in a group of MVPs comprising the MVPs of SEQ ID NOS: 261 to 266 denoted by [CG]; or wherein in step (iii) the methylation pattern is defined to score as methylated each MVP in a group of MVPs consisting of the MVPs of SEQ ID NOS: 261 to 266 denoted by [CG].

128. A method according to claim 125, wherein step (ii) comprises determining the methylation status of a group of MVPs comprising or consisting of all 6 MVPs of SEQ ID NOS: 261 to 266 denoted by [CG]; wherein in step (iii) the methylation pattern is defined to score as methylated all 6 MVPs of SEQ ID NOS: 261 to 266 denoted by [CG]; and wherein in step (v) the DMR methylation pattern frequency threshold value in the sample DNA is 0.0005 or more, preferably 0.0008.

129. A method according to any one of claims 1 to 16, wherein in step (i) the DMR is comprised within the sequence set forth in SEQ ID NO: 267, and in step (ii) the group of linked MVPs are the 10 MVPs of SEQ ID NOS: 268 to 277 denoted by [CG].

130. A method according to claim 129, wherein step (ii) comprises determining the methylation status of at least four or at least five MVPs of SEQ ID NOS: 268 to 277 denoted by [CG]; or wherein step (ii) comprises determining the methylation status of all 10 MVPs of SEQ ID NOS: 268 to 277 denoted by [CG].

131. A method according to any one of claims 129 to 130, wherein in step (iii) the methylation pattern is defined to score as methylated each MVP in a group of MVPs comprising the MVPs of SEQ ID NOS: 268 to 277 denoted by [CG]; or wherein in step (iii) the methylation pattern is defined to score as methylated each MVP in a group of MVPs consisting of the MVPs of SEQ ID NOS: 268 to 277 denoted by [CG].

132. A method according to claim 129, wherein step (ii) comprises determining the methylation status of a group of MVPs comprising or consisting of all 10 MVPs of SEQ ID NOS: 268 to 277 denoted by [CG]; wherein in step (iii) the methylation pattern is defined to score as methylated all 10 MVPs of SEQ ID NOS: 268 to 277 denoted by [CG]; and wherein in step (v) the DMR methylation pattern frequency threshold value in the sample DNA is 0.0005 or more, preferably 0.0008.

133. A method according to any one of claims 1 to 16, wherein in step (i) the DMR is comprised within the sequence set forth in SEQ ID NO: 278, and in step (ii) the group of linked MVPs are the 6 MVPs of SEQ ID NOS: 279 to 284 denoted by [CG].

134. A method according to claim 133, wherein step (ii) comprises determining the methylation status of at least four or at least five MVPs of SEQ ID NOS: 279 to 284 denoted by [CG]; or wherein step (ii) comprises determining the methylation status of all 6 MVPs of SEQ ID NOS: 279 to 284 denoted by [CG].

135. A method according to any one of claims 133 to 134, wherein in step (iii) the methylation pattern is defined to score as methylated each MVP in a group of MVPs comprising the MVPs of SEQ ID NOS: 279 to 284 denoted by [CG]; or wherein in step (iii) the methylation pattern is defined to score as methylated each MVP in a group of MVPs consisting of the MVPs of SEQ ID NOS: 279 to 284 denoted by [CG].

136. A method according to claim 133, wherein step (ii) comprises determining the methylation status of a group of MVPs comprising or consisting of all 6 MVPs of SEQ ID NOS: 279 to 284 denoted by [CG]; wherein in step (iii) the methylation pattern is defined to score as methylated all 6 MVPs of SEQ ID NOS: 279 to 284 denoted by [CG]; and wherein in step (v) the DMR methylation pattern frequency threshold value in the sample DNA is 0.0005 or more, preferably 0.0008.

137. A method according to any one of claims 1 to 16, wherein in step (i) the DMR is comprised within the sequence set forth in SEQ ID NO: 285, and in step (ii) the group of linked MVPs are the 6 MVPs of SEQ ID NOS: 286 to 291 denoted by [CG].

138. A method according to claim 137, wherein step (ii) comprises determining the methylation status of at least four or at least five MVPs of SEQ ID NOS: 286 to 291 denoted by [CG]; or wherein step (ii) comprises determining the methylation status of all 6 MVPs of SEQ ID NOS: 286 to 291 denoted by [CG].

139. A method according to any one of claims 137 to 138, wherein in step (iii) the methylation pattern is defined to score as methylated each MVP in a group of MVPs comprising the MVPs of SEQ ID NOS: 286 to 291 denoted by [CG]; or wherein in step (iii) the methylation pattern is defined to score as methylated each MVP in a group of MVPs consisting of the MVPs of SEQ ID NOS: 286 to 291 denoted by [CG].

140. A method according to claim 137, wherein step (ii) comprises determining the methylation status of a group of MVPs comprising or consisting of all 6 MVPs of SEQ ID NOS: 286 to 291 denoted by [CG]; wherein in step (iii) the methylation pattern is defined to score as methylated all 6 MVPs of SEQ ID NOS: 286 to 291 denoted by [CG]; and wherein in step (v) the DMR methylation pattern frequency threshold value in the sample DNA is 0.0005 or more, preferably 0.0008.

141. A method according to any one of claims 1 to 16, wherein in step (i) the DMR is comprised within the sequence set forth in SEQ ID NO: 292, and in step (ii) the group of linked MVPs are the 4 MVPs of SEQ ID NOS: 293 to 296 denoted by [CG].

142. A method according to claim 141, wherein step (ii) comprises determining the methylation status of at least four or at least five MVPs of SEQ ID NOS: 293 to 296 denoted by [CG]; or wherein step (ii) comprises determining the methylation status of all 4 MVPs of SEQ ID NOS: 293 to 296 denoted by [CG].

143. A method according to any one of claims 141 to 142, wherein in step (iii) the methylation pattern is defined to score as methylated each MVP in a group of MVPs comprising the MVPs of SEQ ID NOS: 293 to 296 denoted by [CG]; or wherein in step (iii) the methylation pattern is defined to score as methylated each MVP in a group of MVPs consisting of the MVPs of SEQ ID NOS: 293 to 296 denoted by [CG].

144. A method according to claim 141, wherein step (ii) comprises determining the methylation status of a group of MVPs comprising or consisting of all 4 MVPs of SEQ ID NOS: 293 to 296 denoted by [CG]; wherein in step (iii) the methylation pattern is defined to score as methylated all 4 MVPs of SEQ ID NOS: 293 to 296 denoted by [CG]; and wherein in step (v) the DMR methylation pattern frequency threshold value in the sample DNA is 0.0005 or more, preferably 0.0008.

145. A method according to any one of the preceding claims, wherein for a given DMR the step of determining the methylation status of MVPs and the step of selecting a DMR methylation pattern for MVPs is performed by a single process, the process comprising the steps of:
a) amplifying bisulphite converted sample DNA to form methylation pattern amplicons comprising DMRs or sub-regions of DMRs, preferably wherein the amplifying step is performed using PCR; and
b) simultaneously determining the methylation status of MVPs and the DMR methylation pattern within DMRs or within sub-regions of DMRs by detecting the formation of methylation pattern amplicons.

146. A method according to claim 145, wherein step (a) comprises amplifying using forward and reverse primers which are designed to anneal to sites which flank regions of MVPs to be analysed within DMRs or within sub-regions of DMRs, and wherein in step (b) the formation of methylation pattern amplicons is detected using one or more detection probes, wherein the one or more detection probes are designed to anneal to sites comprising MVPs to be analysed, wherein annealing is dependent upon the methylation status of MVPs, and wherein sequence-dependent annealing of the one or more detection probes is detected during or after the amplification step.

147. A method according to claim 146, further comprising the use of forward blocker oligonucleotides and/or reverse blocker oligonucleotides, wherein blocker oligonucleotides are designed to anneal to sites comprising MVPs to be analysed, provided that blocker oligonucleotides are designed not to anneal to a site comprising a sequence which prior to bisulphite conversion comprised MVPs whose methylation status matched the status of MVPs in a selected pre-defined DMR methylation pattern, wherein the annealing site for a forward blocker oligonucleotide and the annealing site for a reverse blocker oligonucleotide overlaps with the annealing site for forward and reverse primers respectively, and wherein annealing of a blocker oligonucleotide prevents annealing of a respective primer whereupon amplification is prevented.

148. A method according to claim 146, further comprising the use of a forward blocker oligonucleotide and/or a reverse blocker oligonucleotide, wherein blocker oligonucleotides are designed to anneal to sites comprising MVPs to be analysed and to anneal only when each MVP within the site was unmethylated prior to bisulphite conversion, wherein the annealing site for a forward blocker oligonucleotide and the annealing site for a reverse blocker oligonucleotide overlaps with the annealing site for forward and reverse primers respectively, and wherein annealing of a blocker oligonucleotide prevents annealing of a respective primer whereupon amplification prevented.

149. A method according to claim 146, wherein step (a) comprises amplifying using forward and reverse primers which are designed to anneal to sites comprising MVPs to be analysed, wherein annealing is dependent upon the methylation status of MVPs, and wherein in step (b) the formation of methylation pattern amplicons is detected using one or more detection probes, wherein the one or more detection probes are designed to anneal to sites between MVPs to be analysed, and wherein sequence-dependent annealing of the one or more detection probes is detected during or after the amplification step.

150. A method according to claim 146, wherein step (a) comprises amplifying using forward and reverse primers which are designed to anneal to sites comprising MVPs to be analysed, wherein annealing is dependent upon the methylation status of MVPs, and wherein in step (b) the formation of methylation pattern amplicons is detected using one or more detection probes, wherein the one or more detection probes are designed to anneal to sites comprising MVPs to be analysed, wherein annealing is dependent upon the methylation status of MVPs, and wherein sequence-dependent annealing of the one or more detection probes is detected during or after the amplification step.

151. A method according to claim 149 or claim 150, further comprising the use of forward blocker oligonucleotides and/or reverse blocker oligonucleotides, wherein forward and reverse blocker oligonucleotides are designed to anneal to sites comprising MVPs to be analysed, and wherein the MVPs to be analysed are the same MVPs comprised respectively within forward and reverse primer binding sites, provided that a blocker oligonucleotide is designed not to anneal to a site wherein prior to bisulphite conversion the methylation status of MVPs within the site matched the status of MVPs within a selected pre-defined DMR methylation pattern, and wherein annealing of a blocker oligonucleotide prevents annealing of a respective primer whereupon amplification is prevented.

152. A method according to claim 149 or claim 150, further comprising the use of forward blocker oligonucleotides and/or reverse blocker oligonucleotides, wherein forward and reverse blocker oligonucleotides are designed to anneal to sites comprising MVPs to be analysed, and wherein the MVPs to be analysed are the same MVPs comprised respectively within forward and reverse primer binding sites, provided that a blocker oligonucleotide is designed to anneal only when each MVP within the site was unmethylated prior to bisulphite conversion, and wherein annealing of a blocker oligonucleotide prevents annealing of a respective primer whereupon amplification is prevented.

153. A method according to any one of claim 147, 148, 151 or 152, wherein both forward and reverse blocker oligonucleotides are used.

154. A method according to any one of claims 145 to 153, wherein the step of determining a pattern frequency for the DMR methylation pattern within the sample DNA comprises quantifying methylation pattern amplicons produced during a number of amplification cycles, quantifying control amplicons produced during the same number of amplification cycles and determining the ratio of methylation pattern amplicons to control amplicons.

155. A method according to claim 154, wherein control amplicons are produced by a process comprising amplifying bisulphite converted sample DNA to form amplicons comprising the DMR or a sub-region of the DMR, wherein amplification is performed using forward and reverse primers which are designed to anneal to DMR sequences which exclude MVPs to be analysed, preferably wherein the quantity of sample DNA amplified to produce control amplicons is the same as the quantity of sample DNA amplified to produce methylation pattern amplicons, more preferably wherein the control amplicons and methylation pattern amplicons are produced from the same sample DNA in the same reaction vessel during the same amplification cycles.

156. A method according to claim 155, wherein the formation of control amplicons is detected using one or more detection probes, wherein the one or more detection probes are designed to anneal to sequences which exclude MVPs to be analysed and which are located between forward and reverse primer annealing sites.

157. A method according to any one of claims 146 to 155, wherein the one or more detection probes is an oligonucleotide comprising a fluorophore and a quencher and wherein quenching occurs by fluorescence resonance energy transfer (FRET) or by static/contact quenching.

158. A method according to claim 157, wherein when the one or more detection probes is annealed, fluorescence from the fluorophore is quenched.

159. A method according to claim 158, wherein quenching of fluorescence is disrupted by the exonuclease action of DNA polymerase during the step of amplification.

160. A method according to claim 157, wherein quenching of fluorescence is disrupted when the one or more detection probes is annealed.

161. A method according to any one claims 1 to 144, wherein for a given DMR the step of determining the methylation status of MVPs comprises the steps of:
a) amplifying bisulphite converted sample DNA, preferably by PCR; and
b) analysing MVPs within DMRs or within sub-regions of DMRs.

162. A method according to claim 161, wherein the step of analysing MVPs within DMRs or within sub-regions of DMRs comprises sequencing the DMRs or sub-regions of DMRs or portions thereof.

163. A method according to claim 162, wherein adaptor sequences to facilitate DNA sequencing are incorporated into amplicons during the step of amplifying sample DNA or wherein adaptor sequences to facilitate DNA sequencing are ligated to amplicons after the step of amplifying sample DNA.

164. A method according to claim 163, wherein unique index sequences (barcode sequences) are incorporated into amplicons during the step of amplifying sample DNA, or wherein barcode sequences are ligated to amplicons after the step of amplifying sample DNA; wherein each barcode sequence is designed to be specific for a given sample from an individual.

165. A method according to claim 164, wherein prior to the sequencing step two or more populations of amplicons are pooled to form a library of amplicons, wherein amplicons from different populations have different barcode sequences and amplicons from the same population have the same barcode sequence.

166. A method according to any one of claims 161 to 164, wherein the step of analysing MVPs within DMRs or within sub-regions of DMRs to determine the methylation status of MVPs within DMRs or within sub-regions of DMRs comprises analysing sequencing reads, preferably wherein sequencing reads are analysed using a sequence analysis software program.

167. A method according to claim 166, wherein the step of determining a pattern frequency for the pre-defined DMR methylation pattern within the sample DNA comprises determining the proportion of sequencing reads which score positive for the selected pre-defined DMR methylation pattern, determining the proportion of sequencing reads which score negative for the selected pre-defined DMR methylation pattern and determining a ratio of positive to negative sequencing reads, preferably wherein sequencing reads are analysed and scored using a computer algorithm.

168. A method according to any one of the preceding claims, wherein the sample from the individual is a sample of serum, preferably wherein sample DNA is cell-free DNA obtained following removal of cells from serum.

169. A method of treating a patient having metastatic breast cancer (mBC) comprising identifying mBC DNA within a sample from the individual by performing the method of any one of the preceding claims and providing one or more cancer treatments to the patient.

170. A method according to claim 169, wherein the one or more cancer treatments comprise one or more surgical procedures, one or more chemotherapeutic agents, one or more cytotoxic chemotherapeutic agents, one or more radiotherapeutic agents, one or more immunotherapeutic agents or any combination thereof.

171. A detection probe comprising an isolated oligonucleotide molecule and a detection system, wherein the probe is designed to anneal to a site comprising one or more of the MVPs of or corresponding to SEQ ID NOS: 2 to 12 denoted by [CG], and wherein annealing is dependent on the methylation status of the one or more MVPs.

172. A detection probe comprising an isolated oligonucleotide molecule and a detection system, wherein the probe is designed to anneal to a site comprising one or more of the MVPs of or corresponding to SEQ ID NOS: 14 to 24 denoted by [CG], or to a site comprising one or more of the MVPs of or corresponding to SEQ ID NOS: 26 to 36 denoted by [CG], or to a site comprising one or more of the MVPs of or corresponding to SEQ ID NOS: 38 to 53 denoted by [CG], or to a site comprising one or more of the MVPs of or corresponding to SEQ ID NOS: 55 to 66 denoted by [CG], or to a site comprising one or more of the MVPs of or corresponding to SEQ ID NOS: 68 to 74 denoted by [CG], or to a site comprising one or more of the MVPs of or corresponding to SEQ ID NOS: 76 to 82 denoted by [CG], or to a site comprising one or more of the MVPs of or corresponding to SEQ ID NOS: 84 to 88 denoted by [CG], or to a site comprising one or more of the MVPs of or corresponding to SEQ ID NOS: 90 to 96 denoted by [CG], or to a site comprising one or more of the MVPs of or corresponding to SEQ ID NOS: 98 to 111 denoted by [CG], or to a site comprising one or more of the MVPs of or corresponding to SEQ ID NOS: 113 to 119 denoted by [CG], or to a site comprising one or more of the MVPs of or corresponding to SEQ ID NOS: 121 to 128 denoted by [CG], or to a site comprising one or more of the MVPs of or corresponding to SEQ ID NOS: 130 to 136 denoted by [CG], or to a site comprising one or more of the MVPs of or corresponding to SEQ ID NOS: 138 to 143 denoted by [CG], or to a site comprising one or more of the MVPs of or corresponding to SEQ ID NOS: 145 to 153 denoted by [CG], or to a site comprising one or more of the MVPs of or corresponding to SEQ ID NOS: 155 to 161 denoted by [CG], or to a site comprising one or more of the MVPs of or corresponding to SEQ ID NOS: 163 to 167 denoted by [CG], or to a site comprising one or more of the MVPs of or corresponding to SEQ ID NOS: 169 to 180 denoted by [CG], or to a site comprising one or more of the MVPs of or corresponding to SEQ ID NOS: 182 to 192 denoted by [CG], or to a site comprising one or more of the MVPs of or corresponding to SEQ ID NOS: 194 to 200 denoted by [CG], or to a site comprising one or more of the MVPs of or corresponding to SEQ ID NOS: 202 to 212 denoted by [CG], or to a site comprising one or more of the MVPs of or corresponding to SEQ ID NOS: 214 to 223 denoted by [CG], or to a site comprising one or more of the MVPs of or corresponding to SEQ ID NOS: 225 to 234 denoted by [CG], or to a site comprising one or more of the MVPs of or corresponding to SEQ ID NOS: 236 to 241 denoted by [CG], or to a site comprising one or more of the MVPs of or corresponding to SEQ ID NOS: 243 to 251 denoted by [CG], or to a site comprising one or more of the MVPs of or corresponding to SEQ ID NOS: 253 to 259 denoted by [CG], or to a site comprising one or more of the MVPs of or corresponding to SEQ ID NOS: 261 to 266 denoted by [CG], or to a site comprising one or more of the MVPs of or corresponding to SEQ ID NOS: 268 to 277 denoted by [CG], or to a site comprising one or more of the MVPs of or corresponding to SEQ ID NOS: 279 to 284 denoted by [CG], or to a site comprising one or more of the MVPs of or corresponding to SEQ ID NOS: 286 to 291 denoted by [CG], and wherein in each case annealing of the probe is dependent on the methylation status of the one or more MVPs.

173. A detection probe according to claim 171 or claim 172, wherein the detection system comprises one or more fluorophore and quencher pairs, wherein the quencher of a pair is capable of quenching the fluorescence of the fluorophore of the pair.

174. A detection probe according to claim 173, wherein the quencher is capable of quenching the fluorescence of the fluorophore by fluorescence resonance energy transfer (FRET) or by static/contact quenching.

175. An isolated oligonucleotide molecule for use as an amplification primer, wherein the molecule is designed to anneal to a site comprising one or more of the MVPs of or corresponding to SEQ ID NOS: 2 to 12 denoted by [CG], and wherein annealing is dependent on the methylation status of the one or more MVPs.

176. An isolated oligonucleotide molecule for use as an amplification primer, wherein the molecule is designed to anneal to a site comprising one or more of the MVPs of or corresponding to SEQ ID NOS: 14 to 24 denoted by [CG], or to a site comprising one or more of the MVPs of or corresponding to SEQ ID NOS: 26 to 36 denoted by [CG], or to a site comprising one or more of the MVPs of or corresponding to SEQ ID NOS: 38 to 53 denoted by [CG], or to a site comprising one or more of the MVPs of or corresponding to SEQ ID NOS: 55 to 66 denoted by [CG], or to a site comprising one or more of the MVPs of or corresponding to SEQ ID NOS: 68 to 74 denoted by [CG], or to a site comprising one or more of the MVPs of or corresponding to SEQ ID NOS: 76 to 82 denoted by [CG], or to a site comprising one or more of the MVPs of or corresponding to SEQ ID NOS: 84 to 88 denoted by [CG], or to a site comprising one or more of the MVPs of or corresponding to SEQ ID NOS: 90 to 96 denoted by [CG], or to a site comprising one or more of the MVPs of or corresponding to SEQ ID NOS: 98 to 111 denoted by [CG], or to a site comprising one or more of the MVPs of or corresponding to SEQ ID NOS: 113 to 119 denoted by [CG], or to a site comprising one or more of the MVPs of or corresponding to SEQ ID NOS: 121 to 128 denoted by [CG], or to a site comprising one or more of the MVPs of or corresponding to SEQ ID NOS: 130 to 136 denoted by [CG], or to a site comprising one or more of the MVPs of or corresponding to SEQ ID NOS: 138 to 143 denoted by [CG], or to a site comprising one or more of the MVPs of or corresponding to SEQ ID NOS: 145 to 153 denoted by [CG], or to a site comprising one or more of the MVPs of or corresponding to SEQ ID NOS: 155 to 161 denoted by [CG], or to a site comprising one or more of the MVPs of or corresponding to SEQ ID NOS: 163 to 167 denoted by [CG], or to a site comprising one or more of the MVPs of or corresponding to SEQ ID NOS: 169 to 180 denoted by [CG], or to a site comprising one or more of the MVPs of or corresponding to SEQ ID NOS: 182 to 192 denoted by [CG], or to a site comprising one or more of the MVPs of or corresponding to SEQ ID NOS: 194 to 200 denoted by [CG], or to a site comprising one or more of the MVPs of or corresponding to SEQ ID NOS: 202 to 212 denoted by [CG], or to a site comprising one or more of the MVPs of or corresponding to SEQ ID NOS: 214 to 223 denoted by [CG], or to a site comprising one or more of the MVPs of or corresponding to SEQ ID NOS: 225 to 234 denoted by [CG], or to a site comprising one or more of the MVPs of or corresponding to SEQ ID NOS: 236 to 241 denoted by [CG], or to a site comprising one or more of the MVPs of or corresponding to SEQ ID NOS: 243 to 251 denoted by [CG], or to a site comprising one or more of the MVPs of or corresponding to SEQ ID NOS: 253 to 259 denoted by [CG], or to a site comprising one or more of the MVPs of or corresponding to SEQ ID NOS: 261 to 266 denoted by [CG], or to a site comprising one or more of the MVPs of or corresponding to SEQ ID NOS: 268 to 277 denoted by [CG], or to a site comprising one or more of the MVPs of or corresponding to SEQ ID NOS: 279 to 284 denoted by [CG], or to a site comprising one or more of the MVPs of or corresponding to SEQ ID NOS: 286 to 291 denoted by [CG], and wherein in each case annealing of the oligonucleotide is dependent on the methylation status of the one or more MVPs.

177. A pair of isolated oligonucleotide molecules for use as forward and reverse amplification primers, wherein one or both of the molecules are designed to anneal to a site comprising one or more of the MVPs of or corresponding to SEQ ID NOS: 2 to 12 denoted by [CG], and wherein annealing is dependent on the methylation status of the one or more MVPs.

178. A pair of isolated oligonucleotide molecules for use as forward and reverse amplification primers, wherein one or both of the molecules are designed to anneal to a site comprising one or more of the MVPs of or corresponding to SEQ ID NOS: 14 to 24 denoted by [CG], or to a site comprising one or more of the MVPs of or corresponding to SEQ ID NOS: 26 to 36 denoted by [CG], or to a site comprising one or more of the MVPs of or corresponding to SEQ ID NOS: 38 to 53 denoted by [CG], or to a site comprising one or more of the MVPs of or corresponding to SEQ ID NOS: 55 to 66 denoted by [CG], or to a site comprising one or more of the MVPs of or corresponding to SEQ ID NOS: 68 to 74 denoted by [CG], or to a site comprising one or more of the MVPs of or corresponding to SEQ ID NOS: 76 to 82 denoted by [CG], or to a site comprising one or more of the MVPs of or corresponding to SEQ ID NOS: 84 to 88 denoted by [CG], or to a site comprising one or more of the MVPs of or corresponding to SEQ ID NOS: 90 to 96 denoted by [CG], or to a site comprising one or more of the MVPs of or corresponding to SEQ ID NOS: 98 to 111 denoted by [CG], or to a site comprising one or more of the MVPs of or corresponding to SEQ ID NOS: 113 to 119 denoted by [CG], or to a site comprising one or more of the MVPs of or corresponding to SEQ ID NOS: 121 to 128 denoted by [CG], or to a site comprising one or more of the MVPs of or corresponding to SEQ ID NOS: 130 to 136 denoted by [CG], or to a site comprising one or more of the MVPs of or corresponding to SEQ ID NOS: 138 to 143 denoted by [CG], or to a site comprising one or more of the MVPs of or corresponding to SEQ ID NOS: 145 to 153 denoted by [CG], or to a site comprising one or more of the MVPs of or corresponding to SEQ ID NOS: 155 to 161 denoted by [CG], or to a site comprising one or more of the MVPs of or corresponding to SEQ ID NOS: 163 to 167 denoted by [CG], or to a site comprising one or more of the MVPs of or corresponding to SEQ ID NOS: 169 to 180 denoted by [CG], or to a site comprising one or more of the MVPs of or corresponding to SEQ ID NOS: 182 to 192 denoted by [CG], or to a site comprising one or more of the MVPs of or corresponding to SEQ ID NOS: 194 to 200 denoted by [CG], or to a site comprising one or more of the MVPs of or corresponding to SEQ ID NOS: 202 to 212 denoted by [CG], or to a site comprising one or more of the MVPs of or corresponding to SEQ ID NOS: 214 to 223 denoted by [CG], or to a site comprising one or more of the MVPs of or corresponding to SEQ ID NOS: 225 to 234 denoted by [CG], or to a site comprising one or more of the MVPs of or corresponding to SEQ ID NOS: 236 to 241 denoted by [CG], or to a site comprising one or more of the MVPs of or corresponding to SEQ ID NOS: 243 to 251 denoted by [CG], or to a site comprising one or more of the MVPs of or corresponding to SEQ ID NOS: 253 to 259 denoted by [CG], or to a site comprising one or more of the MVPs of or corresponding to SEQ ID NOS: 261 to 266 denoted by [CG], or to a site comprising one or more of the MVPs of or corresponding to SEQ ID NOS: 268 to 277 denoted by [CG], or to a site comprising one or more of the MVPs of or corresponding to SEQ ID NOS: 279 to 284 denoted by [CG], or to a site comprising one or more of the MVPs of or corresponding to SEQ ID NOS: 286 to 291 denoted by [CG], and wherein in each case annealing of the oligonucleotide is dependent on the methylation status of the one or more MVPs.

179. An isolated oligonucleotide molecule for use as an amplification primer, wherein the molecule is designed to anneal to a site adjacent to a DNA region comprising one or more of the MVPs of or corresponding to SEQ ID NOS: 2 to 12 denoted by [CG].

180. An isolated oligonucleotide molecule for use as an amplification primer, wherein the molecule is designed to anneal to a site adjacent to a DNA region comprising one or more of the MVPs of or corresponding to SEQ ID NOS: 14 to 24 denoted by [CG], or to a site comprising one or more of the MVPs of or corresponding to SEQ ID NOS: 26 to 36 denoted by [CG], or to a site comprising one or more of the MVPs of or corresponding to SEQ ID NOS: 38 to 53 denoted by [CG], or to a site comprising one or more of the MVPs of or corresponding to SEQ ID NOS: 55 to 66 denoted by [CG], or to a site comprising one or more of the MVPs of or corresponding to SEQ ID NOS: 68 to 74 denoted by [CG], or to a site comprising one or more of the MVPs of or corresponding to SEQ ID NOS: 76 to 82 denoted by [CG], or to a site comprising one or more of the MVPs of or corresponding to SEQ ID NOS: 84 to 88 denoted by [CG], or to a site comprising one or more of the MVPs of or corresponding to SEQ ID NOS: 90 to 96 denoted by [CG], or to a site comprising one or more of the MVPs of or corresponding to SEQ ID NOS: 98 to 111 denoted by [CG], or to a site comprising one or more of the MVPs of or corresponding to SEQ ID NOS: 113 to 119 denoted by [CG], or to a site comprising one or more of the MVPs of or corresponding to SEQ ID NOS: 121 to 128 denoted by [CG], or to a site comprising one or more of the MVPs of or corresponding to SEQ ID NOS: 130 to 136 denoted by [CG], or to a site comprising one or more of the MVPs of or corresponding to SEQ ID NOS: 138 to 143 denoted by [CG], or to a site comprising one or more of the MVPs of or corresponding to SEQ ID NOS: 145 to 153 denoted by [CG], or to a site comprising one or more of the MVPs of or corresponding to SEQ ID NOS: 155 to 161 denoted by [CG], or to a site comprising one or more of the MVPs of or corresponding to SEQ ID NOS: 163 to 167 denoted by [CG], or to a site comprising one or more of the MVPs of or corresponding to SEQ ID NOS: 169 to 180 denoted by [CG], or to a site comprising one or more of the MVPs of or corresponding to SEQ ID NOS: 182 to 192 denoted by [CG], or to a site comprising one or more of the MVPs of or corresponding to SEQ ID NOS: 194 to 200 denoted by [CG], or to a site comprising one or more of the MVPs of or corresponding to SEQ ID NOS: 202 to 212 denoted by [CG], or to a site comprising one or more of the MVPs of or corresponding to SEQ ID NOS: 214 to 223 denoted by [CG], or to a site comprising one or more of the MVPs of or corresponding to SEQ ID NOS: 225 to 234 denoted by [CG], or to a site comprising one or more of the MVPs of or corresponding to SEQ ID NOS: 236 to 241 denoted by [CG], or to a site comprising one or more of the MVPs of or corresponding to SEQ ID NOS: 243 to 251 denoted by [CG], or to a site comprising one or more of the MVPs of or corresponding to SEQ ID NOS: 253 to 259 denoted by [CG], or to a site comprising one or more of the MVPs of or corresponding to SEQ ID NOS: 261 to 266 denoted by [CG], or to a site comprising one or more of the MVPs of or corresponding to SEQ ID NOS: 268 to 277 denoted by [CG], or to a site comprising one or more of the MVPs of or corresponding to SEQ ID NOS: 279 to 284 denoted by [CG], or to a site comprising one or more of the MVPs of or corresponding to SEQ ID NOS: 286 to 291 denoted by [CG].

181. A pair of isolated oligonucleotide molecules for use as forward and reverse amplification primers, wherein one or both of the molecules are designed to anneal to a site adjacent to a DNA region comprising one or more of the MVPs of or corresponding to SEQ ID NOS: 2 to 12 denoted by [CG].

182. A pair of isolated oligonucleotide molecules for use as forward and reverse amplification primers, wherein one or both of the molecules are designed to anneal to a site adjacent to a DNA region comprising one or more of the MVPs of or corresponding to SEQ ID NOS: 14 to 24 denoted by [CG], or to a site comprising one or more of the MVPs of or corresponding to SEQ ID NOS: 26 to 36 denoted by [CG], or to a site comprising one or more of the MVPs of or corresponding to SEQ ID NOS: 38 to 53 denoted by [CG], or to a site comprising one or more of the MVPs of or corresponding to SEQ ID NOS: 55 to 66 denoted by [CG], or to a site comprising one or more of the MVPs of or corresponding to SEQ ID NOS: 68 to 74 denoted by [CG], or to a site comprising one or more of the MVPs of or corresponding to SEQ ID NOS: 76 to 82 denoted by [CG], or to a site comprising one or more of the MVPs of or corresponding to SEQ ID NOS: 84 to 88 denoted by [CG], or to a site comprising one or more of the MVPs of or corresponding to SEQ ID NOS: 90 to 96 denoted by [CG], or to a site comprising one or more of the MVPs of or corresponding to SEQ ID NOS: 98 to 111 denoted by [CG], or to a site comprising one or more of the MVPs of or corresponding to SEQ ID NOS: 113 to 119 denoted by [CG], or to a site comprising one or more of the MVPs of or corresponding to SEQ ID NOS: 121 to 128 denoted by [CG], or to a site comprising one or more of the MVPs of or corresponding to SEQ ID NOS: 130 to 136 denoted by [CG], or to a site comprising one or more of the MVPs of or corresponding to SEQ ID NOS: 138 to 143 denoted by [CG], or to a site comprising one or more of the MVPs of or corresponding to SEQ ID NOS: 145 to 153 denoted by [CG], or to a site comprising one or more of the MVPs of or corresponding to SEQ ID NOS: 155 to 161 denoted by [CG], or to a site comprising one or more of the MVPs of or corresponding to SEQ ID NOS: 163 to 167 denoted by [CG], or to a site comprising one or more of the MVPs of or corresponding to SEQ ID NOS: 169 to 180 denoted by [CG], or to a site comprising one or more of the MVPs of or corresponding to SEQ ID NOS: 182 to 192 denoted by [CG], or to a site comprising one or more of the MVPs of or corresponding to SEQ ID NOS: 194 to 200 denoted by [CG], or to a site comprising one or more of the MVPs of or corresponding to SEQ ID NOS: 202 to 212 denoted by [CG], or to a site comprising one or more of the MVPs of or corresponding to SEQ ID NOS: 214 to 223 denoted by [CG], or to a site comprising one or more of the MVPs of or corresponding to SEQ ID NOS: 225 to 234 denoted by [CG], or to a site comprising one or more of the MVPs of or corresponding to SEQ ID NOS: 236 to 241 denoted by [CG], or to a site comprising one or more of the MVPs of or corresponding to SEQ ID NOS: 243 to 251 denoted by [CG], or to a site comprising one or more of the MVPs of or corresponding to SEQ ID NOS: 253 to 259 denoted by [CG], or to a site comprising one or more of the MVPs of or corresponding to SEQ ID NOS: 261 to 266 denoted by [CG], or to a site comprising one or more of the MVPs of or corresponding to SEQ ID NOS: 268 to 277 denoted by [CG], or to a site comprising one or more of the MVPs of or corresponding to SEQ ID NOS: 279 to 284 denoted by [CG], or to a site comprising one or more of the MVPs of or corresponding to SEQ ID NOS: 286 to 291 denoted by [CG].

183. An isolated oligonucleotide molecule for use as a forward or reverse amplification primer, wherein the molecule is designed to anneal to a site in a DMR and to be capable of amplifying the DMR or a region of the DMR when used with a corresponding reverse or forward amplification primer, wherein the DMR is a DMR having a sequence set forth in any one of SEQ ID NOS: 1, 13, 25, 37, 54, 67, 75, 83, 89, 97, 112, 120, 129, 137, 144, 154, 162, 168, 181, 193, 201, 213, 224, 235, 242, 252, 260, 267, 278, 285 and 292.

184. An isolated oligonucleotide molecule according to claim 180, wherein the molecule has a nucleic acid sequence set forth in any one of SEQ ID NOS: 297 to 358.

185. A pair of isolated oligonucleotide molecules for use as forward and reverse amplification primers, wherein the molecules are designed to amplify a DMR or wherein the molecules are designed to amplify a region of a DMR, wherein the DMR is a DMR having a sequence set forth in any one of SEQ ID NOS: 1, 13, 25, 37, 54, 67, 75, 83, 89, 97, 112, 120, 129, 137, 144, 154, 162, 168, 181, 193, 201, 213, 224, 235, 242, 252, 260, 267, 278, 285 and 292.

186. A pair of isolated oligonucleotide molecules according to claim 182, wherein:
1. the forward primer has a nucleic acid sequence set forth in SEQ ID NO: 297 and the reverse primer has a nucleic acid sequence set forth in SEQ ID NO: 298; or
2. the forward primer has a nucleic acid sequence set forth in SEQ ID NO: 299 and the reverse primer has a nucleic acid sequence set forth in SEQ ID NO: 300; or
3. the forward primer has a nucleic acid sequence set forth in SEQ ID NO: 301 and the reverse primer has a nucleic acid sequence set forth in SEQ ID NO: 302; or
4. the forward primer has a nucleic acid sequence set forth in SEQ ID NO: 303 and the reverse primer has a nucleic acid sequence set forth in SEQ ID NO: 304; or
5. the forward primer has a nucleic acid sequence set forth in SEQ ID NO: 305 and the reverse primer has a nucleic acid sequence set forth in SEQ ID NO: 306; or
6. the forward primer has a nucleic acid sequence set forth in SEQ ID NO: 307 and the reverse primer has a nucleic acid sequence set forth in SEQ ID NO: 308; or
7. the forward primer has a nucleic acid sequence set forth in SEQ ID NO: 309 and the reverse primer has a nucleic acid sequence set forth in SEQ ID NO: 310; or
8. the forward primer has a nucleic acid sequence set forth in SEQ ID NO: 311 and the reverse primer has a nucleic acid sequence set forth in SEQ ID NO: 312; or
9. the forward primer has a nucleic acid sequence set forth in SEQ ID NO: 313 and the reverse primer has a nucleic acid sequence set forth in SEQ ID NO: 314; or
10. the forward primer has a nucleic acid sequence set forth in SEQ ID NO: 315 and the reverse primer has a nucleic acid sequence set forth in SEQ ID NO: 316; or
11. the forward primer has a nucleic acid sequence set forth in SEQ ID NO: 317 and the reverse primer has a nucleic acid sequence set forth in SEQ ID NO: 318; or
12. the forward primer has a nucleic acid sequence set forth in SEQ ID NO: 319 and the reverse primer has a nucleic acid sequence set forth in SEQ ID NO: 320; or
13. the forward primer has a nucleic acid sequence set forth in SEQ ID NO: 321 and the reverse primer has a nucleic acid sequence set forth in SEQ ID NO: 322; or
14. the forward primer has a nucleic acid sequence set forth in SEQ ID NO: 323 and the reverse primer has a nucleic acid sequence set forth in SEQ ID NO: 324; or
15. the forward primer has a nucleic acid sequence set forth in SEQ ID NO: 325 and the reverse primer has a nucleic acid sequence set forth in SEQ ID NO: 326; or
16. the forward primer has a nucleic acid sequence set forth in SEQ ID NO: 327 and the reverse primer has a nucleic acid sequence set forth in SEQ ID NO: 328; or
17. the forward primer has a nucleic acid sequence set forth in SEQ ID NO: 329 and the reverse primer has a nucleic acid sequence set forth in SEQ ID NO: 330; or
18. the forward primer has a nucleic acid sequence set forth in SEQ ID NO: 331 and the reverse primer has a nucleic acid sequence set forth in SEQ ID NO: 332; or
19. the forward primer has a nucleic acid sequence set forth in SEQ ID NO: 333 and the reverse primer has a nucleic acid sequence set forth in SEQ ID NO: 334; or
20. the forward primer has a nucleic acid sequence set forth in SEQ ID NO: 335 and the reverse primer has a nucleic acid sequence set forth in SEQ ID NO: 336; or
21. the forward primer has a nucleic acid sequence set forth in SEQ ID NO: 337 and the reverse primer has a nucleic acid sequence set forth in SEQ ID NO: 338; or
22. the forward primer has a nucleic acid sequence set forth in SEQ ID NO: 339 and the reverse primer has a nucleic acid sequence set forth in SEQ ID NO: 340; or
23. the forward primer has a nucleic acid sequence set forth in SEQ ID NO: 341 and the reverse primer has a nucleic acid sequence set forth in SEQ ID NO: 342; or
24. the forward primer has a nucleic acid sequence set forth in SEQ ID NO: 343 and the reverse primer has a nucleic acid sequence set forth in SEQ ID NO: 344; or
25. the forward primer has a nucleic acid sequence set forth in SEQ ID NO: 345 and the reverse primer has a nucleic acid sequence set forth in SEQ ID NO: 346; or
26. the forward primer has a nucleic acid sequence set forth in SEQ ID NO: 347 and the reverse primer has a nucleic acid sequence set forth in SEQ ID NO: 348; or
27. the forward primer has a nucleic acid sequence set forth in SEQ ID NO: 349 and the reverse primer has a nucleic acid sequence set forth in SEQ ID NO: 350; or
28. the forward primer has a nucleic acid sequence set forth in SEQ ID NO: 351 and the reverse primer has a nucleic acid sequence set forth in SEQ ID NO: 352; or
29. the forward primer has a nucleic acid sequence set forth in SEQ ID NO: 353 and the reverse primer has a nucleic acid sequence set forth in SEQ ID NO: 354; or
30. the forward primer has a nucleic acid sequence set forth in SEQ ID NO: 355 and the reverse primer has a nucleic acid sequence set forth in SEQ ID NO: 356; or
31. the forward primer has a nucleic acid sequence set forth in SEQ ID NO: 357 and the reverse primer has a nucleic acid sequence set forth in SEQ ID NO: 358;
and wherein the sequence of each primer as defined in each SEQ ID NO is read in the 5′ to 3′ direction.

187. An isolated oligonucleotide molecule or pair of isolated oligonucleotide molecules according to any one of claims 175 to 186, wherein the molecule or molecules further comprise adaptor sequences configured to facilitate DNA sequencing.

188. An isolated oligonucleotide molecule or pair of isolated oligonucleotide molecules according to any one of claims 175 to 187, wherein the molecule or molecules further comprise unique index sequences (barcode sequences), wherein each barcode sequence is designed to be specific for a given sample from an individual.

189. A kit comprising a pair of isolated oligonucleotide molecules according to any one of claims 177, 178, 181, 182 and 185 to 188.

190. A kit according to claim 63, further comprising a detection probe according to any one of claims 171 to 174.

191. A kit according to claim 189 or 190, further comprising reagents for amplifying DNA.
